(19) United States
(12) Patent Application Publication
(10) Pub. No.: US 2025/0171404 A1
(43) Pub. Date: May 29, 2025

(54) PCNA INHIBITORS AND USES THEREOF

(71) Applicant: CITY OF HOPE, Duarte, CA (US)

(72) Inventors: Robert J. HICKEY, Lakeview Terrace, CA (US); Linda H. MALKAS, Lakeview Terrace, CA (US); Pouya HARATIPOUR, Chino Hills, CA (US); Long GU, Arcadia, CA (US); Gerald WUENSCHELL, Arcadia, CA (US)

(21) Appl. No.: 18/842,728

(22) PCT Filed: Mar. 6, 2023
(86) PCT No.: PCT/US2023/063800

§ 371 Date: Aug. 29, 2024

Related U.S. Application Data
(60) Provisional application No. 63/317,430, filed on Mar. 7, 2022.

(51) Int. Cl. C07D 217/26; A61K 31/167; A61K 31/4409; A61K 31/4725; A61P 35/00; C07C 237/48; C07D 213/68; C07D 401/12

(52) U.S. Cl. CPC C07D 217/26; A61K 31/167; A61K 31/4409; A61K 31/4725; A61P 35/00; C07C 237/48; C07D 213/68; C07D 401/12

(57) ABSTRACT

Described herein, inter alia, are PCNA inhibitors and uses thereof.

### CROSS-REFERENCE TO RELATED APPLICATIONS

This application claims the benefit of U.S. Provisional Application No. 63/317,430 filed Mar. 7, 2022, the contents of which is hereby incorporated herein in its entirety and for all purposes.

### STATEMENT AS TO RIGHTS TO INVENTIONS MADE UNDER FEDERALLY SPONSORED RESEARCH AND DEVELOPMENT

This invention was made with government support under W81XWH-19-1-0326 awarded by the Medical Research and Development Command. The government has certain rights in the invention.

### REFERENCE TO AN ELECTRONIC SEQUENCE LISTING

The contents of the electronic sequence listing (048440-826001WO_Sequence_Listing_ST26.xml; Size 15,199 bytes; and Date of Creation: Feb. 13, 2023) are hereby incorporated by reference in their entirety.

### BACKGROUND

Proliferating cell nuclear antigen (PCNA) is critical to DNA replication and repair processes and it is also a proliferation biomarker in a variety of human tumors. A unique cancer-associated isoform of the protein, caPCNA, has been previously identified that potentially allows for selective therapeutic targeting of cancer cells. A number of strategies have been employed to develop agents targeting caPCNA, including peptide and small molecule-based inhibitors, but the success in developing therapeutically tractable compounds has been limited. Disclosed herein, inter alia, are solutions to these and other problems in the art.

### BRIEF SUMMARY

In an aspect is provided a compound, or a pharmaceutically acceptable salt thereof, having the formula:

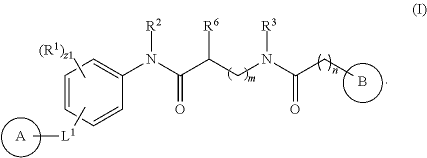

L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—.

R⁷, R⁸, and R⁹ are independently hydrogen, halogen, —OH, —N₃, or substituted or unsubstituted alkyl.

Ring A is substituted or unsubstituted phenyl or substituted or unsubstituted 5 to 6 membered heteroaryl.

Ring B is substituted or unsubstituted phenyl, substituted or unsubstituted naphthyl, substituted or unsubstituted quinolinyl, or substituted or unsubstituted isoquinolinyl.

R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

R² is hydrogen, halogen, —CX²₃, —CHX²₂, CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ and R⁶ may optionally be joined to form a substituted or unsubstituted heterocycloalkyl or substituted or unsubstituted heteroaryl.

R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl.

The symbol z1 is an integer from 0 to 4. The symbols m1, m6, v1, and v6 are independently 1 or 2. The symbols n1 and n6 are independently an integer from 0 to 4.

X, X¹, X², X³, and X⁶ are independently —Cl, —Br, —I, or —F.

The symbol m is an integer from 0 to 5. The symbol n is an integer from 0 to 10.

In an aspect is provided a compound, or a pharmaceutically acceptable salt thereof, having the formula:

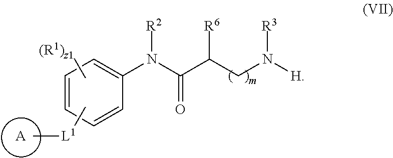

L¹, Ring A, R¹, z1, R², R³, R⁶, and m are as described herein, including in embodiments.

In an aspect is provided a pharmaceutical composition including a compound described herein, or a pharmaceutically acceptable salt thereof, and a pharmaceutically acceptable excipient.

In an aspect is provided a method of treating a disease associated with PCNA activity in a subject in need thereof, the method including administering to the subject in need thereof a therapeutically effective amount of a compound described herein, or a pharmaceutically acceptable salt thereof.

In an aspect is provided a method of treating cancer in a subject in need thereof, the method including administering to the subject in need thereof a therapeutically effective amount of a compound described herein, or a pharmaceutically acceptable salt thereof.

In an aspect is provided a method of inhibiting PCNA activity, the method including contacting PCNA with an effective amount of a compound described herein, or a pharmaceutically acceptable salt thereof.

In an aspect is provided a method of making compound (I), or a pharmaceutically acceptable salt thereof, the method including mixing compound (VII) and compound (X) together in a reaction vessel. Compound (I) has the formula:

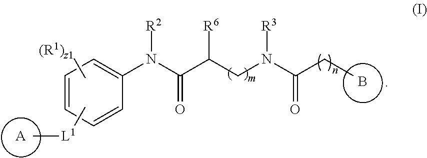

Compound (VII) has the formula:

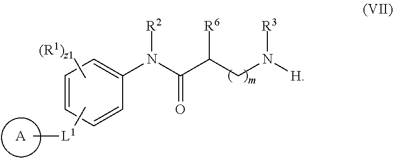

Compound (X) has the formula:

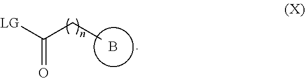

L¹, Ring A, R¹, z1, R², R³, R⁶, m, and n are as described herein, including in embodiments. LG is a leaving group.

### BRIEF DESCRIPTION OF THE DRAWINGS

FIGS. 1A-1E. AOH1160 analog interactions with PCNA. FIG. 1A: Thermal shift assay: Normalized inverse derivative thermal denaturation plots of 9 μM apo-PCNA with DMSO control is depicted with a black dashed line and PCNA in the presence of 10 μM AOH1160LE is shown in light grey and 30 μM AOH1160LE in dark grey. ΔTₘ values are provided. FIG. 1B: As described in FIG. 1A, in the presence of 10 μM AOH1996 depicted in light grey and 30 μM AOH1996 depicted in dark grey. FIG. 1C: Four monomers (chains A-D) of PCNA are present in the asymmetric unit of the crystal. Chains A, B, and C form the homotrimer biological unit, while chain Dis orientated perpendicular to the plane of the ring, below the interface of chains B and C and it belongs to an adjacent ring. Inset: Three molecules of AOH1160LE bind in and adjacent to the PIP box cavity of each of the PCNA ring subunits. The omit map, gray mesh, is contoured to 1.56. Two PCNA monomer subunits from adjacent rings stack against each other, placing the PIP box and IDCLs of each subunit in opposing directions. FIG. 1D: The three AOH1160LE molecules are shown as stick figure representations. Two of the compounds, left and central, bind into the PIP box cavity at known PCNA-partner/compound interaction sites. FIG. 1E: Superimposition of the PCNA:T2AA and the PCNA:AOH1160LE complexes, centered on the PIP box cavity, with T2AA carbons in dark grey. The electrostatic surface maps are depicted with Poisson-Boltzmann electrostatic surface potentials shown in dark grey and light grey, corresponding to −10 to +10 kT/e respectively.

FIGS. 2A-2D. Interaction of AOH1996 with PCNA. FIG. 2A: The PCNA gene was mutated by CRISPR resulting in codon substitution of the Leucine 47 residue by a Valine. Shown are the DNA sequencing results of cell lines heterozygous or homozygous of the mutated gene. Original Seq: ACGTCTCTTTGGTGCAGCTCACC (SEQ ID NO:1); Donor Seq: ACGTCTCTGTCGTGCAGCTCACC (SEQ ID NO:2); Homozygote: ACGTCTCTGTCGTGCAGCTCACCC (SEQ ID NO:3). FIGS. 2B-2C: Cell lines heterozygous or homozygous of the mutated PCNA gene were treated by the indicated concentrations of AOH1996 or R⁹-caPep, respectively, for 72 hrs. The parent SK-N-AS cells were used as control. Relative cell growth in triplicate was averaged and graphed ±S.D. FIG. 2D: Expression of γH2A.X was measured by Western blot in cell lines heterozygous (#25H and #37H) or homozygous (#33 and #35) of the mutated PCNA allele after cells were treated by 500 nM AOH1996 or 30 μM R⁹-caPep for the indicated time.

FIGS. 3A-3D. Therapeutic properties of AOH1996. FIG. 3A: Normal cells (7SM0032) or cancer cells (SH-SY5Y and SK-N-BE(2)c) cells were fixed, stained with PI, and analyzed by flow cytometry following treatment with 500 nM AOH1996 for the indicated time. FIG. 3B: SK-N-DZ neuroblastoma cells and nonmalignant 7SM0032 stem cells were incubated with 500 nM AOH1996 for 24 h. Then, after being fixed on slides, cell apoptosis were analyzed by a TUNEL assay. Left: TMR fluorophore attached to the free ends of DNA indicates cells undergoing apoptosis. DAPI stained nuclei also shown. Right: Average abundance ±S.D. of apoptotic 7SM0032 (black histogram) and SK-N-DZ (grey histogram) cells relative to the total number of cells are shown in 5 randomly selected fields. FIG. 3C: Human SK-N-DZ neuroblastoma cells were treated with or without the indicated concentrations of cisplatin in the absence or presence of 500 nM of AOH1996 for 18 hours. Cells were washed twice with growth medium and cultured in fresh media for 18 d to allow colony formation. The colony counts in dishes treated with cisplatin but not AOH1996 (black) were normalized to the colony counts in dishes untreated by either agent. The colony counts in dishes treated by both cisplatin and AOH1996 (grey) were normalized to the colony counts in dishes treated with 500 nM AOH1996 alone. The relative number of colonies determined in triplicates for each treatment condition were averaged and graphed ±SDs (*, P<0.01). FIG. 3D: SK-N-AS cells were treated by the indicated concentrations of AOH1996, topotecan, or both agents in combination. Cell growth was measured as the percentage of cell confluence by imaging every 6 h for a total of 48 h.

FIGS. 4A-4H. Pharmacokinetics and anti-tumor growth activity of AOH1996 in vivo. FIG. 4A: After oral administration, the plasma concentrations of AOH1996 from three male and three female ES1^{e}/SCID mice at the indicated time points were averaged and graphed ±S.D. The inset contains PK parameters determined by a standard noncompartmental method. FIG. 4B: A similar PK study of AOH1996 was performed in dogs. FIGS. 4C-4E: Mice bearing the xenograft tumors of neuroblastoma (FIG. 4C: SK-N-BE(2)c), breast cancer (FIG. 4D: MDA-MB-468), and small-cell lung cancer (FIG. 4E: H82) were given vehicle only (black) or 40 mg/kg of AOH1996 (grey) twice daily immediately after the first measurement. Tumor sizes were measured by a dial caliper each week. Tumor volumes (0.4×L×W²) were averaged and graphed ±S.E. (*, P<0.01). FIG. 4F: Animal body weight was monitored throughout the studies as an indicator of toxicity. Shown is a typical study results from the study of the SK-N-BE(2)c tumor model described in (FIG. 4C). FIG. 4G: The levels of phosphor-Chk1 (pChk1) and γH2A.X in SK-N-BE(2)c derived tumor samples were analyzed by IHC. Shown are representative images taken from tumors treated by vehicle only or by 40 mg/kg AOH1996. FIG. 4H: ES1^{e}/SCID mice bearing SK-N-AS derived xenograft tumors were treated with either 80 mg/kg of AOH1996 (black, n=7) for 8 d beginning 8 d after tumor implantation, 15 mg/kg of CPT-11 (n=8) for 3 d beginning 12 d after tumor implantation, or both agents (combination, n=8) under the same dosages and schedules as they were dosed alone. Mice implanted with the same tumor and left untreated were used as control (n=8). Shown are the survival graphs. The p values determined by the Log-rank (Mantel-Cox) test between combination treatment and each of the control groups are p=0.0003 (Combination vs. NoRx), p=0.005 (Combination vs. AOH1996 alone), and p=0.024 (Combination vs. CPT-11 alone).

FIGS. 5A-5E. Modulation of PCNA interaction with RNA polymerase II. FIG. 5A: Chromatin-bound (CB) proteins were fractioned from HEK293T cells expressing FLAG-tagged PCNA after the cells were treated with or without 500 nM AOH1996. Proteins in complex with FLAG-PCNA were immune-precipitated and analyzed by mass spectrometry. Shown are numbers of proteins whose abundances were altered (grey) or unaltered (black) by more than 2-fold by AOH1996 treatment. FIG. 5B: SK-N-AS cells exogenously expressing a FLAG-tagged RPB1 gene were fractioned before and after being treated with 500 nM AOH1996 overnight. PCNA in complex with chromatin-bound (CB) FLAG-RBP1 were analyzed by western blotting. FIG. 5C: Human SK-N-AS cells were treated with UV in the presence or absence of AOH1996 (AOH) or R9-caPep (caPep). Whole cell extracts were analyzed by western blotting. FIG. 5D: Cells exogenously expressing FLAG-tagged wildtype RPB1 or FLAG-tagged APIM-mutant RPB1 gene were fractioned. PCNA in complex with chromatin-bound (CB) FLAG-RBP1 were analyzed by western blotting. FIG. 5E: HEK293T cells were transiently transfected with a FLAG-tagged wildtype RPB1 (APIM WT) gene or mutant RPB1 gene (APIM mutant). The intracellular MCM7 and RBP1 (both the hypo-phosphorylated RNAPIIa and hyper-phosphorylated RNAPIIo forms) were analyzed by western blot after cells were treated by the indicated agents and/or UV.

FIGS. 6A-6C. The effect of AOH1996 is mediated through PCNA interaction with RPB1. FIG. 6A: Cell lines heterozygous or homozygous of the APIM-mutant RPB1 gene were treated by the indicated concentrations of AOH1996 for 72 hrs. The parent SK-N-AS cells were used as control. Relative cell growth in triplicate was averaged and graphed ±S.D. FIG. 6B: Whole cell proteome from SK-N-AS cells homozygous of the APIM-mutant RPB1 gene were analyzed by mass spectrum before and after the cells were treated by 500 nM AOH1996 overnight in quadruplicates. To average out any clonal difference unrelated to the RPB1 mutation, the quadruplicated samples were derived from 2 independent RPB1 mutant clones. The parent SK-N-AS cells in quadruplicates were used as control. The enrichment of proteins whose expressions were altered by AOH1996 by at least 2 fold and with a p-value less than 0.05 in cells of either genotype was analyzed by MetaCore's (Clarivate Analytics, Philadelphia, PA) gene ontology program. Shown in the cricos diagram are the enriched GO processes these proteins associated with and the average fold change of their expression induced by AOH1996 treatment. FIG. 6C: The fold of changes in the expression of the proteins identified in FIG. 6B was calculated for each AOH1996-treated sample relative to the average expression level in untreated corresponding cells and visualized in the dot plot heatmap. Also shown in the dot plot heatmap is the statistical significance expressed in-log₁₀(p-value) between treated and untreated samples of the same genotype.

FIGS. 7A-7E. Transcription dependent effect on DNA replication and damage. FIG. 7A: Left: Schematic of the cell fractionation procedure. Right: MDA-MB-468 cells were treated by increasing concentrations of AOH1996 (5, 50, or 500 nM) for 24 h. Cells treated with DMSO were used as control. Whole cell extract (WCE) and protein fractions associated with actively transcribed chromatin (CB:RNA+) or with low or non-transcribed chromatin (CB:RNA−) were analyzed by Western blot using antibodies against PCNA, CAF-1, and MCM7. FIG. 7B: Synchronized cancer cells were sequentially incubated in the presence of CldU (light grey) and IdU (dark grey) before and after AOH1996 treatment, respectively. Cells sequentially incubated with the same two nucleotide analogues but without AOH1996 were used as control. Left: Representative images of labeled DNA strands from cells treated with or without AOH1996. Middle and Right: Lengths of CldU (light grey) and IdU (dark grey) incorporated DNA segments measured for more than 30 independent DNA strands from the indicated cancer cell type were averaged and graphed ±S.D. FIG. 7C: Whole cell lysates were extracted from SK-N-AS cells homozygous of the wildtype RPB1 allele or the APIM-mutant allele. Histone H2A.X and γH2A.X was analyzed by Western blot after treatment with or without AOH1996 overnight. FIG. 7D: Histone H2A.X and γH2A.X in whole cell lysates from SK-N-AS cells were analyzed by Western blot after treatment with 500 nM AOH1996 and/or 50 μM DRB overnight. FIG. 7E: A working model of AOH1996 action mechanism: binding of AOH1996 to PCNA stabilizes PCNA interaction with RNA polymerase II and interferes with TRC resolution leading to dissociation of PCNA from chromatin in a transcription dependent manner. By exploiting this cancer vulnerability, AOH 1996 selectively inhibits tumor growth without causing any discernible side effect.

FIG. 8. Schematic of the interactions of the three AOH1160LE molecules in the PCNA PIP box/APIM binding pockets. Individual AOH1160LE molecules are listed from left to right based on their positioning in the PIP box pocket as shown in FIGS. 1A-1E. Specific protein-ligand interactions are highlighted based on the provided legend.

FIG. 9. Structural superimposition of the central bound AOH1160LE molecule and the PCNA: APIM peptide complex. The central AOH1160LE (dark grey) binds to the same hydrophobic pockets as the ZRANB3 APIM peptide (PDB code: 5MLW) colored in light grey. ZRANB3 Phe1075 side chain is also depicted, where this side chain interacts in a second region of the PCNA APIM binding pocket, in which a separate molecule of AOH1160LE interacts (Left molecule in FIG. 8). Inset: Close up view of the two hydrophobic binding regions of PCNA, with the diphenyl ether of central AOH1160LE positioned where Ile1072 and Val1077 of APIM peptide bind.

FIG. 10. Orientation of AOH1160LE bound into PIP box cavity of PCNA. Chains A-D are depicted by solid boxes, with corresponding symmetry mates in dashes, each labeled accordingly. The orientation of the central AOH1160LE diphenyl ether binding is depicted and further indicated by the direction of the black arrows.

FIG. 11. Chain C central AOH1160LE molecule interactions with PCNA, via the naphthalene group. Specific protein-ligand interactions are highlighted based on the provided legend.

FIGS. 12A-12E. AOH1996 metabolism and in vivo pharmacokinetics. FIG. 12A: Illustration of AOH1160 degradation by carboxyl esterase-mediated cleavage or by hydroxylation. FIG. 12B: Chromatogram of liver microsome reaction mixtures of AOH1160 and AOH1996. The metabolites shown in FIG. 12A are indicated above their corresponding peaks. FIG. 12C: An aliquot of the liver microsome reaction mixture of AOH1996 was taken after various incubation times in the presence or absence of NADPH as the energy source. AOH1996 concentrations, determined by LC/MS-MS, as a percentage of the input concentration were graphed. FIG. 12D: After oral administration, the plasma concentrations of AOH1996 from three male and three female ES1e/SCID mice at the indicated time points were averaged and graphed ±S.D. The inset contains PK parameters determined by a standard noncompartmental method. FIG. 12E: A similar PK study of AOH1996 was performed in dogs.

FIGS. 13A-13E. Selective inhibition of cancer cells. FIG. 13A: Cancer cells of the NCI60 panel were incubated in the presence of various concentrations of AOH1996 for 48 hours. Cells growth was analyzed by a sulforhodamine B (SRB) assay after cells were fixed by ice-cold 10% trichloroacetic acid (TCA). The GI50 in molar for each cell line was calculated by NCI (see Example 2 for details). Cell lines: Leukemia: CCRF-CEM, HL-60 (TB), K-562, MOLT-4, RPMI-8226, SR; Non-Small Cell Lung Cancer: A549/ATCC, EKVX, HOP-62, HOP-92, NCI-H226, NCI-H23, NCI-H322M, NCI-H460, NCI-H522; Colon Cancer: COLO 205, HCC-2998, HCT-116, HCT-15, HT29, KM12, SW-620; CNS Cancer: SF-268, SF-295, SF-539, SNB-19, SNB-75, U251; Melanoma: LOX IMVI, MALME-3M, M14, MDA-MB-435, SK-MEL-2, SK-MEL-28, SK-MEL-5, UACC-257, UACC-62; Ovarian Cancer: IGROV1, OVCAR-3, OVCAR-4, OVCAR-5, OVCAR-8, NCI/ADR-RES, SK-OV-3; Renal Cancer: 786-0, A498, ACHN, CAKI-1, RXF 393, SN12C, TK-10, UO-31; Prostate Cancer: PC-3, DU-145; Breast Cancer: MCF7, MDA-MC-231/ATCC, HS 578T, BT-549, T-47D, MDA-MB-468. FIGS. 13B-13D: Shown in the graph are the LogGI₅₀. As indicated in the figure, small cell lung cancer (FIG. 13B: H-82, H-524, H-526, LX²₂, and LX³₃), neuroblastoma (FIG. 13C: SK-N-BE(2) c, SH-SY5Y, and SK-N-AS), and prostate cancer (FIG. 13D: LN-caP, LN-caP-R, 22RV1, H660, LASCPC, PC3, and DU145) cell lines were treated with various concentrations of AOH1996 for 72 hours. Non-malignant cells (FIG. 13B: hSAEC and PBMC; and FIG. 13C: 7SM0032) were used as control. Cell growth was measured by the CellTiter-Glo® Luminescent assay. Relative cell growth in triplicate was averaged and graphed ±S.D. FIG. 13E: Histone H2A.X and γH2A.X in whole cell lysates from the indicated cells were analyzed by Western blot after treatment with 500 nM AOH1996 for various time.

FIG. 14. Chemical structures of AOH1996 and AOH1160.

FIG. 15. Features of the binding pocket within the PIP box domain of PCNA. A, B: Primarily hydrophobic depressions extending well under the IDCL. Some polarity “deep inside” but hard to access. IDCL is shaded. C: Distinct depression, but with more polarity than A or B. D: “Wall” is hydrophobic but has does have polar character. E, F, G: Other hydrophobic pockets significant to alternative binding configurations. H: Large hydrophobic region, difficult to utilize with compounds of shorter lengths.

FIG. 16. Representation for the lowest energy conformation of AOH1996 (E=−8.7).

FIG. 17. Representation for the lowest energy configuration of fluorinated AOH1996.

FIG. 18. Representation for the lowest energy configuration of AOH1160 containing isonipecotic acid in place of the glycine linker.

FIG. 19. Representation of the conformations for AOH1996 and the D-homophenylalanine docking with the PIP binding domain of caPCNA.

FIG. 20. Representation of the conformations for AOH1996 analogs containing adamantyl amides of D-aspartic acid and D-glutamic acid docking with the PIP domain of caPCNA.

FIGS. 21A-21D. Binary complex models of AOH1990-2 (FIG. 21A), AOH1160NH (FIG. 21B), AOH1160RCHF (FIG. 21C), and AOH1160CF2 (FIG. 21D) inside the PCNA active site.

FIGS. 22A-22B. Binary complex models of AOH1996 (FIG. 22A) and AOH1160 (FIG. 22B) inside the PCNA active site.

FIGS. 23A-23B. Binary complex models of AOH1160eNaph (FIG. 23A) and AOH1996eeNaph (FIG. 23B) inside the PCNA active site.

FIG. 24. TSA data for AOH1160LV. AOH1160LV exhibited very similar ΔTₘ compared to AOH1996.

FIG. 25. IC₅₀ data for AOH1160LA. IC₅₀=˜3 μM.

FIG. 26. IC₅₀ data for AOH1996LA. IC₅₀s were 1.5, 1.6, and 1.8 μM for AK-N-AS, H1752, and H358, respectively.

FIG. 27. IC₅₀ data for AOH1996TMB. IC₅₀ values: Hela: 6.5 μM, A673: 5.5 μM, A549: 4.7 μM.

FIGS. 28A-28D. IC₅₀ data for AOH analogs in MDA-MB-468 cell line. IC₅₀ values for AOH1996: 740 nM (FIG. 28A), AOH1160S: 316 nM (FIG. 28B), AOH1160LA: 4900 nM (FIG. 28C), AOH1996LA: 2300 nM (FIG. 28D).

FIG. 29. IC₅₀ data for select compounds. The indicated cell lines were seeded at 10⁴ cells per well in a 96 well plate. After allowing to attach to the plate overnight, cells were treated with various concentrations of the indicated compounds in triplicates for 72 hrs. The cell growth was measured by an SRB assay. The cell abundances under each treatment condition relative to the baseline were averaged and graphed ±S.D. The baseline is defined as the median value of the cell abundances under the treatment by the two lowest compound concentrations. The IC₅₀ was calculated by the Prism program.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH—CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

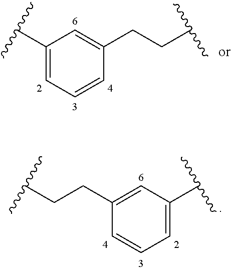 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R″, —OC(O)R′, —C(O)R′, —CO₂R′, —CONR′R″, —OC(O)NR′R″, —NR″C(O)R′, —NR′C(O)NR″R″, —NR″C(O)₂R′, —NRC(NR′R″R″)═NR″, —NRC(NR′R″)═NR″, —S(O)R′, —S(O)₂R′, —S(O)₂NR′R″, —NRSO₂R′, —NR′NR″R″, —ONR′R″, —NR′C(O)NR″NR″R″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R″, —OC(O)R′, —C(O)R′, —CO₂R′, —CONR′R″, —OC(O)NR′R″, —NR″C(O)R′, —NR′C(O)NR″R″, —NR″C(O)₂R′, —NR—C(NR′R″R″)═NR″, —NR—C(NR′R″)═NR″, —S(O)R′, —S(O)₂R′, —S(O)₂NR′R″, —NRSO₂R′, —NR′NR″R″, —ONR′R″, —NR′C(O)NR″NR″R″, —CN, —NO₂, —R′, —N₃, —CH(Ph)₂, fluoro (C₁-C₄)alkoxy, and fluoro (C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″, and R″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3.

Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R″)_{d}—, where s and d are independently integers of from 0 to 3, and X is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, —S(O)₂—, or —S(O)₂NR′—. The substituents R, R′, R″, and R″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur(S), phosphorus (P), selenium (Se), and silicon (Si). In embodiments, the terms “heteroatom” or “ring heteroatom” are meant to include oxygen (O), nitrogen (N), sulfur(S), phosphorus (P), and silicon (Si).

A “substituent group,” as used herein, means a group selected from the following moieties:
(A) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, RIA may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1} respectively. In turn, each first substituent group (e.g., R^{1.1}, R^{2.1}, R^{3.1}, R^{4.1}, R^{5.1}. R^{100.1}, R^{1A.1}, R^{2A.1}, R^{3A.1}, R^{4A.1}, R^{5A.1} . . . R^{100A.1}. R^{L1.1}. R^{L2.1}, R^{L3.1}, R^{L4.1}, R^{L5.1}. R^{L100.1}) may be further substituted with one or more second substituent groups (e.g., R^{1.2}, R^{2.2}, R^{3.2}, R^{4.2}, R^{5.2} . . . . R^{100.2}; R^{1A.2}, R^{2A.2}, R^{3A.2}, R^{4A.2}, R^{5A.2} . . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2}. R^{100.2}, R^{1A.2}, R^{2A.2}, R^{3A.2}, R^{4A.2}, R^{5A.2}. R^{100A.2}; R^{L1.2}, R^{L2.2}, R^{L3.2}, R^{L4.2}, R^{L5.2} . . . . R^{L100.2}) may be further substituted with one or more third substituent groups (e.g., R^{1.3}, R^{2.3}, R^{3.3}, R^{4.3}, R^{5.3}. R^{100.3}. R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3}. R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

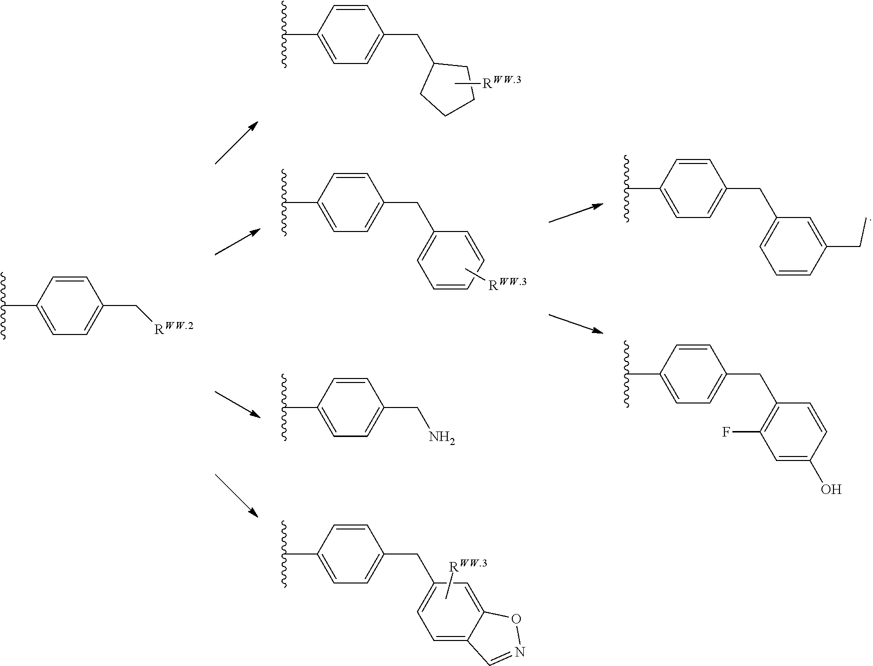

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}. R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group are as defined (1, 2, 3, 1A, 2A, 3A, 1B, 2B, 3B, etc.). R^{LWW.1}, as well as R^{LWW.2} and R^{LWW.3} above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylates, phenoxides, boronic acid, boronate esters, and alkoxides. In embodiments, the leaving group is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. For example, certain methods herein treat diseases associated with PCNA activity. Certain methods described herein may treat diseases associated with PCNA activity (e.g., cancer or neuroblastoma) by inhibiting PCNA activity. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments, contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

“Anti-cancer agent” or “anti-cancer drug” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estroge+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111 In, 90Y, or 1311, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g., tomaymycin), carboplatin, CC-1065 and CC-1065 analogs including amino-CBIs, nitrogen mustards (such as chlorambucil and melphalan), dolastatin and dolastatin analogs (including auristatins: e.g., monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g., mertansine), methotrexate, mitomycin C, taxoids, vinca alkaloids (such as vinblastine and vincristine), epothilones (e.g., epothilone B), camptothecin and its clinical analogs topotecan and irinotecan, or the like.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is cancer (e.g., sarcoma, adenocarcinoma, leukemia, or lymphoma).

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” and “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refers to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., cancer) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease. For example, a disease associated with PCNA activity may be treated with an agent (e.g., compound as described herein) effective for decreasing the level of PCNA activity.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “amino acid side chain” refers to the side chain of an amino acid. For example, if an amino acid has the formula

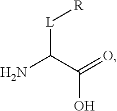

then-L-R is the amino acid side chain. As an example, leucine has the formula

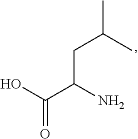

and the L-leucine side chain is

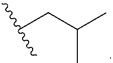 [SMILES: *CC(C)C]

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to His44 of PCNA when the selected residue occupies the same essential spatial or other structural relationship as His44 of PCNA. In some embodiments, where a selected protein is aligned for maximum homology with PCNA, the position in the aligned selected protein aligning with His44 is said to correspond to His44. Instead of a primary sequence alignment, a three dimensional structural alignment can also be used, e.g., where the structure of the selected protein is aligned for maximum correspondence with PCNA and the overall structures compared. In this case, an amino acid that occupies the same essential position as His44 in the structural model is said to correspond to the His44 residue.

The term “Proliferating cell nuclear antigen” or “PCNA” refers to a ˜29 kDa protein that self assembles into a protein complex consisting of 3 subunits of individual PCNA proteins. Together these joined PCNA molecules form a DNA clamp that acts as a processivity factor for DNA polymerase 8 in eukaryotic cells. The term “PCNA” may refer to the nucleotide sequence or protein sequence of human PCNA (e.g., Entrez 5111, Uniprot P12004, RefSeq NM_002592, or RefSeq NP_002583). The term “PCNA” includes both the wild-type form of the nucleotide sequences or proteins as well as any mutants thereof. In some embodiments, “PCNA” is wild-type PCNA. In some embodiments, “PCNA” is one or more mutant forms. The term “PCNA” XYZ refers to a nucleotide sequence or protein of a mutant PCNA wherein the Y numbered amino acid of PCNA that normally has an X amino acid in the wild-type, instead has a Z amino acid in the mutant. In embodiments, a PCNA is the human PCNA. In embodiments, the PCNA has the nucleotide sequence corresponding to reference number GI: 33239449. In embodiments, the PCNA has the nucleotide sequence corresponding to RefSeq NM_002592.2. In embodiments, the PCNA has the protein sequence corresponding to reference number GI: 4505641. In embodiments, the PCNA has the nucleotide sequence corresponding to RefSeq NP_002583.1. In embodiments, the PCNA has the following amino acid sequence:

(SEQ ID NO: 4)
MFEARLVQGSILKKVLEALKDLINEACWDISSSGVNLQSMDSSHVSLVQ
LTLRSEGFDTYRCDRNLAMGVNLTSMSKILKCAGNEDIITLRAEDNADT
LALVFEAPNQEKVSDYEMKLMDLDVEQLGIPEQEYSCVVKMPSGEFARI
CRDLSHIGDAVVISCAKDGVKFSASGELGNGNIKLSQTSNVDKEEEAVT
IEMNEPVQLTFALRYLNFFTKATPLSSTVTLSMSADVPLVVEYKIADMG
HLKYYLAPKIEDEEGS.

In embodiments, the PCNA is a mutant PCNA. In embodiments, the mutant PCNA is associated with a disease that is not associated with wild-type PCNA. In embodiments, the PCNA includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. PCNA may be post-translationally modified. Modifications may include phosphorylation, methylation, methylesters of acidic amino acids, ribosylation, acetylation, glycosylation with a variety of sugars, lipidation with a variety of different lipids, poly(ADP) ribosylation, or other post-translational modifications known in the art. Differences in the extent and type of modification influences the levels (e.g., protein levels) of the ca- and nm-PCNA isoforms. In embodiments, a post-translational modification or plurality of post-translational modifications modify the inhibition of PCNA by a compound described herein or the binding of a compound described herein to PCNA, relative to PCNA without the post-translational modification(s).

The terms “cancer-associated proliferating cell nuclear antigen” or “caPCNA” as used herein refer to an isoform of PCNA having an acidic isoelectric point (e.g., peptide including protonated amine and/or carboxyl groups, acidic isoelectric point compared to a non-cancer-associated PCNA, PCNA in non-cancerous cells, non-malignant PCNA, prevalent PCNA isoform in non-cancerous cells, or less acidic PCNA isoform in non-cancerous cells). In embodiments, the caPCNA protein includes methylated amino acids (e.g., glutamate, aspartic acid). In embodiments, the caPCNA protein is post-translationally modified with a methylester of an acidic amino acid. In embodiments, the methylesterification of the acidic amino acid residues on PCNA exhibit a T_{1/2} of approximately 20 minutes at pH 8.5. In embodiments, caPCNA is post-translationally modified as described in F. Shen, et al. J Cell Biochem. 2011 March; 112 (3): 756-760, which is incorporated by reference in its entirety for all purposes.

The terms “non-malignant Proliferating cell nuclear antigen” or “nmPCNA” as used herein refer to an isoform of PCNA having a basic isoelectric point (e.g., peptide including deprotonated amine and/or carboxyl groups, basic isoelectric point compared to a caPCNA, caPCNA in cancerous cells). In embodiments, nmPCNA is the prevalent PCNA isoform in non-cancerous cells.

### II. Compounds

In an aspect is provided a compound, or a pharmaceutically acceptable salt thereof, having the formula:

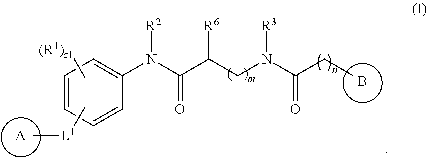

L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—.

R⁷, R⁸, and R⁹ are independently hydrogen, halogen, —OH, —N₃, or substituted or unsubstituted alkyl (e.g., C₁-C₈, C₁-C₆, C₁-C₄, or C₁-C₂).

Ring A is substituted or unsubstituted phenyl or substituted or unsubstituted 5 to 6 membered heteroaryl.

Ring B is substituted or unsubstituted phenyl, substituted or unsubstituted naphthyl, substituted or unsubstituted quinolinyl, or substituted or unsubstituted isoquinolinyl.

R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two adjacent R¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is hydrogen, halogen, —CX²₃, —CHX²₂, —CH₂X², —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ is hydrogen, halogen, —CX³₃, —CHX³₂, —CH₂X³, —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂ROD, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R³ and R⁶ may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); RIA and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{6A} and R^{6B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

The symbol z1 is an integer from 0 to 4.

The symbols m1, m6, v1, and v6 are independently 1 or 2.

The symbols n1 and n6 are independently an integer from 0 to 4.

X, X¹, X², X³, and X⁶ are independently —Cl, —Br, —I, or —F.

The symbol m is an integer from 0 to 5.

The symbol n is an integer from 0 to 10.

In embodiments, the compound, or a pharmaceutically acceptable salt thereof, has the formula:

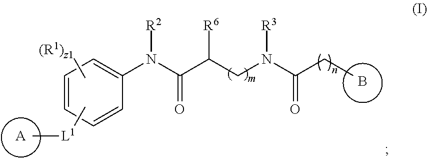

L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—; R⁷, R⁸, and R⁹ are independently hydrogen, halogen, —OH, —N₃, or substituted or unsubstituted alkyl (e.g., C₁-C₈, C₁-C₆, C₁-C₄, or C₁-C₂); Ring A is substituted or unsubstituted phenyl or substituted or unsubstituted 5 to 6 membered heteroaryl; Ring B is substituted or unsubstituted phenyl, substituted or unsubstituted naphthyl, substituted or unsubstituted quinolinyl, or substituted or unsubstituted isoquinolinyl; R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two adjacent R¹ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R² is hydrogen, halogen, —CX²₃, —CHX²₂, —CH₂X², —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R³ is hydrogen, halogen, —CX³₃, —CHX³₂, —CH₂X³, —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂ROD, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R³ and R⁶ may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); RIA and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{6A} and R^{6B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); the symbol z1 is an integer from 0 to 4; the symbols m1, m6, v1, and v6 are independently 1 or 2; the symbols n1 and n6 are independently an integer from 0 to 4; X, X¹, X², X³, and X⁶ are independently —Cl, —Br, —I, or —F; the symbol m is an integer from 0 to 5; the symbol n is an integer from 0 to 10.

In embodiments, when L¹ is —O—, m is 0, n is 0, and Ring B is substituted or unsubstituted naphthyl, substituted or unsubstituted quinolinyl, or substituted or unsubstituted isoquinolinyl, then R⁶ is not hydrogen.

In embodiments, the compound has the formula:

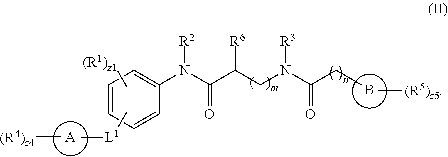

L¹, R¹, z1, R², R³, R⁶, m, and n are as described herein, including in embodiments.

Ring A is phenyl or 5 to 6 membered heteroaryl.

Ring B is phenyl, naphthyl, quinolinyl, or isoquinolinyl.

R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR⁴NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two adjacent R⁴ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R⁵ is independently a halogen, —CX₅³, —CHX⁵₂, —CH₂X⁵, —OCX₅³, —OCHX⁵₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); two adjacent R⁵ substituents may optionally be joined to form a substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{4A} and R^{4B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{5A} and R^{5B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

The symbol z4 is an integer from 0 to 5.

The symbol z5 is an integer from 0 to 7.

The symbols m4, m5, v4, and v5 are independently 1 or 2.

The symbols n4 and n5 are independently an integer from 0 to 4.

X, X⁴, and X⁵ are independently —Cl, —Br, —I, or —F.

In embodiments, the compound has the formula:

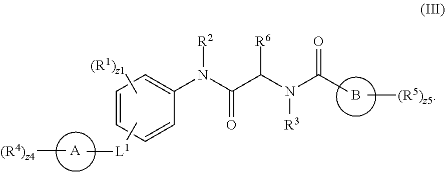

L¹, Ring A, Ring B, R¹, z1, R², R³, R⁴, z4, R⁵, z5, and R⁶ are as described herein, including in embodiments.

In embodiments, the compound has the formula:

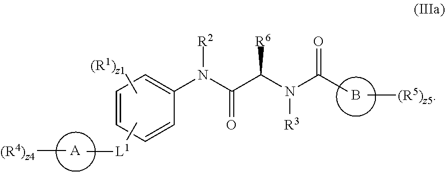

L¹, Ring A, Ring B, R¹, z1, R², R³, R⁴, z4, R⁵, z5, and R⁶ are as described herein, including in embodiments.

In embodiments, the compound has the formula:

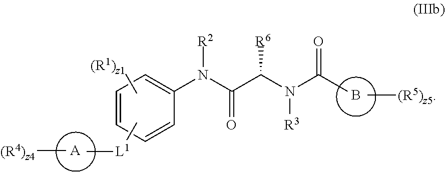

L¹, Ring A, Ring B, R¹, z1, R², R³, R⁴, z4, R⁵, z5, and R⁶ are as described herein, including in embodiments.

In embodiments, the compound has the formula:

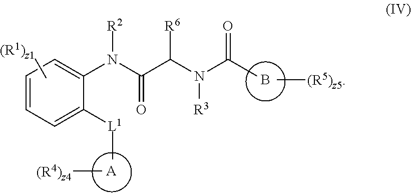

L¹, Ring A, Ring B, R¹, z1, R², R³, R⁴, z4, R⁵, z5, and R⁶ are as described herein, including in embodiments.

In embodiments, the compound has the formula:

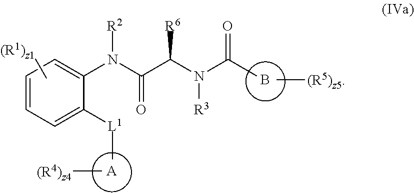

L¹, Ring A, Ring B, R¹, z1, R², R³, R⁴, z4, R⁵, z5, and R⁶ are as described herein, including in embodiments.

In embodiments, the compound has the formula:

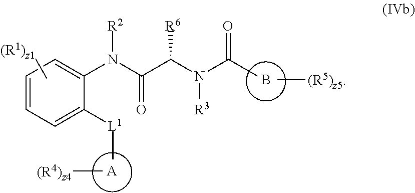

L¹, Ring A, Ring B, R¹, z1, R², R³, R⁴, z4, R⁵, z5, and R⁶ are as described herein, including in embodiments.

In embodiments, the compound has the formula:

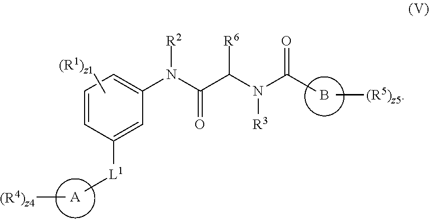

L¹, Ring A, Ring B, R¹, z1, R², R³, R⁴, z4, R⁵, z5, and R⁶ are as described herein, including in embodiments.

In embodiments, the compound has the formula:

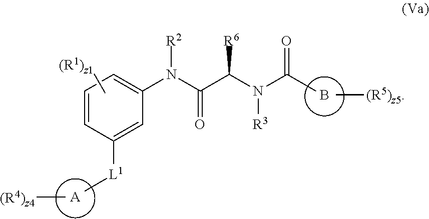

L¹, Ring A, Ring B, R¹, z1, R², R³, R⁴, z4, R⁵, z5, and R⁶ are as described herein, including in embodiments.

In embodiments, the compound has the formula:

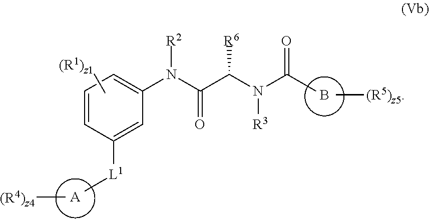

L¹, Ring A, Ring B, R¹, z1, R², R³, R⁴, z4, R⁵, z5, and R⁶ are as described herein, including in embodiments.

In embodiments, the compound has the formula:

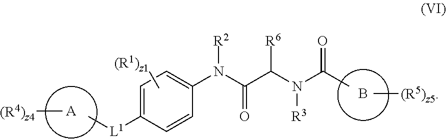

L¹, Ring A, Ring B, R¹, z1, R², R³, R⁴, z4, R⁵, z5, and R⁶ are as described herein, including in embodiments.

In embodiments, the compound has the formula:

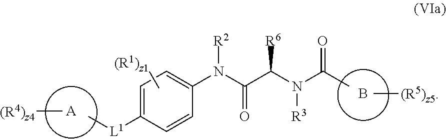

L¹, Ring A, Ring B, R¹, z1, R², R³, R⁴, z4, R⁵, z5, and R⁶ are as described herein, including in embodiments.

In embodiments, the compound has the formula:

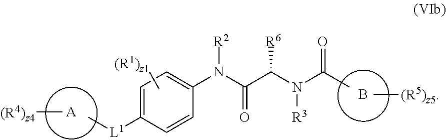

L¹, Ring A, Ring B, R¹, z1, R², R³, R⁴, z4, R⁵, z5, and R⁶ are as described herein, including in embodiments.

In embodiments, L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; wherein R⁸ and R⁹ are as described herein, including in embodiments. In embodiments, L¹ is —O—, —NH—, —NCH₃—, —S—, —C(O)—, —C(O)O—, —OC(O)—, —NHC(O)—, —C(O)NH—, —NHC(O)NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and R⁸ and R⁹ are independently halogen or unsubstituted methyl.

In embodiments, L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; wherein R⁸ and R⁹ are as described herein, including in embodiments. In embodiments, L¹ is —O—, —NH—, —NCH₃—, —S—, —C(O)—, —C(O)O—, —OC(O)—, —NHC(O)—, —C(O)NH—, —NHC(O)NH—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and R⁸ and R⁹ are independently halogen or unsubstituted methyl.

In embodiments, L¹ is —O—. In embodiments, L¹ is —NR⁷—, wherein R⁷ is as described herein, including in embodiments. In embodiments, L¹ is —NH—. In embodiments, L¹ is —NCH₃—. In embodiments, L¹ is —S—. In embodiments, L¹ is —C(O)—. In embodiments, L¹ is —C(O)O—. In embodiments, L¹ is —OC(O)—. In embodiments, L¹ is —NR⁷C(O)—, wherein R⁷ is as described herein, including in embodiments. In embodiments, L¹ is —NHC(O)—. In embodiments, L¹ is —C(O)NR⁷—, wherein R⁷ is as described herein, including in embodiments. In embodiments, L¹ is —C(O)NH—. In embodiments, L¹ is —NR⁷C(O)NR⁸—. In embodiments, L¹ is —NHC(O)NH—. In embodiments, L¹ is —NR⁷S(O)₂O—. In embodiments, L¹ is —NHS(O)₂O—. In embodiments, L¹ is —OS(O)₂NR⁷—. In embodiments, L¹ is —OS(O)₂NH—. In embodiments, L¹ is —NR⁷S(O)₂—. In embodiments, L¹ is —NHS(O)₂—. In embodiments, L¹ is —S(O)₂NR⁷—. In embodiments, L¹ is —S(O)₂NH—. In embodiments, L¹ is —S(O)—. In embodiments, L¹ is —S(O)₂—. In embodiments, L¹ is —OS(O)₂O—. In embodiments, L¹ is —S(O)₂O—. In embodiments, L¹ is —OS(O)₂—. In embodiments, L¹ is —P(O)(OR⁷)—, wherein R⁷ is as described herein, including in embodiments. In embodiments, L¹ is —P(O)(OH)—. In embodiments, L¹ is —OP(O)(OR⁷)O—, wherein R⁷ is as described herein, including in embodiments. In embodiments, L¹ is —OP(O)(OH)O—. In embodiments, L¹ is —OP(O)(OR⁷)—, wherein R⁷ is as described herein, including in embodiments. In embodiments, L¹ is —OP(O)(OH)—. In embodiments, L¹ is —P(O)(OR⁷)O—, wherein R⁷ is as described herein, including in embodiments. In embodiments, L¹ is —P(O)(OH)O—. In embodiments, L¹ is —CHR⁹—, wherein R⁹ is as described herein, including in embodiments. In embodiments, L¹ is —CR⁸R⁹—, wherein R⁸ and R⁹ are as described herein, including in embodiments. In embodiments, L¹ is —CHF—. In embodiments, L¹ is —CF₂—.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted ring formed when two R¹ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R¹ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R¹ substituents are joined is substituted, it is substituted with at least one substituent group. In embodiments, when the substituted ring formed when two R¹ substituents are joined is substituted, it is substituted with at least one size-limited substituent group. In embodiments, when the substituted ring formed when two R¹ substituents are joined is substituted, it is substituted with at least one lower substituent group.

In embodiments, a substituted RIA (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted RIA is substituted with a plurality of groups selected from substituent groups, size-limited substituent groups, and lower substituent groups; each substituent group, size-limited substituent group, and/or lower substituent group may optionally be different. In embodiments, when RIA is substituted, it is substituted with at least one substituent group. In embodiments, when RIA is substituted, it is substituted with at least one size-limited substituent group. In embodiments, when RIA is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when RIA and RIB substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted RID (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted RID is substituted with a plurality of groups selected from substituent groups, size-limited substituent groups, and lower substituent groups; each substituent group, size-limited substituent group, and/or lower substituent group may optionally be different. In embodiments, when RID is substituted, it is substituted with at least one substituent group. In embodiments, when RID is substituted, it is substituted with at least one size-limited substituent group. In embodiments, when RID is substituted, it is substituted with at least one lower substituent group.

In embodiments, R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered); two adjacent R¹ substituents may optionally be joined to form an unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl. In embodiments, R¹ is independently halogen, —OH, —CF₃, —CHF₂, —CH₂F, —OCF₃, —OCHF₂, —OCH₂F, unsubstituted methyl, or unsubstituted methoxy.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CCl₃. In embodiments, R¹ is independently —CBr₃. In embodiments, R¹ is independently —CF₃. In embodiments, R¹ is independently —Cl₃. In embodiments, R¹ is independently —CH₂Cl. In embodiments, R¹ is independently —CH₂Br. In embodiments, R¹ is independently —CH₂F. In embodiments, R¹ is independently —CH₂I. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently —SO₃H. In embodiments, R¹ is independently —OSO₃H. In embodiments, R¹ is independently —SO₂NH₂. In embodiments, R¹ is independently —NHNH₂. In embodiments, R¹ is independently —ONH₂. In embodiments, R¹ is independently —NHC(O)NHNH₂. In embodiments, R¹ is independently —NHC(O)NH₂. In embodiments, R¹ is independently —NHSO₂H. In embodiments, R¹ is independently —NHC(O)H. In embodiments, R¹ is independently —NHC(O)OH. In embodiments, R¹ is independently —NHOH. In embodiments, R¹ is independently —OCCl₃. In embodiments, R¹ is independently —OCBr₃. In embodiments, R¹ is independently —OCF₃. In embodiments, R¹ is independently —OCl₃. In embodiments, R¹ is independently —OCH₂Cl. In embodiments, R¹ is independently —OCH₂Br. In embodiments, R¹ is independently —OCH₂F. In embodiments, R¹ is independently —OCH₂I. In embodiments, R¹ is independently —OCHCl₂. In embodiments, R¹ is independently —OCHBr₂. In embodiments, R¹ is independently —OCHF₂. In embodiments, R¹ is independently —OCHI₂. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 4 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy. In embodiments, R¹ is independently unsubstituted n-butoxy. In embodiments, R¹ is independently unsubstituted isobutoxy. In embodiments, R¹ is independently unsubstituted tert-butoxy.

In embodiments, z1 is 0. In embodiments, z1 is 1. In embodiments, z1 is 2. In embodiments, z1 is 3. In embodiments, z1 is 4.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is hydrogen, halogen, —CX²₃, —CHX²₂, —CH₂X², —CN, —COOH, —CONH₂, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R² is hydrogen, —CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R² is hydrogen, unsubstituted methyl, unsubstituted ethyl, or unsubstituted isopropyl. In embodiments, R² is hydrogen.

In embodiments, R² is hydrogen or unsubstituted C₁-C₄ alkyl. In embodiments, R² is hydrogen. In embodiments, R² is unsubstituted C₁-C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted isobutyl. In embodiments, R² is unsubstituted tert-butyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen, halogen, —CX³₃, —CHX³₂, —CH₂X³, —CN, —COOH, —CONH₂, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R³ is hydrogen, —CX³₃, —CHX³₂, —CH₂X³, —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R³ is hydrogen, unsubstituted methyl, unsubstituted ethyl, or unsubstituted isopropyl. In embodiments, R³ is hydrogen.

In embodiments, R³ is hydrogen or unsubstituted C₁-C₄ alkyl. In embodiments, R³ is hydrogen. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted isobutyl. In embodiments, R³ is unsubstituted tert-butyl.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted ring formed when two R⁴ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R⁴ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R⁴ substituents are joined is substituted, it is substituted with at least one substituent group. In embodiments, when the substituted ring formed when two R⁴ substituents are joined is substituted, it is substituted with at least one size-limited substituent group. In embodiments, when the substituted ring formed when two R⁴ substituents are joined is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered); two adjacent R⁴ substituents may optionally be joined to form an unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁴ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl. In embodiments, R⁴ is independently halogen, —CF₃, —CHF₂, —CH₂F, —OCF₃, —OCHF₂, —OCH₂F, —OH, unsubstituted methyl, or unsubstituted methoxy.

In embodiments, R⁴ is independently halogen. In embodiments, R⁴ is independently —F. In embodiments, R⁴ is independently —Cl. In embodiments, R⁴ is independently —Br. In embodiments, R⁴ is independently —I. In embodiments, R⁴ is independently —CCl₃. In embodiments, R⁴ is independently —CBr₃. In embodiments, R⁴ is independently —CF₃. In embodiments, R⁴ is independently —Cl₃. In embodiments, R⁴ is independently —CH₂Cl. In embodiments, R⁴ is independently —CH₂Br. In embodiments, R⁴ is independently —CH₂F. In embodiments, R⁴ is independently —CH₂I. In embodiments, R⁴ is independently —CHCl₂. In embodiments, R⁴ is independently —CHBr₂. In embodiments, R⁴ is independently —CHF₂. In embodiments, R⁴ is independently —CHI₂. In embodiments, R⁴ is independently —CN. In embodiments, R⁴ is independently —OH. In embodiments, R⁴ is independently —NH₂. In embodiments, R⁴ is independently —COOH. In embodiments, R⁴ is independently —CONH₂. In embodiments, R⁴ is independently —NO₂. In embodiments, R⁴ is independently —SH. In embodiments, R⁴ is independently —SO₃H. In embodiments, R⁴ is independently —OSO₃H. In embodiments, R⁴ is independently —SO₂NH₂. In embodiments, R⁴ is independently —NHNH₂. In embodiments, R⁴ is independently —ONH₂. In embodiments, R⁴ is independently —NHC(O)NHNH₂. In embodiments, R⁴ is independently —NHC(O)NH₂. In embodiments, R⁴ is independently —NHSO₂H. In embodiments, R⁴ is independently —NHC(O)H. In embodiments, R⁴ is independently —NHC(O)OH. In embodiments, R⁴ is independently —NHOH. In embodiments, R⁴ is independently —OCCl₃. In embodiments, R⁴ is independently —OCBr₃. In embodiments, R⁴ is independently —OCF₃. In embodiments, R⁴ is independently —OCl₃. In embodiments, R⁴ is independently —OCH₂Cl. In embodiments, R⁴ is independently —OCH₂Br. In embodiments, R⁴ is independently —OCH₂F. In embodiments, R⁴ is independently —OCH₂I. In embodiments, R⁴ is independently —OCHCl₂. In embodiments, R⁴ is independently —OCHBr₂. In embodiments, R⁴ is independently —OCHF₂. In embodiments, R⁴ is independently —OCHI₂. In embodiments, R⁴ is independently unsubstituted C₁-C₄ alkyl. In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted n-propyl. In embodiments, R⁴ is independently unsubstituted isopropyl. In embodiments, R⁴ is independently unsubstituted butyl. In embodiments, R⁴ is independently unsubstituted n-butyl. In embodiments, R⁴ is independently unsubstituted isobutyl. In embodiments, R⁴ is independently unsubstituted tert-butyl. In embodiments, R⁴ is independently unsubstituted 2 to 4 membered heteroalkyl. In embodiments, R⁴ is independently unsubstituted methoxy. In embodiments, R⁴ is independently unsubstituted ethoxy. In embodiments, R⁴ is independently unsubstituted propoxy. In embodiments, R⁴ is independently unsubstituted n-propoxy. In embodiments, R⁴ is independently unsubstituted isopropoxy. In embodiments, R⁴ is independently unsubstituted butoxy. In embodiments, R⁴ is independently unsubstituted n-butoxy. In embodiments, R⁴ is independently unsubstituted isobutoxy. In embodiments, R⁴ is independently unsubstituted tert-butoxy.

In embodiments, R⁴ is independently —OR^{4D}, wherein R^{4D} is as described herein, including in embodiments. In embodiments, R^{4D} is hydrogen or substituted or unsubstituted alkyl. In embodiments, R^{4D} is independently hydrogen or unsubstituted alkyl. In embodiments, R^{4D} is independently hydrogen or unsubstituted C₁-C₅ alkyl. In embodiments, R^{4D} is independently hydrogen or unsubstituted methyl. In embodiments, R^{4D} is independently hydrogen. In embodiments, R^{4D} is independently unsubstituted C₁-C₅ alkyl.

In embodiments, R^{4D} is independently unsubstituted methyl. In embodiments, R^{4D} is independently unsubstituted ethyl. In embodiments, R^{4D} is independently unsubstituted propyl. In embodiments, R^{4D} is independently unsubstituted n-propyl. In embodiments, R^{4D} is independently unsubstituted isopropyl. In embodiments, R^{4D} is independently unsubstituted butyl. In embodiments, R^{4D} is independently unsubstituted n-butyl. In embodiments, R^{4D} is independently unsubstituted isobutyl. In embodiments, R^{4D} is independently unsubstituted tert-butyl.

In embodiments, z4 is 0. In embodiments, z4 is 1. In embodiments, z4 is 2. In embodiments, z4 is 3. In embodiments, z4 is 4.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, a substituted ring formed when two R⁵ substituents are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed when two R⁵ substituents are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when two R⁵ substituents are joined is substituted, it is substituted with at least one substituent group. In embodiments, when the substituted ring formed when two R⁵ substituents are joined is substituted, it is substituted with at least one size-limited substituent group. In embodiments, when the substituted ring formed when two R⁵ substituents are joined is substituted, it is substituted with at least one lower substituent group.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted ring formed when R^{5A} and R^{5B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{5A} and R^{5B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{5A} and R^{5B} substituents bonded to the same nitrogen atom are joined is substituted, it is substituted with at least one substituent group. In embodiments, when the substituted ring formed when R^{5A} and R^{5B} substituents bonded to the same nitrogen atom are joined is substituted, it is substituted with at least one size-limited substituent group. In embodiments, when the substituted ring formed when R^{5A} and R^{5B} substituents bonded to the same nitrogen atom are joined is substituted, it is substituted with at least one lower substituent group.

In embodiments, a substituted R^{5C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{5D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5D} is substituted with a plurality of groups selected from substituent groups, size-limited substituent groups, and lower substituent groups; each substituent group, size-limited substituent group, and/or lower substituent group may optionally be different. In embodiments, when R^{5D} is substituted, it is substituted with at least one substituent group. In embodiments, when R^{5D} is substituted, it is substituted with at least one size-limited substituent group. In embodiments, when R^{5D} is substituted, it is substituted with at least one lower substituent group.

In embodiments, R⁵ is independently a halogen, —CX₅³, —CHX⁵₂, —CH₂X⁵, —OCX₅³, —OCHX⁵₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered); two adjacent R⁵ substituents may optionally be joined to form an unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁵ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁵ is independently halogen, —CF₃, —CHF₂, —CH₂F, —OCF₃, —OCHF₂, —OCH₂F, —OH, —NH₂, —SH, unsubstituted C₁-C₄ alkyl, unsubstituted 2 to 4 membered heteroalkyl, or unsubstituted phenyl. In embodiments, R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

In embodiments, R⁵ is independently halogen. In embodiments, R⁵ is independently —F. In embodiments, R⁵ is independently —Cl. In embodiments, R⁵ is independently —Br. In embodiments, R⁵ is independently —I. In embodiments, R⁵ is independently —CCl₃. In embodiments, R⁵ is independently —CBr₃. In embodiments, R⁵ is independently —CF₃. In embodiments, R⁵ is independently —Cl₃. In embodiments, R⁵ is independently —CH₂Cl. In embodiments, R⁵ is independently —CH₂Br. In embodiments, R⁵ is independently —CH₂F. In embodiments, R⁵ is independently —CH₂I. In embodiments, R⁵ is independently —CHCl₂. In embodiments, R⁵ is independently —CHBr₂. In embodiments, R⁵ is independently —CHF₂. In embodiments, R⁵ is independently —CHI₂. In embodiments, R⁵ is independently —CN. In embodiments, R⁵ is independently —OH. In embodiments, R⁵ is independently —NH₂. In embodiments, R⁵ is independently —COOH. In embodiments, R⁵ is independently —CONH₂. In embodiments, R⁵ is independently —NO₂. In embodiments, R⁵ is independently —SH. In embodiments, R⁵ is independently —SO₃H. In embodiments, R⁵ is independently —OSO₃H. In embodiments, R⁵ is independently —SO₂NH₂. In embodiments, R⁵ is independently —NHNH₂. In embodiments, R⁵ is independently —ONH₂. In embodiments, R⁵ is independently —NHC(O)NHNH₂. In embodiments, R⁵ is independently —NHC(O)NH₂. In embodiments, R⁵ is independently —NHSO₂H. In embodiments, R⁵ is independently —NHC(O)H. In embodiments, R⁵ is independently —NHC(O)OH. In embodiments, R⁵ is independently —NHOH. In embodiments, R⁵ is independently —OCCl₃. In embodiments, R⁵ is independently —OCBr₃. In embodiments, R⁵ is independently —OCF₃. In embodiments, R⁵ is independently —OCl₃. In embodiments, R⁵ is independently —OCH₂Cl. In embodiments, R⁵ is independently —OCH₂Br. In embodiments, R⁵ is independently —OCH₂F. In embodiments, R⁵ is independently —OCH₂I. In embodiments, R⁵ is independently —OCHCl₂. In embodiments, R⁵ is independently —OCHBr₂. In embodiments, R⁵ is independently —OCHF₂. In embodiments, R⁵ is independently —OCHI₂. In embodiments, R⁵ is independently unsubstituted C₁-C₄ alkyl. In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted n-propyl. In embodiments, R⁵ is independently unsubstituted isopropyl. In embodiments, R⁵ is independently unsubstituted butyl. In embodiments, R⁵ is independently unsubstituted n-butyl. In embodiments, R⁵ is independently unsubstituted isobutyl. In embodiments, R⁵ is independently unsubstituted tert-butyl. In embodiments, R⁵ is independently unsubstituted 2 to 4 membered heteroalkyl. In embodiments, R⁵ is independently unsubstituted methoxy. In embodiments, R⁵ is independently unsubstituted ethoxy. In embodiments, R⁵ is independently unsubstituted propoxy. In embodiments, R⁵ is independently unsubstituted n-propoxy. In embodiments, R⁵ is independently unsubstituted isopropoxy. In embodiments, R⁵ is independently unsubstituted butoxy. In embodiments, R⁵ is independently unsubstituted n-butoxy. In embodiments, R⁵ is independently unsubstituted isobutoxy. In embodiments, R⁵ is independently unsubstituted tert-butoxy. In embodiments, R⁵ is independently substituted or unsubstituted phenyl. In embodiments, R⁵ is independently substituted phenyl. In embodiments, R⁵ is independently unsubstituted phenyl.

In embodiments, z5 is 0. In embodiments, z5 is 1. In embodiments, z5 is 2. In embodiments, z5 is 3. In embodiments, z5 is 4. In embodiments, z5 is 5. In embodiments, z5 is 6. In embodiments, z5 is 7.

In embodiments,

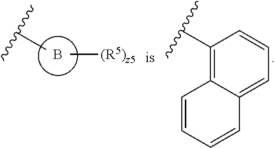

In embodiments,

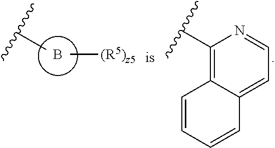

In embodiments,

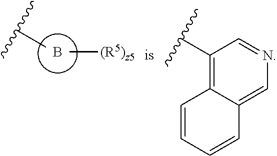

In embodiments,

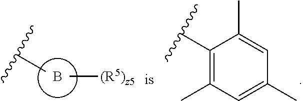

In embodiments,

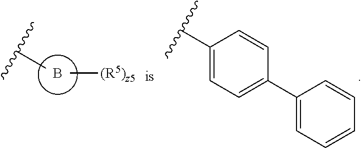

In embodiments,

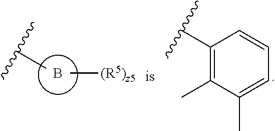

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted ring formed when R^{6A} and R^{6B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{6A} and R^{6B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{6A} and R^{6B} substituents bonded to the same nitrogen atom are joined is substituted, it is substituted with at least one substituent group. In embodiments, when the substituted ring formed when R^{6A} and R^{6B} substituents bonded to the same nitrogen atom are joined is substituted, it is substituted with at least one size-limited substituent group. In embodiments, when the substituted ring formed when R^{6A} and R^{6B} substituents bonded to the same nitrogen atom are joined is substituted, it is substituted with at least one lower substituent group.

In embodiments, a substituted R^{6C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted R^{6D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6D} is substituted with a plurality of groups selected from substituent groups, size-limited substituent groups, and lower substituent groups; each substituent group, size-limited substituent group, and/or lower substituent group may optionally be different. In embodiments, when R^{6D} is substituted, it is substituted with at least one substituent group. In embodiments, when R^{6D} is substituted, it is substituted with at least one size-limited substituent group. In embodiments, when R^{6D} is substituted, it is substituted with at least one lower substituent group.

In embodiments, R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered).

In embodiments, R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl. In embodiments, R⁶ is substituted or unsubstituted C₁-C₆ alkyl or substituted or unsubstituted 2 to 6 membered heteroalkyl.

In embodiments, R⁶ is hydrogen. In embodiments, R⁶ is halogen. In embodiments, R⁶ is —F. In embodiments, R⁶ is —Cl. In embodiments, R⁶ is —Br. In embodiments, R⁶ is —I.

In embodiments, R⁶ is —CCl₃. In embodiments, R⁶ is —CBr₃. In embodiments, R⁶ is —CF₃. In embodiments, R⁶ is —Cl₃. In embodiments, R⁶ is —CH₂Cl. In embodiments, R⁶ is —CH₂Br. In embodiments, R⁶ is —CH₂F. In embodiments, R⁶ is —CH₂I. In embodiments, R⁶ is —CHCl₂. In embodiments, R⁶ is —CHBr₂. In embodiments, R⁶ is —CHF₂. In embodiments, R⁶ is —CHI₂. In embodiments, R⁶ is —CN. In embodiments, R⁶ is —OH. In embodiments, R⁶ is —NH₂. In embodiments, R⁶ is —COOH. In embodiments, R⁶ is —CONH₂. In embodiments, R⁶ is —NO₂. In embodiments, R⁶ is —SH. In embodiments, R⁶ is —SO₃H. In embodiments, R⁶ is —OSO₃H. In embodiments, R⁶ is —SO₂NH₂. In embodiments, R⁶ is —NHNH₂. In embodiments, R⁶ is —ONH₂. In embodiments, R⁶ is —NHC(O)NHNH₂. In embodiments, R⁶ is —NHC(O)NH₂. In embodiments, R⁶ is —NHSO₂H. In embodiments, R⁶ is —NHC(O)H. In embodiments, R⁶ is —NHC(O)OH. In embodiments, R⁶ is —NHOH. In embodiments, R⁶ is —OCCl₃. In embodiments, R⁶ is —OCBr₃. In embodiments, R⁶ is —OCF₃. In embodiments, R⁶ is —OCl₃. In embodiments, R⁶ is —OCH₂Cl. In embodiments, R⁶ is —OCH₂Br. In embodiments, R⁶ is —OCH₂F. In embodiments, R⁶ is —OCH₂I. In embodiments, R⁶ is —OCHCl₂. In embodiments, R⁶ is —OCHBr₂. In embodiments, R⁶ is —OCHF₂. In embodiments, R⁶ is —OCHI₂. In embodiments, R⁶ is substituted or unsubstituted C₁-C₄ alkyl. In embodiments, R⁶ is substituted C₁-C₄ alkyl. In embodiments, R⁶ is substituted methyl. In embodiments, R⁶ is substituted ethyl. In embodiments, R⁶ is substituted propyl. In embodiments, R⁶ is substituted n-propyl. In embodiments, R⁶ is substituted isopropyl. In embodiments, R⁶ is substituted butyl. In embodiments, R⁶ is substituted n-butyl. In embodiments, R⁶ is substituted isobutyl. In embodiments, R⁶ is substituted tert-butyl. In embodiments, R⁶ is unsubstituted C₁-C₄ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted n-propyl. In embodiments, R⁶ is unsubstituted isopropyl. In embodiments, R⁶ is unsubstituted butyl. In embodiments, R⁶ is unsubstituted n-butyl. In embodiments, R⁶ is unsubstituted isobutyl. In embodiments, R⁶ is unsubstituted tert-butyl. In embodiments, R⁶ is substituted or unsubstituted 2 to 4 membered heteroalkyl. In embodiments, R⁶ is substituted 2 to 4 membered heteroalkyl. In embodiments, R⁶ is unsubstituted 2 to 4 membered heteroalkyl. In embodiments, R⁶ is unsubstituted methoxy. In embodiments, R⁶ is unsubstituted ethoxy. In embodiments, R⁶ is unsubstituted propoxy. In embodiments, R⁶ is unsubstituted n-propoxy. In embodiments, R⁶ is unsubstituted isopropoxy. In embodiments, R⁶ is unsubstituted butoxy. In embodiments, R⁶ is unsubstituted n-butoxy. In embodiments, R⁶ is unsubstituted isobutoxy. In embodiments, R⁶ is unsubstituted tert-butoxy.

In embodiments, R⁶ is an amino acid side chain. In embodiments, R⁶ is a glycine side chain. In embodiments, R⁶ is an alanine side chain. In embodiments, R⁶ is a valine side chain. In embodiments, R⁶ is a leucine side chain. In embodiments, R⁶ is an isoleucine side chain. In embodiments, R⁶ is a methionine side chain. In embodiments, R⁶ is a serine side chain. In embodiments, R⁶ is a threonine side chain. In embodiments, R⁶ is a cysteine side chain. In embodiments, R⁶ is an aspartic acid side chain. In embodiments, R⁶ is a glutamic acid side chain. In embodiments, R⁶ is an asparagine side chain. In embodiments, R⁶ is a glutamine side chain. In embodiments, R⁶ is a histidine side chain. In embodiments, R⁶ is a phenylalanine side chain. In embodiments, R⁶ is a tyrosine side chain. In embodiments, R⁶ is a tryptophan side chain. In embodiments, R⁶ is an arginine side chain. In embodiments, R⁶ is a lysine side chain.

In embodiments, R⁶ is an amino acid side chain. In embodiments, R⁶ is an L-glycine side chain. In embodiments, R⁶ is an L-alanine side chain. In embodiments, R⁶ is an L-valine side chain. In embodiments, R⁶ is an L-leucine side chain. In embodiments, R⁶ is an L-isoleucine side chain. In embodiments, R⁶ is an L-methionine side chain. In embodiments, R⁶ is an L-serine side chain. In embodiments, R⁶ is an L-threonine side chain. In embodiments, R⁶ is an L-cysteine side chain. In embodiments, R⁶ is an L-aspartic acid side chain. In embodiments, R⁶ is an L-glutamic acid side chain. In embodiments, R⁶ is an L-asparagine side chain. In embodiments, R⁶ is an L-glutamine side chain. In embodiments, R⁶ is an L-histidine side chain. In embodiments, R⁶ is an L-phenylalanine side chain. In embodiments, R⁶ is an L-tyrosine side chain. In embodiments, R⁶ is an L-tryptophan side chain. In embodiments, R⁶ is an L-arginine side chain. In embodiments, R⁶ is an L-lysine side chain.

In embodiments, R⁶ is an amino acid side chain. In embodiments, R⁶ is a D-glycine side chain. In embodiments, R⁶ is a D-alanine side chain. In embodiments, R⁶ is a D-valine side chain. In embodiments, R⁶ is a D-leucine side chain. In embodiments, R⁶ is a D-isoleucine side chain. In embodiments, R⁶ is a D-methionine side chain. In embodiments, R⁶ is a D-serine side chain. In embodiments, R⁶ is a D-threonine side chain. In embodiments, R⁶ is a D-cysteine side chain. In embodiments, R⁶ is a D-aspartic acid side chain. In embodiments, R⁶ is a D-glutamic acid side chain. In embodiments, R⁶ is a D-asparagine side chain. In embodiments, R⁶ is a D-glutamine side chain. In embodiments, R⁶ is a D-histidine side chain. In embodiments, R⁶ is a D-phenylalanine side chain. In embodiments, R⁶ is a D-tyrosine side chain. In embodiments, R⁶ is a D-tryptophan side chain. In embodiments, R⁶ is a D-arginine side chain. In embodiments, R⁶ is a D-lysine side chain.

In embodiments, R⁶ is hydrogen, unsubstituted methyl, unsubstituted isopropyl,

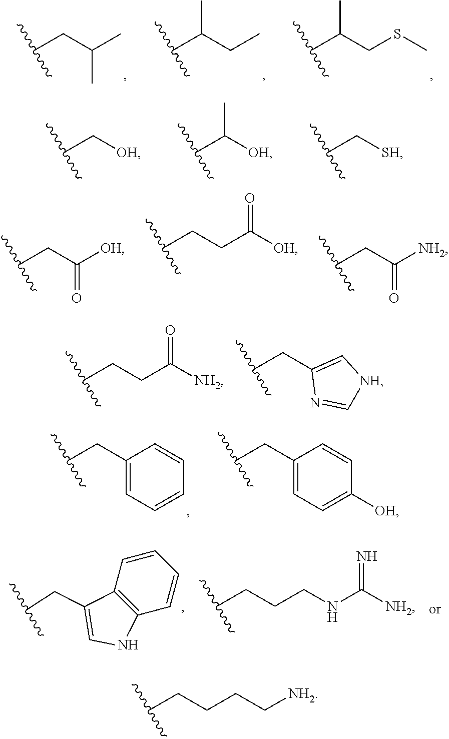

In embodiments, a substituted ring formed when R³ and R⁶ substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R³ and R⁶ substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R³ and R⁶ substituents bonded to the same nitrogen atom are joined is substituted, it is substituted with at least one substituent group. In embodiments, when the substituted ring formed when R³ and R⁶ substituents bonded to the same nitrogen atom are joined is substituted, it is substituted with at least one size-limited substituent group. In embodiments, when the substituted ring formed when R³ and R⁶ substituents bonded to the same nitrogen atom are joined is substituted, it is substituted with at least one lower substituent group.

In embodiments, R³ and R⁶ may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered).

In embodiments, R³ and R⁶ are joined to form a substituted or unsubstituted heterocycloalkyl or substituted or unsubstituted heteroaryl. In embodiments, R³ and R⁶ are joined to form a substituted or unsubstituted 4 to 8 membered heterocycloalkyl. In embodiments, R³ and R⁶ are joined to form an unsubstituted pyrrolidinyl. In embodiments, R³ and R⁶ are joined to form an unsubstituted piperidinyl.

R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered); R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered); R^{6A} and R^{6B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered).

R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered); R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered); R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered).

In embodiments, a substituted R⁷ (e.g., substituted alkyl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁷ is hydrogen, halogen, —OH, —N₃, or substituted or unsubstituted alkyl (e.g., C₁-C₈, C₁-C₆, C₁-C₄, or C₁-C₂). In embodiments, R⁷ is hydrogen, halogen, —OH, —N₃, or unsubstituted alkyl (e.g., C₁-C₈, C₁-C₆, C₁-C₄, or C₁-C₂). In embodiments, R⁷ is hydrogen. In embodiments, R⁷ is halogen. In embodiments, R⁷ is —F. In embodiments, R⁷ is —Cl. In embodiments, R⁷ is —Br. In embodiments, R⁷ is —I. In embodiments, R⁷ is —OH. In embodiments, R⁷ is —N₃. In embodiments, R⁷ is substituted or unsubstituted C₁-C₄ alkyl. In embodiments, R⁷ is unsubstituted C₁-C₄ alkyl. In embodiments, R⁷ is unsubstituted methyl. In embodiments, R⁷ is unsubstituted ethyl. In embodiments, R⁷ is unsubstituted propyl. In embodiments, R⁷ is unsubstituted n-propyl. In embodiments, R⁷ is unsubstituted isopropyl. In embodiments, R⁷ is unsubstituted butyl. In embodiments, R⁷ is unsubstituted n-butyl. In embodiments, R⁷ is unsubstituted isobutyl. In embodiments, R⁷ is unsubstituted tert-butyl.

In embodiments, a substituted R⁸ (e.g., substituted alkyl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R⁸ is hydrogen, halogen, —OH, —N₃, or substituted or unsubstituted alkyl (e.g., C₁-C₈, C₁-C₆, C₁-C₄, or C₁-C₂). In embodiments, R⁸ is hydrogen, halogen, —OH, —N₃, or unsubstituted alkyl (e.g., C₁-C₈, C₁-C₆, C₁-C₄, or C₁-C₂). In embodiments, R⁸ is hydrogen. In embodiments, R⁸ is halogen. In embodiments, R⁸ is —F. In embodiments, R⁸ is —Cl. In embodiments, R⁸ is —Br. In embodiments, R⁸ is —I. In embodiments, R⁸ is —OH. In embodiments, R⁸ is —N₃. In embodiments, R⁸ is substituted or unsubstituted C₁-C₄ alkyl. In embodiments, R⁸ is unsubstituted C₁-C₄ alkyl. In embodiments, R⁸ is unsubstituted methyl. In embodiments, R⁸ is unsubstituted ethyl. In embodiments, R⁸ is unsubstituted propyl. In embodiments, R⁸ is unsubstituted n-propyl. In embodiments, R⁸ is unsubstituted isopropyl. In embodiments, R⁸ is unsubstituted butyl. In embodiments, R⁸ is unsubstituted n-butyl. In embodiments, R⁸ is unsubstituted isobutyl. In embodiments, R⁸ is unsubstituted tert-butyl.

In embodiments, a substituted R⁹ (e.g., substituted alkyl) is substituted with at least one substituent group, size-limited substituent group, or lower substituent group; wherein if the substituted R⁹ is substituted with a plurality of groups selected from substituent groups, size-limited substituent groups, and lower substituent groups; each substituent group, size-limited substituent group, and/or lower substituent group may optionally be different. In embodiments, when R⁹ is substituted, it is substituted with at least one substituent group. In embodiments, when R⁹ is substituted, it is substituted with at least one size-limited substituent group. In embodiments, when R⁹ is substituted, it is substituted with at least one lower substituent group.

In embodiments, R⁹ is hydrogen, halogen, —OH, —N₃, or substituted or unsubstituted alkyl (e.g., C₁-C₈, C₁-C₆, C₁-C₄, or C₁-C₂). In embodiments, R⁹ is hydrogen, halogen, —OH, —N₃, or unsubstituted alkyl (e.g., C₁-C₈, C₁-C₆, C₁-C₄, or C₁-C₂). In embodiments, R⁹ is hydrogen. In embodiments, R⁹ is halogen. In embodiments, R⁹ is —F. In embodiments, R⁹ is —Cl. In embodiments, R⁹ is —Br. In embodiments, R⁹ is —I. In embodiments, R⁹ is —OH. In embodiments, R⁹ is —N₃. In embodiments, R⁹ is substituted or unsubstituted C₁-C₄ alkyl. In embodiments, R⁹ is unsubstituted C₁-C₄ alkyl. In embodiments, R⁹ is unsubstituted methyl. In embodiments, R⁹ is unsubstituted ethyl. In embodiments, R⁹ is unsubstituted propyl. In embodiments, R⁹ is unsubstituted n-propyl. In embodiments, R⁹ is unsubstituted isopropyl. In embodiments, R⁹ is unsubstituted butyl. In embodiments, R⁹ is unsubstituted n-butyl. In embodiments, R⁹ is unsubstituted isobutyl. In embodiments, R⁹ is unsubstituted tert-butyl.

In embodiments, a substituted Ring A (e.g., substituted phenyl and/or substituted 5 to 6 membered heteroaryl) is substituted with at least one substituent group, size-limited substituent group, or lower substituent group; wherein if the substituted Ring A is substituted with a plurality of groups selected from substituent groups, size-limited substituent groups, and lower substituent groups; each substituent group, size-limited substituent group, and/or lower substituent group may optionally be different. In embodiments, when Ring A is substituted, it is substituted with at least one substituent group. In embodiments, when Ring A is substituted, it is substituted with at least one size-limited substituent group. In embodiments, when Ring A is substituted, it is substituted with at least one lower substituent group.

In embodiments, Ring A is unsubstituted phenyl or unsubstituted 5 to 6 membered heteroaryl. In embodiments, Ring A is a substituted phenyl. In embodiments, Ring A is an unsubstituted phenyl. In embodiments, Ring A is a substituted 5 to 6 membered heteroaryl. In embodiments, Ring A is an unsubstituted 5 to 6 membered heteroaryl. In embodiments, Ring A is a substituted thienyl. In embodiments, Ring A is an unsubstituted thienyl. In embodiments, Ring A is a substituted 2-thienyl. In embodiments, Ring A is an unsubstituted 2-thienyl. In embodiments, Ring A is a substituted 3-thienyl. In embodiments, Ring A is an unsubstituted 3-thienyl. In embodiments, Ring A is a substituted pyridyl. In embodiments, Ring A is an unsubstituted pyridyl. In embodiments, Ring A is a substituted 2-pyridyl. In embodiments, Ring A is an unsubstituted 2-pyridyl. In embodiments, Ring A is a substituted 3-pyridyl. In embodiments, Ring A is an unsubstituted 3-pyridyl. In embodiments, Ring A is a substituted 4-pyridyl. In embodiments, Ring A is an unsubstituted 4-pyridyl. In embodiments, Ring A is a substituted pyrrolyl. In embodiments, Ring A is an unsubstituted pyrrolyl. In embodiments, Ring A is a substituted furanyl. In embodiments, Ring A is an unsubstituted furanyl. In embodiments, Ring A is a substituted pyrazolyl. In embodiments, Ring A is an unsubstituted pyrazolyl. In embodiments, Ring A is a substituted imidazolyl. In embodiments, Ring A is an unsubstituted imidazolyl. In embodiments, Ring A is a substituted oxazolyl. In embodiments, Ring A is an unsubstituted oxazolyl. In embodiments, Ring A is a substituted isoxazolyl. In embodiments, Ring A is an unsubstituted isoxazolyl. In embodiments, Ring A is a substituted thiazolyl. In embodiments, Ring A is an unsubstituted thiazolyl. In embodiments, Ring A is a substituted triazolyl. In embodiments, Ring A is an unsubstituted triazolyl.

In embodiments, Ring A is phenyl. In embodiments, Ring A is 5 to 6 membered heteroaryl. In embodiments, Ring A is thienyl. In embodiments, Ring A is 2-thienyl. In embodiments, Ring A is 3-thienyl. In embodiments, Ring A is pyridyl. In embodiments, Ring A is 2-pyridyl. In embodiments, Ring A is 3-pyridyl. In embodiments, Ring A is 4-pyridyl. In embodiments, Ring A is pyrrolyl. In embodiments, Ring A is furanyl. In embodiments, Ring A is pyrazolyl. In embodiments, Ring A is imidazolyl. In embodiments, Ring A is oxazolyl. In embodiments, Ring A is isoxazolyl. In embodiments, Ring A is thiazolyl. In embodiments, Ring A is triazolyl.

In embodiments, Ring A is

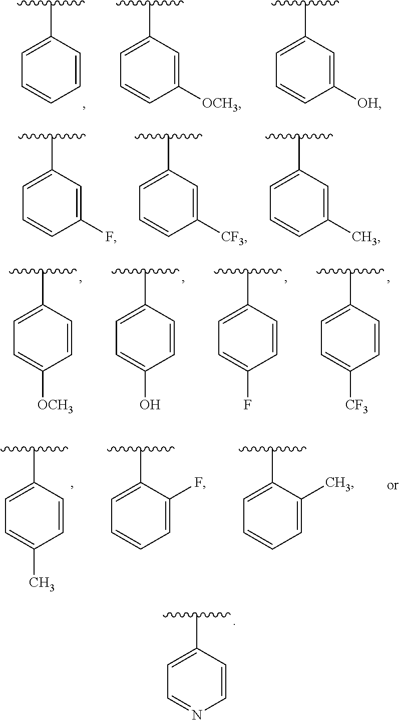

In embodiments, Ring A is

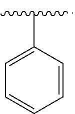 [SMILES: *c1ccccc1]

In embodiments, Ring A is

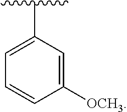 [SMILES: *c1cccc(OC)c1]

In embodiments, Ring A is

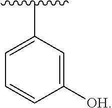 [SMILES: *c1cccc(O)c1]

In embodiments, Ring A is

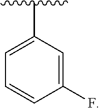 [SMILES: *c1cccc(F)c1]

In embodiments, Ring A is

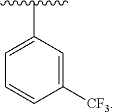 [SMILES: *c1cccc(C(F)(F)F)c1]

In embodiments, Ring A is

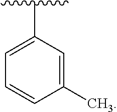 [SMILES: *c1cccc(C)c1]

In embodiments, Ring A is

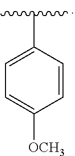 [SMILES: *c1ccc(OC)cc1]

In embodiments, Ring A is

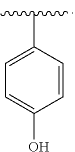 [SMILES: *c1ccc(O)cc1]

In embodiments, Ring A is

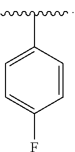 [SMILES: *c1ccc(F)cc1]

In embodiments, Ring A is

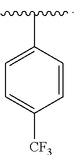 [SMILES: *c1ccc(C(F)(F)F)cc1]

In embodiments, Ring A is

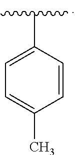 [SMILES: *c1ccc(C)cc1]

In embodiments, Ring A is

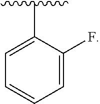 [SMILES: *c1ccccc1F]

In embodiments, Ring A is

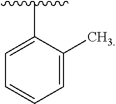 [SMILES: *c1ccccc1C]

In embodiments, Ring A is

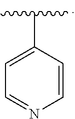 [SMILES: *c1ccncc1]

In embodiments, a substituted Ring B (e.g., substituted phenyl, substituted naphthyl, substituted quinolinyl, and/or substituted isoquinolinyl) is substituted with at least one substituent group, size-limited substituent group, or lower substituent group; wherein if the substituted Ring B is substituted with a plurality of groups selected from substituent groups, size-limited substituent groups, and lower substituent groups; each substituent group, size-limited substituent group, and/or lower substituent group may optionally be different. In embodiments, when Ring B is substituted, it is substituted with at least one substituent group. In embodiments, when Ring B is substituted, it is substituted with at least one size-limited substituent group. In embodiments, when Ring B is substituted, it is substituted with at least one lower substituent group.

In embodiments, Ring B is unsubstituted phenyl, unsubstituted naphthyl, unsubstituted quinolinyl, or unsubstituted isoquinolinyl. In embodiments, Ring B is a substituted phenyl. In embodiments, Ring B is an unsubstituted phenyl. In embodiments, Ring B is a substituted naphthyl. In embodiments, Ring B is an unsubstituted naphthyl. In embodiments, Ring B is a substituted 1-naphthyl. In embodiments, Ring B is an unsubstituted 1-naphthyl. In embodiments, Ring B is a substituted 2-naphthyl. In embodiments, Ring B is an unsubstituted 2-naphthyl. In embodiments, Ring B is a substituted quinolinyl. In embodiments, Ring B is an unsubstituted quinolinyl. In embodiments, Ring B is a substituted 2-quinolinyl. In embodiments, Ring B is an unsubstituted 2-quinolinyl. In embodiments, Ring B is a substituted 3-quinolinyl. In embodiments, Ring B is an unsubstituted 3-quinolinyl. In embodiments, Ring B is a substituted 4-quinolinyl. In embodiments, Ring B is an unsubstituted 4-quinolinyl. In embodiments, Ring B is a substituted isoquinolinyl. In embodiments, Ring B is an unsubstituted isoquinolinyl. In embodiments, Ring B is a substituted 1-isoquinolinyl. In embodiments, Ring B is an unsubstituted 1-isoquinolinyl. In embodiments, Ring B is a substituted 3-isoquinolinyl. In embodiments, Ring B is an unsubstituted 3-isoquinolinyl. In embodiments, Ring B is a substituted 4-isoquinolinyl. In embodiments, Ring B is an unsubstituted 4-isoquinolinyl.

In embodiments, Ring B is phenyl. In embodiments, Ring B is naphthyl. In embodiments, Ring B is 1-naphthyl. In embodiments, Ring B is 2-naphthyl. In embodiments, Ring B is quinolinyl. In embodiments, Ring B is 2-quinolinyl. In embodiments, Ring B is 3-quinolinyl. In embodiments, Ring B is 4-quinolinyl. In embodiments, Ring B is isoquinolinyl. In embodiments, Ring B is 1-isoquinolinyl. In embodiments, Ring B is 3-isoquinolinyl. In embodiments, Ring B is 4-isoquinolinyl.

In embodiments, Ring B is

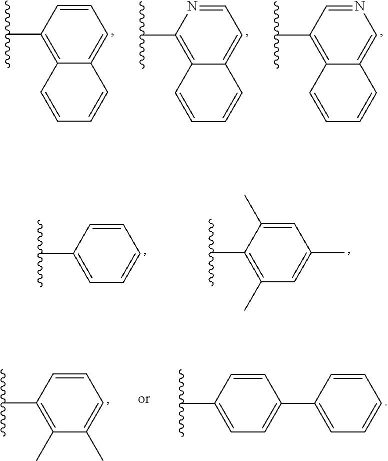

In embodiments, Ring B is

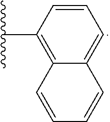 [SMILES: *c1cccc2ccccc12]

In embodiments, Ring B is

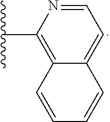 [SMILES: *c1nccc2ccccc12]

In embodiments, Ring B is

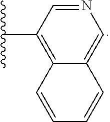 [SMILES: *c1cncc2ccccc12]

In embodiments, Ring B is

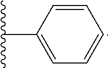 [SMILES: *c1ccccc1]

In embodiments, Ring B is

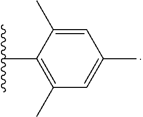 [SMILES: *c1c(C)cc(C)cc1C]

In embodiments, Ring B is

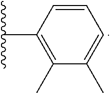 [SMILES: *c1cccc(C)c1C]

In embodiments, Ring B is

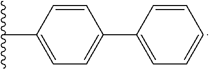 [SMILES: *c1ccc(-c2ccccc2)cc1]

In embodiments, m is 0. In embodiments, m is 1. In embodiments, m is 2. In embodiments, m is 3. In embodiments, m is 4. In embodiments, m is 5.

In embodiments, n is 0. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10.

In embodiments, the compound has the formula:

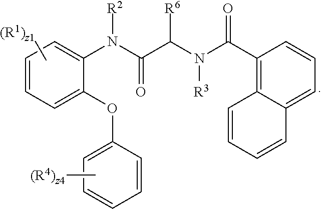

R¹, z1, R², R³, R⁴, z4, and R⁶ are as described herein, including in embodiments.

In embodiments, the compound has the formula:

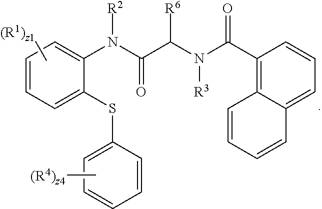

R¹, z1, R², R³, R⁴, z4, and R⁶ are as described herein, including in embodiments.

In embodiments, the compound has the formula:

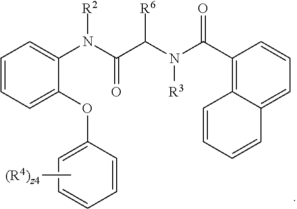

R², R³, R⁴, z4, and R⁶ are as described herein, including in embodiments.

In embodiments, the compound has the formula:

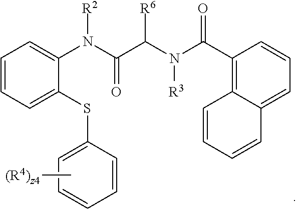

R², R³, R⁴, z4, and R⁶ are as described herein, including in embodiments.

In embodiments, the compound has the formula:

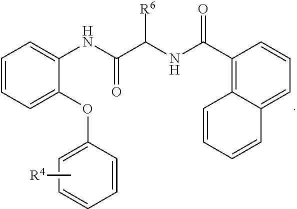

R⁴, z4, and R⁶ are as described herein, including in embodiments.

In embodiments, the compound has the formula:

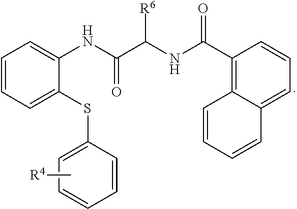

R⁴, z4, and R⁶ are as described herein, including in embodiments.

In embodiments, the compound has the formula:

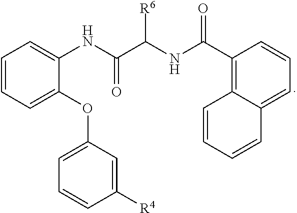

R⁴, z4, and R⁶ are as described herein, including in embodiments.

In embodiments, the compound has the formula:

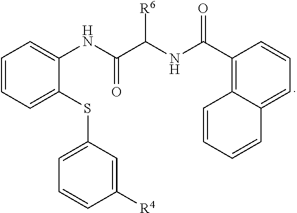

R⁴, z4, and R⁶ are as described herein, including in embodiments.

In embodiments, the compound has the formula:

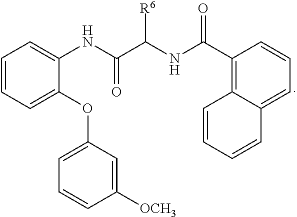

R⁶ is as described herein, including in embodiments.

In embodiments, the compound has the formula:

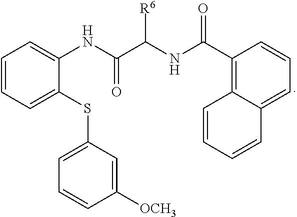

R⁶ is as described herein, including in embodiments.

In embodiments, the compound has the formula:

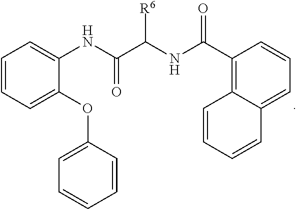

R⁶ is as described herein, including in embodiments.

In embodiments, the compound has the formula:

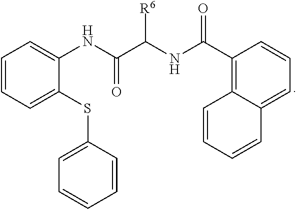

R⁶ is as described herein, including in embodiments.

In embodiments, the compound has the formula:

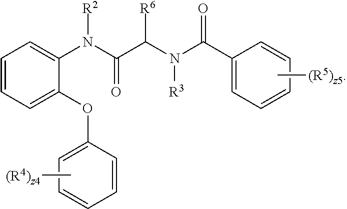

R², R³, R⁴, z4, R⁵, z5, and R⁶ are as described herein, including in embodiments.

In embodiments, the compound has the formula:

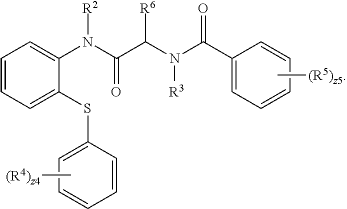

R², R³, R⁴, z4, R⁵, z5, and R⁶ are as described herein, including in embodiments.

In embodiments, the compound has the formula:

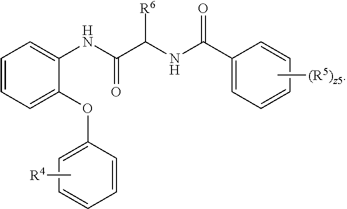

R⁴, R⁵, z5, and R⁶ are as described herein, including in embodiments.

In embodiments, the compound has the formula:

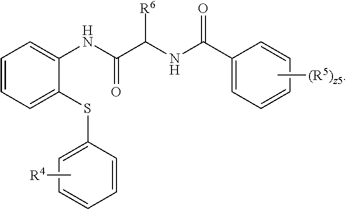

R⁴, R⁵, z5, and R⁶ are as described herein, including in embodiments.

In embodiments, the compound has the formula:

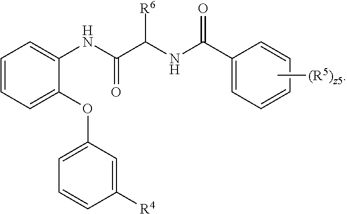

R⁴, R⁵, z5, and R⁶ are as described herein, including in embodiments.

In embodiments, the compound has the formula:

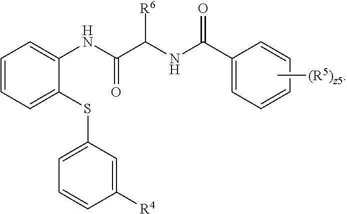

R⁴, R⁵, z5, and R⁶ are as described herein, including in embodiments.

In embodiments, the compound has the formula:

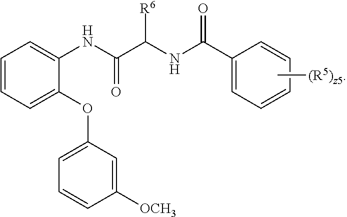

R⁵, z5, and R⁶ are as described herein, including in embodiments.

In embodiments, the compound has the formula:

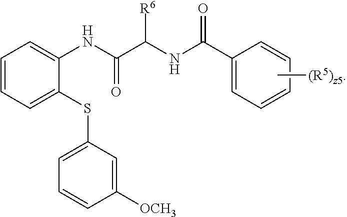

R³, z5, and R⁶ are as described herein, including in embodiments.

In embodiments, the compound has the formula:

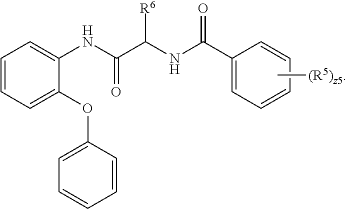

R⁵, z5, and R⁶ are as described herein, including in embodiments.

In embodiments, the compound has the formula:

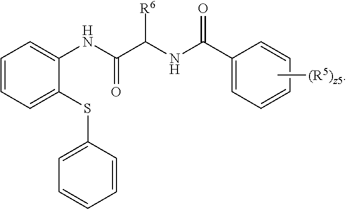

R⁵, z5, and R⁶ are as described herein, including in embodiments.

In embodiments, the compound has the formula:

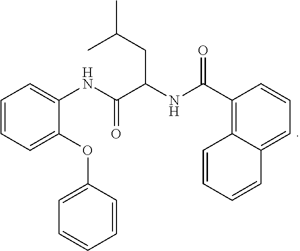 [SMILES: CC(C)CC(NC(=O)c1cccc2ccccc12)C(=O)Nc1ccccc1Oc1ccccc1]

In embodiments, the compound has the formula:

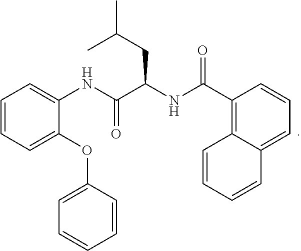 [SMILES: CC(C)C[C@@H](NC(=O)c1cccc2ccccc12)C(=O)Nc1ccccc1Oc1ccccc1]

In embodiments, the compound has the formula:

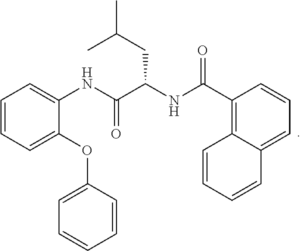 [SMILES: CC(C)C[C@H](NC(=O)c1cccc2ccccc12)C(=O)Nc1ccccc1Oc1ccccc1]

In embodiments, the compound has the formula:

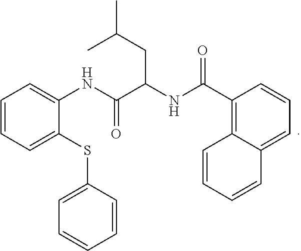 [SMILES: CC(C)CC(NC(=O)c1cccc2ccccc12)C(=O)Nc1ccccc1Sc1ccccc1]

In embodiments, the compound has the formula:

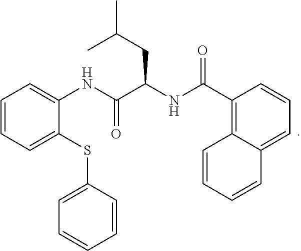 [SMILES: CC(C)C[C@@H](NC(=O)c1cccc2ccccc12)C(=O)Nc1ccccc1Sc1ccccc1]

In embodiments, the compound has the formula:

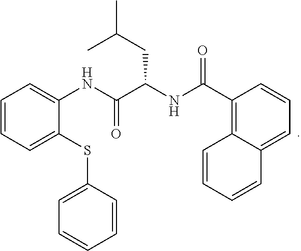 [SMILES: CC(C)C[C@H](NC(=O)c1cccc2ccccc12)C(=O)Nc1ccccc1Sc1ccccc1]

In embodiments, the compound has the formula:

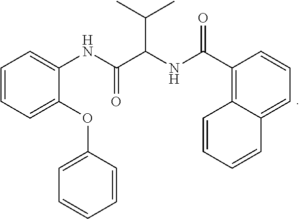 [SMILES: CC(C)C(NC(=O)c1cccc2ccccc12)C(=O)Nc1ccccc1Oc1ccccc1]

In embodiments, the compound has the formula:

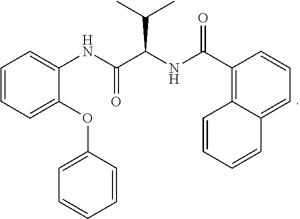 [SMILES: CC(C)[C@@H](NC(=O)c1cccc2ccccc12)C(=O)Nc1ccccc1Oc1ccccc1]

In embodiments, the compound has the formula:

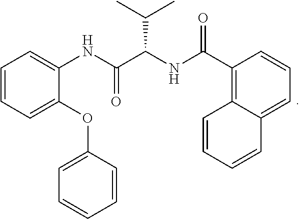 [SMILES: CC(C)[C@H](NC(=O)c1cccc2ccccc12)C(=O)Nc1ccccc1Oc1ccccc1]

In embodiments, the compound has the formula:

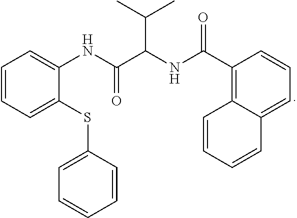 [SMILES: CC(C)C(NC(=O)c1cccc2ccccc12)C(=O)Nc1ccccc1Sc1ccccc1]

In embodiments, the compound has the formula:

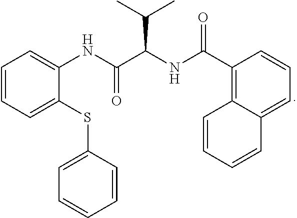 [SMILES: CC(C)[C@@H](NC(=O)c1cccc2ccccc12)C(=O)Nc1ccccc1Sc1ccccc1]

In embodiments, the compound has the formula:

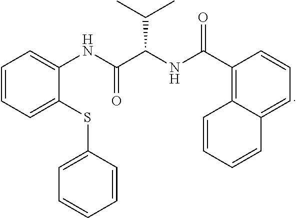 [SMILES: CC(C)[C@H](NC(=O)c1cccc2ccccc12)C(=O)Nc1ccccc1Sc1ccccc1]

In embodiments, the compound has the formula:

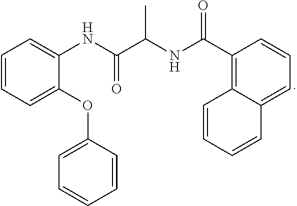 [SMILES: CC(NC(=O)c1cccc2ccccc12)C(=O)Nc1ccccc1Oc1ccccc1]

In embodiments, the compound has the formula:

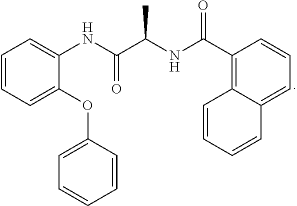 [SMILES: C[C@@H](NC(=O)c1cccc2ccccc12)C(=O)Nc1ccccc1Oc1ccccc1]

In embodiments, the compound has the formula:

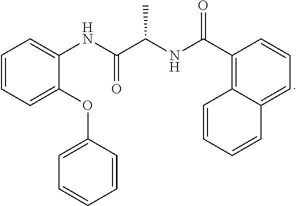 [SMILES: C[C@H](NC(=O)c1cccc2ccccc12)C(=O)Nc1ccccc1Oc1ccccc1]

In embodiments, the compound has the formula:

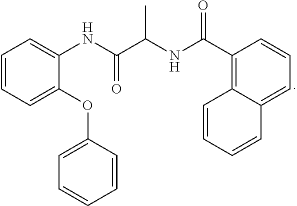 [SMILES: CC(NC(=O)c1cccc2ccccc12)C(=O)Nc1ccccc1Oc1ccccc1]

In embodiments, the compound has the formula:

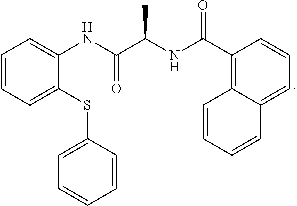 [SMILES: C[C@@H](NC(=O)c1cccc2ccccc12)C(=O)Nc1ccccc1Sc1ccccc1]

In embodiments, the compound has the formula:

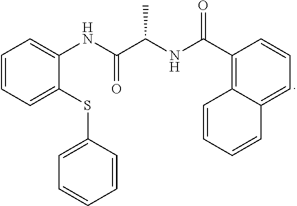 [SMILES: C[C@H](NC(=O)c1cccc2ccccc12)C(=O)Nc1ccccc1Sc1ccccc1]

In embodiments, the compound has the formula:

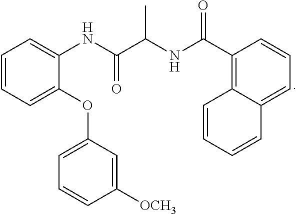 [SMILES: COc1cccc(Oc2ccccc2NC(=O)C(C)NC(=O)c2cccc3ccccc23)c1]

In embodiments, the compound has the formula:

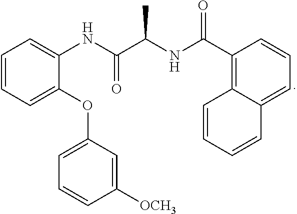 [SMILES: COc1cccc(Oc2ccccc2NC(=O)[C@@H](C)NC(=O)c2cccc3ccccc23)c1]

In embodiments, the compound has the formula:

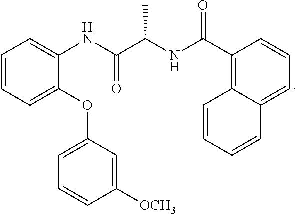 [SMILES: COc1cccc(Oc2ccccc2NC(=O)[C@H](C)NC(=O)c2cccc3ccccc23)c1]

In embodiments, the compound has the formula:

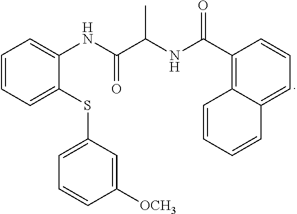 [SMILES: COc1cccc(Sc2ccccc2NC(=O)C(C)NC(=O)c2cccc3ccccc23)c1]

In embodiments, the compound has the formula:

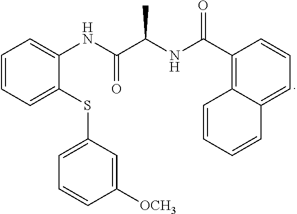 [SMILES: COc1cccc(Sc2ccccc2NC(=O)[C@@H](C)NC(=O)c2cccc3ccccc23)c1]

In embodiments, the compound has the formula:

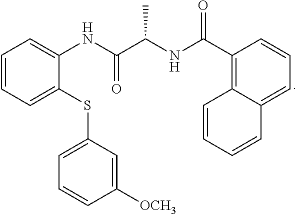 [SMILES: COc1cccc(Sc2ccccc2NC(=O)[C@H](C)NC(=O)c2cccc3ccccc23)c1]

In embodiments, the compound has the formula:

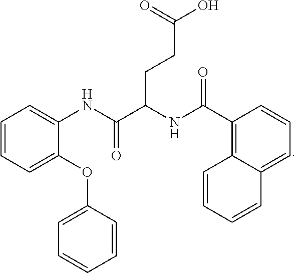 [SMILES: O=C(O)CCC(NC(=O)c1cccc2ccccc12)C(=O)Nc1ccccc1Oc1ccccc1]

In embodiments, the compound has the formula:

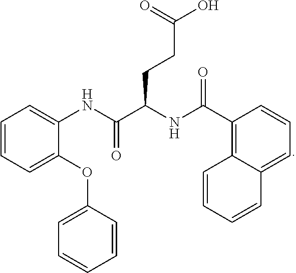 [SMILES: O=C(O)CC[C@@H](NC(=O)c1cccc2ccccc12)C(=O)Nc1ccccc1Oc1ccccc1]

In embodiments, the compound has the formula:

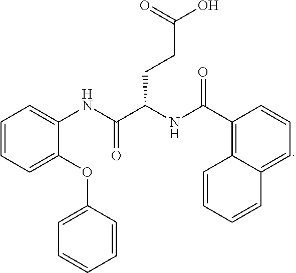 [SMILES: O=C(O)CC[C@H](NC(=O)c1cccc2ccccc12)C(=O)Nc1ccccc1Oc1ccccc1]

In embodiments, the compound has the formula:

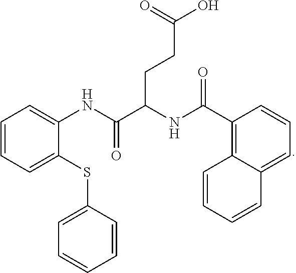 [SMILES: O=C(O)CCC(NC(=O)c1cccc2ccccc12)C(=O)Nc1ccccc1Sc1ccccc1]

In embodiments, the compound has the formula:

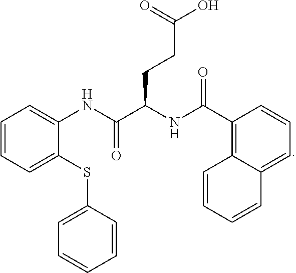 [SMILES: O=C(O)CC[C@@H](NC(=O)c1cccc2ccccc12)C(=O)Nc1ccccc1Sc1ccccc1]

In embodiments, the compound has the formula:

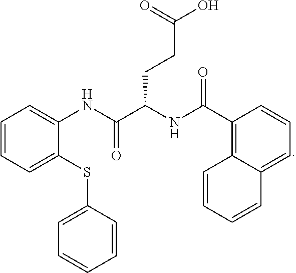 [SMILES: O=C(O)CC[C@H](NC(=O)c1cccc2ccccc12)C(=O)Nc1ccccc1Sc1ccccc1]

In embodiments, the compound has the formula:

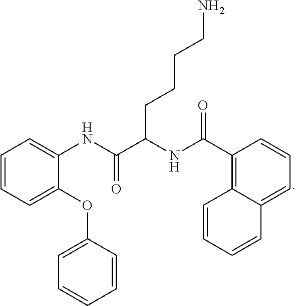 [SMILES: NCCCCC(NC(=O)c1cccc2ccccc12)C(=O)Nc1ccccc1Oc1ccccc1]

In embodiments, the compound has the formula:

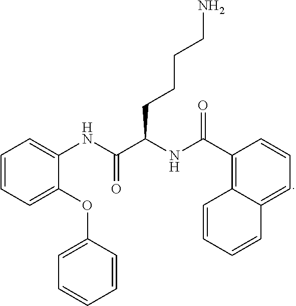 [SMILES: NCCCC[C@@H](NC(=O)c1cccc2ccccc12)C(=O)Nc1ccccc1Oc1ccccc1]

In embodiments, the compound has the formula:

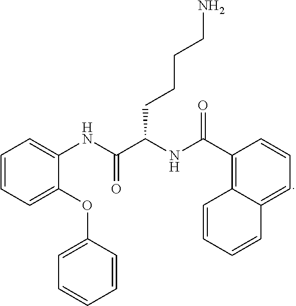 [SMILES: NCCCC[C@H](NC(=O)c1cccc2ccccc12)C(=O)Nc1ccccc1Oc1ccccc1]

In embodiments, the compound has the formula:

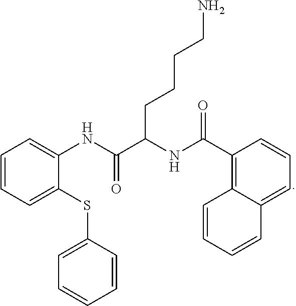 [SMILES: NCCCCC(NC(=O)c1cccc2ccccc12)C(=O)Nc1ccccc1Sc1ccccc1]

In embodiments, the compound has the formula:

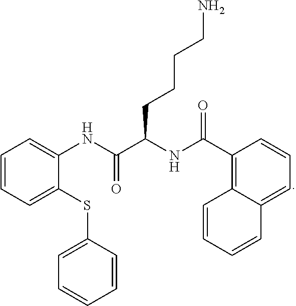 [SMILES: NCCCC[C@@H](NC(=O)c1cccc2ccccc12)C(=O)Nc1ccccc1Sc1ccccc1]

In embodiments, the compound has the formula:

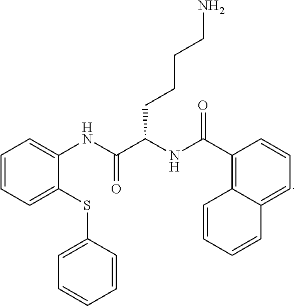 [SMILES: NCCCC[C@H](NC(=O)c1cccc2ccccc12)C(=O)Nc1ccccc1Sc1ccccc1]

In embodiments, the compound has the formula:

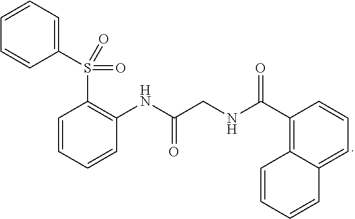 [SMILES: O=C(CNC(=O)c1cccc2ccccc12)Nc1ccccc1S(=O)(=O)c1ccccc1]

In embodiments, the compound has the formula:

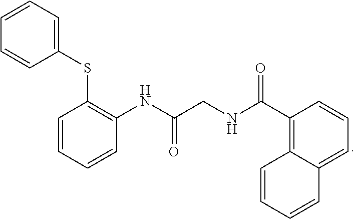 [SMILES: O=C(CNC(=O)c1cccc2ccccc12)Nc1ccccc1Sc1ccccc1]

In embodiments, the compound has the formula:

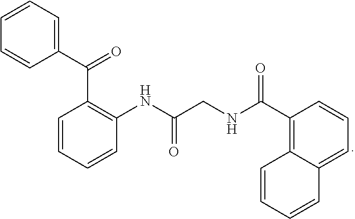 [SMILES: O=C(CNC(=O)c1cccc2ccccc12)Nc1ccccc1C(=O)c1ccccc1]

In embodiments, the compound has the formula:

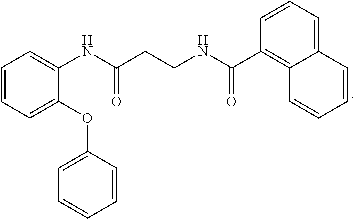 [SMILES: O=C(CCNC(=O)c1cccc2ccccc12)Nc1ccccc1Oc1ccccc1]

In embodiments, the compound has the formula:

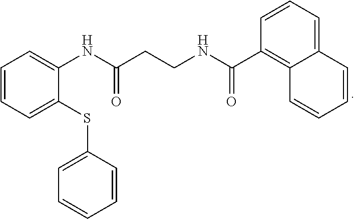 [SMILES: O=C(CCNC(=O)c1cccc2ccccc12)Nc1ccccc1Sc1ccccc1]

In embodiments, the compound has the formula:

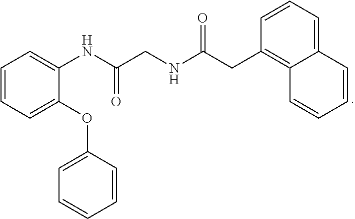 [SMILES: O=C(Cc1cccc2ccccc12)NCC(=O)Nc1ccccc1Oc1ccccc1]

In embodiments, the compound has the formula:

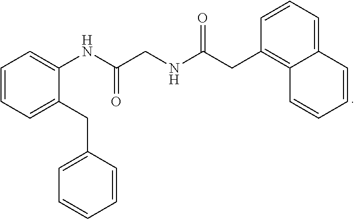 [SMILES: O=C(Cc1cccc2ccccc12)NCC(=O)Nc1ccccc1Cc1ccccc1]

In embodiments, the compound has the formula:

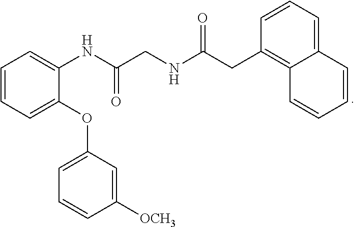 [SMILES: COc1cccc(Oc2ccccc2NC(=O)CNC(=O)Cc2cccc3ccccc23)c1]

In embodiments, the compound has the formula:

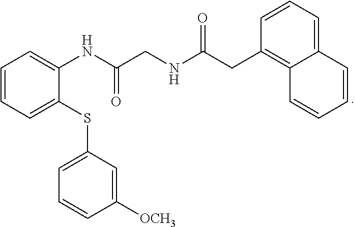 [SMILES: COc1cccc(Sc2ccccc2NC(=O)CNC(=O)Cc2cccc3ccccc23)c1]

In embodiments, the compound has the formula:

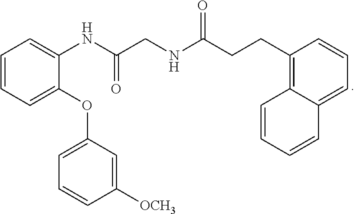 [SMILES: COc1cccc(Oc2ccccc2NC(=O)CNC(=O)CCc2cccc3ccccc23)c1]

In embodiments, the compound has the formula:

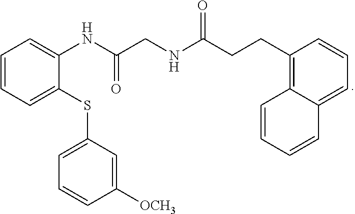 [SMILES: COc1cccc(Sc2ccccc2NC(=O)CNC(=O)CCc2cccc3ccccc23)c1]

In embodiments, the compound has the formula:

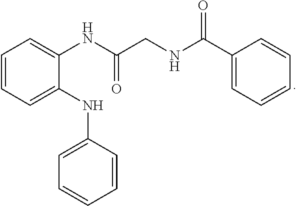 [SMILES: O=C(CNC(=O)c1ccccc1)Nc1ccccc1Nc1ccccc1]

In embodiments, the compound has the formula:

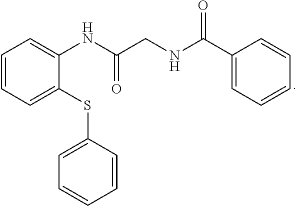 [SMILES: O=C(CNC(=O)c1ccccc1)Nc1ccccc1Sc1ccccc1]

In embodiments, the compound has the formula:

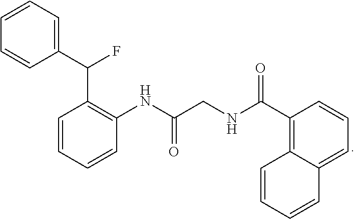 [SMILES: O=C(CNC(=O)c1cccc2ccccc12)Nc1ccccc1C(F)c1ccccc1]

In embodiments, the compound has the formula:

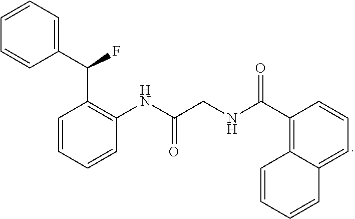 [SMILES: O=C(CNC(=O)c1cccc2ccccc12)Nc1ccccc1[C@H](F)c1ccccc1]

In embodiments, the compound has the formula:

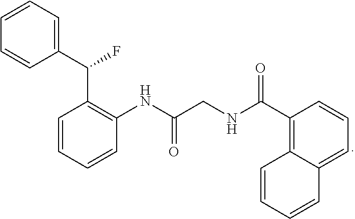 [SMILES: O=C(CNC(=O)c1cccc2ccccc12)Nc1ccccc1[C@@H](F)c1ccccc1]

In embodiments, the compound has the formula:

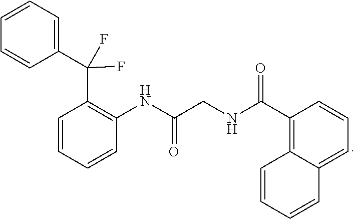 [SMILES: O=C(CNC(=O)c1cccc2ccccc12)Nc1ccccc1C(F)(F)c1ccccc1]

In embodiments, the compound has the formula:

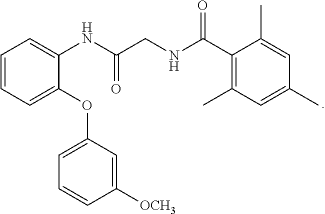 [SMILES: COc1cccc(Oc2ccccc2NC(=O)CNC(=O)c2c(C)cc(C)cc2C)c1]

In embodiments, the compound has the formula:

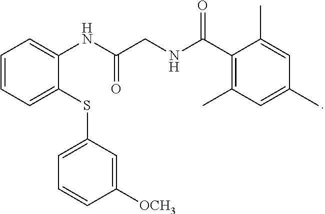 [SMILES: COc1cccc(Sc2ccccc2NC(=O)CNC(=O)c2c(C)cc(C)cc2C)c1]

In embodiments, the compound has the formula:

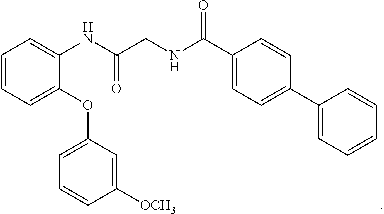 [SMILES: COc1cccc(Oc2ccccc2NC(=O)CNC(=O)c2ccc(-c3ccccc3)cc2)c1]

In embodiments, the compound has the formula:

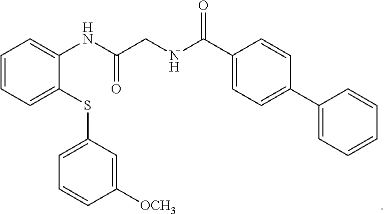 [SMILES: COc1cccc(Sc2ccccc2NC(=O)CNC(=O)c2ccc(-c3ccccc3)cc2)c1]

In embodiments, the compound has the formula:

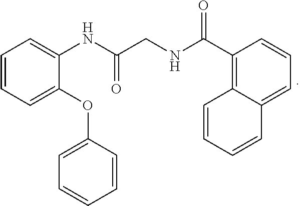 [SMILES: O=C(CNC(=O)c1cccc2ccccc12)Nc1ccccc1Oc1ccccc1]

In embodiments, the compound has the formula:

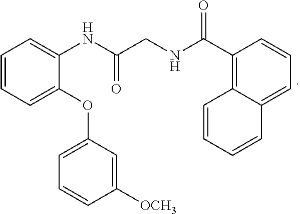 [SMILES: COc1cccc(Oc2ccccc2NC(=O)CNC(=O)c2cccc3ccccc23)c1]

In embodiments, the compound has the formula:

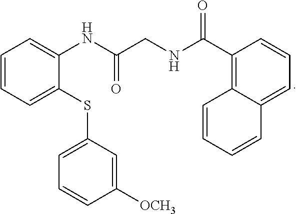 [SMILES: COc1cccc(Sc2ccccc2NC(=O)CNC(=O)c2cccc3ccccc23)c1]

In embodiments, the compound has the formula:

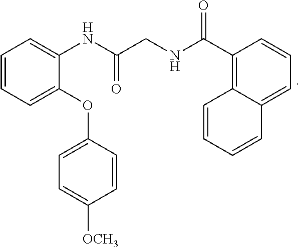 [SMILES: COc1ccc(Oc2ccccc2NC(=O)CNC(=O)c2cccc3ccccc23)cc1]

In embodiments, the compound has the formula:

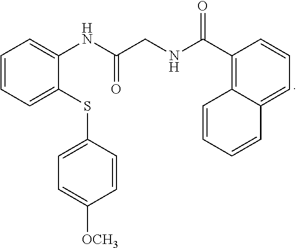 [SMILES: COc1ccc(Sc2ccccc2NC(=O)CNC(=O)c2cccc3ccccc23)cc1]

In embodiments, the compound has the formula:

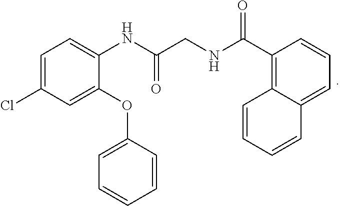 [SMILES: O=C(CNC(=O)c1cccc2ccccc12)Nc1ccc(Cl)cc1Oc1ccccc1]

In embodiments, the compound has the formula:

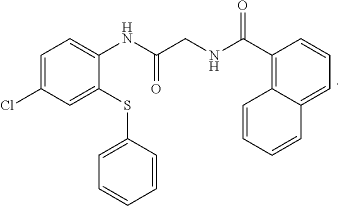 [SMILES: O=C(CNC(=O)c1cccc2ccccc12)Nc1ccc(Cl)cc1Sc1ccccc1]

In embodiments, the compound has the formula:

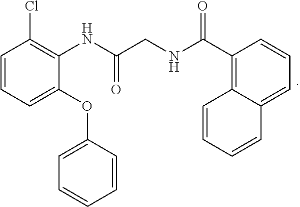 [SMILES: O=C(CNC(=O)c1cccc2ccccc12)Nc1c(Cl)cccc1Oc1ccccc1]

In embodiments, the compound has the formula:

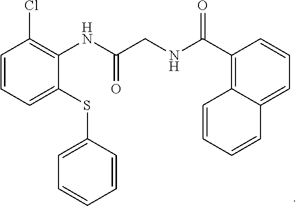 [SMILES: O=C(CNC(=O)c1cccc2ccccc12)Nc1c(Cl)cccc1Sc1ccccc1]

In embodiments, the compound has the formula:

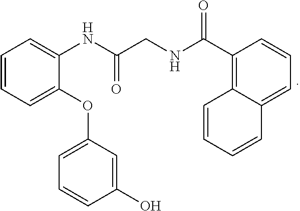 [SMILES: O=C(CNC(=O)c1cccc2ccccc12)Nc1ccccc1Oc1cccc(O)c1]

In embodiments, the compound has the formula:

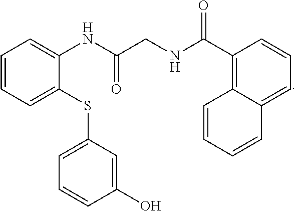 [SMILES: O=C(CNC(=O)c1cccc2ccccc12)Nc1ccccc1Sc1cccc(O)c1]

In embodiments, the compound has the formula:

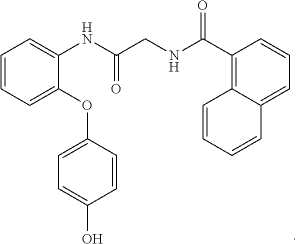 [SMILES: O=C(CNC(=O)c1cccc2ccccc12)Nc1ccccc1Oc1ccc(O)cc1]

In embodiments, the compound has the formula:

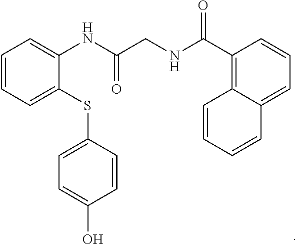 [SMILES: O=C(CNC(=O)c1cccc2ccccc12)Nc1ccccc1Sc1ccc(O)cc1]

In embodiments, the compound has the formula:

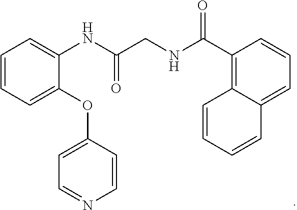 [SMILES: O=C(CNC(=O)c1cccc2ccccc12)Nc1ccccc1Oc1ccncc1]

In embodiments, the compound has the formula:

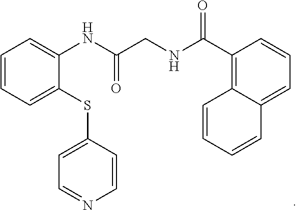 [SMILES: O=C(CNC(=O)c1cccc2ccccc12)Nc1ccccc1Sc1ccncc1]

In embodiments, the compound has the formula:

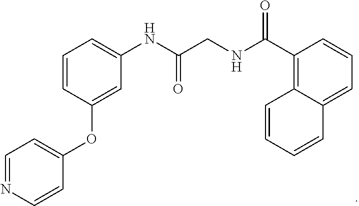 [SMILES: O=C(CNC(=O)c1cccc2ccccc12)Nc1cccc(Oc2ccncc2)c1]

In embodiments, the compound has the formula:

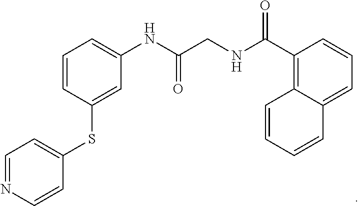 [SMILES: O=C(CNC(=O)c1cccc2ccccc12)Nc1cccc(Sc2ccncc2)c1]

In embodiments, the compound has the formula:

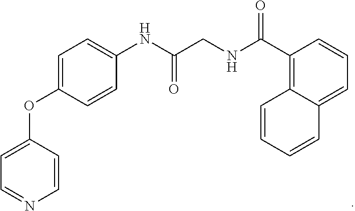 [SMILES: O=C(CNC(=O)c1cccc2ccccc12)Nc1ccc(Oc2ccncc2)cc1]

In embodiments, the compound has the formula:

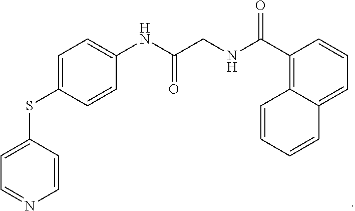 [SMILES: O=C(CNC(=O)c1cccc2ccccc12)Nc1ccc(Sc2ccncc2)cc1]

In embodiments, the compound has the formula:

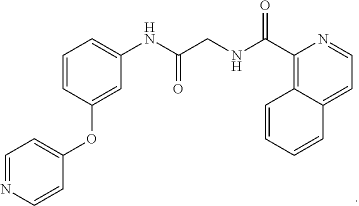 [SMILES: O=C(CNC(=O)c1nccc2ccccc12)Nc1cccc(Oc2ccncc2)c1]

In embodiments, the compound has the formula:

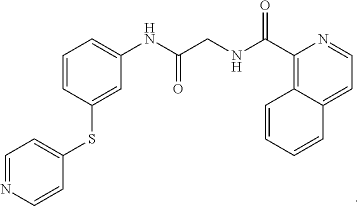 [SMILES: O=C(CNC(=O)c1nccc2ccccc12)Nc1cccc(Sc2ccncc2)c1]

In embodiments, the compound has the formula:

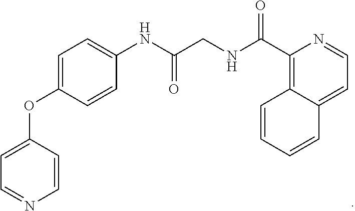 [SMILES: O=C(CNC(=O)c1nccc2ccccc12)Nc1ccc(Oc2ccncc2)cc1]

In embodiments, the compound has the formula:

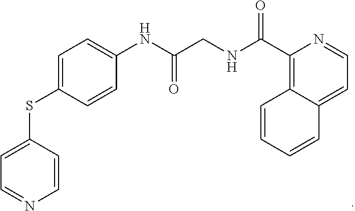 [SMILES: O=C(CNC(=O)c1nccc2ccccc12)Nc1ccc(Sc2ccncc2)cc1]

In embodiments, the compound has the formula:

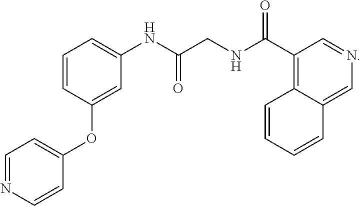 [SMILES: O=C(CNC(=O)c1cncc2ccccc12)Nc1cccc(Oc2ccncc2)c1]

In embodiments, the compound has the formula:

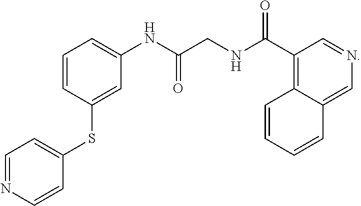 [SMILES: O=C(CNC(=O)c1cncc2ccccc12)Nc1cccc(Sc2ccncc2)c1]

In embodiments, the compound has the formula:

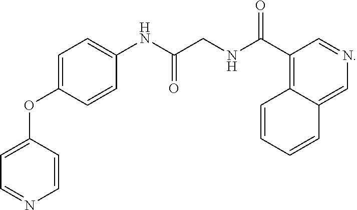 [SMILES: O=C(CNC(=O)c1cncc2ccccc12)Nc1ccc(Oc2ccncc2)cc1]

In embodiments, the compound has the formula:

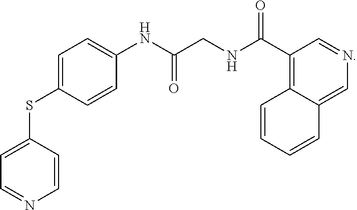 [SMILES: O=C(CNC(=O)c1cncc2ccccc12)Nc1ccc(Sc2ccncc2)cc1]

In embodiments, the compound has the formula:

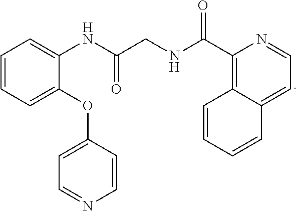 [SMILES: O=C(CNC(=O)c1nccc2ccccc12)Nc1ccccc1Oc1ccncc1]

In embodiments, the compound has the formula:

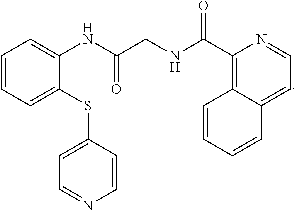 [SMILES: O=C(CNC(=O)c1nccc2ccccc12)Nc1ccccc1Sc1ccncc1]

In embodiments, the compound has the formula:

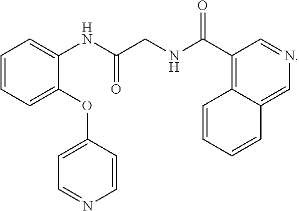 [SMILES: O=C(CNC(=O)c1cncc2ccccc12)Nc1ccccc1Oc1ccncc1]

In embodiments, the compound has the formula:

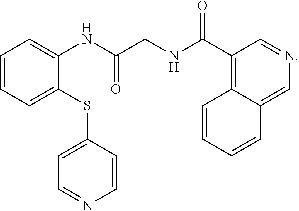 [SMILES: O=C(CNC(=O)c1cncc2ccccc12)Nc1ccccc1Sc1ccncc1]

In embodiments, the compound has the formula:

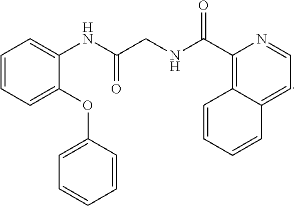 [SMILES: O=C(CNC(=O)c1nccc2ccccc12)Nc1ccccc1Oc1ccccc1]

In embodiments, the compound has the formula:

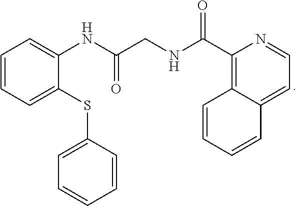 [SMILES: O=C(CNC(=O)c1nccc2ccccc12)Nc1ccccc1Sc1ccccc1]

In embodiments, the compound has the formula:

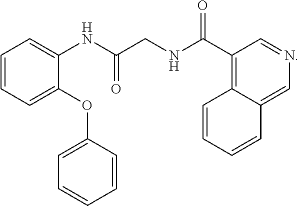 [SMILES: O=C(CNC(=O)c1cncc2ccccc12)Nc1ccccc1Oc1ccccc1]

In embodiments, the compound has the formula:

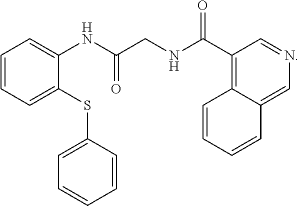 [SMILES: O=C(CNC(=O)c1cncc2ccccc12)Nc1ccccc1Sc1ccccc1]

In embodiments, the compound has the formula:

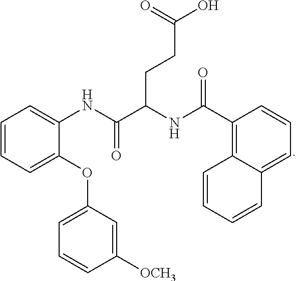 [SMILES: COc1cccc(Oc2ccccc2NC(=O)C(CCC(=O)O)NC(=O)c2cccc3ccccc23)c1]

In embodiments, the compound has the formula:

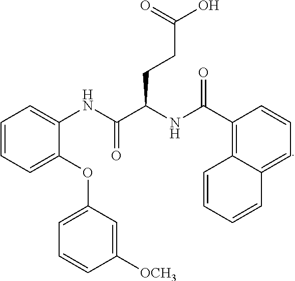 [SMILES: COc1cccc(Oc2ccccc2NC(=O)[C@@H](CCC(=O)O)NC(=O)c2cccc3ccccc23)c1]

In embodiments, the compound has the formula:

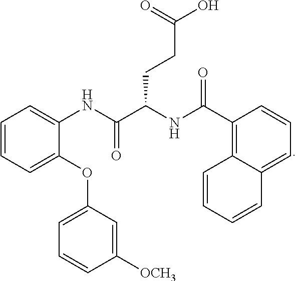 [SMILES: COc1cccc(Oc2ccccc2NC(=O)[C@H](CCC(=O)O)NC(=O)c2cccc3ccccc23)c1]

In embodiments, the compound has the formula:

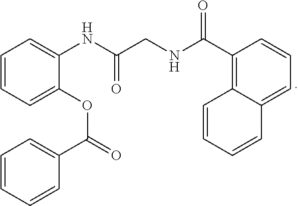 [SMILES: O=C(CNC(=O)c1cccc2ccccc12)Nc1ccccc1OC(=O)c1ccccc1]

In embodiments, the compound has the formula:

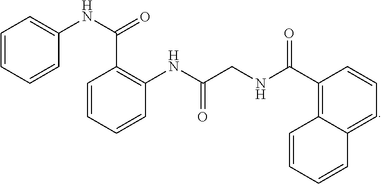 [SMILES: O=C(CNC(=O)c1cccc2ccccc12)Nc1ccccc1C(=O)Nc1ccccc1]

In embodiments, the compound has the formula:

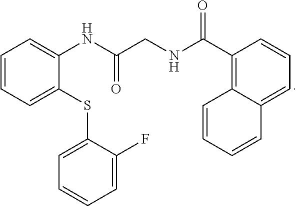 [SMILES: O=C(CNC(=O)c1cccc2ccccc12)Nc1ccccc1Sc1ccccc1F]

In embodiments, the compound has the formula:

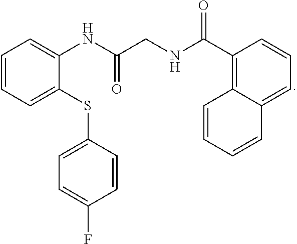 [SMILES: O=C(CNC(=O)c1cccc2ccccc12)Nc1ccccc1Sc1ccc(F)cc1]

In embodiments, the compound has the formula:

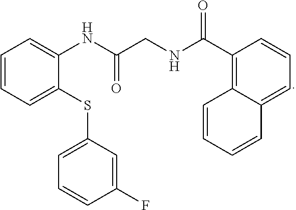 [SMILES: O=C(CNC(=O)c1cccc2ccccc12)Nc1ccccc1Sc1cccc(F)c1]

In embodiments, the compound has the formula:

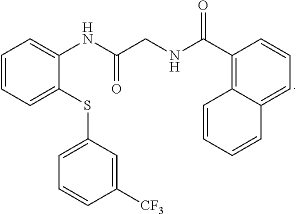 [SMILES: O=C(CNC(=O)c1cccc2ccccc12)Nc1ccccc1Sc1cccc(C(F)(F)F)c1]

In embodiments, the compound has the formula:

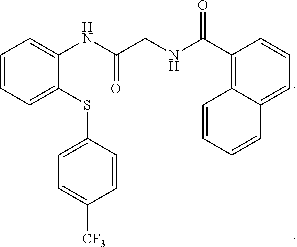 [SMILES: O=C(CNC(=O)c1cccc2ccccc12)Nc1ccccc1Sc1ccc(C(F)(F)F)cc1]

In embodiments, the compound has the formula:

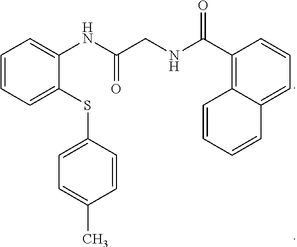 [SMILES: Cc1ccc(Sc2ccccc2NC(=O)CNC(=O)c2cccc3ccccc23)cc1]

In embodiments, the compound has the formula:

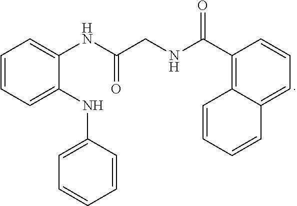 [SMILES: O=C(CNC(=O)c1cccc2ccccc12)Nc1ccccc1Nc1ccccc1]

In embodiments, the compound has the formula:

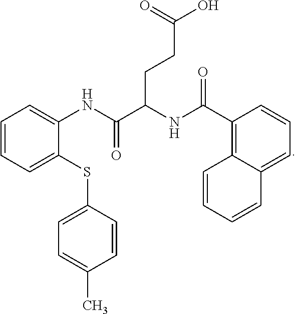 [SMILES: Cc1ccc(Sc2ccccc2NC(=O)C(CCC(=O)O)NC(=O)c2cccc3ccccc23)cc1]

In embodiments, the compound has the formula:

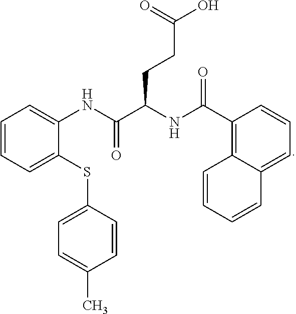 [SMILES: Cc1ccc(Sc2ccccc2NC(=O)[C@@H](CCC(=O)O)NC(=O)c2cccc3ccccc23)cc1]

In embodiments, the compound has the formula:

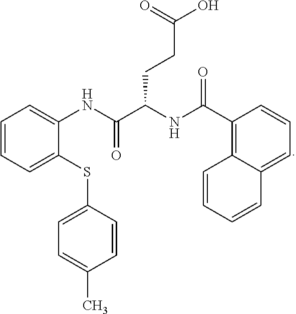 [SMILES: Cc1ccc(Sc2ccccc2NC(=O)[C@H](CCC(=O)O)NC(=O)c2cccc3ccccc23)cc1]

In embodiments, the compound has the formula:

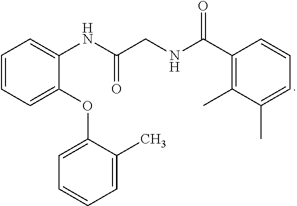 [SMILES: Cc1ccccc1Oc1ccccc1NC(=O)CNC(=O)c1cccc(C)c1C]

In embodiments, the compound is a compound in this paragraph, wherein the R² position is methyl instead of hydrogen. In embodiments, the compound is a compound in this paragraph, wherein the R³ position is methyl instead of hydrogen. In embodiments, the compound is a compound in this paragraph, wherein the R² and R³ positions are each methyl instead of hydrogen.

In embodiments, the compound has the formula:

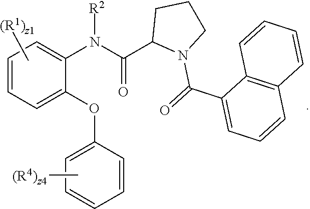

R¹, z1, R², R⁴, and z4 are as described herein, including in embodiments.

In embodiments, the compound has the formula:

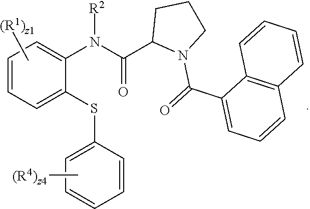

R¹, z1, R², R⁴, and z4 are as described herein, including in embodiments.

In embodiments, the compound has the formula:

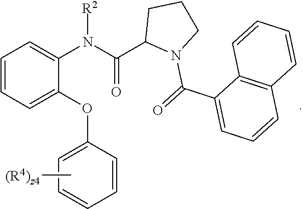

R², R⁴, and z4 are as described herein, including in embodiments.

In embodiments, the compound has the formula:

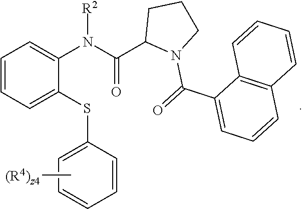

R², R⁴, and z4 are as described herein, including in embodiments.

In embodiments, the compound has the formula:

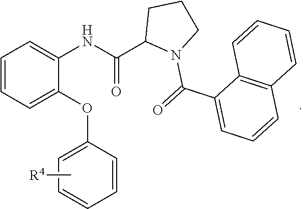

R⁴ is as described herein, including in embodiments.

In embodiments, the compound has the formula:

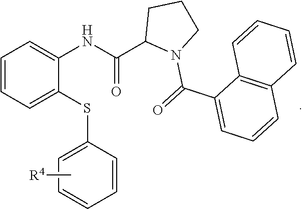

R⁴ is as described herein, including in embodiments.

In embodiments, the compound has the formula:

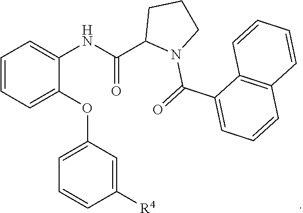

R⁴ is as described herein, including in embodiments.

In embodiments, the compound has the formula:

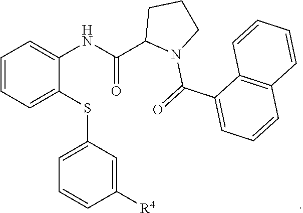

R⁴ is as described herein, including in embodiments.

In embodiments, the compound has the formula:

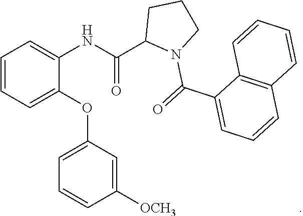 [SMILES: COc1cccc(Oc2ccccc2NC(=O)C2CCCN2C(=O)c2cccc3ccccc23)c1]

In embodiments, the compound has the formula:

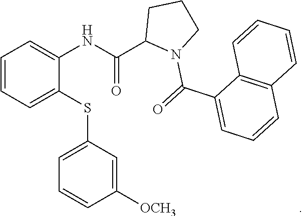 [SMILES: COc1cccc(Sc2ccccc2NC(=O)C2CCCN2C(=O)c2cccc3ccccc23)c1]

In embodiments, the compound has the formula:

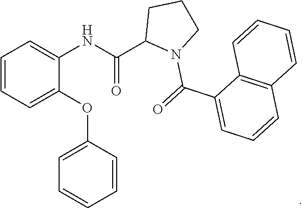 [SMILES: O=C(Nc1ccccc1Oc1ccccc1)C1CCCN1C(=O)c1cccc2ccccc12]

In embodiments, the compound has the formula:

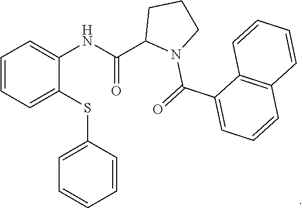 [SMILES: O=C(Nc1ccccc1Sc1ccccc1)C1CCCN1C(=O)c1cccc2ccccc12]

In embodiments, the compound has the formula:

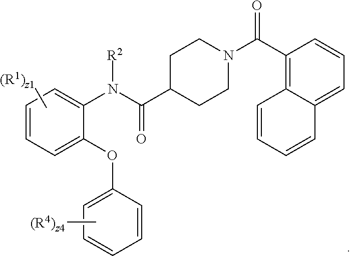

R¹, z1, R², R⁴, and z4 are as described herein, including in embodiments.

In embodiments, the compound has the formula:

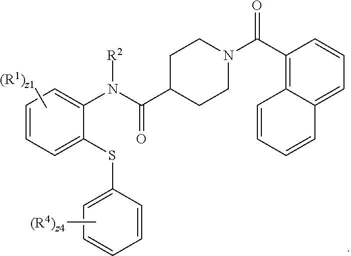

R¹, z1, R², R⁴, and z4 are as described herein, including in embodiments.

In embodiments, the compound has the formula:

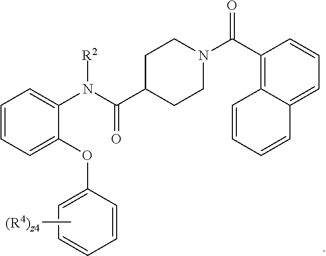

R², R⁴, and z4 are as described herein, including in embodiments.

In embodiments, the compound has the formula:

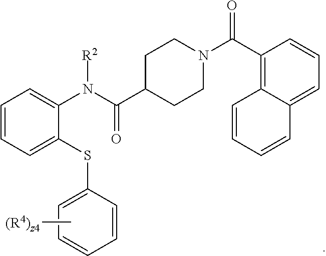

R², R⁴, and z4 are as described herein, including in embodiments.

In embodiments, the compound has the formula:

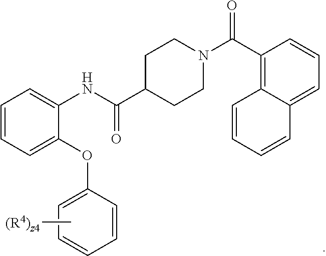

R⁴ is as described herein, including in embodiments.

In embodiments, the compound has the formula:

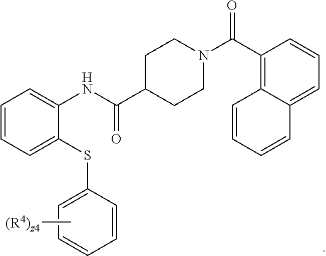

R⁴ is as described herein, including in embodiments.

In embodiments, the compound has the formula:

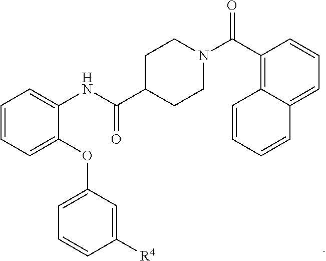

R⁴ is as described herein, including in embodiments.

In embodiments, the compound has the formula:

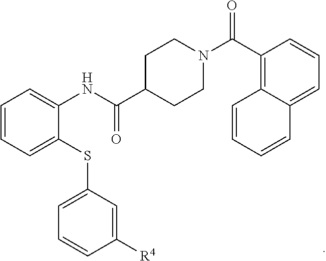

R⁴ is as described herein, including in embodiments.

In embodiments, the compound has the formula:

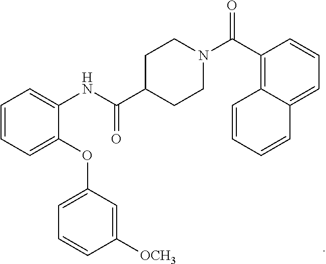 [SMILES: COc1cccc(Oc2ccccc2NC(=O)C2CCN(C(=O)c3cccc4ccccc34)CC2)c1]

In embodiments, the compound has the formula:

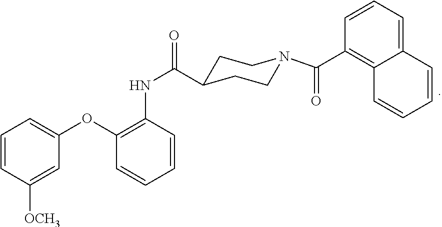 [SMILES: COc1cccc(Oc2ccccc2NC(=O)C2CCN(C(=O)c3cccc4ccccc34)CC2)c1]

In embodiments, the compound has the formula:

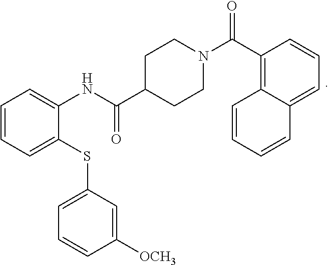 [SMILES: COc1cccc(Sc2ccccc2NC(=O)C2CCN(C(=O)c3cccc4ccccc34)CC2)c1]

In embodiments, the compound has the formula:

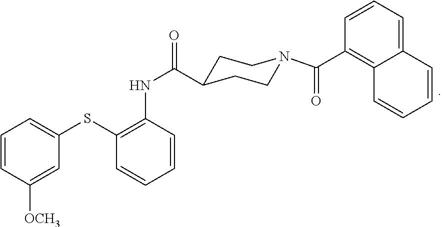 [SMILES: COc1cccc(Sc2ccccc2NC(=O)C2CCN(C(=O)c3cccc4ccccc34)CC2)c1]

In embodiments, the compound has the formula:

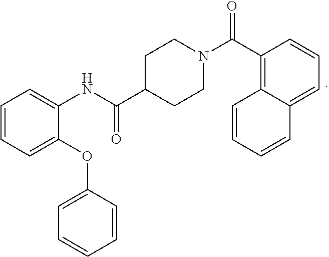 [SMILES: O=C(Nc1ccccc1Oc1ccccc1)C1CCN(C(=O)c2cccc3ccccc23)CC1]

In embodiments, the compound has the formula:

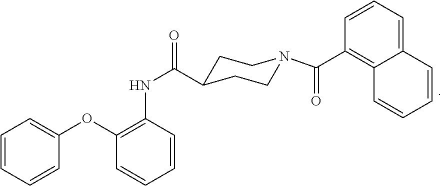 [SMILES: O=C(Nc1ccccc1Oc1ccccc1)C1CCN(C(=O)c2cccc3ccccc23)CC1]

In embodiments, the compound has the formula:

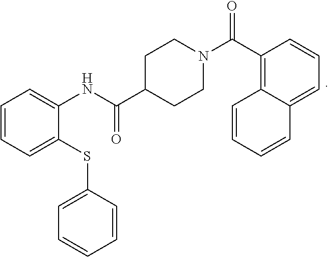 [SMILES: O=C(Nc1ccccc1Sc1ccccc1)C1CCN(C(=O)c2cccc3ccccc23)CC1]

In embodiments, the compound has the formula:

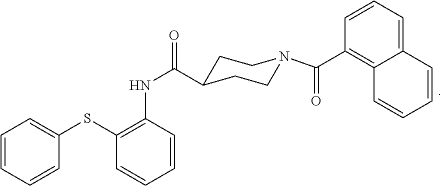 [SMILES: O=C(Nc1ccccc1Sc1ccccc1)C1CCN(C(=O)c2cccc3ccccc23)CC1]

In an aspect is provided a compound, or a pharmaceutically acceptable salt thereof, having the formula:

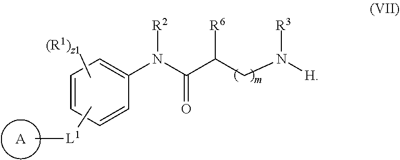

L¹, Ring A, R¹, z1, R², R³, R⁶, and m are as described herein, including in embodiments.

In embodiments, the compound has the formula:

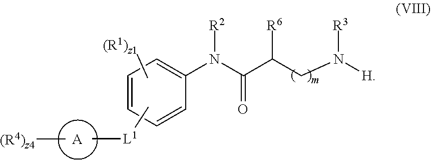

L¹, Ring A, R¹, z1, R², R³, R⁴, z4, R⁶, and m are as described herein, including in embodiments.

In embodiments, the compound has the formula:

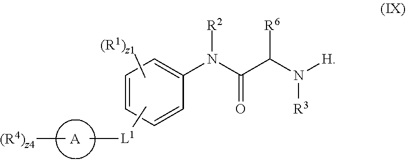

L¹, Ring A, R¹, z1, R², R³, R⁴, z4, and R⁶ are as described herein, including in embodiments.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2} and R^{WW.3} correspond to R¹, R^{1.1}, R^{1.2}, and R^{1.3}, respectively.

In embodiments, when two adjacent R¹ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹, R^{1.1}, R^{1.2}, and R^{1.3}, respectively.

In embodiments, when RIA is substituted, RIA is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1B}, R^{1B.1}, R^{1B.2}, and R^{1B.3}, respectively.

In embodiments, when RIA and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when RIA and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1C}, R^{1C.1}, R^{1C.2}, and R^{1C.3}, respectively.

In embodiments, when R^{1D} is substituted, RID is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R², R^{2.1}, R^{2.2}, and R^{2.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R³, R^{3.1}, R^{3.2}, and R^{3.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2} and R^{WW.3} correspond to R⁴, R^{4.1}, R^{4.2}, and R^{4.3}, respectively.

In embodiments, when two adjacent R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{4B}, R^{4B.1}, R^{4B.2}, and R^{4B.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A.1}, R^{4A.2}, and R^{4A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4A.1}, R^{4A.2}, and R^{4A.3}, respectively.

In embodiments, when R^{4A} and R^{4B} substituents bonded to the same nitrogen atom are optionally joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B.1}, R^{4B.2}, and R^{4B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{1C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{4D}, R^{4D.1}, R^{4D.2}, and R^{4D.3}, respectively.

In embodiments, when R⁵ is substituted, R⁵ is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁵, R^{5.1}, R^{5.2}, and R^{5.3}, respectively.

In embodiments, when two adjacent R⁵ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{5.1} as explained in the definitions section above in the description of “first substituent group(s)”. In embodiments, when an R^{5.1} substituent group is substituted, the R^{5.1} substituent group is substituted with one or more second substituent groups denoted by R^{5.2} as explained in the definitions section above in the description of “first substituent group(s)”. In embodiments, when an R^{5.2} substituent group is substituted, the R^{5.2} substituent group is substituted with one or more third substituent groups denoted by R^{5.3} as explained in the definitions section above in the description of “first substituent group(s)”. In the above embodiments, R⁵, R^{5.1}, R^{5.2}, and R^{5.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁵, R^{5.1}, R^{5.2}, and R^{5.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2} and R^{WW.3} correspond to R^{5A}, R^{5A.1}, R^{5A.2}, and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{5B}, R^{5B.1}, R^{5B.2}, and R^{5B.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A.1}, R^{5A.2}, and R^{5A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{5A.1}, R^{5A.2}, and R^{5A.3}, respectively.

In embodiments, when R^{5A} and R^{5B} substituents bonded to the same nitrogen atom are optionally joined to form a moiety that is substituted (e.g., a substituted heterocycloalkyl or substituted heteroaryl), the moiety is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} explained in the definitions section above in the description of “first substituent group(s)”. In the above embodiments, R^{5B.1}, R^{5B.2}, and R^{5B.3} have values corresponding to the values of R^{WW.1}. R^{WW.2}, and R^{WW.3}, respectively, as explained in the definitions section above in the description of “first substituent group(s)”, wherein R^{WW.1}, R^{WW.2}, and R^{WW.3} correspond to R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2} and R^{WW.3} correspond to R^{5C}, R^{5C.1}, R^{5C.2}, and R^{5C.3}, respectively.

In embodiments, when R^{5D} is substituted, R^{5D} is substituted with one or more first substituent groups denoted by R^{5D.1} as explained in the definitions section above in the description of “first substituent group(s)”. In embodiments, when an R^{5D.1} substituent group is substituted, the R^{5D.1} substituent group is substituted with one or more second substituent groups denoted by R^{5D.2} as explained in the definitions section above in the description of “first substituent group(s)”. In embodiments, when an R^{5D.2} substituent group is substituted, the R^{5D.2} substituent group is substituted with one or more third substituent groups denoted by R^{5D.3} as explained in the definitions section above in the description of “first substituent group(s)”. In the above embodiments, R^{5D}, R^{5D.1}, R^{5D.2}, and R^{5D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{5D}, R^{5D.1}, R^{5D.2}, and R^{5D.3}, respectively.

In embodiments, when R⁶ is substituted, R⁶ is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R⁶, R^{6.1}, R^{6.2}, and R^{6.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2} and R^{WW.3} correspond to R⁶, R^{6.1}, R^{6.2}, and R^{6.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{6B}, R^{6B.1}, R^{6B.2}, and R^{6B.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A.1}, R^{6A.2}, and R^{6A.3} have values corresponding to the values of R^{WW.1}, R^{WW.2}, and R^{WW.3} respectively, as explained in the definitions section above in the description of “first substituent group(s)”, wherein R^{WW.1}, R^{WW.2}, and R^{WW.3} correspond to R^{6A.1}, R^{6A.2}, and R^{6A.3}, respectively.

In embodiments, when R^{6A} and R^{6B} substituents bonded to the same nitrogen atom are optionally joined to form a moiety that is substituted (e.g., a substituted heterocycloalkyl or substituted heteroaryl), the moiety is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B.1}, R^{6B.2}, and R^{6B.3} have values corresponding to the values of R^{WW.1}. R^{WW.2}, and R^{WW.3}, respectively, as explained in the definitions section above in the description of “first substituent group(s)”, wherein R^{WW.1}, R^{WW.2}, and R^{WW.3} correspond to R^{6B.1}, R^{6B.2}, and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R^{6C}, R^{6C.1}, R^{6C.2}, and R^{6C.3}, respectively.

In embodiments, when R^{6D} is substituted, R^{6D} is substituted with one or more first substituent groups denoted by R^{6D.1} as explained in the definitions section above in the description of “first substituent group(s)”. In embodiments, when an R^{6D.1} substituent group is substituted, the R^{6D.1} substituent group is substituted with one or more second substituent groups denoted by R^{6D.2} as explained in the definitions section above in the description of “first substituent group(s)”. In embodiments, when an R^{6D.2} substituent group is substituted, the R^{6D.2} substituent group is substituted with one or more third substituent groups denoted by R^{6D.3} as explained in the definitions section above in the description of “first substituent group(s)”. In the above embodiments, R^{6D}, R^{6D.1}, R^{6D.2}, and R^{6D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2}, and R^{WW.3} correspond to R^{6D}, R^{6D.1}, R^{6D.2}, and R^{6D.3}, respectively.

In embodiments, when R³ and R⁶ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above, embodiments, R^{3.1}, R^{3.2}, and R^{3.3} have values corresponding to the values of R^{WW.1}, R^{WW.2} and R^{WW.3}, respectively, as explained in the definitions section above in the description of “first substituent group(s)”, wherein R^{WW.1}, R^{WW.2}, and R^{WW.3} correspond to R^{3.1}, R^{3.2}, and R^{3.3}, respectively.

In embodiments, when R³ and R⁶ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{6.1} as explained in the definitions section above in the description of “first substituent group(s)”. In embodiments, when an R^{6.1} substituent group is substituted, the R^{6.1} substituent group is substituted with one or more second substituent groups denoted by R^{6.2} as explained in the definitions section above in the description of “first substituent group(s)”. In embodiments, when an R^{6.2} substituent group is substituted, the R^{6.2} substituent group is substituted with one or more third substituent groups denoted by R^{6.3} as explained in the definitions section above in the description of “first substituent group(s)”. In the above embodiments, R^{6.1}, R^{6.2}, and R^{6.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{6.1}, R^{6.2}, and R^{6.3}, respectively.

In embodiments, when R⁷ is substituted, R⁷ is substituted with one or more first substituent groups denoted by R^{7.1} as explained in the definitions section above in the description of “first substituent group(s)”. In embodiments, when an R^{7.1} substituent group is substituted, the R^{7.1} substituent group is substituted with one or more second substituent groups denoted by R^{7.2} as explained in the definitions section above in the description of “first substituent group(s)”. In embodiments, when an R^{7.2} substituent group is substituted, the R^{7.2} substituent group is substituted with one or more third substituent groups denoted by R^{7.3} as explained in the definitions section above in the description of “first substituent group(s)”. In the above embodiments, R⁷, R^{7.1}, R^{7.2}, and R^{7.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R⁷, R^{7.1}, R^{7.2}, and R^{7.3}, respectively.

In embodiments, when R⁸ is substituted, R⁸ is substituted with one or more first substituent groups denoted by R^{8.1} as explained in the definitions section above in the description of “first substituent group(s)”. In embodiments, when an R^{8.1} substituent group is substituted, the R^{8.1} substituent group is substituted with one or more second substituent groups denoted by R^{8.2} as explained in the definitions section above in the description of “first substituent group(s)”. In embodiments, when an R^{8.2} substituent group is substituted, the R^{8.2} substituent group is substituted with one or more third substituent groups denoted by R^{8.3} as explained in the definitions section above in the description of “first substituent group(s)”. In the above embodiments, R⁸, R^{8.1}, R^{8.2}, and R^{8.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}. R^{WW.2} and R^{WW.3} correspond to R⁸, R^{8.1}, R^{8.2}, and R^{8.3}, respectively.

In embodiments, when R⁹ is substituted, R⁹ is substituted with one or more first substituent groups denoted by R^{9.1} as explained in the definitions section above in the description of “first substituent group(s)”. In embodiments, when an R^{9.1} substituent group is substituted, the R^{9.1} substituent group is substituted with one or more second substituent groups denoted by R^{9.2} as explained in the definitions section above in the description of “first substituent group(s)”. In embodiments, when an R^{9.2} substituent group is substituted, the R^{9.2} substituent group is substituted with one or more third substituent groups denoted by R^{9.3} as explained in the definitions section above in the description of “first substituent group(s)”. In the above embodiments, R⁹, R^{9.1}, R^{9.2}, and R^{9.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R⁹, R^{9.1}, R^{9.2}, and R^{9.3}, respectively.

In embodiments, when Ring A is substituted, Ring A is substituted with one or more first substituent groups denoted by R^{A.1} as explained in the definitions section above in the description of “first substituent group(s)”. In embodiments, when an R^{A.1} substituent group is substituted, the R^{A.1} substituent group is substituted with one or more second substituent groups denoted by R^{A.2} as explained in the definitions section above in the description of “first substituent group(s)”. In embodiments, when an R^{A.2} substituent group is substituted, the R^{A.2} substituent group is substituted with one or more third substituent groups denoted by R^{A.3} as explained in the definitions section above in the description of “first substituent group(s)”. In the above embodiments, Ring A, R^{A.1}, R^{A.2}, and R^{A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to Ring A, R^{A.1}, R^{A.2}, and R^{A.3}, respectively.

In embodiments, when Ring B is substituted, Ring B is substituted with one or more first substituent groups denoted by R^{B.1} as explained in the definitions section above in the description of “first substituent group(s)”. In embodiments, when an R^{B.1} substituent group is substituted, the R^{B.1} substituent group is substituted with one or more second substituent groups denoted by R^{B.2} as explained in the definitions section above in the description of “first substituent group(s)”. In embodiments, when an R^{B.2} substituent group is substituted, the R^{B.2} substituent group is substituted with one or more third substituent groups denoted by R^{B.3} as explained in the definitions section above in the description of “first substituent group(s)”. In the above embodiments, Ring B, R^{B.1}, R^{B.2}, and R^{B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to Ring B, R^{B.1}, R^{B.2}, and R^{B.3}, respectively.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

In embodiments, R⁶ is not hydrogen. In embodiments, L¹ is not —O—. In embodiments, m is not 0. In embodiments, n is not 0.

In embodiments, the compound is not

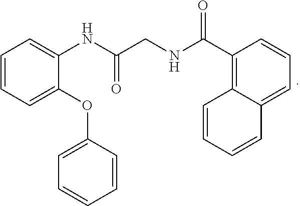 [SMILES: O=C(CNC(=O)c1cccc2ccccc12)Nc1ccccc1Oc1ccccc1]

In embodiments, the compound is not

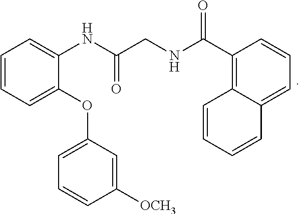 [SMILES: COc1cccc(Oc2ccccc2NC(=O)CNC(=O)c2cccc3ccccc23)c1]

In embodiments, the compound is not

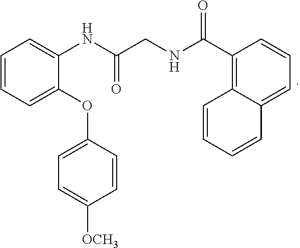 [SMILES: COc1ccc(Oc2ccccc2NC(=O)CNC(=O)c2cccc3ccccc23)cc1]

In embodiments, the compound is not

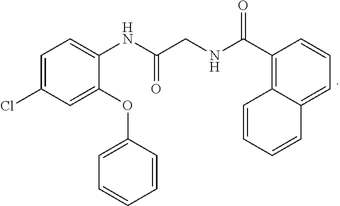 [SMILES: O=C(CNC(=O)c1cccc2ccccc12)Nc1ccc(Cl)cc1Oc1ccccc1]

In embodiments, the compound is not

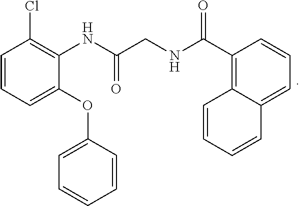 [SMILES: O=C(CNC(=O)c1cccc2ccccc12)Nc1c(Cl)cccc1Oc1ccccc1]

In embodiments, the compound is not

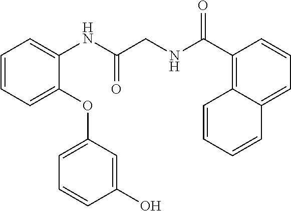 [SMILES: O=C(CNC(=O)c1cccc2ccccc12)Nc1ccccc1Oc1cccc(O)c1]

In embodiments, the compound is not

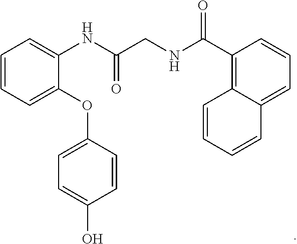 [SMILES: O=C(CNC(=O)c1cccc2ccccc12)Nc1ccccc1Oc1ccc(O)cc1]

In embodiments, the compound is not

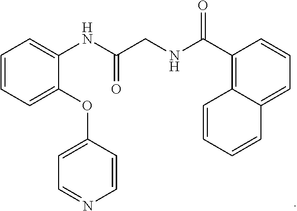 [SMILES: O=C(CNC(=O)c1cccc2ccccc12)Nc1ccccc1Oc1ccncc1]

In embodiments, the compound is not

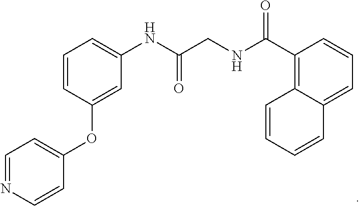 [SMILES: O=C(CNC(=O)c1cccc2ccccc12)Nc1cccc(Oc2ccncc2)c1]

In embodiments, the compound is not

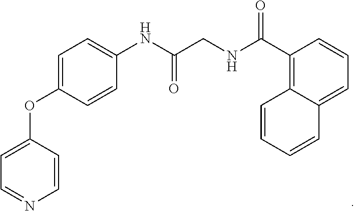 [SMILES: O=C(CNC(=O)c1cccc2ccccc12)Nc1ccc(Oc2ccncc2)cc1]

In embodiments, the compound is not

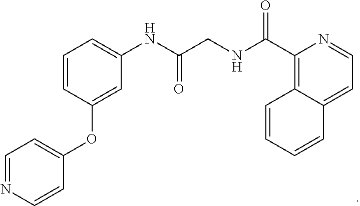 [SMILES: O=C(CNC(=O)c1nccc2ccccc12)Nc1cccc(Oc2ccncc2)c1]

In embodiments, the compound is not

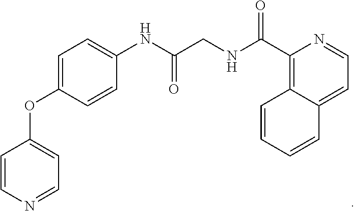 [SMILES: O=C(CNC(=O)c1nccc2ccccc12)Nc1ccc(Oc2ccncc2)cc1]

In embodiments, the compound is not

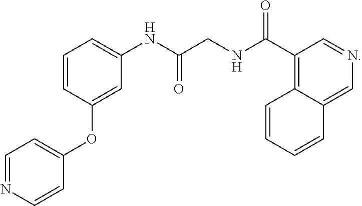 [SMILES: O=C(CNC(=O)c1cncc2ccccc12)Nc1cccc(Oc2ccncc2)c1]

In embodiments, the compound is not

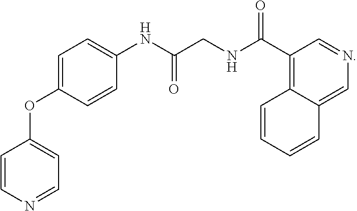 [SMILES: O=C(CNC(=O)c1cncc2ccccc12)Nc1ccc(Oc2ccncc2)cc1]

In embodiments, the compound is not

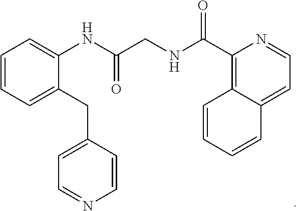 [SMILES: O=C(CNC(=O)c1nccc2ccccc12)Nc1ccccc1Cc1ccncc1]

In embodiments, the compound is not

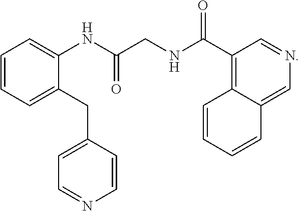 [SMILES: O=C(CNC(=O)c1cncc2ccccc12)Nc1ccccc1Cc1ccncc1]

In embodiments, the compound is not

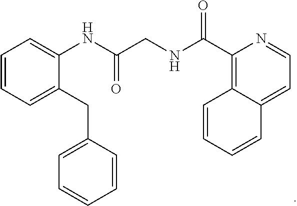 [SMILES: O=C(CNC(=O)c1nccc2ccccc12)Nc1ccccc1Cc1ccccc1]

In embodiments, the compound is not

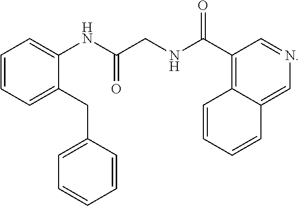 [SMILES: O=C(CNC(=O)c1cncc2ccccc12)Nc1ccccc1Cc1ccccc1]

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound. In embodiments, the compound is a compound of formula (I), (II), (III), (IIIa), (IIIb), (IV), (IVa), (IVb), (V), (Va), (Vb), (VI), (VIa), or (VIb), including embodiments thereof. In embodiments, the compound is a compound of formula (I), (II), (III), (IIIa), (IIIb), (IV), (IVa), (IVb), (V), (Va), (Vb), (VI), (VIa), or (VIb).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid in a mixture with the finely divided active component (e.g., a compound provided herein). In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragees cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active component (e.g., compounds described herein) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In embodiments, the pharmaceutical composition further includes an anti-cancer agent. In embodiments, the anti-cancer agent is a platinum-based compound, topoisomerase inhibitor, or Chk1 inhibitor. In embodiments, the anti-cancer agent is cisplatin. In embodiments, the anti-cancer agent is oxaloplatin. In embodiments, the anti-cancer agent is carboplatin. In embodiments, the anti-cancer agent is etoposide. In embodiments, the anti-cancer agent is SN-38. In embodiments, the anti-cancer agent is camptothecin. In embodiments, the anti-cancer agent is gemcitabine. In embodiments, the anti-cancer agent is CHIR-124. In embodiments, the anti-cancer agent is debromohymenialdisine. In embodiments, the anti-cancer agent is SB 218078. In embodiments, the anti-cancer agent is LY2603618. In embodiments, the anti-cancer agent is SCH900776. In embodiments, the anti-cancer agent is TCS 2312. In embodiments, the anti-cancer agent is PF 477736. In embodiments, the anti-cancer agent is UCN-01. In embodiments, the anti-cancer agent is AZD7762.

### IV. Methods of Use

In an aspect is provided a method of treating a disease associated with PCNA activity in a subject in need thereof, the method including administering to the subject in need thereof a therapeutically effective amount of a compound described herein, or a pharmaceutically acceptable salt thereof.

In an aspect is provided a method of treating cancer in a subject in need thereof, the method including administering to the subject in need thereof a therapeutically effective amount of a compound described herein, or a pharmaceutically acceptable salt thereof.

In embodiments, the cancer is a sarcoma, adenocarcinoma, leukemia, or lymphoma. In embodiments, the cancer is a lung cancer, colon cancer, central nervous system cancer, brain cancer, neuroblastoma, skin cancer, head and neck cancer, melanoma, ovarian cancer, renal cancer, prostate cancer, breast cancer, mesothelioma, liver cancer, stomach cancer, esophageal cancer, bladder cancer, cervical cancer, osteosarcoma, pancreatic cancer, adrenal cortical cancer, adrenal gland cancer, colorectal cancer, testicular cancer, myeloma, B-acute lymphoblastic lymphoma, non-Hodgkin's lymphoma, Hodgkin's lymphoma, chronic leukemia, acute leukemia, glandular carcinoma, or hematoid carcinoma. In embodiments, the cancer is a sarcoma, In embodiments, the cancer is adenocarcinoma. In embodiments, the cancer is leukemia. In embodiments, the cancer is lymphoma. In embodiments, the cancer is a CNS cancer. In embodiments, the cancer is melanoma. In embodiments, the cancer is renal cancer. In embodiments, the cancer is metastatic cancer. In embodiments, the cancer is breast cancer. In embodiments, the cancer is triple negative breast cancer. In embodiments, the cancer is metastatic breast cancer. In embodiments, the cancer is brain cancer. In embodiments, the cancer is neuroblastoma. In embodiments, the cancer is glioblastoma. In embodiments, the cancer is astrocytoma. In embodiments, the cancer is glioma. In embodiments, the cancer is pancreatic cancer. In embodiments, the cancer is chronic lymphoid leukemia (CLL). In embodiments, the cancer is non-Hodgkin's lymphoma. In embodiments, the cancer is skin cancer. In embodiments, the cancer is squamous cell carcinoma. In embodiments, the cancer is T lymphotrophic leukemia. In embodiments, the cancer is malignant melanoma. In embodiments, the cancer is lung cancer. In embodiments, the cancer is non-small cell lung cancer. In embodiments, the cancer is small-cell lung cancer. In embodiments, the cancer is colon cancer. In embodiments, the cancer is prostate cancer. In embodiments, the cancer is ovarian cancer. In embodiments, the cancer is kidney cancer. In embodiments, the cancer may be prostate, thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples may include, but are not limited to Hodgkin's Disease, Non-Hodgkin's Lymphoma, multiple myeloma, neuroblastoma, glioma, glioblastoma multiforme, ovarian cancer, neuroblastoma, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is leukemia, myeloma, non-small cell lung cancer, colon cancer, central nervous system cancer, melanoma, ovarian cancer, renal cancer, prostate cancer, or breast cancer. In embodiments, the cancer is triple negative breast cancer. In embodiments, the cancer is a central nervous system (CNS) cancer. In embodiments, the cancer is a sympathetic nervous system (SNS) cancer. In embodiments, the cancer is an adrenal gland cancer. In embodiments, the cancer is a cancer of a neuron in the neck, chest, abdomen, or pelvis. In embodiments, the cancer is an esthesioneuroblastoma. In embodiments, the cancer is a stage 1 neuroblastoma (e.g., localized tumor confined to an area near the origin). In embodiments, the cancer is a a stage 2A neuroblastoma (e.g., Unilateral tumor with incomplete gross resection and/or identifiable ipsilateral and contralateral lymph node negative for tumor). In embodiments, the cancer is a a stage 2B neuroblastoma (e.g., Unilateral tumor with complete or incomplete gross resection; with ipsilateral lymph node positive for tumor; identifiable contralateral lymph node negative for tumor). In embodiments, the cancer is a a stage 3 neuroblastoma (e.g., Tumor infiltrating across midline with or without regional lymph node involvement; or unilateral tumor with contralateral lymph node involvement; or midline tumor with bilateral lymph node involvement). In embodiments, the cancer is a a stage 4 neuroblastoma (e.g., Dissemination of tumor to distant lymph nodes, bone marrow, bone, liver, or other organs except as defined by Stage 4S). In embodiments, the cancer is a a stage 4S neuroblastoma (e.g., Age <1 year old with localized primary tumor as described in Stage 1 or Stage 2 above, with dissemination limited to liver, skin, or bone marrow (less than 10 percent of nucleated bone marrow cells are tumors). In embodiments, the cancer is a stage L1 neuroblastoma (e.g., localized cancer without image-defined risk factors) according to the International Neuroblastoma Risk Group (INRG) staging system. In embodiments, the cancer is a stage L2 neuroblastoma (e.g., localized cancer with image-defined risk factors) according to the International Neuroblastoma Risk Group (INRG) staging system. In embodiments, the cancer is a stage M neuroblastoma (e.g., metastatic cancer) according to the International Neuroblastoma Risk Group (INRG) staging system. In embodiments, the cancer is a stage MS neuroblastoma (e.g., metastatic cancer “special” where MS is equivalent to stage 4S as described above) according to the International Neuroblastoma Risk Group (INRG) staging system. In embodiments, the cancer is a neuroblastoma risk stratification pre-treatment group, according to the International Neuroblastoma Risk Group (INRG) staging system, of very low. In embodiments, the cancer is a neuroblastoma risk stratification pre-treatment group, according to the International Neuroblastoma Risk Group (INRG) staging system, of low. In embodiments, the cancer is a neuroblastoma risk stratification pre-treatment group, according to the International Neuroblastoma Risk Group (INRG) staging system, of intermediate. In embodiments, the cancer is a neuroblastoma risk stratification pre-treatment group, according to the International Neuroblastoma Risk Group (INRG) staging system, of high risk.

In embodiments, the cancer is cervical cancer, colon cancer, thyroid cancer, gastric cancer, ovarian cancer, breast cancer, lung cancer, uterine cancer, or Ductal carcinoma in situ (DCIS). In embodiments, the cancer is cervical cancer. In embodiments, the cancer is colon cancer. In embodiments, the cancer is thyroid cancer. In embodiments, the cancer is gastric cancer. In embodiments, the cancer is ovarian cancer. In embodiments, the cancer is breast cancer. In embodiments, the cancer is lung cancer. In embodiments, the cancer is uterine cancer. In embodiments, the cancer is Ductal carcinoma in situ (DCIS).

In embodiments, the cancer is esophageal adenocarcinoma. In embodiments, the cancer is stage 0 esophageal cancer. In embodiments, the cancer is stage I esophageal cancer. In embodiments, the cancer is stage IA esophageal cancer. In embodiments, the cancer is stage IB esophageal cancer. In embodiments, the cancer is stage IIA esophageal cancer. In embodiments, the cancer is stage IIB esophageal cancer. In embodiments, the cancer is stage IIIA esophageal cancer. In embodiments, the cancer is stage IIIB esophageal cancer. In embodiments, the cancer is stage IIIC esophageal cancer. In embodiments, the cancer is stage IV esophageal cancer. In embodiments, the cancer is stage I esophageal adenocarcinoma. In embodiments, the cancer is colorectal cancer. In embodiments, the cancer is prostate cancer (e.g., prostatic adenocarcinoma). In embodiments, the cancer is high-grade prostatic intraepithelial neoplasia (PIN). In embodiments, the cancer is associated with Barrett's esophagus. In embodiments, the cancer is associated with Barrett's esophagus without epithelial dysplasia. In embodiments, the cancer is associated with Barrett's esophagus with low grade epithelial dysplasia. In embodiments, the cancer is associated with Barrett's esophagus with high-grade epithelial dysplasia. In embodiments, the cancer is oesophagogastric junctional adenocarcinoma. In embodiments, the cancer is described in Hammoud et al. (Z. T. Hammoud, et al. Journal of Thoracic & Cardiovascular Surgery 2006; 133 (1): 82-87); Wang X., et al. Prostate. 2011 May 15; 71 (7): 748-54; or Shen F., et al. J Cell Biochem. 2011 March; 112 (3): 756-60, which are incorporated by reference in their entirety for all purposes.

In embodiments, the method includes administering a second agent (e.g., therapeutic agent). In embodiments, the second agent is an anti-cancer agent. In embodiments, the anti-cancer agent is a platinum-based compound. In embodiments, the anti-cancer agent is cisplatin. In embodiments, the anti-cancer agent is oxaloplatin. In embodiments, the anti-cancer agent is carboplatin. In embodiments, the anti-cancer agent is a DNA damaging agent or cytotoxic agent in routine clinical use for treating cancer. In embodiments, the method includes administration of high-dose chemotherapy. In embodiments, the method includes stem cell transplantation (HDCT/autoSCT). In embodiments, the method includes administration of 13-cis-retinoid acid. In embodiments, the method includes administration of immunotherapy. In embodiments, the method includes administration of radiation. In embodiments, the second agent is a chemotherapeutic agent. In embodiments, the second agent is included in a therapeutically effective amount.

In an aspect is provided a method of inhibiting PCNA activity, the method including contacting PCNA with an effective amount of a compound described herein, or a pharmaceutically acceptable salt thereof. In embodiments, the PCNA is a human PCNA.

In embodiments, the compound binds to His44, Val45, Leu47, Pro234, Tyr250, Leu251, Ala252, Met40, Leu47, Leu126, Leu128, Val233, Pro234, Ala252, Pro253, or Asp 232 of PCNA. In embodiments, the compound binds noncovalently to His44, Val45, Leu47, Pro234, Tyr250, Leu251, Ala252, Met40, Leu47, Leu126, Leu128, Val233, Pro234, Ala252, Pro253, or Asp 232 of PCNA.

### V. Methods of Making

In an aspect is provided a method of making compound (I), or a pharmaceutically acceptable salt thereof, the method including mixing compound (VII) and compound (X) together in a reaction vessel. Compound (I) has the formula:

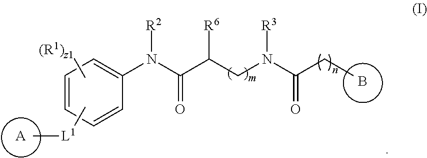

Compound (VII) has the formula:

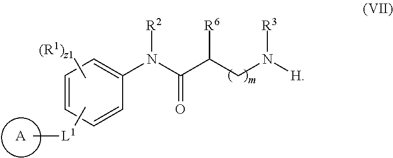

Compound (X) has the formula:

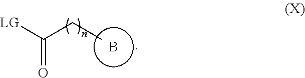

L¹, Ring A, R¹, z1, R², R³, R⁶, m, and n are as described herein, including in embodiments. LG is a leaving group.

In embodiments, LG is halogen. In embodiments, LG is —F. In embodiments, LG is —Cl. In embodiments, LG is —Br. In embodiments, LG is —I. In embodiments, LG is —OH. In embodiments, LG is

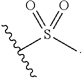 [SMILES: *S(C)(=O)=O]

In embodiments, LG is

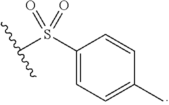 [SMILES: *S(=O)(=O)c1ccc(C)cc1]

In embodiments, LG is

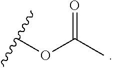 [SMILES: *OC(C)=O]

In embodiments, LG is

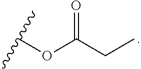 [SMILES: *OC(=O)CC]

In embodiments, LG is

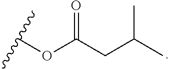 [SMILES: *OC(=O)CC(C)C]

In embodiments, LG is

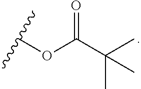 [SMILES: *OC(=O)C(C)(C)C]

In embodiments, LG is

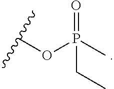 [SMILES: *OP(C)(=O)CC]

In embodiments, LG is

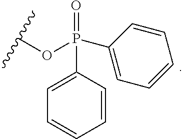 [SMILES: *OP(=O)(c1ccccc1)c1ccccc1]

In embodiments, LG is

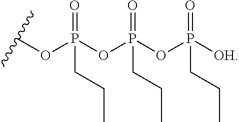 [SMILES: *OP(=O)(CCC)OP(=O)(CCC)OP(=O)(O)CCC]

In embodiments, the method further comprises a base. In embodiments, the base is N,N-diisopropylethylamine. In embodiments, the base is triethylamine. In embodiments, the base is N-methylpiperidine.

In embodiments, the method further comprises a peptide coupling agent. In embodiments, the peptide coupling agent is dicyclohexylcarbodiimide. In embodiments, the peptide coupling agent is HBTU. In embodiments, the peptide coupling agent is HOBt. In embodiments, the peptide coupling agent is PyBOP. In embodiments, the peptide coupling agent is BOP. In embodiments, the peptide coupling agent is COMU. In embodiments, the peptide coupling agent is HATU. In embodiments, the peptide coupling agent is HCTU. In embodiments, the peptide coupling agent isPyAOP. In embodiments, the peptide coupling agent is PyClock. In embodiments, the peptide coupling agent is PyOxim. In embodiments, the peptide coupling agent is TOTU.

In embodiments, the method further comprises a peptide coupling agent. In embodiments, the method further includes mixing compound (X) with a peptide coupling agent to form an activated imidazolide compound. In embodiments, the peptide coupling agent is

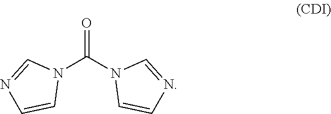 [SMILES: O=C(n1ccnc1)n1ccnc1]

In embodiments, the method further includes mixing compound (X) with a peptide coupling agent to form an activated O-acylisourea ester compound. In embodiments, the peptide coupling agent is

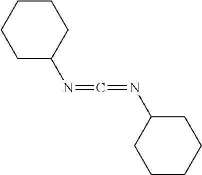 [SMILES: C(=NC1CCCCC1)=NC1CCCCC1]

(DCC or dicyclohexylcarbodiimide). In embodiments, the peptide coupling agent is

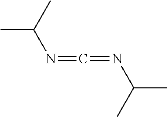

(DIC). In embodiments, the peptide coupling agent is

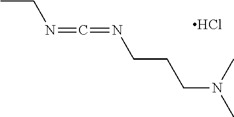

(EDCI, EDC, EDAC, or WSC). In embodiments, the method further includes mixing compound (X) with a peptide coupling agent to form an activated benzotriazole ester compound. In embodiments, the peptide coupling agent is

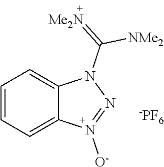

(HBTU). In embodiments, the peptide coupling agent is HOBt. In embodiments, the peptide coupling agent is

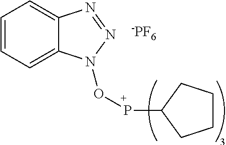

(PyBOP). In embodiments, the peptide coupling agent is

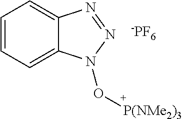

(BOP). In embodiments, the peptide coupling agent is COMU. In embodiments, the peptide coupling agent is

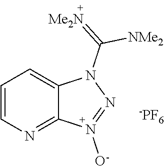

(HATU). In embodiments, the peptide coupling agent is

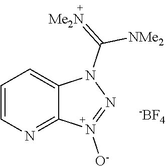

(TBTU). In embodiments, the peptide coupling agent is HCTU. In embodiments, the peptide coupling agent is PyAOP. In embodiments, the peptide coupling agent is PyClock. In embodiments, the peptide coupling agent is PyOxim. In embodiments, the method further includes mixing compound (X) with a peptide coupling agent to form an activated N-oxide ester compound. In embodiments, the peptide coupling agent is

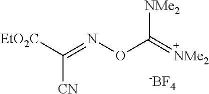

(TOTU). In embodiments, the peptide coupling agent is

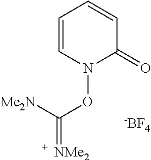

(TPTU). In embodiments, the method further includes mixing compound (X) with a peptide coupling agent to form an activated triazine ester compound. In embodiments, the peptide coupling agent is

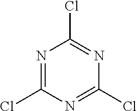

(TCT). In embodiments, the peptide coupling agent is

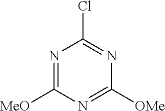 [SMILES: COc1nc(Cl)nc(OC)n1]

(CDMT). In embodiments, the peptide coupling agent is

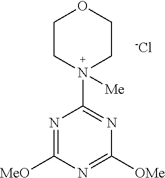

(DMTMM). In embodiments, the method further includes mixing compound (X) with a peptide coupling agent to form an activated boron-derived mixed anhydride compound. In embodiments, the peptide coupling agent is B(OH)₃ (boric acid). In embodiments, the peptide coupling agent is

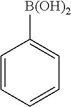

(phenylboronic acid). In embodiments, the peptide coupling agent is

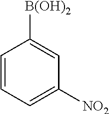 [SMILES: O=[N+]([O-])c1cccc(B(O)O)c1]

(3-nitrophenylboronic acid). In embodiments, the method further includes mixing compound (X) with a peptide coupling agent to form an activated silyl ester compound. In embodiments, the peptide coupling agent is

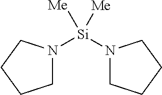 [SMILES: C[Si](C)(N1CCCC1)N1CCCC1]

(dimethylbis(pyrrolidin-1-yl) silane).

In embodiments, compound (I) is a compound of formula (II):

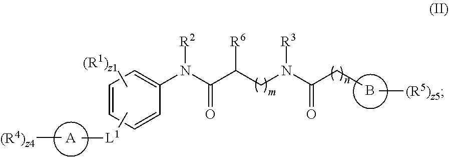

compound (VII) is a compound of formula (VIII);

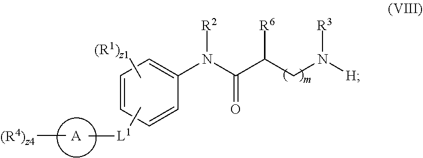

and compound (X) is a compound of formula (XI):

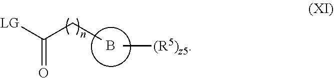

L¹, Ring A, R¹, z1, R², R³, R⁴, z4, R⁵, z5, R⁶, m, n, and LG are as described herein, including in embodiments.

In embodiments, the method further includes mixing compound (XII) with an acid to make compound (VII), wherein compound (XII) has the formula:

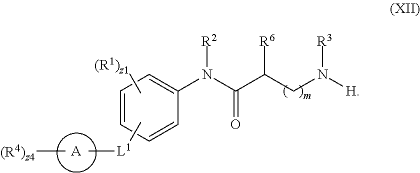

L¹, Ring A, R¹, z1, R², R³, R⁴, z4, R⁶, and m are as described herein, including in embodiments. PG is a protecting group.

In embodiments, PG is tert-butyloxycarbonyl (Boc). In embodiments, PG is fluorenylmethyloxycarbonyl (Fmoc). In embodiments, PG is benzyloxycarbonyl (Cbz). In embodiments, PG is —C(O)CH₃ (also denoted Ac). In embodiments, PG is —CH₂Ph (also denoted Bn). In embodiments, PG is —C(Ph)₃ (also denoted trityl). In embodiments, PG is toluenesulfonyl (also denoted Ts). In embodiments, PG is —C(O)CF₃. In embodiments, PG is phthalimide. In embodiments, PG is benzylideneamine.

In embodiments, the acid is trifluoroacetic acid (TFA).

In embodiments, the method further includes mixing compound (XIII), compound (XIV), and a peptide coupling agent to make compound (XII); wherein compound (XIII) has the formula:

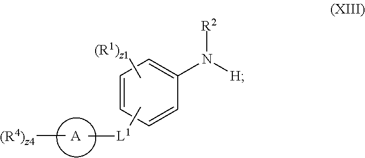

and compound (XIV) has the formula:

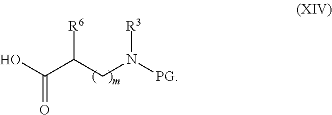

L¹, Ring A, R¹, z1, R², R³, R⁴, z4, R⁶, m, and PG are as described herein, including in embodiments.

In embodiments, the peptide coupling agent is dicyclohexylcarbodiimide. In embodiments, the peptide coupling agent is HBTU. In embodiments, the peptide coupling agent is HOBt. In embodiments, the peptide coupling agent is PyBOP.

### VI. Embodiments

Embodiment P1. A compound, or a pharmaceutically acceptable salt thereof, having the formula:

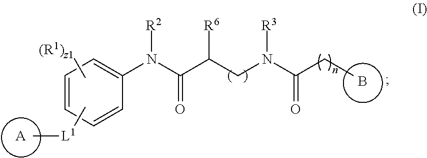

wherein
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
Ring B is substituted or unsubstituted phenyl, substituted or unsubstituted naphthyl, substituted or unsubstituted quinolinyl, or substituted or unsubstituted isoquinolinyl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F;
m is an integer from 0 to 5; and
n is an integer from 0 to 10;
provided when L¹ is —O—, m is 0, n is 0, and Ring B is substituted or unsubstituted naphthyl, substituted or unsubstituted quinolinyl, or substituted or unsubstituted isoquinolinyl, then R⁶ is not hydrogen.

Embodiment P2. The compound of embodiment P1, having the formula:

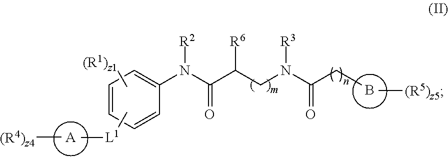

wherein
Ring A is phenyl or 5 to 6 membered heteroaryl;
Ring B is phenyl, naphthyl, quinolinyl, or isoquinolinyl;
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX₅³, —CHX⁵₂, —CH₂X⁵, —OCX₅³, —OCHX⁵₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
z4 is an integer from 0 to 5;
z5 is an integer from 0 to 7;
m4, m5, v4, and v5 are independently 1 or 2;
n4 and n5 are independently an integer from 0 to 4; and
X⁴ and X⁵ are independently —Cl, —Br, —I, or —F.

Embodiment P3. The compound of embodiment P2, having the formula:

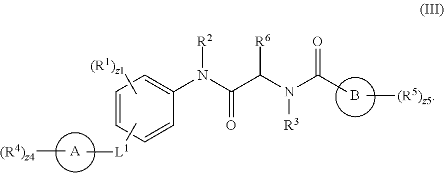

Embodiment P4. The compound of embodiment P3, having the formula:

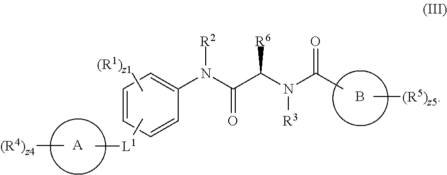

Embodiment P5. The compound of embodiment P3, having the formula:

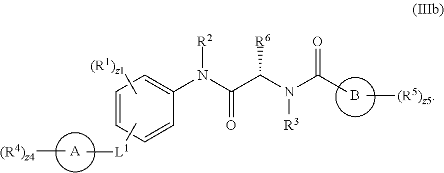

Embodiment P6. The compound of embodiment P3, having the formula:

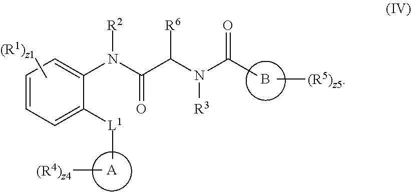

Embodiment P7. The compound of embodiment P3, having the formula:

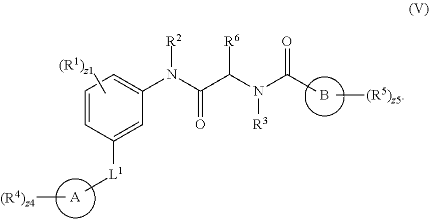

Embodiment P8. The compound of embodiment P3, having the formula:

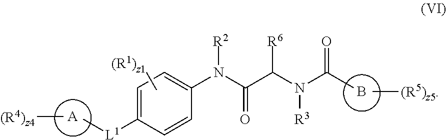

Embodiment P9. The compound of one of embodiments P1 to P8, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

Embodiment P10. The compound of one of embodiments P1 to P8, wherein L¹ is —O—.

Embodiment P11. The compound of one of embodiments P1 to P8, wherein L¹ is —S—.

Embodiment P12. The compound of one of embodiments P1 to P8, wherein L¹ is —S(O)₂—.

Embodiment P13. The compound of one of embodiments P1 to P12, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P14. The compound of one of embodiments P1 to P12, wherein R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P15. The compound of one of embodiments P1 to P12, wherein R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment P16. The compound of one of embodiments P1 to P12, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment P17. The compound of one of embodiments P1 to P16, wherein z1 is 1.

Embodiment P18. The compound of one of embodiments P1 to P12, wherein z1 is 0.

Embodiment P19. The compound of one of embodiments P1 to P18, wherein R² is hydrogen, —CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P20. The compound of one of embodiments P1 to P18, wherein R² is hydrogen, unsubstituted methyl, unsubstituted ethyl, or unsubstituted isopropyl.

Embodiment P21. The compound of one of embodiments P1 to P18, wherein R² is hydrogen.

Embodiment P22. The compound of one of embodiments P1 to P21, wherein R³ is hydrogen, —CX³₃, —CHX³₂, —CH₂X³, —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P23. The compound of one of embodiments P1 to P21, wherein R³ is hydrogen, unsubstituted methyl, unsubstituted ethyl, or unsubstituted isopropyl.

Embodiment P24. The compound of one of embodiments P1 to P21, wherein R³ is hydrogen.

Embodiment P25. The compound of one of embodiments P1 to P24, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P26. The compound of one of embodiments P1 to P24, wherein R⁶ is substituted or unsubstituted C₁-C₆ alkyl or substituted or unsubstituted 2 to 6 membered heteroalkyl.

Embodiment P27. The compound of one of embodiments P1 to P24, wherein R⁶ is hydrogen, unsubstituted methyl, unsubstituted isopropyl,

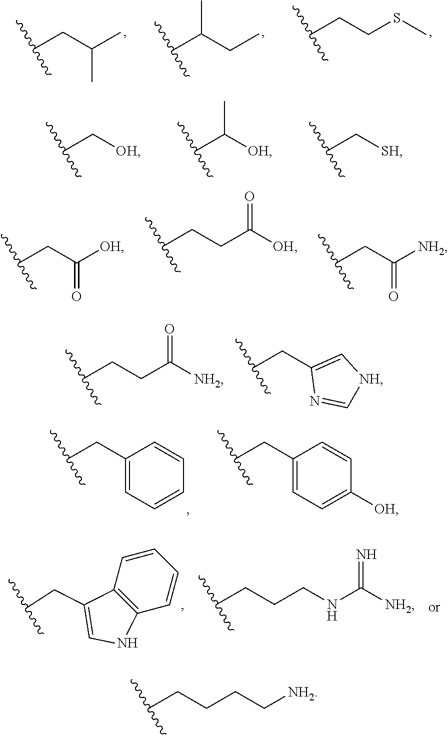

Embodiment P28. The compound of one of embodiments P1 to P21, wherein R³ and R⁶ are joined to form a substituted or unsubstituted heterocycloalkyl or substituted or unsubstituted heteroaryl.

Embodiment P29. The compound of one of embodiments P1 to P21, wherein R³ and R⁶ are joined to form a substituted or unsubstituted 4 to 8 membered heterocycloalkyl.

Embodiment P30. The compound of one of embodiments P1 to P21, wherein R³ and R⁶ are joined to form an unsubstituted pyrrolidinyl.

Embodiment P31. The compound of one of embodiments P1 to P21, wherein R³ and R⁶ are joined to form an unsubstituted piperidinyl.

Embodiment P32. The compound of one of embodiments P2 to P31, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P33. The compound of one of embodiments P2 to P31, wherein R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P34. The compound of one of embodiments P2 to P31, wherein R⁴ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment P35. The compound of one of embodiments P2 to P31, wherein R⁴ is independently halogen, —CF₃, —CHF₂, —CH₂F, —OCF₃, —OCHF₂, —OCH₂F, —OH, unsubstituted methyl, or unsubstituted methoxy.

Embodiment P36. The compound of one of embodiments P2 to P31, wherein R⁴ is independently —OR^{4D}.

Embodiment P37. The compound of embodiment P36, wherein R^{4D} is hydrogen or substituted or unsubstituted alkyl.

Embodiment P38. The compound of embodiment P36, wherein R^{4D} is hydrogen or unsubstituted alkyl.

Embodiment P39. The compound of embodiment P36, wherein R^{4D} is hydrogen or unsubstituted C₁-C₅ alkyl.

Embodiment P40. The compound of embodiment P36, wherein R^{4D} is hydrogen or unsubstituted methyl.

Embodiment P41. The compound of embodiment P36, wherein R^{4D} is unsubstituted methyl.

Embodiment P42. The compound of one of embodiments P2 to P41, wherein z4 is 1.

Embodiment P43. The compound of one of embodiments P2 to P31, wherein z4 is 0.

Embodiment P44. The compound of one of embodiments P2 to P43, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P45. The compound of one of embodiments P2 to P43, wherein R⁵ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P46. The compound of one of embodiments P2 to P43, wherein R⁵ is independently halogen, —CF₃, —CHF₂, —CH₂F, —OCF₃, —OCHF₂, —OCH₂F, —OH, —NH₂, —SH, unsubstituted C₁-C₄ alkyl, unsubstituted 2 to 4 membered heteroalkyl, or unsubstituted phenyl.

Embodiment P47. The compound of one of embodiments P2 to P43, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

Embodiment P48. The compound of one of embodiments P2 to P47, wherein z5 is 1.

Embodiment P49. The compound of one of embodiments P2 to P43, wherein z5 is 0.

Embodiment P50. The compound of embodiment P1, wherein Ring A is a substituted or unsubstituted phenyl.

Embodiment P51. The compound of embodiment P1, wherein Ring A is a substituted or unsubstituted 5 to 6 membered heteroaryl.

Embodiment P52. The compound of embodiment P1, wherein Ring A is a substituted or unsubstituted thienyl.

Embodiment P53. The compound of embodiment P1, wherein Ring A is a substituted or unsubstituted 2-thienyl.

Embodiment P54. The compound of embodiment P1, wherein Ring A is a substituted or unsubstituted 3-thienyl.

Embodiment P55. The compound of embodiment P1, wherein Ring A is a substituted or unsubstituted pyridyl.

Embodiment P56. The compound of embodiment P1, wherein Ring A is a substituted or unsubstituted 2-pyridyl.

Embodiment P57. The compound of embodiment P1, wherein Ring A is a substituted or unsubstituted 3-pyridyl.

Embodiment P58. The compound of embodiment P1, wherein Ring A is a substituted or unsubstituted 4-pyridyl.

Embodiment P59. The compound of embodiment P1, wherein Ring B is a substituted or unsubstituted phenyl.

Embodiment P60. The compound of embodiment P1, wherein Ring B is a substituted or unsubstituted naphthyl.

Embodiment P61. The compound of embodiment P1, wherein Ring B is a substituted or unsubstituted 1-naphthyl.

Embodiment P62. The compound of embodiment P1, wherein Ring B is a substituted or unsubstituted 2-naphthyl.

Embodiment P63. The compound of embodiment P1, wherein Ring B is a substituted or unsubstituted quinolinyl.

Embodiment P64. The compound of embodiment P1, wherein Ring B is a substituted or unsubstituted 2-quinolinyl.

Embodiment P65. The compound of embodiment P1, wherein Ring B is a substituted or unsubstituted 3-quinolinyl.

Embodiment P66. The compound of embodiment P1, wherein Ring B is a substituted or unsubstituted 4-quinolinyl.

Embodiment P67. The compound of embodiment P1, wherein Ring B is a substituted or unsubstituted isoquinolinyl.

Embodiment P68. The compound of embodiment P1, wherein Ring B is a substituted or unsubstituted 1-isoquinolinyl.

Embodiment P69. The compound of embodiment P1, wherein Ring B is a substituted or unsubstituted 3-isoquinolinyl.

Embodiment P70. The compound of embodiment P1, wherein Ring B is a substituted or unsubstituted 4-isoquinolinyl.

Embodiment P71. The compound of embodiment P2, having the formula:

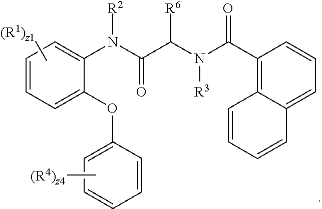

Embodiment P72. The compound of embodiment P2, having the formula:

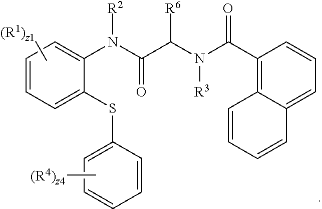

Embodiment P73. The compound of embodiment P2, having the formula:

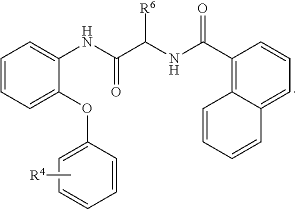

Embodiment P74. The compound of embodiment P2, having the formula:

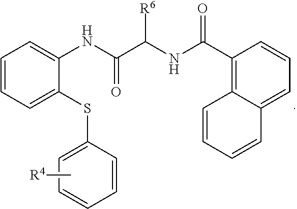

Embodiment P75. The compound of embodiment P1, having the formula:

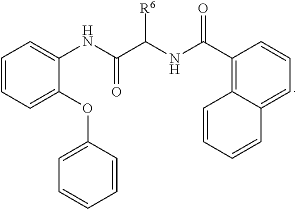

Embodiment P76. The compound of embodiment P1, having the formula:

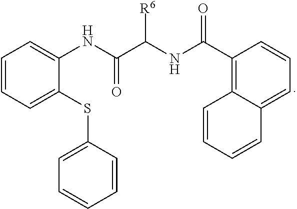

Embodiment P77. The compound of embodiment P2, having the formula:

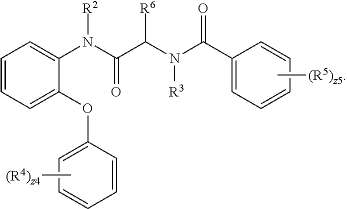

Embodiment P78. The compound of embodiment P2, having the formula:

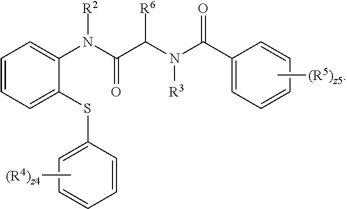

Embodiment P79. The compound of embodiment P2, having the formula:

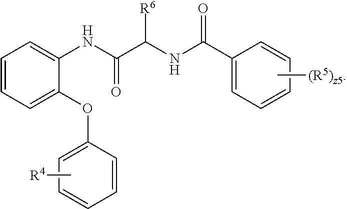

Embodiment P80. The compound of embodiment P1, having the formula:

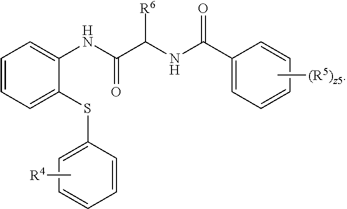

Embodiment P81. The compound of embodiment P2, having the formula:

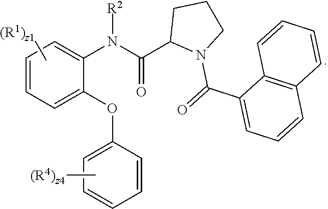

Embodiment P82. The compound of embodiment P2, having the formula:

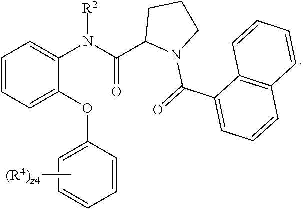

Embodiment P83. The compound of embodiment P2, having the formula:

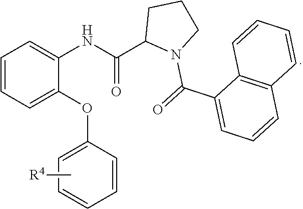

Embodiment P84. The compound of embodiment P2, having the formula:

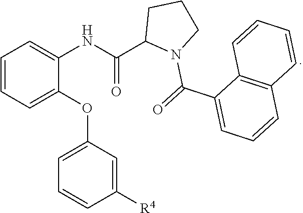

Embodiment P85. The compound of embodiment P1, having the formula:

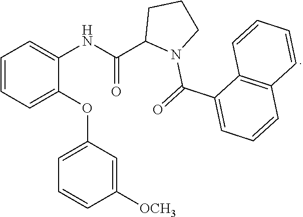 [SMILES: COc1cccc(Oc2ccccc2NC(=O)C2CCCN2C(=O)c2cccc3ccccc23)c1]

Embodiment P86. The compound of embodiment P1, having the formula:

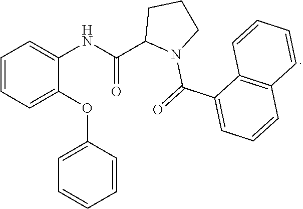 [SMILES: O=C(Nc1ccccc1Oc1ccccc1)C1CCCN1C(=O)c1cccc2ccccc12]

Embodiment P87. The compound embodiment P1, having the formula:

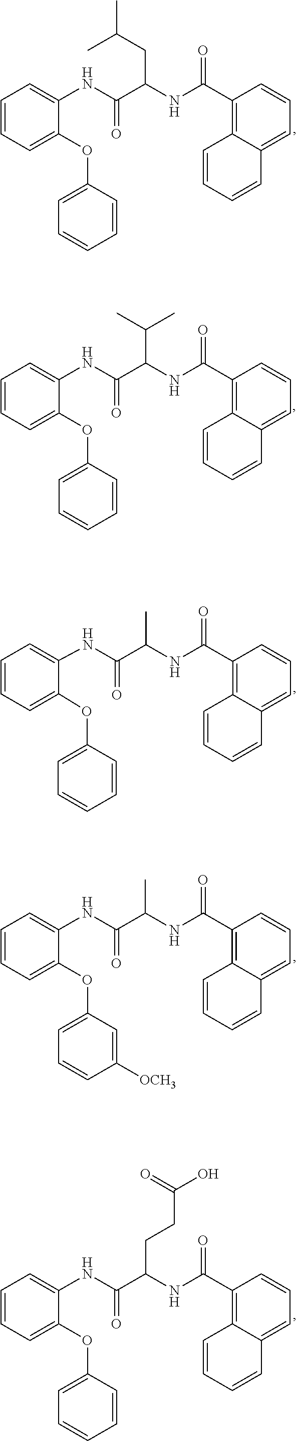 [SMILES: CC(C)CC(NC(=O)c1cccc2ccccc12)C(=O)Nc1ccccc1Oc1ccccc1 ; CC(C)C(NC(=O)c1cccc2ccccc12)C(=O)Nc1ccccc1Oc1ccccc1 ; CC(NC(=O)c1cccc2ccccc12)C(=O)Nc1ccccc1Oc1ccccc1 ; COc1cccc(Oc2ccccc2NC(=O)C(C)NC(=O)c2cccc3ccccc23)c1 ; O=C(O)CCC(NC(=O)c1cccc2ccccc12)C(=O)Nc1ccccc1Oc1ccccc1]

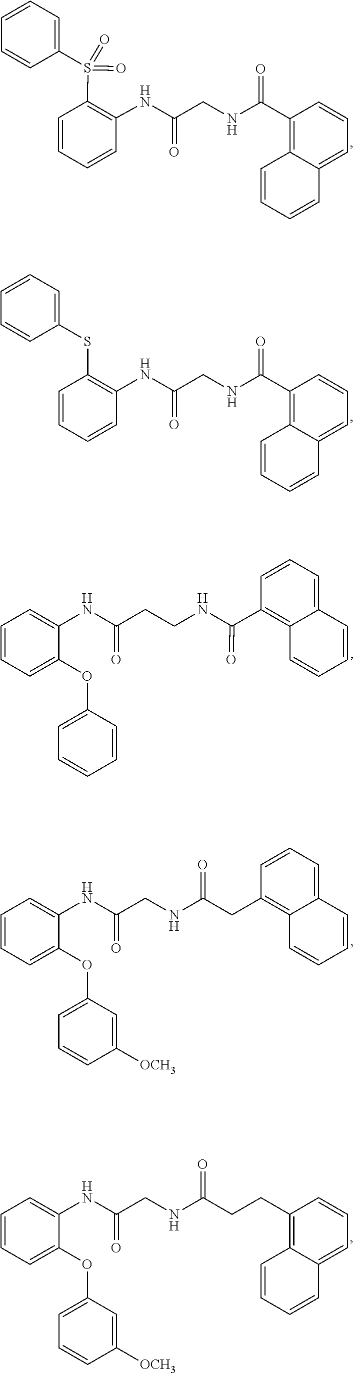 [SMILES: O=C(CNC(=O)c1cccc2ccccc12)Nc1ccccc1S(=O)(=O)c1ccccc1 ; O=C(CNC(=O)c1cccc2ccccc12)Nc1ccccc1Sc1ccccc1 ; O=C(CCNC(=O)c1cccc2ccccc12)Nc1ccccc1Oc1ccccc1 ; COc1cccc(Oc2ccccc2NC(=O)CNC(=O)Cc2cccc3ccccc23)c1 ; COc1cccc(Oc2ccccc2NC(=O)CNC(=O)CCc2cccc3ccccc23)c1]

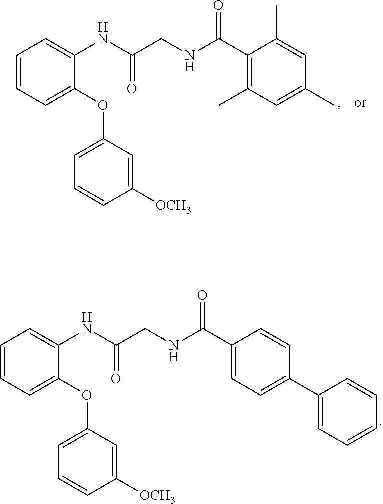 [SMILES: COc1cccc(Oc2ccccc2NC(=O)CNC(=O)c2c(C)cc(C)cc2C)c1 ; COc1cccc(Oc2ccccc2NC(=O)CNC(=O)c2ccc(-c3ccccc3)cc2)c1]

Embodiment P88. A pharmaceutical composition comprising a compound of one of embodiments P1 to P87 or a pharmaceutically acceptable salt thereof, and a pharmaceutically acceptable excipient.

Embodiment P89. The pharmaceutical composition of embodiment P88, further comprising an anti-cancer agent.

Embodiment P90. The pharmaceutical composition of embodiment P89, wherein the anti-cancer agent is a platinum-based compound, topoisomerase inhibitor, or Chk1 inhibitor.

Embodiment P91. The pharmaceutical composition of embodiment P89, wherein the anti-cancer agent is a cisplatin.

Embodiment P92. The pharmaceutical composition of embodiment P89, wherein the anti-cancer agent is etoposide, SN-38, camptothecin, or gemcitabine.

Embodiment P93. The pharmaceutical composition of embodiment P89, wherein the anti-cancer agent is CHIR-124, debromohymenialdisine, SB 218078, LY2603618, SCH900776, TCS 2312, PF 477736, UCN-01, or AZD7762.

Embodiment P94. A method of treating a disease associated with PCNA activity in a subject in need of such treatment, said method comprising administering a therapeutically effective amount of a compound of one of embodiments P1 to P87, or a pharmaceutically acceptable salt thereof.

Embodiment P95. A method of treating cancer in a subject in need of such treatment, said method comprising administering a therapeutically effective amount of a compound of one of embodiments P1 to P87, or a pharmaceutically acceptable salt thereof.

Embodiment P96. The method of embodiment P95, further comprising administering radiation.

Embodiment P97. The method of one of embodiments P95 to P96, wherein said cancer is a sarcoma, adenocarcinoma, leukemia, or lymphoma.

Embodiment P98. The method of one of embodiments P95 to P96, wherein said cancer is a lung cancer, colon cancer, central nervous system cancer, brain cancer, neuroblastoma, skin cancer, head and neck cancer, melanoma, ovarian cancer, renal cancer, prostate cancer, breast cancer, mesothelioma, liver cancer, stomach cancer, esophageal cancer, bladder cancer, cervical cancer, osteosarcoma, pancreatic cancer, adrenal cortical cancer, adrenal gland cancer, colorectal cancer, testicular cancer, myeloma, B-acute lymphoblastic lymphoma, non-Hodgkin's lymphoma, Hodgkin's lymphoma, chronic leukemia, acute leukemia, glandular carcinoma, or hematoid carcinoma.

Embodiment P99. A method of inhibiting PCNA activity, said method comprising contacting PCNA with an effective amount of a compound of one of embodiments P1 to P87, or a pharmaceutically acceptable salt thereof.

Embodiment P100. The method of embodiment P99, wherein the compound binds to His44, Val45, Leu47, Pro234, Tyr250, Leu251, Ala252, Met40, Leu47, Leu126, Leu128, Val233, Pro234, Ala252, Pro253, or Asp 232 of PCNA.

Embodiment P101. The method of embodiment P99, wherein the compound binds noncovalently to His44, Val45, Leu47, Pro234, Tyr250, Leu251, Ala252, Met40, Leu47, Leu126, Leu128, Val233, Pro234, Ala252, Pro253, or Asp 232 of PCNA.

Embodiment P102. A compound, or a pharmaceutically acceptable salt thereof, having the formula:

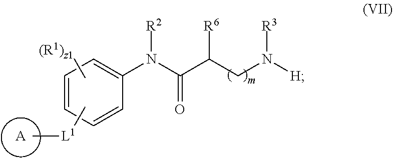

wherein
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F; and
m is an integer from 0 to 5.

Embodiment P103. The compound of embodiment P102, having the formula:

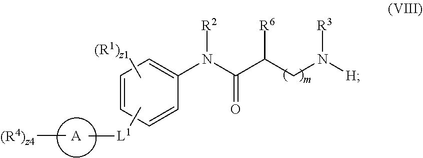

wherein
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, and R^{4D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
z4 is an integer from 0 to 5;
m4 and v4 are independently 1 or 2;
n4 is an integer from 0 to 4; and
X⁴ is independently —Cl, —Br, —I, or —F.

Embodiment P104. The compound of embodiment P103, having the formula:

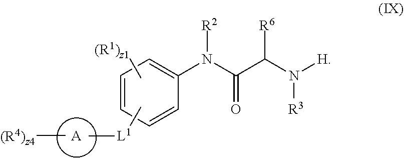

Embodiment P105. The compound of one of embodiments P102 to P103, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

Embodiment P106. The compound of one of embodiments P102 to P103, wherein L¹ is —O—.

Embodiment P107. The compound of one of embodiments P102 to P103, wherein L¹ is —S—.

Embodiment P108. The compound of one of embodiments P102 to P103, wherein L¹ is —S(O)₂—.

Embodiment P109. The compound of one of embodiments P102 to P108, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P110. The compound of one of embodiments P102 to P108, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment P111. The compound of one of embodiments P102 to P110, wherein z1 is 1.

Embodiment P112. The compound of one of embodiments P102 to P108, wherein z1 is 0.

Embodiment P113. The compound of one of embodiments P102 to P112, wherein R² is hydrogen, unsubstituted methyl, unsubstituted ethyl, or unsubstituted isopropyl.

Embodiment P114. The compound of one of embodiments P102 to P113, wherein R³ is hydrogen, unsubstituted methyl, unsubstituted ethyl, or unsubstituted isopropyl.

Embodiment P115. The compound of one of embodiments P102 to P114, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P116. The compound of one of embodiments P102 to P114, wherein R⁶ is substituted or unsubstituted C₁-C₆ alkyl or substituted or unsubstituted 2 to 6 membered heteroalkyl.

Embodiment P117. The compound of one of embodiments P102 to P114, wherein R⁶ is hydrogen, unsubstituted methyl, unsubstituted isopropyl,

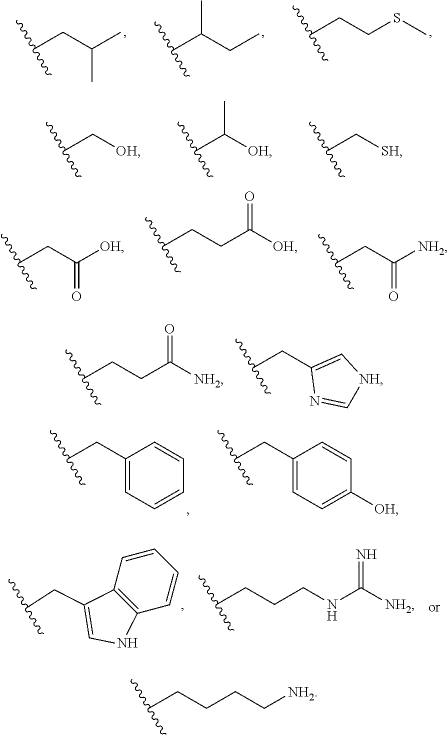

Embodiment P118. The compound of one of embodiments P103 to P117, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P119. The compound of one of embodiments P103 to P117, wherein R⁴ is independently halogen, —CF₃, CHF₂, —CH₂F, —OCF₃, —OCHF₂, —OCH₂F, —OH, unsubstituted methyl, or unsubstituted methoxy.

Embodiment P120. The compound of one of embodiments P103 to P117, wherein R⁴ is independently —OR^{4D}.

Embodiment P121. The compound of embodiment P120, wherein R^{4D} is hydrogen or substituted or unsubstituted alkyl.

Embodiment P122. The compound of embodiment P120, wherein R^{4D} is hydrogen or unsubstituted C₁-C₅ alkyl.

Embodiment P123. The compound of embodiment P120, wherein R^{4D} is unsubstituted methyl.

Embodiment P124. The compound of one of embodiments P103 to P123, wherein z4 is 1.

Embodiment P125. The compound of one of embodiments P103 to P117, wherein z4 is 0.

Embodiment P126. The compound of one of embodiments P103 to P125, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P127. The compound of one of embodiments P103 to P125, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

Embodiment P128. The compound of one of embodiments P103 to P127, wherein z5 is 1.

Embodiment P129. The compound of one of embodiments P103 to P125, wherein z5 is 0.

Embodiment P130. The compound of embodiment P102, wherein Ring A is a substituted or unsubstituted phenyl.

Embodiment P131. The compound of embodiment P102, wherein Ring A is a substituted or unsubstituted 5 to 6 membered heteroaryl.

Embodiment P132. The compound of embodiment P102, wherein Ring A is a substituted or unsubstituted thienyl.

Embodiment P133. The compound of embodiment P102, wherein Ring A is a substituted or unsubstituted pyridyl.

Embodiment P134. A method of making compound (I), or a pharmaceutically acceptable salt thereof, said method comprising mixing compound (VII) and compound (X) together in a reaction vessel; wherein
compound (I) has the formula:

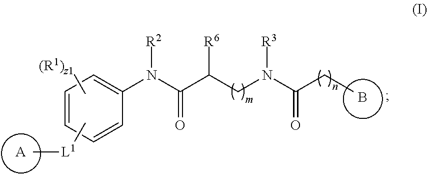

compound (VII) has the formula:

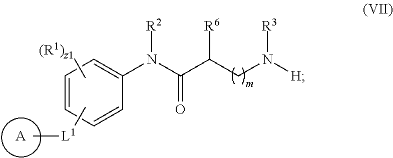

compound (X) has the formula:

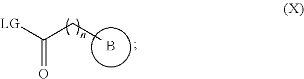

L¹ is —O—, —NR⁷—, —S—, —C(O)—, —C(O)O—, —OC(O)—, —NR¹C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
Ring B is substituted or unsubstituted phenyl, substituted or unsubstituted naphthyl, substituted or unsubstituted quinolinyl, or substituted or unsubstituted isoquinolinyl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F;
m is an integer from 0 to 5;
n is an integer from 0 to 10; and
LG is a leaving group.

Embodiment P135. The method of embodiment P134, wherein LG is halogen.

Embodiment P136. The method of embodiment P134, wherein LG is —Cl.

Embodiment P137. The method of one of embodiments P134 to P137, further comprising a base.

Embodiment P138. The method of embodiment P137, wherein the base is N,N-diisopropylethylamine.

Embodiment P139. The method of embodiment P134, wherein LG is —OH.

Embodiment P140. The method of embodiment P134 or P139, further comprising a peptide coupling agent.

Embodiment P141. The method of embodiment P140, wherein the peptide coupling agent is dicyclohexylcarbodiimide.

Embodiment P142. The method of embodiment P140, wherein the peptide coupling agent is HBTU.

Embodiment P143. The method of embodiment P140, wherein the peptide coupling agent is HOBt.

Embodiment P144. The method of embodiment P140, wherein the peptide coupling agent is PyBOP.

Embodiment P145. The method of one of embodiments P134 to P142, wherein
compound (I) is a compound of formula (II):

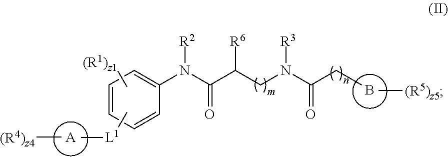

compound (VII) is a compound of formula (VIII):

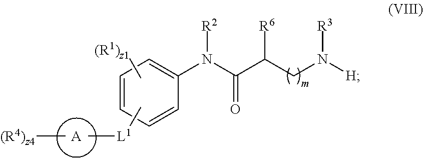

compound (X) is a compound of formula (XI):

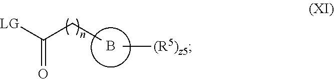

R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX₅³, —CHX⁵₂, —CH₂X⁵, —OCX₅³, —OCHX³₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
z4 is an integer from 0 to 5;
z5 is an integer from 0 to 7;
m4, m5, v4, and v5 are independently 1 or 2;
n4 and n5 are independently an integer from 0 to 4; and
X⁴ and X⁵ are independently —Cl, —Br, —I, or —F.

Embodiment P146. The method of one of embodiments P134 to P145, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

Embodiment P147. The method of one of embodiments P134 to P145, wherein L¹ is —O—.

Embodiment P148. The method of one of embodiments P134 to P145, wherein L¹ is —S—.

Embodiment P149. The method of one of embodiments P134 to P145, wherein L¹ is —S(O)₂—.

Embodiment P150. The method of one of embodiments P134 to P149, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P151. The method of one of embodiments P134 to P149, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment P152. The method of one of embodiments P134 to P151, wherein z1 is 1.

Embodiment P153. The method of one of embodiments P134 to P149, wherein z1 is 0.

Embodiment P154. The method of one of embodiments P134 to P153, wherein R² is hydrogen, unsubstituted methyl, unsubstituted ethyl, or unsubstituted isopropyl.

Embodiment P155. The method of one of embodiments P134 to P153, wherein R³ is hydrogen, unsubstituted methyl, unsubstituted ethyl, or unsubstituted isopropyl.

Embodiment P156. The method of one of embodiments P134 to P155, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P157. The method of one of embodiments P134 to P155, wherein R⁶ is substituted or unsubstituted C₁-C₆ alkyl or substituted or unsubstituted 2 to 6 membered heteroalkyl.

Embodiment P158. The method of one of embodiments P134 to P155, wherein R⁶ is hydrogen, unsubstituted methyl, unsubstituted isopropyl,

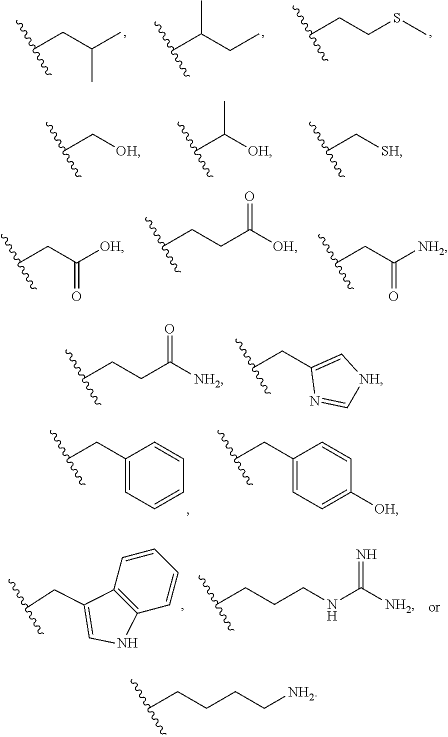

Embodiment P159. The method of one of embodiments P134 to P158, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P160. The method of one of embodiments P134 to P158, wherein R⁴ is independently halogen, —CF₃, —CHF₂, —CH₂F, —OCF₃, —OCHF₂, —OCH₂F, —OH, unsubstituted methyl, or unsubstituted methoxy.

Embodiment P161. The method of one of embodiments P134 to P158, wherein R⁴ is independently —OR^{4D}.

Embodiment P162. The method of embodiment P161, wherein R^{4D} is hydrogen or substituted or unsubstituted alkyl.

Embodiment P163. The method of embodiment P161, wherein R^{4D} is hydrogen or unsubstituted C₁-C₅ alkyl.

Embodiment P164. The method of embodiment P161, wherein R^{4D} is unsubstituted methyl.

Embodiment P165. The method of one of embodiments P134 to P164, wherein z4 is 1.

Embodiment P166. The method of one of embodiments P134 to P158, wherein z4 is 0.

Embodiment P167. The method of one of embodiments P134 to P166, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment P168. The method of one of embodiments P134 to P166, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

Embodiment P169. The method of one of embodiments P134 to P168, wherein z5 is 1.

Embodiment P170. The method of one of embodiments P134 to P166, wherein z5 is 0.

Embodiment P171. The method of embodiment P134, wherein Ring A is a substituted or unsubstituted phenyl.

Embodiment P172. The method of embodiment P134, wherein Ring A is a substituted or unsubstituted 5 to 6 membered heteroaryl.

Embodiment P173. The method of embodiment P134, wherein Ring A is a substituted or unsubstituted thienyl.

Embodiment P174. The method of embodiment P134, wherein Ring A is a substituted or unsubstituted pyridyl.

Embodiment P175. The method of embodiment P134, wherein Ring B is a substituted or unsubstituted phenyl.

Embodiment P176. The method of embodiment P134, wherein Ring B is a substituted or unsubstituted naphthyl.

Embodiment P177. The method of embodiment P134, wherein Ring B is a substituted or unsubstituted quinolinyl.

Embodiment P178. The method of embodiment P134, wherein Ring B is a substituted or unsubstituted isoquinolinyl.

### VII. Additional Embodiments

Embodiment 1. A compound, or a pharmaceutically acceptable salt thereof, having the formula:

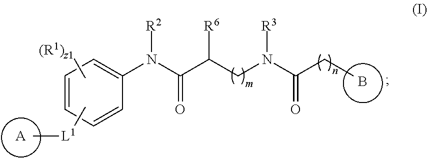

wherein
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
Ring B is substituted or unsubstituted phenyl, substituted or unsubstituted naphthyl, substituted or unsubstituted quinolinyl, or substituted or unsubstituted isoquinolinyl;
R¹ is independently halogen, —CX¹₃, —CHX¹₂, —CH₂X¹, —OCX¹₃, —OCHX¹₂, —OCH₂X¹, —CN, —SOⁿ¹R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F;
m is an integer from 0 to 5; and
n is an integer from 0 to 10;
provided when L¹ is —O—, m is 0, n is 0, and Ring B is substituted or unsubstituted naphthyl, substituted or unsubstituted quinolinyl, or substituted or unsubstituted isoquinolinyl, then R⁶ is not hydrogen.

Embodiment 2. The compound of embodiment 1, having the formula:

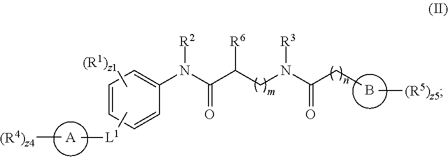

wherein
Ring A is phenyl or 5 to 6 membered heteroaryl;
Ring B is phenyl, naphthyl, quinolinyl, or isoquinolinyl;
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX₅³, —CHX⁵₂, —CH₂X⁵, —OCX₅³, —OCHX³₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
z4 is an integer from 0 to 5;
z5 is an integer from 0 to 7;
m4, m5, v4, and v5 are independently 1 or 2;
n4 and n5 are independently an integer from 0 to 4; and
X⁴ and X⁵ are independently —Cl, —Br, —I, or —F.

Embodiment 3. The compound of embodiment 2, having the formula:

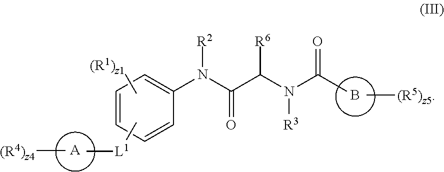

Embodiment 4. The compound of embodiment 3, having the formula:

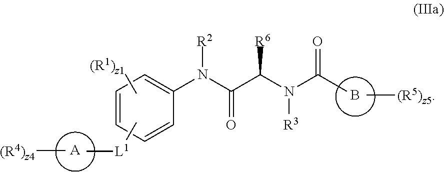

Embodiment 5. The compound of embodiment 3, having the formula:

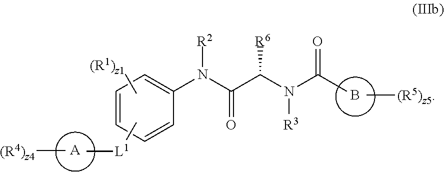

Embodiment 6. The compound of embodiment 3, having the formula:

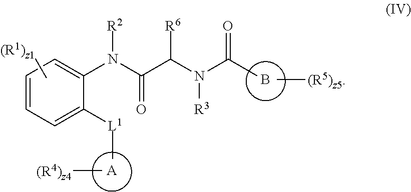

Embodiment 7. The compound of embodiment 3, having the formula:

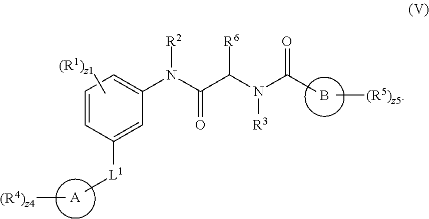

Embodiment 8. The compound of embodiment 3, having the formula:

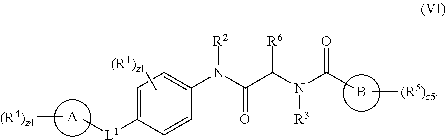

Embodiment 9. The compound of one of embodiments 1 to 8, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

Embodiment 10. The compound of one of embodiments 1 to 8, wherein L¹ is —O—.

Embodiment 11. The compound of one of embodiments 1 to 8, wherein L¹ is —S—.

Embodiment 12. The compound of one of embodiments 1 to 8, wherein L¹ is —S(O)₂—.

Embodiment 13. The compound of one of embodiments 1 to 12, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 14. The compound of one of embodiments 1 to 12, wherein R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 15. The compound of one of embodiments 1 to 12, wherein R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment 16. The compound of one of embodiments 1 to 12, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment 17. The compound of one of embodiments 1 to 16, wherein z1 is 1.

Embodiment 18. The compound of one of embodiments 1 to 12, wherein z1 is 0.

Embodiment 19. The compound of one of embodiments 1 to 18, wherein R² is hydrogen, —CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 20. The compound of one of embodiments 1 to 18, wherein R² is hydrogen, unsubstituted methyl, unsubstituted ethyl, or unsubstituted isopropyl.

Embodiment 21. The compound of one of embodiments 1 to 18, wherein R² is hydrogen.

Embodiment 22. The compound of one of embodiments 1 to 21, wherein R³ is hydrogen, —CX³₃, —CHX³₂, —CH₂X³, —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 23. The compound of one of embodiments 1 to 21, wherein R³ is hydrogen, unsubstituted methyl, unsubstituted ethyl, or unsubstituted isopropyl.

Embodiment 24. The compound of one of embodiments 1 to 21, wherein R³ is hydrogen.

Embodiment 25. The compound of one of embodiments 1 to 24, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 26. The compound of one of embodiments 1 to 24, wherein R⁶ is substituted or unsubstituted C₁-C₆ alkyl or substituted or unsubstituted 2 to 6 membered heteroalkyl.

Embodiment 27. The compound of one of embodiments 1 to 24, wherein R⁶ is hydrogen, unsubstituted methyl, unsubstituted isopropyl,

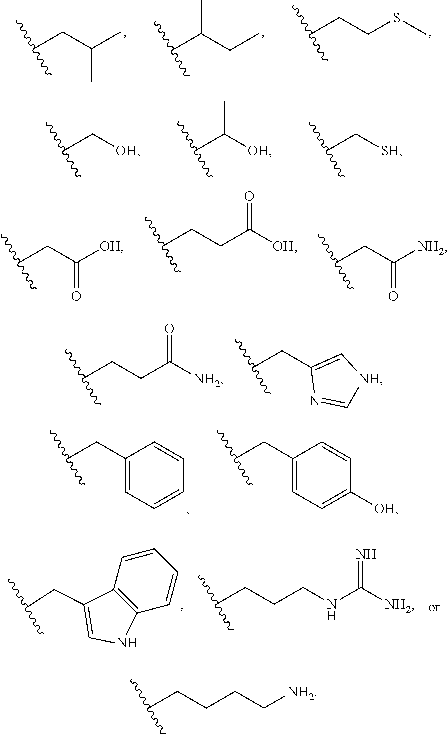

Embodiment 28. The compound of one of embodiments 1 to 21, wherein R³ and R⁶ are joined to form a substituted or unsubstituted heterocycloalkyl or substituted or unsubstituted heteroaryl.

Embodiment 29. The compound of one of embodiments 1 to 21, wherein R³ and R⁶ are joined to form a substituted or unsubstituted 4 to 8 membered heterocycloalkyl.

Embodiment 30. The compound of one of embodiments 1 to 21, wherein R³ and R⁶ are joined to form an unsubstituted pyrrolidinyl.

Embodiment 31. The compound of one of embodiments 1 to 21, wherein R³ and R⁶ are joined to form an unsubstituted piperidinyl.

Embodiment 32. The compound of one of embodiments 2 to 31, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 33. The compound of one of embodiments 2 to 31, wherein R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 34. The compound of one of embodiments 2 to 31, wherein R⁴ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

Embodiment 35. The compound of one of embodiments 2 to 31, wherein R⁴ is independently halogen, —CF₃, —CHF₂, —CH₂F, —OCF₃, —OCHF₂, —OCH₂F, —OH, unsubstituted methyl, or unsubstituted methoxy.

Embodiment 36. The compound of one of embodiments 2 to 31, wherein R⁴ is independently —OR^{4D}.

Embodiment 37. The compound of embodiment 36, wherein R^{4D} is hydrogen or substituted or unsubstituted alkyl.

Embodiment 38. The compound of embodiment 36, wherein R^{4D} is hydrogen or unsubstituted alkyl.

Embodiment 39. The compound of embodiment 36, wherein R^{4D} is hydrogen or unsubstituted C₁-C₅ alkyl.

Embodiment 40. The compound of embodiment 36, wherein R^{4D} is hydrogen or unsubstituted methyl.

Embodiment 41. The compound of embodiment 36, wherein R^{4D} is unsubstituted methyl.

Embodiment 42. The compound of one of embodiments 2 to 41, wherein z4 is 1.

Embodiment 43. The compound of one of embodiments 2 to 31, wherein z4 is 0.

Embodiment 44. The compound of one of embodiments 2 to 43, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 45. The compound of one of embodiments 2 to 43, wherein R⁵ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

Embodiment 46. The compound of one of embodiments 2 to 43, wherein R⁵ is independently halogen, —CF₃, —CHF₂, —CH₂F, —OCF₃, —OCHF₂, —OCH₂F, —OH, —NH₂, —SH, unsubstituted C₁-C₄ alkyl, unsubstituted 2 to 4 membered heteroalkyl, or unsubstituted phenyl.

Embodiment 47. The compound of one of embodiments 2 to 43, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

Embodiment 48. The compound of one of embodiments 2 to 47, wherein z5 is 1.

Embodiment 49. The compound of one of embodiments 2 to 43, wherein z5 is 0.

Embodiment 50. The compound of embodiment 1, wherein Ring A is a substituted or unsubstituted phenyl.

Embodiment 51. The compound of embodiment 1, wherein Ring A is a substituted or unsubstituted 5 to 6 membered heteroaryl.

Embodiment 52. The compound of embodiment 1, wherein Ring A is a substituted or unsubstituted thienyl.

Embodiment 53. The compound of embodiment 1, wherein Ring A is a substituted or unsubstituted 2-thienyl.

Embodiment 54. The compound of embodiment 1, wherein Ring A is a substituted or unsubstituted 3-thienyl.

Embodiment 55. The compound of embodiment 1, wherein Ring A is a substituted or unsubstituted pyridyl.

Embodiment 56. The compound of embodiment 1, wherein Ring A is a substituted or unsubstituted 2-pyridyl.

Embodiment 57. The compound of embodiment 1, wherein Ring A is a substituted or unsubstituted 3-pyridyl.

Embodiment 58. The compound of embodiment 1, wherein Ring A is a substituted or unsubstituted 4-pyridyl.

Embodiment 59. The compound of embodiment 1, wherein Ring B is a substituted or unsubstituted phenyl.

Embodiment 60. The compound of embodiment 1, wherein Ring B is a substituted or unsubstituted naphthyl.

Embodiment 61. The compound of embodiment 1, wherein Ring B is a substituted or unsubstituted 1-naphthyl.

Embodiment 62. The compound of embodiment 1, wherein Ring B is a substituted or unsubstituted 2-naphthyl.

Embodiment 63. The compound of embodiment 1, wherein Ring B is a substituted or unsubstituted quinolinyl.

Embodiment 64. The compound of embodiment 1, wherein Ring B is a substituted or unsubstituted 2-quinolinyl.

Embodiment 65. The compound of embodiment 1, wherein Ring B is a substituted or unsubstituted 3-quinolinyl.

Embodiment 66. The compound of embodiment 1, wherein Ring B is a substituted or unsubstituted 4-quinolinyl.

Embodiment 67. The compound of embodiment 1, wherein Ring B is a substituted or unsubstituted isoquinolinyl.

Embodiment 68. The compound of embodiment 1, wherein Ring B is a substituted or unsubstituted 1-isoquinolinyl.

Embodiment 69. The compound of embodiment 1, wherein Ring B is a substituted or unsubstituted 3-isoquinolinyl.

Embodiment 70. The compound of embodiment 1, wherein Ring B is a substituted or unsubstituted 4-isoquinolinyl.

Embodiment 71. The compound of embodiment 2, having the formula:

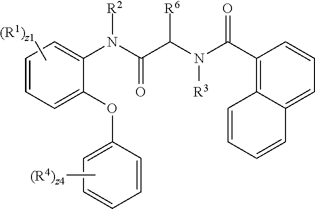

Embodiment 72. The compound of embodiment 2, having the formula:

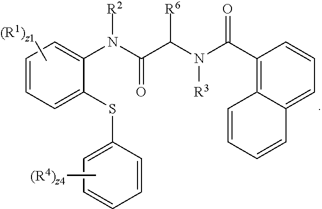

Embodiment 73. The compound of embodiment 2, having the formula:

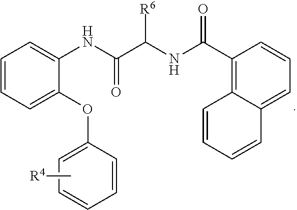

Embodiment 74. The compound of embodiment 2, having the formula:

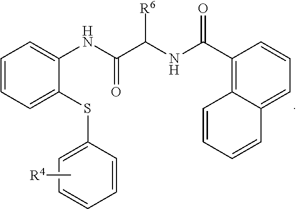

Embodiment 75. The compound of embodiment 1, having the formula:

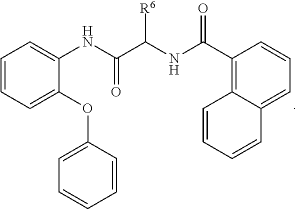

Embodiment 76. The compound of embodiment 1, having the formula:

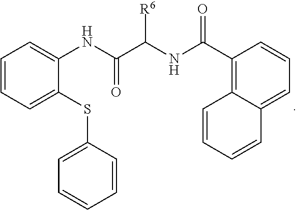

Embodiment 77. The compound of embodiment 2, having the formula:

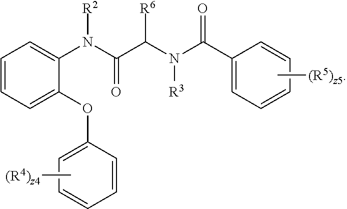

Embodiment 78. The compound of embodiment 2, having the formula:

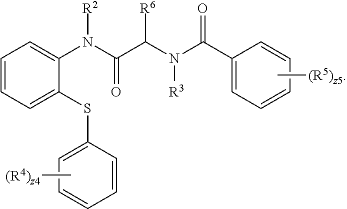

Embodiment 79. The compound of embodiment 2, having the formula:

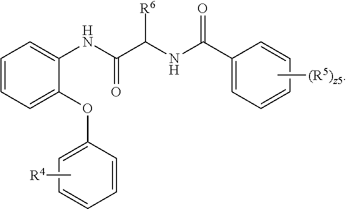

Embodiment 80. The compound of embodiment 1, having the formula:

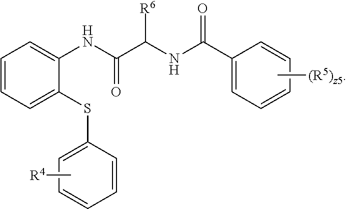

Embodiment 81. The compound of embodiment 2, having the formula:

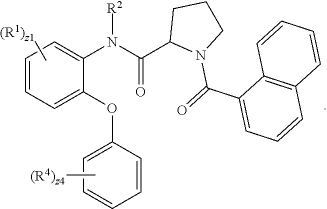

Embodiment 82. The compound of embodiment 2, having the formula:

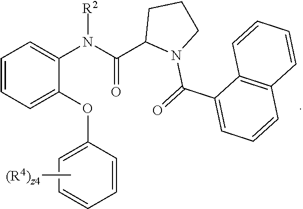

Embodiment 83. The compound of embodiment 2, having the formula:

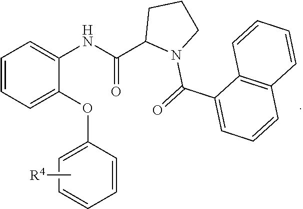

Embodiment 84. The compound of embodiment 2, having the formula:

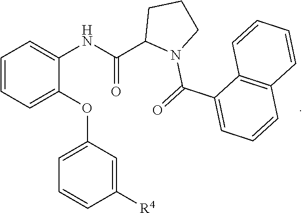

Embodiment 85. The compound of embodiment 1, having the formula:

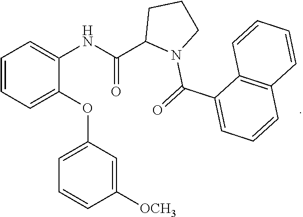 [SMILES: COc1cccc(Oc2ccccc2NC(=O)C2CCCN2C(=O)c2cccc3ccccc23)c1]

Embodiment 86. The compound of embodiment 1, having the formula:

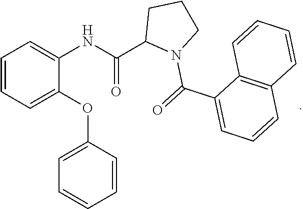 [SMILES: O=C(Nc1ccccc1Oc1ccccc1)C1CCCN1C(=O)c1cccc2ccccc12]

Embodiment 87. The compound embodiment 1, having the formula:

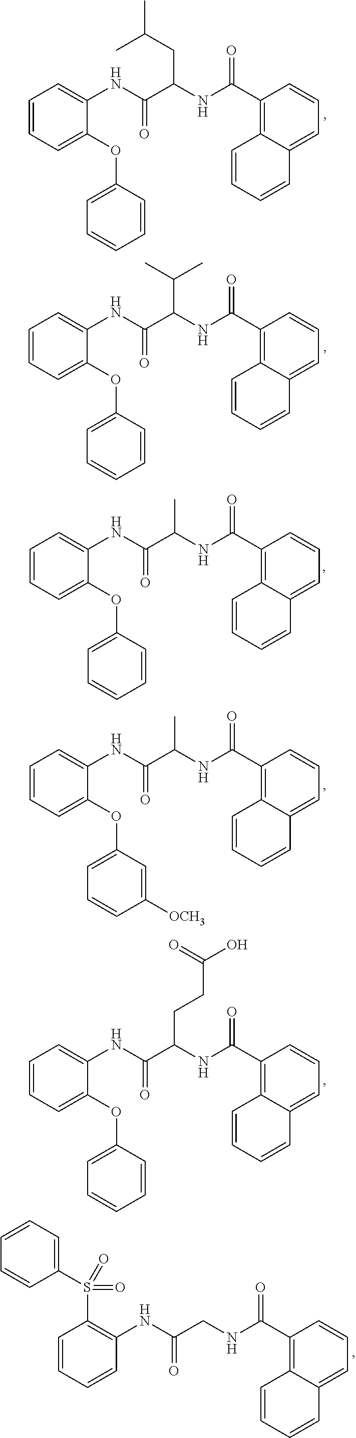

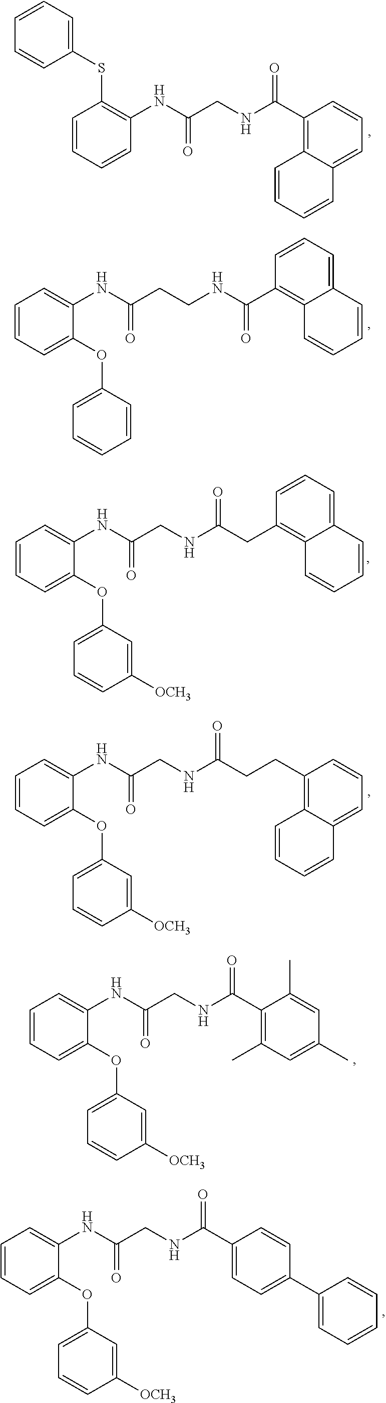

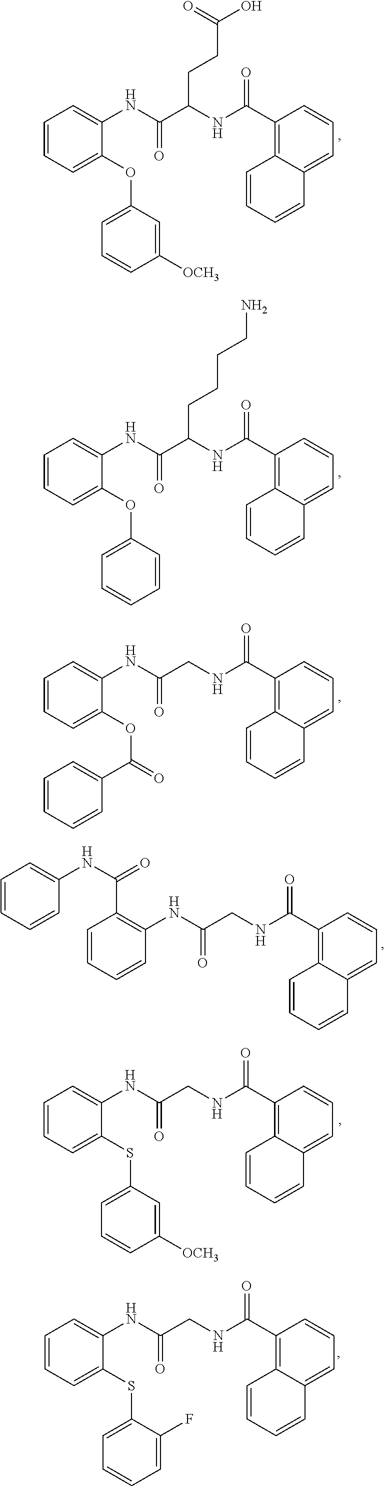

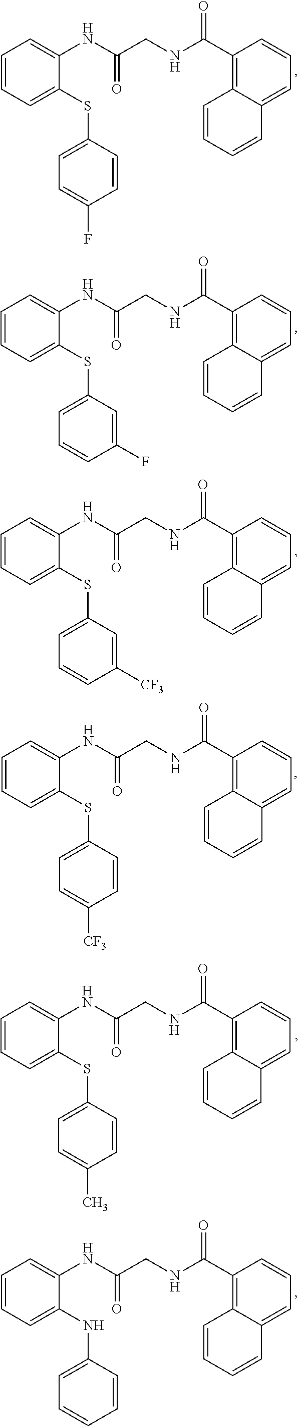 [SMILES: O=C(CNC(=O)c1cccc2ccccc12)Nc1ccccc1Sc1ccc(F)cc1 ; O=C(CNC(=O)c1cccc2ccccc12)Nc1ccccc1Sc1cccc(F)c1 ; O=C(CNC(=O)c1cccc2ccccc12)Nc1ccccc1Sc1cccc(C(F)(F)F)c1 ; O=C(CNC(=O)c1cccc2ccccc12)Nc1ccccc1Sc1ccc(C(F)(F)F)cc1 ; Cc1ccc(Sc2ccccc2NC(=O)CNC(=O)c2cccc3ccccc23)cc1 ; O=C(CNC(=O)c1cccc2ccccc12)Nc1ccccc1Nc1ccccc1]

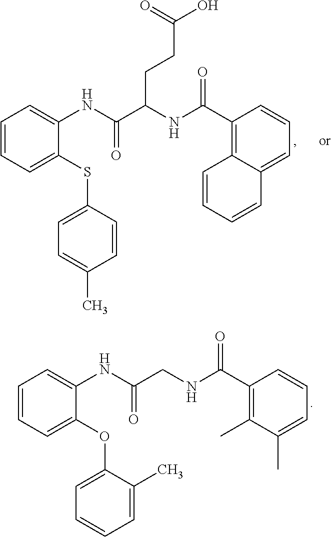 [SMILES: Cc1ccc(Sc2ccccc2NC(=O)C(CCC(=O)O)NC(=O)c2cccc3ccccc23)cc1 ; Cc1ccccc1Oc1ccccc1NC(=O)CNC(=O)c1cccc(C)c1C]

Embodiment 88. A pharmaceutical composition comprising a compound of one of embodiments 1 to 87 or a pharmaceutically acceptable salt thereof, and a pharmaceutically acceptable excipient.

Embodiment 89. The pharmaceutical composition of embodiment 88, further comprising an anti-cancer agent.

Embodiment 90. The pharmaceutical composition of embodiment 89, wherein the anti-cancer agent is a platinum-based compound, topoisomerase inhibitor, or Chk1 inhibitor.

Embodiment 91. The pharmaceutical composition of embodiment 89, wherein the anti-cancer agent is a cisplatin.

Embodiment 92. The pharmaceutical composition of embodiment 89, wherein the anti-cancer agent is etoposide, SN-38, camptothecin, or gemcitabine.

Embodiment 93. The pharmaceutical composition of embodiment 89, wherein the anti-cancer agent is CHIR-124, debromohymenialdisine, SB 218078, LY2603618, SCH900776, TCS 2312, PF 477736, UCN-01, or AZD7762.

Embodiment 94. A method of treating a disease associated with PCNA activity in a subject in need of such treatment, said method comprising administering a therapeutically effective amount of a compound of one of embodiments 1 to 87, or a pharmaceutically acceptable salt thereof.

Embodiment 95. A method of treating cancer in a subject in need of such treatment, said method comprising administering a therapeutically effective amount of a compound of one of embodiments 1 to 87, or a pharmaceutically acceptable salt thereof.

Embodiment 96. The method of embodiment 95, further comprising administering radiation.

Embodiment 97. The method of one of embodiments 95 to 96, wherein said cancer is a sarcoma, adenocarcinoma, leukemia, or lymphoma.

Embodiment 98. The method of one of embodiments 95 to 96, wherein said cancer is a lung cancer, colon cancer, central nervous system cancer, brain cancer, neuroblastoma, skin cancer, head and neck cancer, melanoma, ovarian cancer, renal cancer, prostate cancer, breast cancer, mesothelioma, liver cancer, stomach cancer, esophageal cancer, bladder cancer, cervical cancer, osteosarcoma, pancreatic cancer, adrenal cortical cancer, adrenal gland cancer, colorectal cancer, testicular cancer, myeloma, B-acute lymphoblastic lymphoma, non-Hodgkin's lymphoma, Hodgkin's lymphoma, chronic leukemia, acute leukemia, glandular carcinoma, or hematoid carcinoma.

Embodiment 99. A method of inhibiting PCNA activity, said method comprising contacting PCNA with an effective amount of a compound of one of embodiments 1 to 87, or a pharmaceutically acceptable salt thereof.

Embodiment 100. The method of embodiment 99, wherein the compound binds to His44, Val45, Leu47, Pro234, Tyr250, Leu251, Ala252, Met40, Leu47, Leu126, Leu128, Val233, Pro234, Ala252, Pro253, or Asp 232 of PCNA.

Embodiment 101. The method of embodiment 99, wherein the compound binds noncovalently to His44, Val45, Leu47, Pro234, Tyr250, Leu251, Ala252, Met40, Leu47, Leu126, Leu128, Val233, Pro234, Ala252, Pro253, or Asp 232 of PCNA.

Embodiment 102. A compound, or a pharmaceutically acceptable salt thereof, having the formula:

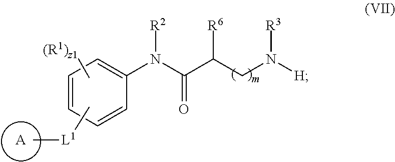

wherein
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; RIA and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F; and
m is an integer from 0 to 5.

Embodiment 103. The compound of embodiment 102, having the formula:

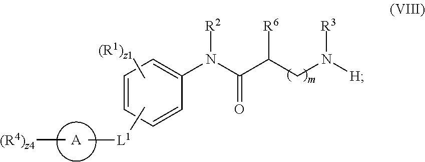

wherein
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, and R^{4D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
z4 is an integer from 0 to 5;
m4 and v4 are independently 1 or 2;
n4 is an integer from 0 to 4; and
X⁴ is independently —Cl, —Br, —I, or —F.

Embodiment 104. The compound of embodiment 103, having the formula:

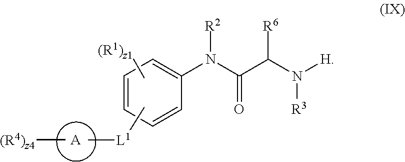

Embodiment 105. The compound of one of embodiments 102 to 103, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

Embodiment 106. The compound of one of embodiments 102 to 103, wherein L¹ is —O—.

Embodiment 107. The compound of one of embodiments 102 to 103, wherein L¹ is —S—.

Embodiment 108. The compound of one of embodiments 102 to 103, wherein L¹ is —S(O)₂—.

Embodiment 109. The compound of one of embodiments 102 to 108, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 110. The compound of one of embodiments 102 to 108, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment 111. The compound of one of embodiments 102 to 110, wherein z1 is 1.

Embodiment 112. The compound of one of embodiments 102 to 108, wherein z1 is 0.

Embodiment 113. The compound of one of embodiments 102 to 112, wherein R² is hydrogen, unsubstituted methyl, unsubstituted ethyl, or unsubstituted isopropyl.

Embodiment 114. The compound of one of embodiments 102 to 113, wherein R³ is hydrogen, unsubstituted methyl, unsubstituted ethyl, or unsubstituted isopropyl.

Embodiment 115. The compound of one of embodiments 102 to 114, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 116. The compound of one of embodiments 102 to 114, wherein R⁶ is substituted or unsubstituted C₁-C₆ alkyl or substituted or unsubstituted 2 to 6 membered heteroalkyl.

Embodiment 117. The compound of one of embodiments 102 to 114, wherein R⁶ is hydrogen, unsubstituted methyl, unsubstituted isopropyl,

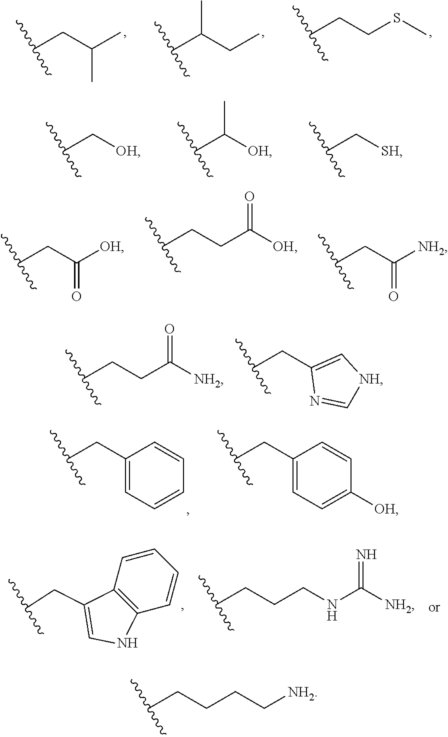

Embodiment 118. The compound of one of embodiments 103 to 117, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 119. The compound of one of embodiments 103 to 117, wherein R⁴ is independently halogen, —CF₃, —CHF₂, —CH₂F, —OCF₃, —OCHF₂, —OCH₂F, —OH, unsubstituted methyl, or unsubstituted methoxy.

Embodiment 120. The compound of one of embodiments 103 to 117, wherein R⁴ is independently —OR^{4D}.

Embodiment 121. The compound of embodiment 120, wherein R^{4D} is hydrogen or substituted or unsubstituted alkyl.

Embodiment 122. The compound of embodiment 120, wherein R^{4D} is hydrogen or unsubstituted C₁-C₅ alkyl.

Embodiment 123. The compound of embodiment 120, wherein R^{4D} is unsubstituted methyl.

Embodiment 124. The compound of one of embodiments 103 to 123, wherein z4 is 1.

Embodiment 125. The compound of one of embodiments 103 to 117, wherein z4 is 0.

Embodiment 126. The compound of one of embodiments 103 to 125, wherein R⁵ is independently halogen, —CF₃, —CHF₂, CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 127. The compound of one of embodiments 103 to 125, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

Embodiment 128. The compound of one of embodiments 103 to 127, wherein z5 is 1.

Embodiment 129. The compound of one of embodiments 103 to 125, wherein z5 is 0.

Embodiment 130. The compound of embodiment 102, wherein Ring A is a substituted or unsubstituted phenyl.

Embodiment 131. The compound of embodiment 102, wherein Ring A is a substituted or unsubstituted 5 to 6 membered heteroaryl.

Embodiment 132. The compound of embodiment 102, wherein Ring A is a substituted or unsubstituted thienyl.

Embodiment 133. The compound of embodiment 102, wherein Ring A is a substituted or unsubstituted pyridyl.

Embodiment 134. A method of making compound (I), or a pharmaceutically acceptable salt thereof, said method comprising mixing compound (VII) and compound (X) together in a reaction vessel; wherein
compound (I) has the formula:

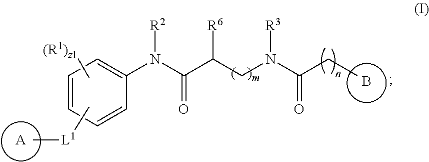

compound (VII) has the formula:

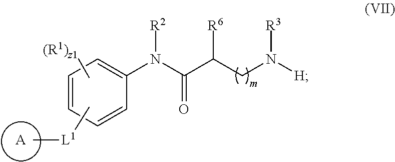

compound (X) has the formula:

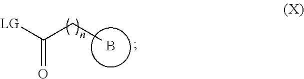

L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
Ring B is substituted or unsubstituted phenyl, substituted or unsubstituted naphthyl, substituted or unsubstituted quinolinyl, or substituted or unsubstituted isoquinolinyl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F;
m is an integer from 0 to 5;
n is an integer from 0 to 10; and
LG is a leaving group.

Embodiment 135. The method of embodiment 134, wherein LG is halogen.

Embodiment 136. The method of embodiment 134, wherein LG is —Cl.

Embodiment 137. The method of one of embodiments 134 to 138, further comprising a base.

Embodiment 138. The method of embodiment 137, wherein the base is N,N-diisopropylethylamine.

Embodiment 139. The method of embodiment 134, wherein LG is —OH.

Embodiment 140. The method of embodiment 134 or 139, further comprising a peptide coupling agent.

Embodiment 141. The method of embodiment 140, wherein the peptide coupling agent is dicyclohexylcarbodiimide.

Embodiment 142. The method of embodiment 140, wherein the peptide coupling agent is HBTU.

Embodiment 143. The method of embodiment 140, wherein the peptide coupling agent is HOBt.

Embodiment 144. The method of embodiment 140, wherein the peptide coupling agent is PyBOP.

Embodiment 145. The method of one of embodiments 134 to 142, wherein
compound (I) is a compound of formula (II):

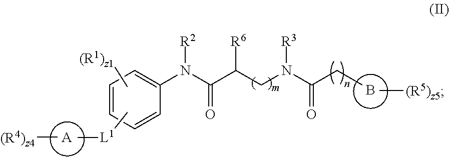

compound (VII) is a compound of formula (VIII):

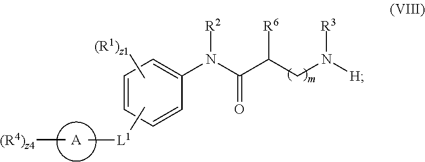

compound (X) is a compound of formula (XI):

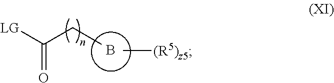

R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX₅³, —CHX⁵₂, —CH₂X⁵, —OCX₅³, —OCHX⁵₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
z4 is an integer from 0 to 5;
z5 is an integer from 0 to 7;
m4, m5, v4, and v5 are independently 1 or 2;
n4 and n5 are independently an integer from 0 to 4; and
X⁴ and X⁵ are independently —Cl, —Br, —I, or —F.

Embodiment 146. The method of one of embodiments 134 to 145, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

Embodiment 147. The method of one of embodiments 134 to 145, wherein L¹ is —O—.

Embodiment 148. The method of one of embodiments 134 to 145, wherein L¹ is —S—.

Embodiment 149. The method of one of embodiments 134 to 145, wherein L¹ is —S(O)₂—.

Embodiment 150. The method of one of embodiments 134 to 149, wherein R¹ is independently halogen, —CF₃, CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 151. The method of one of embodiments 134 to 149, wherein R¹ is independently halogen, —OH, —CF₃, —CHF₂, —CH₂F, —OCF₃, —OCHF₂, —OCH₂F, unsubstituted methyl, or unsubstituted methoxy.

Embodiment 152. The method of one of embodiments 134 to 151, wherein z1 is 1.

Embodiment 153. The method of one of embodiments 134 to 149, wherein z1 is 0.

Embodiment 154. The method of one of embodiments 134 to 153, wherein R² is hydrogen, unsubstituted methyl, unsubstituted ethyl, or unsubstituted isopropyl.

Embodiment 155. The method of one of embodiments 134 to 153, wherein R³ is hydrogen, unsubstituted methyl, unsubstituted ethyl, or unsubstituted isopropyl.

Embodiment 156. The method of one of embodiments 134 to 155, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 157. The method of one of embodiments 134 to 155, wherein R⁶ is substituted or unsubstituted C₁-C₆ alkyl or substituted or unsubstituted 2 to 6 membered heteroalkyl.

Embodiment 158. The method of one of embodiments 134 to 155, wherein R⁶ is hydrogen, unsubstituted methyl, unsubstituted isopropyl,

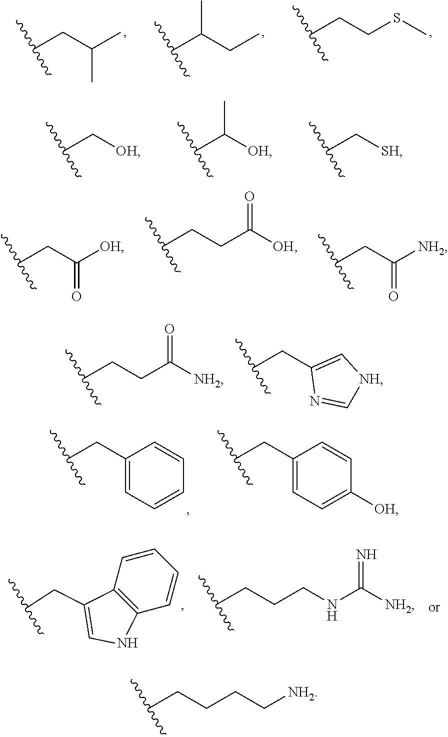

Embodiment 159. The method of one of embodiments 145 to 158, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 160. The method of one of embodiments 145 to 158, wherein R⁴ is independently halogen, —CF₃, —CHF₂, —CH₂F, —OCF₃, —OCHF₂, —OCH₂F, —OH, unsubstituted methyl, or unsubstituted methoxy.

Embodiment 161. The method of one of embodiments 145 to 158, wherein R⁴ is independently —OR^{4D}.

Embodiment 162. The method of embodiment 161, wherein R^{4D} is hydrogen or substituted or unsubstituted alkyl.

Embodiment 163. The method of embodiment 161, wherein R^{4D} is hydrogen or unsubstituted C₁-C₅ alkyl.

Embodiment 164. The method of embodiment 161, wherein R^{4D} is unsubstituted methyl.

Embodiment 165. The method of one of embodiments 145 to 164, wherein z4 is 1.

Embodiment 166. The method of one of embodiments 145 to 158, wherein z4 is 0.

Embodiment 167. The method of one of embodiments 145 to 166, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

Embodiment 168. The method of one of embodiments 145 to 166, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

Embodiment 169. The method of one of embodiments 145 to 168, wherein z5 is 1.

Embodiment 170. The method of one of embodiments 145 to 166, wherein z5 is 0.

Embodiment 171. The method of embodiment 134, wherein Ring A is a substituted or unsubstituted phenyl.

Embodiment 172. The method of embodiment 134, wherein Ring A is a substituted or unsubstituted 5 to 6 membered heteroaryl.

Embodiment 173. The method of embodiment 134, wherein Ring A is a substituted or unsubstituted thienyl.

Embodiment 174. The method of embodiment 134, wherein Ring A is a substituted or unsubstituted pyridyl.

Embodiment 175. The method of embodiment 134, wherein Ring B is a substituted or unsubstituted phenyl.

Embodiment 176. The method of embodiment 134, wherein Ring B is a substituted or unsubstituted naphthyl.

Embodiment 177. The method of embodiment 134, wherein Ring B is a substituted or unsubstituted quinolinyl.

Embodiment 178. The method of embodiment 134, wherein Ring B is a substituted or unsubstituted isoquinolinyl.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Compounds Targeting Cancer-Associated PCNA

We present herein, inter alia, a novel small molecule-based caPCNA inhibitor, AOH1996, which obtained from a comprehensive SAR study in the lead optimization step and appears suitable for clinical studies. AOH1996 selectively kills cancer cells, and it appears to induce replication stress, promotes apoptosis and increases cancer cell sensitivity to genotoxic agents, while these effects are not observed in nonmalignant cell controls. AOH1996 is orally administrable, metabolic stable and it suppresses tumor growth as a monotherapy or as a combination treatment, but it causes no discernable side effects at more than six times its effective dose. In studying the molecular mechanisms of the compound, we determined that it binds into the PIP-box binding pocket of PCNA. This binding enhances the interaction between PCNA and the largest subunit of RNA polymerase II, RPB1, and thereby dissociating PCNA from actively transcribed chromatin regions, and inducing DNA double stranded breaks in a transcription dependent manner in cancer cells. Attenuation of RPB1 interaction with PCNA by a single amino acid mutation in RPB1's PCNA-binding APIM motif confers resistance to AOH1996. PCNA plays a critical role in temporarily dislodging RNA polymerase II from transcription replication conflict sites, to enable the replication fork to proceed. Thus, small molecule inhibitors of transcription replication conflict resolution, such as AOH1996, may provide a novel therapeutic avenue for exploiting this cancer-selective vulnerability.

Proliferating cell nuclear antigen (PCNA) is an evolutionarily conserved multifaceted protein found in all eukaryotic cells, and it plays a critical role in DNA synthesis and in DNA repair. PCNA forms a homotrimeric ring structure encircling DNA (Krishna et al., 1994) and it acts as a central “hub” of the replisome, to provide an anchorage for the many proteins involved in the replication and repair pathways. The cellular functions of PCNA can be modulated through post-translational modifications on the surface of the protein, altering partner interactions (Tsutakawa et al., 2011; Tsutakawa et al., 2015) that occur predominantly through the outer hydrophobic surface of PCNA, adjacent to its inter-domain connector loop (IDCL) (Chapados et al., 2004; Sebesta et al., 2017). Historically, PCNA has been widely used as tumor progression marker and more recent studies have demonstrated that PCNA can play a mitogenic role, to distantly rejuvenate senescent cells via extracellular vesicles (Lei et al., 2021).

DNA replication stress is a hallmark of cancer cells (Hanahan and Weinberg, 2000; Hanahan and Weinberg, 2011) and a major anti-cancer therapeutic strategy is to exploit this cancer-associated feature by introducing further DNA damage in a catastrophic manner. Due to its central roles in replication and repair, PCNA is a potential target for this anti-cancer strategy. Moreover, the identification of a distinct isoform of PCNA associated with cancer cells has potentially opened a novel avenue for the development of new chemotherapeutics. Early effects in targeting PCNA have identified several molecules of interest, both small molecule and peptide-based, which have indicated that directly targeting PCNA for cancer therapy may be a viable approach (Gu et al., 2014; Muller et al., 2013; Punchihewa et al., 2012; Tan et al., 2012; Waga et al., 1994; Yu et al., 2013; Zhao et al., 2011).

Here, we describe both the identification and detailed molecular characterization of AOH1996 that exhibits remarkable therapeutic properties. It is orally administrable in a formulation compatible to clinical use, and it nearly completely inhibits the growth of xenograft tumors and sensitizes them to topoisomerase inhibition in animal studies. In studies that follow the good laboratory practice (GLP) guidelines of the US Food and Drug Administration (FDA) AOH1996 causes no discernible toxicity under at least 6 times the effective dose in mice and dogs.

Our molecular characterizations include the structure of PCNA in complex with a more soluble analog suitable for crystallization experiments, AOH1160LE, which revealed that this compound binds into the PCNA PIP box. In cells, AOH1996 was observed to stabilize the interaction between chromatin-bound PCNA and the largest subunit (RBP1) of RNAPII, leading to degradation of the intracellular RPB1. AOH1996 also dissociates PCNA from actively transcribed chromatin and causes DSB accumulation without affecting the presence of PCNA in the heterochromatin region, suggesting a transcription-associated collapse of DNA replication. Both transcription inhibition and point mutation in the APIM domain (Gilljam et al., 2009) of RPB1 that weakens its interaction with PCNA confer resistance to AOH1996.

Transcription-replication conflicts (TRC) constitute a major intrinsic cause of DSB and genome instability (Gaillard and Aguilera, 2016; Helmrich et al., 2013). Given that transcription and replication are essential cellular processes, and that cancer cells likely enhance encounters between the transcription and replication machineries, this may make cancer cells more susceptible to disruption of TRC resolution. Accumulating evidence indicates that TRC resolution involves removing RNA polymerase II (RNAPII) from the conflict sites, by backtracking or degradation of RNAPII, to allow the replication fork to go through (Helmrich et al., 2013; Li et al., 2018). Therefore, our results demonstrate that PCNA and RBP1 interaction is cancer-selective vulnerability in preclinical models. Our results also demonstrate the therapeutic potential of AOH1996 as a monotherapy, as well as in combination with existing chemotherapies, and its potential usefulness as a chemical tool to further define TRCs in cells.

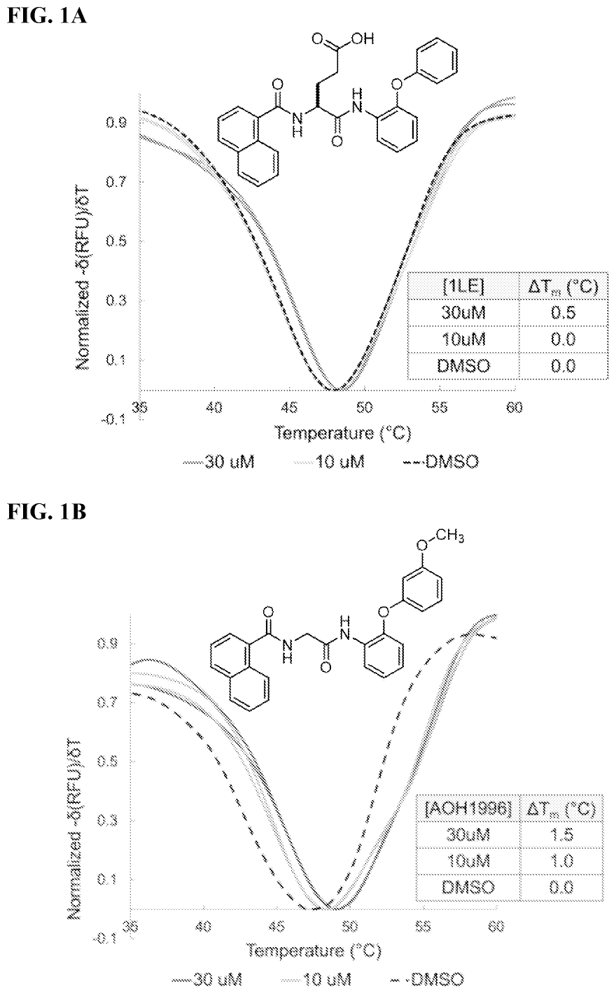

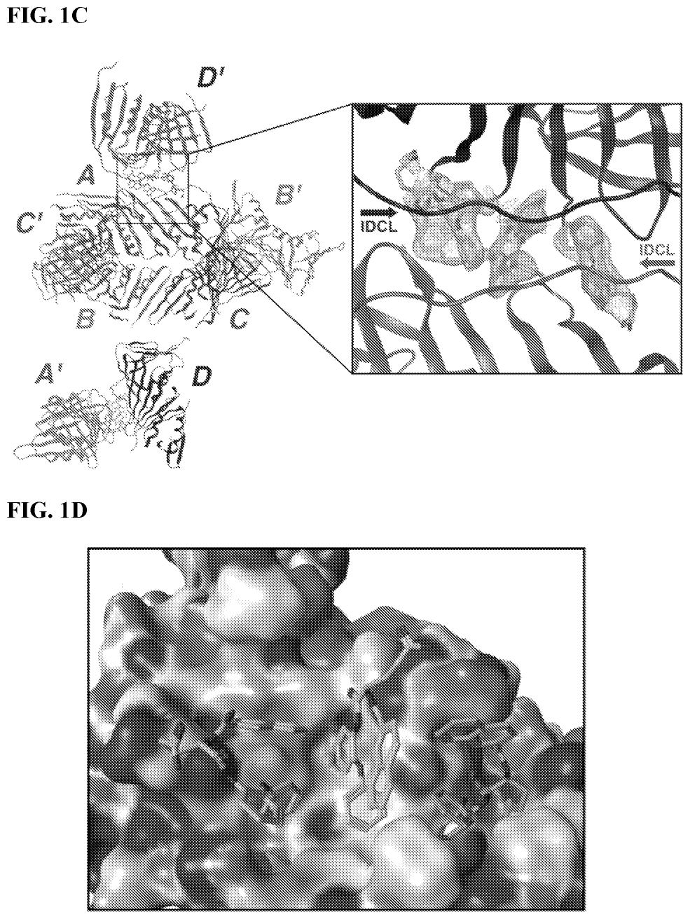

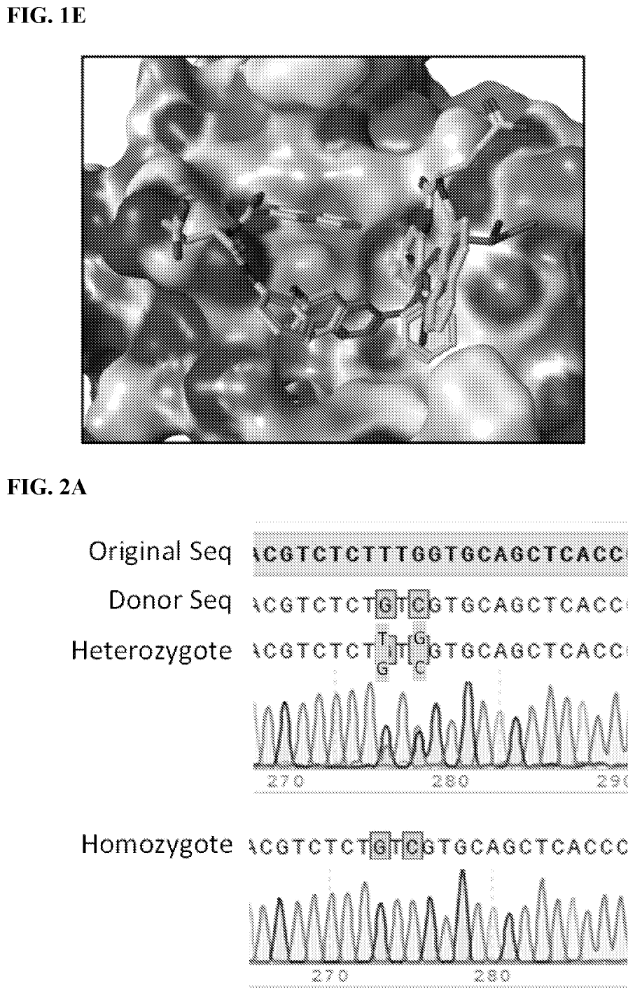

Targeting the PIP box and APIM binding domain of PCNA. AOH1160, a small molecule PCNA ligand, targets the cancer-distinct L126-Y133 region of PCNA (Malkas et al., 2006) and is selectively toxic to cancer cells (Gu et al., 2018). By modeling the detailed molecular interactions between PCNA and its potential ligands using the All-Around-Docking methodology (Friesner et al., 2006), we rationally designed ˜70 drug-like AOH1160 analogs in the lead optimization step that all three components of the parent molecule (1-naphthoyl, diphenyl ether, and glycine linker) were systematically modified for structure-activity relationship (SAR) study. In the case of 1-naphthoyl group, we performed a nitrogen walk (using isoquinoline-1-carbonyl, isoquinoline-4-carbonyl) and also replaced 1-naphthoyl with other monocyclic and bicyclic aromatic groups such as 2,4,6-trimethylbenzoyl, 2-(naphthalen-1-yl) acetyl, 3-(naphthalen-1-yl) propanoyl, and [1,1′-biphenyl]-4-carbonyl. Similarly, a nitrogen atom was introduced to the para position of the terminal phenyl ring and different substituents such as chlorine, hydroxy and methoxy groups were introduced to both rings of diphenyl ether. Also, the bridging oxygen were replaced by sulfur and sulfonyl groups. Moreover, the glycine linker was replaced with different natural and unnatural amino acids. After chemical synthesis (Schemes 1-3 in Example 2) and screening of the potent drug-like lead analogs (Table 1, Table 2) we identified AOH1160LE (FIG. 1A), with a predicted significant increase in solubility and AOH1996 (FIG. 1B), which is derived by adding a methoxy group to the meta position of the terminal phenyl ring of AOH1160 and is thereby metabolically more stable than AOH1160. To confirm binding of AOH1160LE and AOH1996 to PCNA, we performed a thermal denaturation analysis, which is based on the principle that the binding of low molecular weight ligands can increase the thermal stability of their target proteins (Koshland, 1958). The dose dependent protein melting curves shifted by as much as 0.5° C. for AOH1160LE and 1.5° C. for AOH1996 (FIGS. 1A-1B), indicating stabilizing interactions with PCNA.

TABLE 1
Screening of the potent AOH1160 analogs.

| Compound | Chemical structure | IC50 values |
|---|---|---|
| AOH1160 | 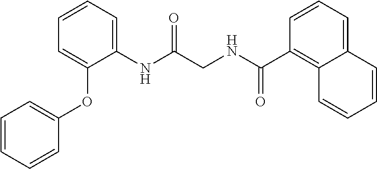 [SMILES: O=C(CNC(=O)c1cccc2ccccc12)Nc1ccccc1Oc1ccccc1] | SK-N-AS: 107 nM SK-N-DZ: 237 nM SK-N-BE(2)c: 325 nM |
| AOH1996 | 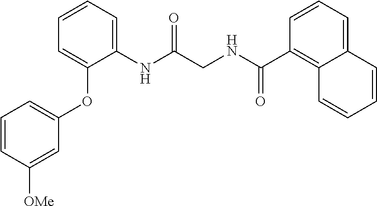 [SMILES: COc1cccc(Oc2ccccc2NC(=O)CNC(=O)c2cccc3ccccc23)c1] | SK-N-AS: 125 nM SK-N-DZ: 288 nM SK-N-BE(2)c: 236 nM MDA-MB-468: 740 nM HTC-116: 250 nM OVACAR8: 549 nM RKO: 255 nM HCT-116: 786 nM |
| AOH1996-4′OMe | 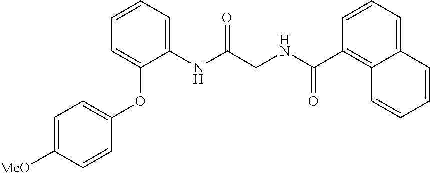 [SMILES: COc1ccc(Oc2ccccc2NC(=O)CNC(=O)c2cccc3ccccc23)cc1] | SK-N-AS: 91 nM SK-N-DZ: 271 nM SK-N-BE(2)c: 234 nM |
| AOH1160-4-C1 | 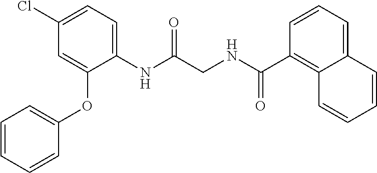 [SMILES: O=C(CNC(=O)c1cccc2ccccc12)Nc1ccc(Cl)cc1Oc1ccccc1] | SK-N-AS: 185 nM SK-N-DZ: 447 nM SK-N-BE(2)c: 500 nM |
| AOH1160-2-C1 | 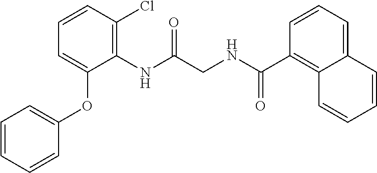 [SMILES: O=C(CNC(=O)c1cccc2ccccc12)Nc1c(Cl)cccc1Oc1ccccc1] | SK-N-AS: >10 uM SK-N-DZ: >10 uM SK-N-BE(2)c: >10 uM |
| AOH1160-3′-OH | 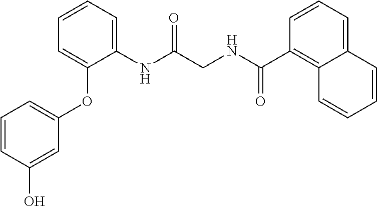 [SMILES: O=C(CNC(=O)c1cccc2ccccc12)Nc1ccccc1Oc1cccc(O)c1] | SK-N-AS: 192 nM SK-N-DZ: 539 nM SK-N-BE(2)c: 485 nM |
| AOH1160-4′-OH | 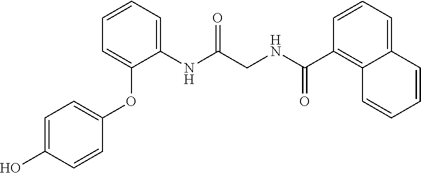 [SMILES: O=C(CNC(=O)c1cccc2ccccc12)Nc1ccccc1Oc1ccc(O)cc1] | SK-N-AS: 156 nM SK-N-DZ: 467 nM SK-N-BE(2)c: 490 nM |
| AOH1162 | 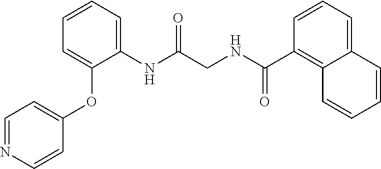 [SMILES: O=C(CNC(=O)c1cccc2ccccc12)Nc1ccccc1Oc1ccncc1] | >50 uM |
| AOH1161 | 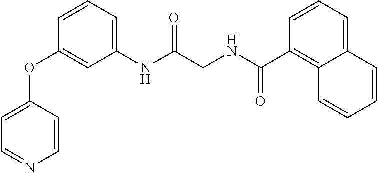 [SMILES: O=C(CNC(=O)c1cccc2ccccc12)Nc1cccc(Oc2ccncc2)c1] | 21.6 uM |
| AOH1178 | 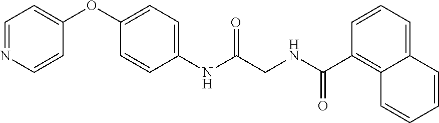 [SMILES: O=C(CNC(=O)c1cccc2ccccc12)Nc1ccc(Oc2ccncc2)cc1] | 1.1 uM |
| AOH1166 | 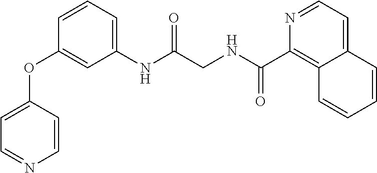 [SMILES: O=C(CNC(=O)c1nccc2ccccc12)Nc1cccc(Oc2ccncc2)c1] | 34 uM |
| AOH1179 | 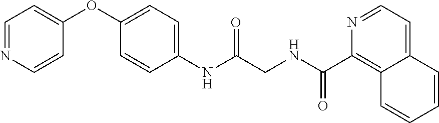 [SMILES: O=C(CNC(=O)c1nccc2ccccc12)Nc1ccc(Oc2ccncc2)cc1] | >30 uM |
| AOH1176 | 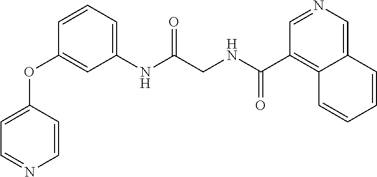 [SMILES: O=C(CNC(=O)c1cncc2ccccc12)Nc1cccc(Oc2ccncc2)c1] | >30 uM |
| AOH1180 | 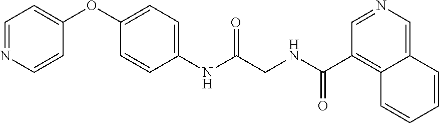 [SMILES: O=C(CNC(=O)c1cncc2ccccc12)Nc1ccc(Oc2ccncc2)cc1] | >30 uM |
| AOH1167 | 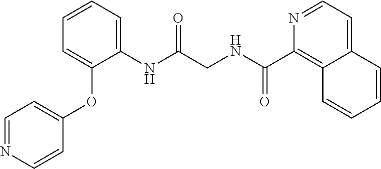 [SMILES: O=C(CNC(=O)c1nccc2ccccc12)Nc1ccccc1Oc1ccncc1] | 42 uM |
| AOH1177 | 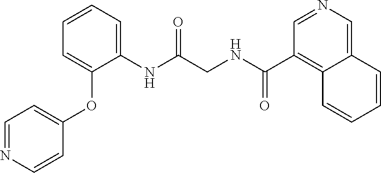 [SMILES: O=C(CNC(=O)c1cncc2ccccc12)Nc1ccccc1Oc1ccncc1] | >30 uM |
| AOH1165 | 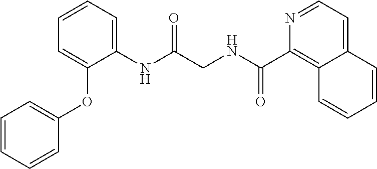 [SMILES: O=C(CNC(=O)c1nccc2ccccc12)Nc1ccccc1Oc1ccccc1] | 35 uM |
| AOH1175 | 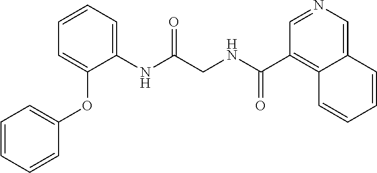 [SMILES: O=C(CNC(=O)c1cncc2ccccc12)Nc1ccccc1Oc1ccccc1] | >30 uM |
| AOH1996eNph | 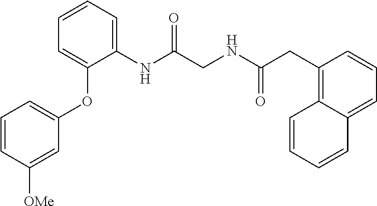 [SMILES: COc1cccc(Oc2ccccc2NC(=O)CNC(=O)Cc2cccc3ccccc23)c1] | >10 uM |
| AOH1996eeNph | 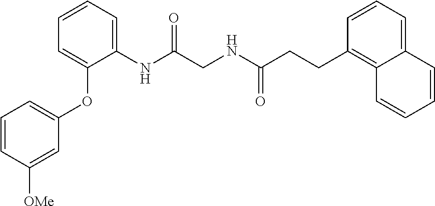 [SMILES: COc1cccc(Oc2ccccc2NC(=O)CNC(=O)CCc2cccc3ccccc23)c1] | >10 uM |
| AOH1996TMB | 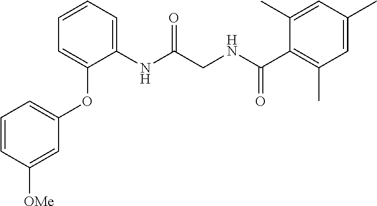 [SMILES: COc1cccc(Oc2ccccc2NC(=O)CNC(=O)c2c(C)cc(C)cc2C)c1] | Hela: 6.5 uM A673: 5.5 uM A549: 4.7 uM |
| AOH1996BiNph | 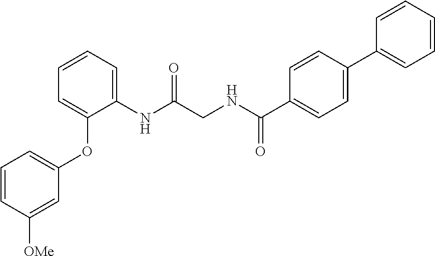 [SMILES: COc1cccc(Oc2ccccc2NC(=O)CNC(=O)c2ccc(-c3ccccc3)cc2)c1] | >10 uM |
| AOH1996t | 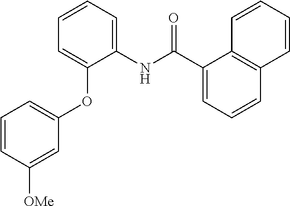 [SMILES: COc1cccc(Oc2ccccc2NC(=O)c2cccc3ccccc23)c1] | >10 uM |
| AOH1160t | 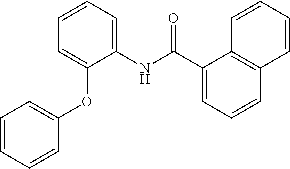 [SMILES: O=C(Nc1ccccc1Oc1ccccc1)c1cccc2ccccc12] | >10 uM |
| AOH1160SO2 | 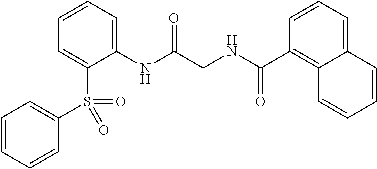 [SMILES: O=C(CNC(=O)c1cccc2ccccc12)Nc1ccccc1S(=O)(=O)c1ccccc1] | >10 uM |
| AOH1160S | 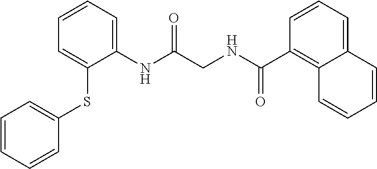 [SMILES: O=C(CNC(=O)c1cccc2ccccc12)Nc1ccccc1Sc1ccccc1] | MDA-MB-468: 316 nM Hela: 0.29 uM SK-MEL-5: 0.61 uM SK-MEL-28: 0.34 uM |
| AOH1160LA | 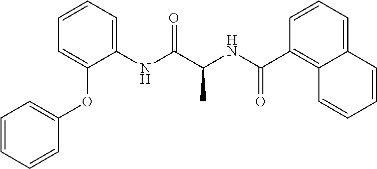 [SMILES: C[C@H](NC(=O)c1cccc2ccccc12)C(=O)Nc1ccccc1Oc1ccccc1] | MDA-MB-468: 4.9 uM SK-N-FI: 3 uM SH-SY5Y: 4 uM |
| AOH1996LA | 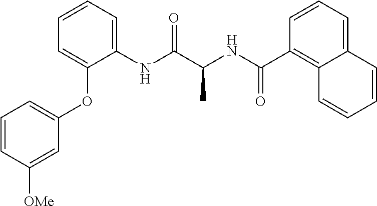 [SMILES: COc1cccc(Oc2ccccc2NC(=O)[C@H](C)NC(=O)c2cccc3ccccc23)c1] | MDA-MB-468: 2.3 uM SK-N-AS: 1.5 uM H1753: 1.6 uM H358: 1.8 uM |
| AOH1160e | 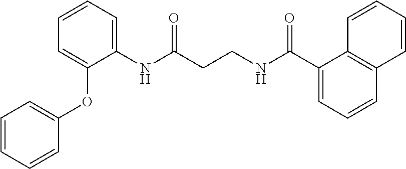 [SMILES: O=C(CCNC(=O)c1cccc2ccccc12)Nc1ccccc1Oc1ccccc1] | >10 uM |
| AOH1160LV | 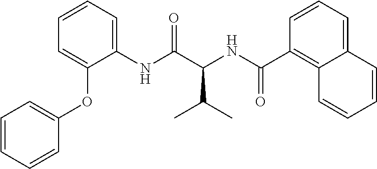 [SMILES: CC(C)[C@H](NC(=O)c1cccc2ccccc12)C(=O)Nc1ccccc1Oc1ccccc1] | >10 uM |
| AOH1160DV | 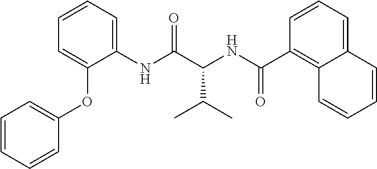 [SMILES: CC(C)[C@@H](NC(=O)c1cccc2ccccc12)C(=O)Nc1ccccc1Oc1ccccc1] | >10 uM |
| AOH1160LE | 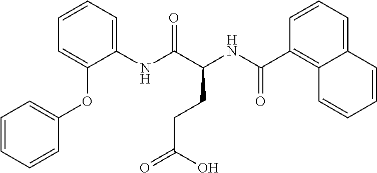 [SMILES: O=C(O)CC[C@H](NC(=O)c1cccc2ccccc12)C(=O)Nc1ccccc1Oc1ccccc1] | >10 uM |
| AOH1160DE | 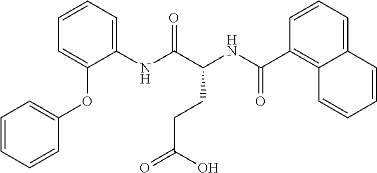 [SMILES: O=C(O)CC[C@@H](NC(=O)c1cccc2ccccc12)C(=O)Nc1ccccc1Oc1ccccc1] | >10 uM |
| AOH1160LK | 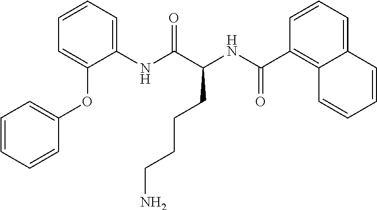 [SMILES: NCCCC[C@H](NC(=O)c1cccc2ccccc12)C(=O)Nc1ccccc1Oc1ccccc1] | >10 uM |
| AOH1160DK | 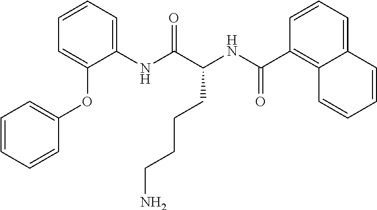 [SMILES: NCCCC[C@@H](NC(=O)c1cccc2ccccc12)C(=O)Nc1ccccc1Oc1ccccc1] | >10 uM |
| AOH1996LE | 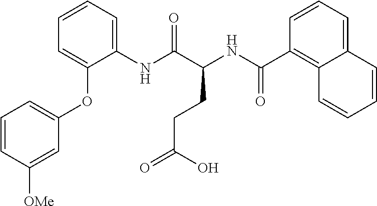 [SMILES: COc1cccc(Oc2ccccc2NC(=O)[C@H](CCC(=O)O)NC(=O)c2cccc3ccccc23)c1] | >10 uM |
| AOH1160-2AB | 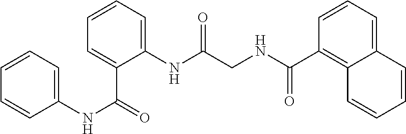 [SMILES: O=C(CNC(=O)c1cccc2ccccc12)Nc1ccccc1C(=O)Nc1ccccc1] | >10 uM |
| AOH1996S | 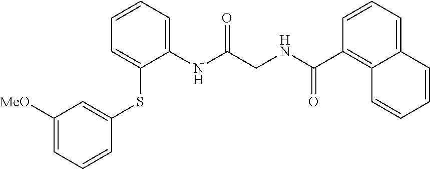 [SMILES: COc1cccc(Sc2ccccc2NC(=O)CNC(=O)c2cccc3ccccc23)c1] | Hela: 0.37 uM A375: 0.17 uM |
| AOH1996S-2F | 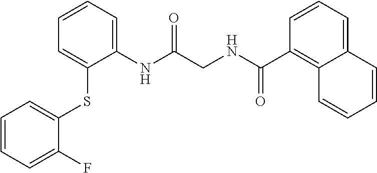 [SMILES: O=C(CNC(=O)c1cccc2ccccc12)Nc1ccccc1Sc1ccccc1F] | OVCAR8: 0.41 uM MDA-MB-231: 0.22 uM |
| AOH1996S-4F | 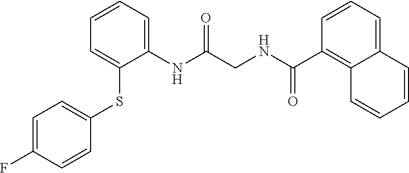 [SMILES: O=C(CNC(=O)c1cccc2ccccc12)Nc1ccccc1Sc1ccc(F)cc1] | SK-N-AS: 0.37 uM A375: 0.35 uM |
| AOH1996S-3F | 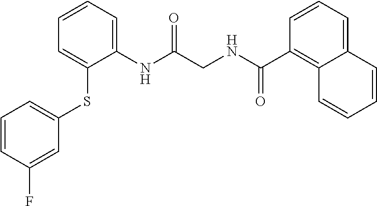 [SMILES: O=C(CNC(=O)c1cccc2ccccc12)Nc1ccccc1Sc1cccc(F)c1] | RKO: 2.32 uM HCT-116: 2.21 uM OVCAR8: 1.6 uM |
| AOH1996S-3CF3 | 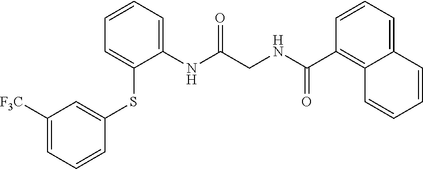 [SMILES: O=C(CNC(=O)c1cccc2ccccc12)Nc1ccccc1Sc1cccc(C(F)(F)F)c1] | OVCAR8: 2.6 uM RKO: 8 uM HTC-116: 4.6 uM |
| AOH1996S-4CF3 | 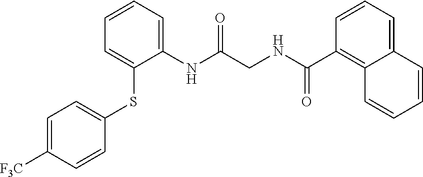 [SMILES: O=C(CNC(=O)c1cccc2ccccc12)Nc1ccccc1Sc1ccc(C(F)(F)F)cc1] | OVCAR8: 3.4 uM RKO: 10 uM HTC-116: 4.6 uM |
| AOH1996S-4CH3 | 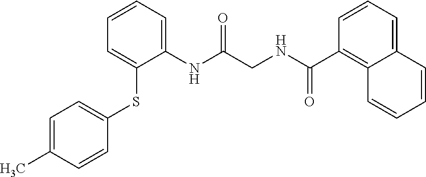 [SMILES: Cc1ccc(Sc2ccccc2NC(=O)CNC(=O)c2cccc3ccccc23)cc1] | OVCAR8: 291 nM RKO: 503 nM HTC-116: 345 nM |
| AOH1160NH | 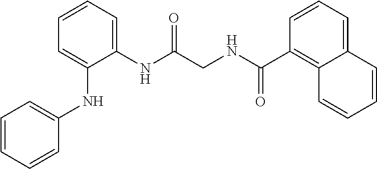 [SMILES: O=C(CNC(=O)c1cccc2ccccc12)Nc1ccccc1Nc1ccccc1] | OVCAR8: >10 uM RKO: >10 uM HTC-116: >10 uM |
| AOH1996SLE-4CH3 | 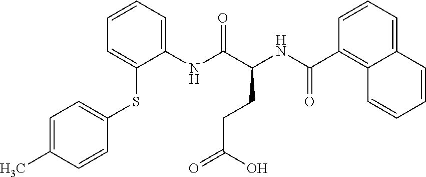 [SMILES: Cc1ccc(Sc2ccccc2NC(=O)[C@H](CCC(=O)O)NC(=O)c2cccc3ccccc23)cc1] | >10 uM |
| AOH-DMB-2CH3 | 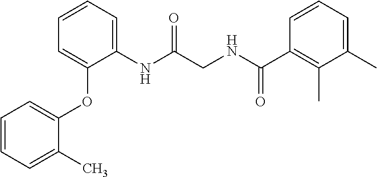 [SMILES: Cc1ccccc1Oc1ccccc1NC(=O)CNC(=O)c1cccc(C)c1C] | SK-N-AS: 1118 nM SK-N-BE(2)c: 1032 nM |

TABLE 2
A comprehensive cell culture study of 3 potent
AOH analogs (AOH1996, AOH1160, AOH1996-4′OMe).

| | Log10(GI₅₀) | Log10(GI₅₀) | Log10(GI₅₀) |
|---|---|---|---|
| Cell type | AOH1996 | AOH1160 | AOH1996-4′OMe |
| CCRF-CEM | −6.44 | −6.46 | −6.04 |
| HL-60(TB) | −7.09 | −6.57 | −6.21 |
| K-562 | −6.89 | −7.15 | −6.84 |
| MOLT-4 | −6.18 | −6.46 | −60.2 |
| RPMI-8226 | −6.02 | −6.50 | −5.95 |
| SR | −6.94 | −7.08 | −6.78 |
| A549/ATCC | −6.50 | −6.34 | −5.90 |
| EKVX | −5.89 | −6.11 | −5.86 |
| HOP-62 | −6.52 | −6.45 | −6.14 |
| HOP-92 | −6.69 | −5.65 | −4.60 |
| NCI-H226 | −5.60 | −5.66 | −5.37 |
| NCI-H23 | −6.13 | −6.18 | −5.89 |
| NCI-H322M | −5.89 | −6.20 | −5.91 |
| NCI-H460 | −6.53 | −6.48 | −6.07 |
| NCI-H522 | −6.75 | −6.82 | −6.71 |
| COLO 205 | −6.55 | −6.51 | −6.19 |
| HCC-2998 | −5.90 | −6.15 | −5.85 |
| HCT-116 | −7.02 | −6.49 | −6.21 |
| HCT-15 | −6.89 | −6.61 | −6.23 |
| HT29 | −6.96 | −6.54 | −6.24 |
| KM12 | −6.72 | −6.54 | −6.24 |
| SW-620 | −6.78 | −6.55 | −6.17 |
| SF-268 | −6.26 | −6.36 | −5.69 |
| SF-295 | −6.95 | −6.60 | −6.40 |
| SF-539 | −6.65 | −6.62 | −6.24 |
| SNB-19 | −6.21 | −6.34 | −5.91 |
| U251 | −6.62 | −6.48 | −5.98 |
| LOX IMVI | −6.57 | −6.51 | −5.87 |
| MALME-3M | −4.77 | −5.00 | −4.60 |
| M14 | −6.80 | −6.56 | −6.26 |
| MDA-MB-435 | −7.15 | −7.37 | −7.08 |
| SK-MEL-2 | −6.74 | −6.57 | 0.00 |
| SK-MEL-28 | −5.36 | −6.09 | −5.64 |
| SK-MEL-5 | −6.66 | −6.58 | −6.42 |
| UACC-62 | −6.68 | −6.75 | −6.25 |
| IGROV1 | −5.98 | −6.04 | −5.80 |
| OVCAR-3 | −7.00 | −6.72 | −6.41 |
| OVCAR-4 | −6.11 | −5.34 | −5.65 |
| OVCAR-5 | −5.95 | −6.28 | −5.70 |
| OVCAR-8 | −6.16 | −6.50 | −5.94 |
| NCI/ADR-RES | −6.62 | −6.62 | −6.37 |
| SK-OV-3 | −6.31 | −6.40 | −5.99 |
| 786-0 | −6.31 | −6.40 | −5.94 |
| A498 | −6.94 | −6.59 | −6.25 |
| ACHN | −6.40 | −6.08 | −5.82 |
| RXF 393 | −6.78 | −6.73 | −6.31 |
| SN12C | −5.84 | −6.23 | −5.64 |
| TK-10 | −5.80 | −6.21 | −5.67 |
| UO-31 | −5.74 | −6.19 | −5.83 |
| PC-3 | −6.55 | −6.52 | −5.98 |
| DU-145 | −6.15 | −6.49 | −5.99 |
| MCF7 | −7.00 | −6.51 | −6.59 |
| MDA-MB- | −6.11 | −6.01 | −5.65 |
| 231/ATCC | | | |
| HS 578T | −6.42 | −6.44 | −5.77 |
| BT-549 | −6.53 | −6.39 | −5.95 |
| T-47D | −6.86 | −6.27 | −5.60 |

AOH1160LE was soluble at a 4 mM concentration in aqueous buffer with 10% DMSO, which enabled co-crystallization studies on this analog. A PCNA:AOH1160LE co-crystal diffracted to 2.85 Å resolution at the synchrotron source, and phasing was provided by molecular replacement. Four PCNA subunits were present in the asymmetric unit, with three, chains A, B, C belonging to the homotrimeric ring structure, and the fourth, chain D (FIG. 1C) forms part of an adjacent ring in the unit cell that consists solely of D chains. Packing within the unit cell also places each monomer subunit of the PCNA ring against a subunit from another ring; these two stacked subunits interact via their PIP box binding pockets and 10 IDCLs that are orientated in directly opposing directions (FIG. 1D). Clearly observed within the electron density maps are three molecules of AOH1160LE, which bind in and adjacent to the PIP box cavity of each of the PCNA ring subunits, and these compounds have further interactions with the PIP box pocket of the stacked PCNA subunit (FIG. 1D).

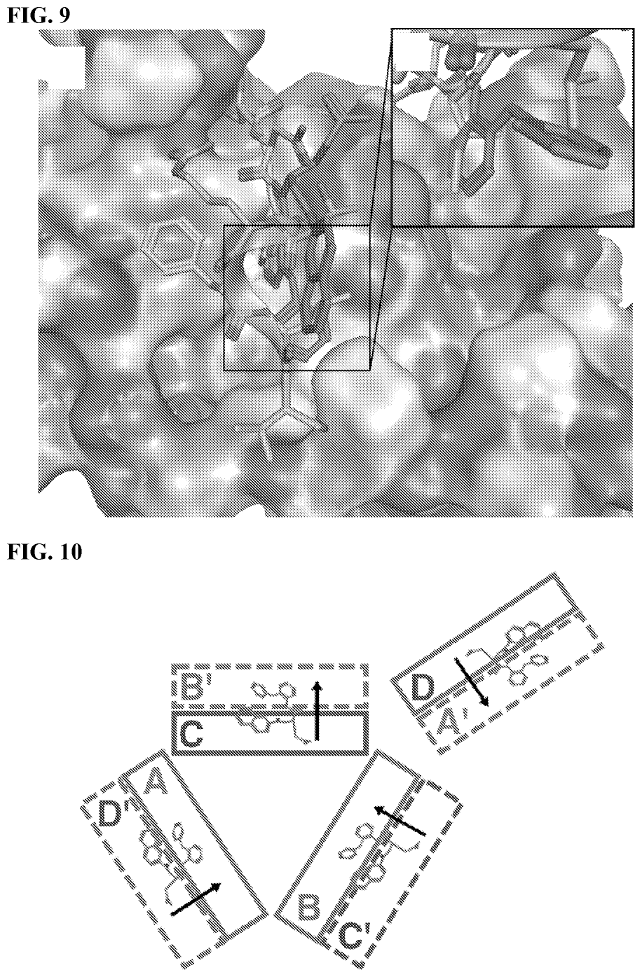

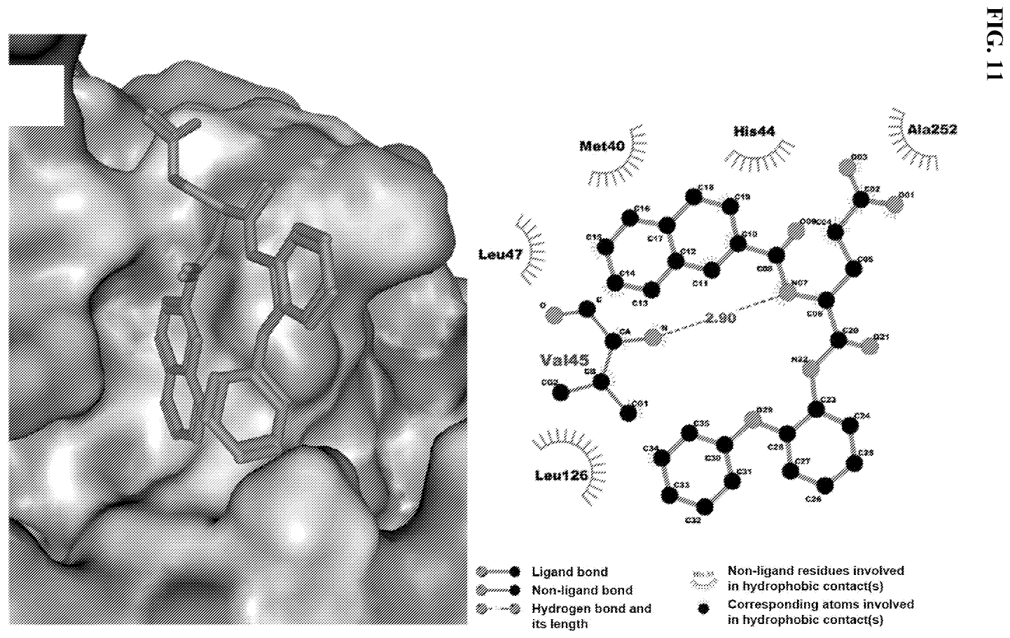

In chains A and B of the PCNA homotrimer, the central AOH1160LE molecule binds the PIP box binding cavity in an approximately perpendicular orientation to the binding pocket (FIG. 1E), similar to triiodothyronine (T3) (Punchihewa et al., 2012) or T2 amino alcohol (T2AA) bound to PCNA (Inoue et al., 2014) (FIG. 1F). One phenyl group of this central AOH1160LE compound binds into a largely hydrophobic region of PCNA consisting of residues His44, Val45, Leu47, Pro234, Tyr250, Leu251 and Ala252, and the second phenyl ether moiety binds into the region formed by PCNA residues Met40, Leu47, Leu126 and Leu128 (FIG. 1D, FIG. 8). Multiple PIP box pocket binders also interact with these two hydrophobic regions, including T2AA and T3 via iodo groups, and APIM peptides, e.g., ZRANB3 APIM motif (PDB code 5MLW) (Sebesta et al., 2017) (FIG. 9) and PIP box peptides, e.g., ZRANB3 PIP box peptide (PDB code: 5MLO) (Sebesta et al., 2017), via hydrophobic side chains. The central AOH1160LE binds in the opposite direction in the chain C and D subunits, with the naphthalene group binding to the two hydrophobic regions of the PIP box cavity (FIGS. 10-11). This is due to symmetry within the crystal, as chain C stacks against Chain B′, which is the symmetry mate of Chain B of the ring, while the D subunit stacks against Chain A′ (FIG. 1C, FIGS. 10-11).

The second AOH1160LE moiety binds, via its naphthalene ring, into a region consisting of PCNA residues Val233, Pro234, Ala252 and Pro253 (FIGS. 1E-IF, FIG. 8), in addition to this compound forming a hydrogen bond to the adjacent side chain of Asp232. This region of PCNA is bound by an aromatic group from the APIM peptide (FIG. 9) and by PIP box peptides, which bind via their first aromatic side chain of the PIP box motif. One phenyl ether group of the second AOH1160LE compound binds into a pocket formed between Pro234 and Gln 131, where an aromatic group of T3 and of T2AA were observed to also bind (FIG. 1F, FIG. 8). Other interactions of this second compound include a potential T-shaped x-interaction between its naphthalene group and a phenyl of the diphenyl ether group of the centrally bound AOH1160LE (FIG. 1E). This second compound also interacts with the stacking PCNA subunit, binding into a pocket that is immediately adjacent to the PIP box cavity of this subunit. Here, the glutamate side chain of this compound extends into this adjacent pocket to form a hydrogen bond to Ser39 of PCNA, and the remaining phenyl ether moiety binds near residues Met40, Ser42, Val123 and Leu126. The third compound bound in the PIP box cavity region binds diametrically opposite to the second: its glutamate side chain and one of the phenyl ether groups binds into the pocket that is immediately adjacent to the PIP box cavity (FIG. 1E, FIG. 8). Also, the naphthalene group and second phenyl group extend into the stacking chain's PIP box pocket, to bind in the same manner as the second compound binds to its PIP box pocket. Thus, this symmetry suggests that the structure is representative of the binding of two AOH1160LE compounds, the central and second compound as described here, which interact with residues of the PIP box cavity that are also known to be critical for binding of PIP box and APIM peptides, and for T2AA and T3.

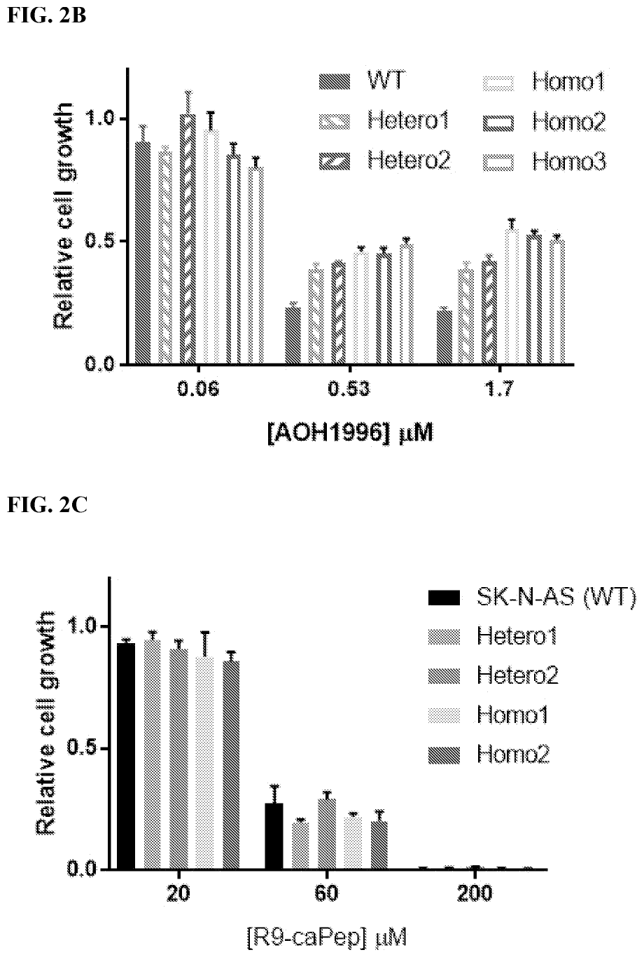

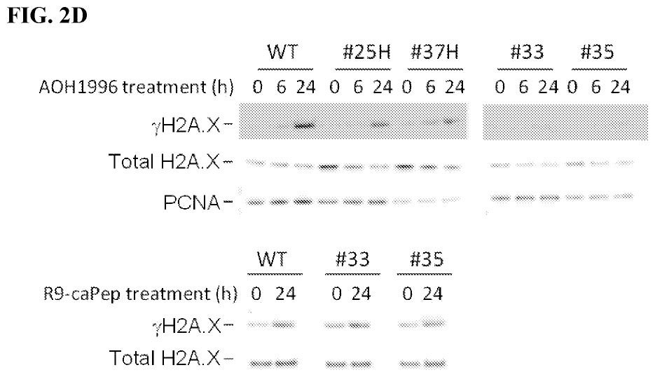

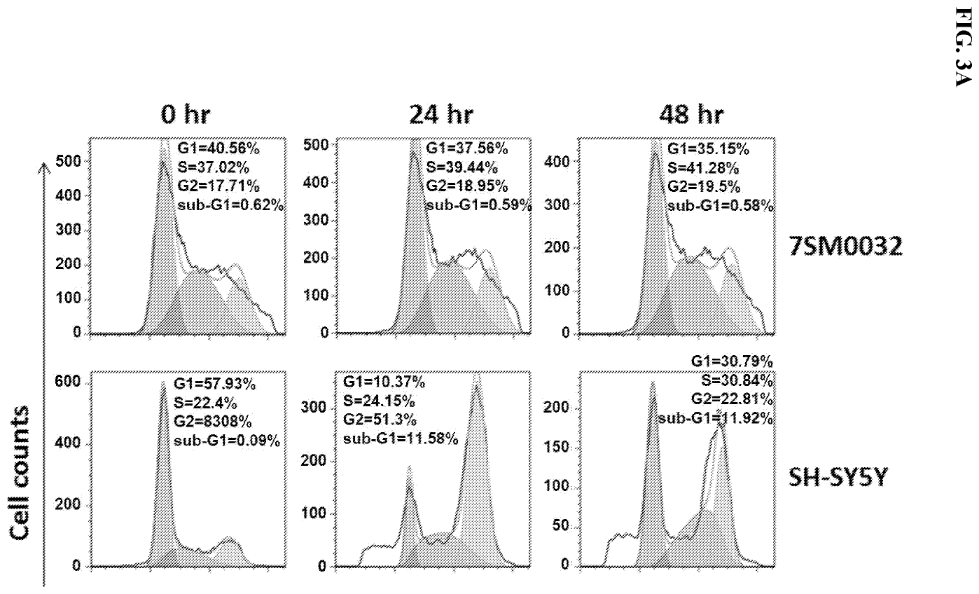

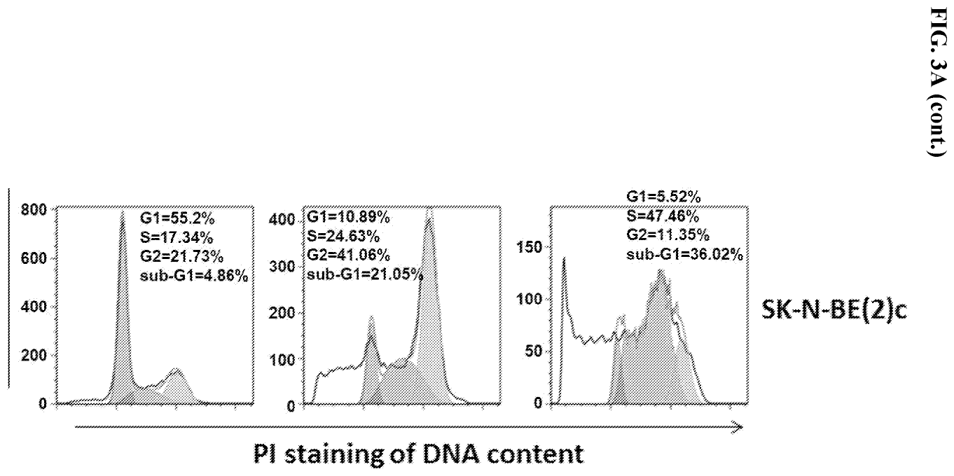

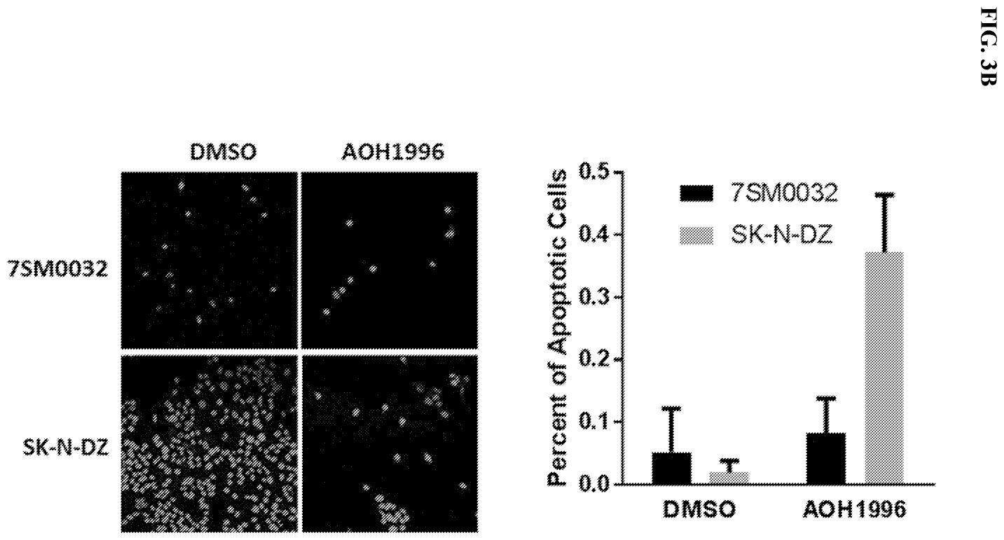

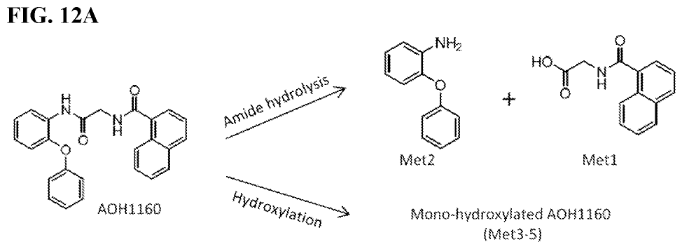

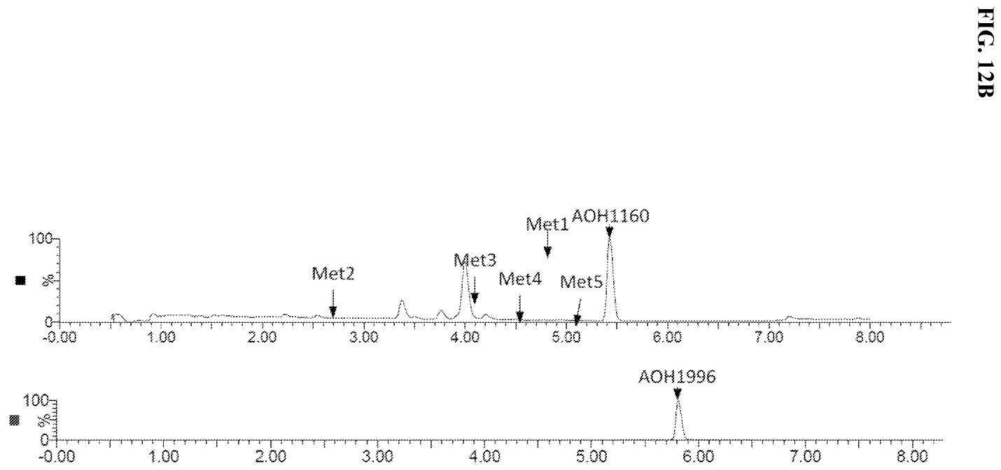

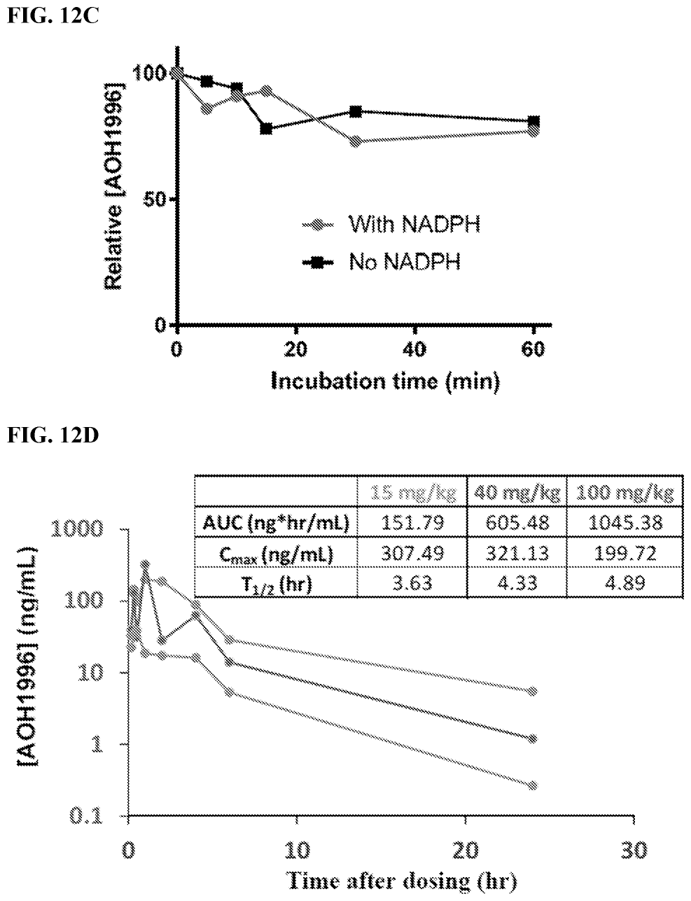

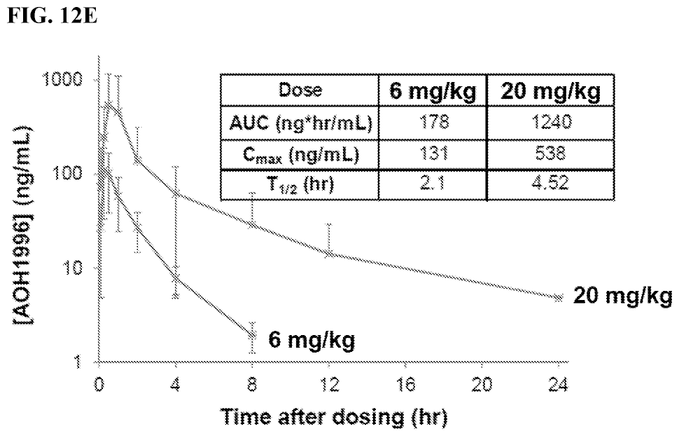

To verify the binding to the same site of AOH1996, a stable analog of AOH1160 (FIGS. 12A-12E), we created PCNA mutant cell lines by CRISPR (de la Fuente-Nunez and Lu, 2017; Hsu et al., 2014), in which the leucine 47 residue (L⁴⁷), one of the amino acids contouring the compound binding pocket (FIG. 8, FIG. 11), is substituted by a valine (FIG. 2A) and tested their sensitivity to AOH1996 treatment. Compared to wildtype parent cells, all mutant cell lines were less sensitive to growth inhibition by AOH1996, with the homozygous mutants being the least sensitive cell lines (FIG. 2B). In contrast, the L⁴⁷V mutation does not seem to affect sensitivity to inhibition of growth by R⁹-caPep (FIG. 2C), a cell permeable peptide that harbors the L126-Y133 sequence of PCNA's IDCL region and inhibits PCNA interaction with its binding partners presumably by acting as a “decoy” to the PIP box and APIM motif proteins (Gu et al., 2015; Gu et al., 2014; Smith et al., 2015). These results demonstrate that the L⁴⁷V mutation affects the binding of AOH1996 to PCNA without significantly changing the outer surface of PCNA that interacts with its binding partners. Similarly, the L⁴⁷V mutation confers resistance to induction of the DNA damage marker γH2A.X by AOH1996, but not by the R⁹-caPep (FIG. 2D). These studies support our model that AOH1996 binds to the same PCNA pocket as AOH1160LE.

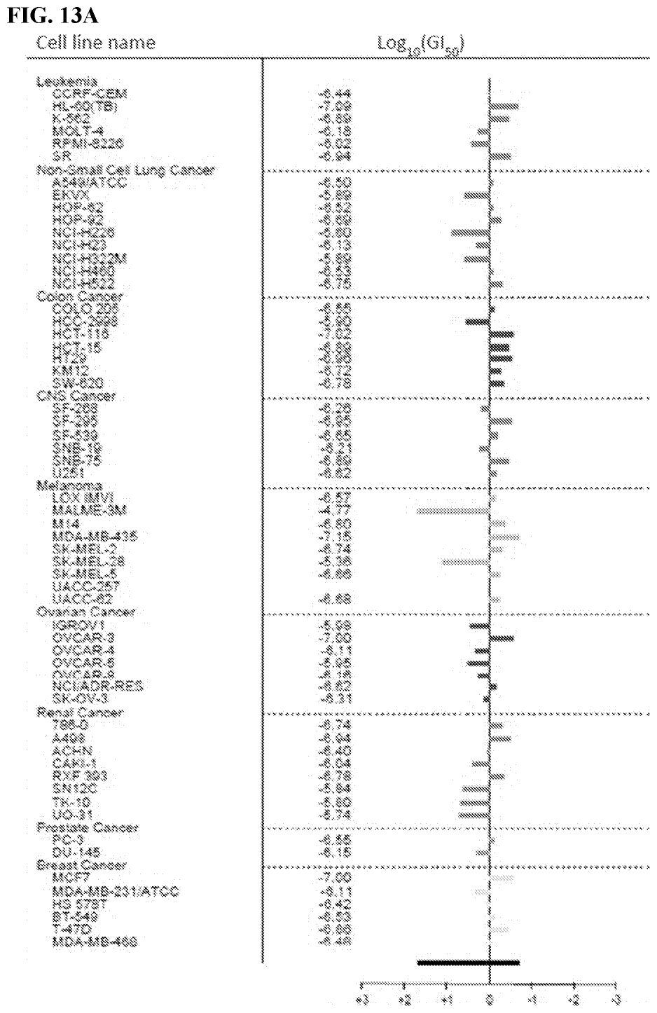

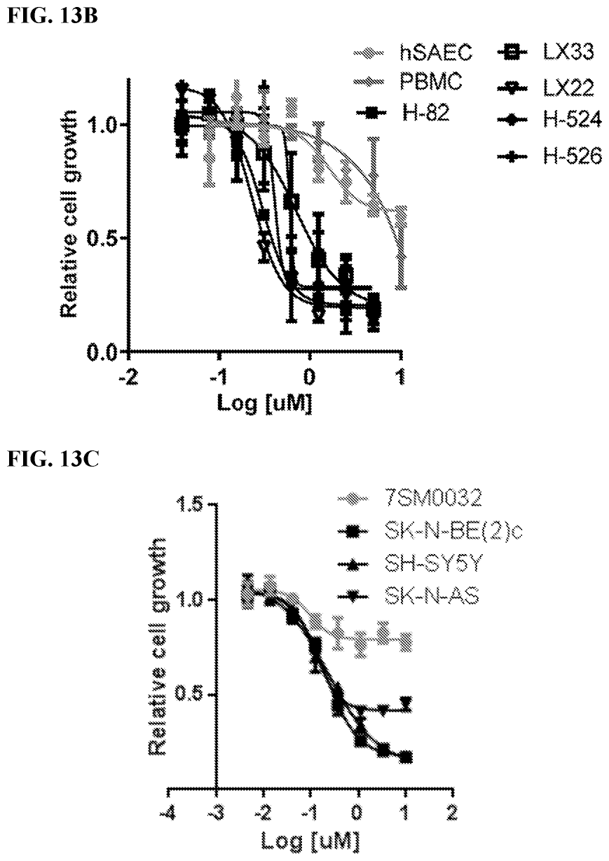

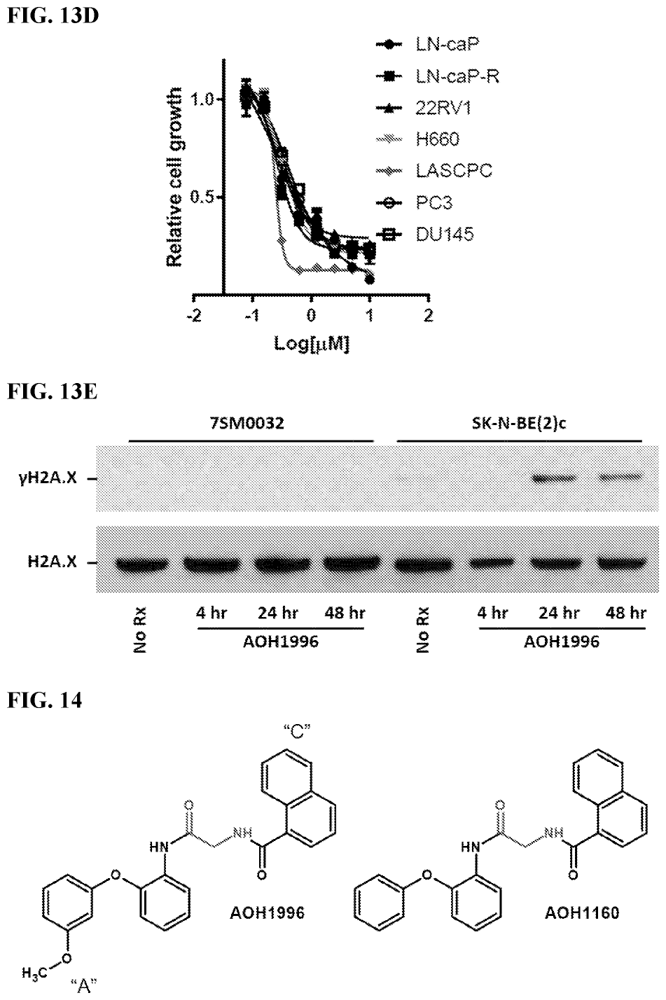

Superior therapeutic properties of AOH1996. AOH1996 selectively kills cancer cells; the median concentration to achieve 50% growth inhibition (GI₅₀) was approximately 300 nM across more than 70 cancer cell lines tested (FIGS. 13A-13E). In contrast, AOH1996 is not significantly toxic to nonmalignant cells, including human PBMCs, small airway epithelial cells (hSEAC), and neural crest stem cells (7SM0032), up to a concentration of at least 10 μM (FIGS. 13A-13E), demonstrating a potential 30-fold difference in sensitivity between cancer and normal cells. Consistent with these findings, AOH1996 treatment caused accumulation of DNA damages as measured by γH2A.X levels in the SK-N-BE(2)c cancer cells, but not in non-malignant cells (FIGS. 13A-13E).

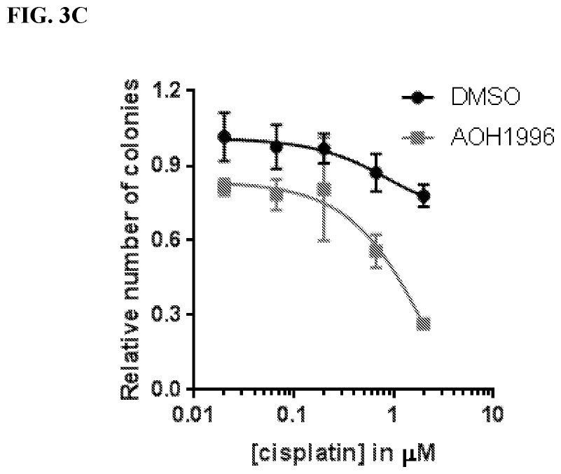

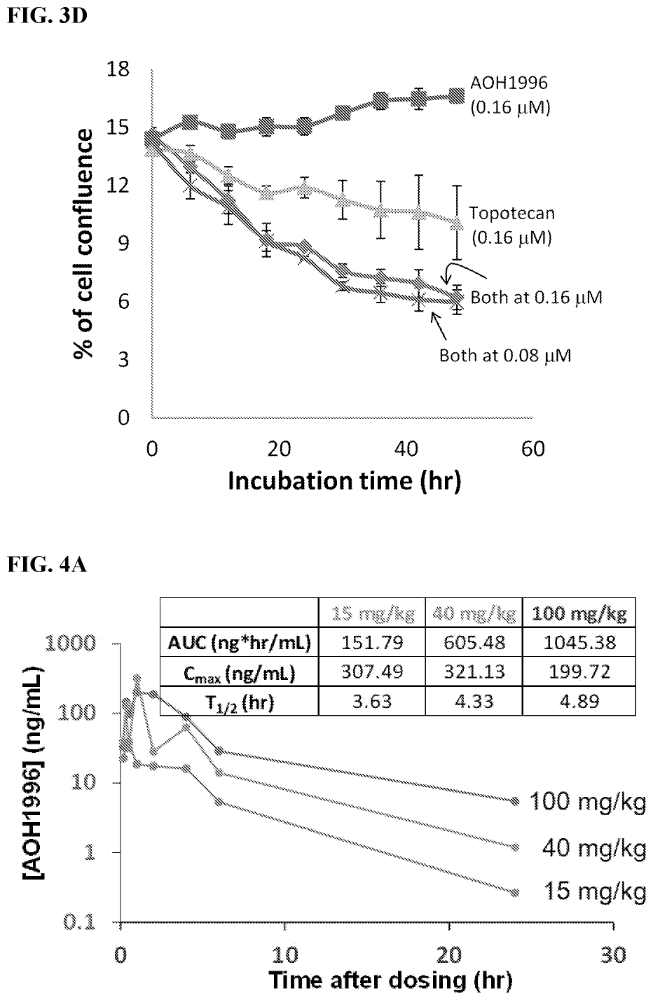

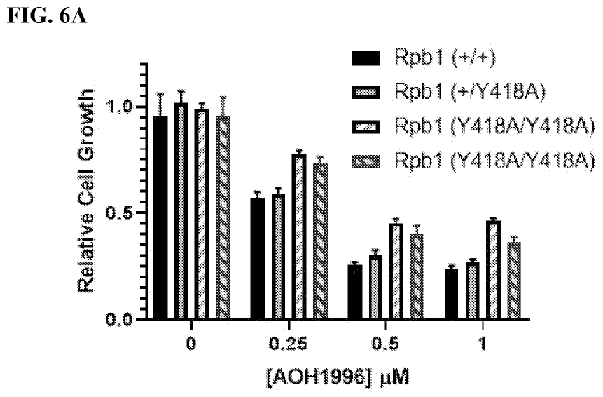

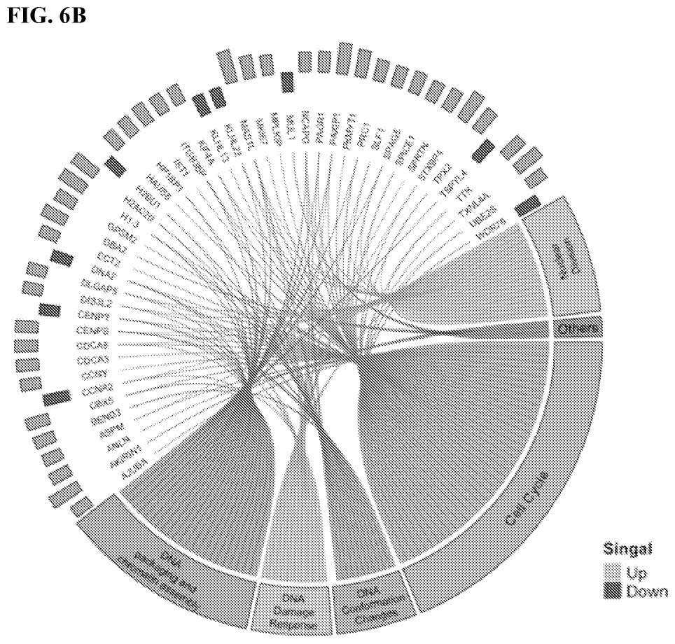

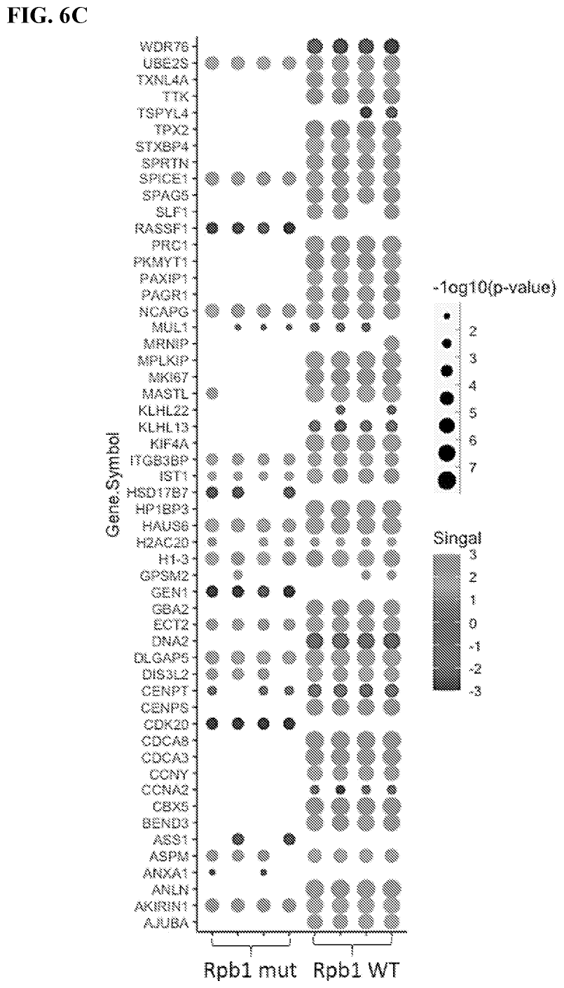

AOH1996 induced a substantial change in cell-cycle profile that indicates G2/M and/or S phase arrest in cancer cells, but not non-malignant stem cells (FIG. 3A), suggesting selective induction of replication stress in cancer cells. In addition, it induced apoptosis as indicated by the increase in the sub-G1 population (FIG. 6A) and TUNEL positivity (FIG. 3B) in cancer cells. Consistent with its lack of toxicity to nonmalignant cells (FIGS. 13A-13E), AOH1996 does not significantly change the cell-cycle profiles of the nonmalignant 7SM0032 cells (FIG. 6A). Nor does it induce apoptosis in 7SM0032 cells (FIG. 3B). AOH1996 increased the sensitivity of cancer cells to genotoxic agents, including cisplatin, which predominantly causes Pt-GG adducts (62-75%) (Dijt et al., 1988) in open chromatin areas (Han et al., 2016) (FIG. 3C). Similar synergy was also observed between AOH1996 and topotecan (FIG. 3D), a topoisomerase I inhibitor, which prevents topoisomerase I from re-ligating the nicked DNA strand and causes DSB during DNA replication (Pommier, 2006).

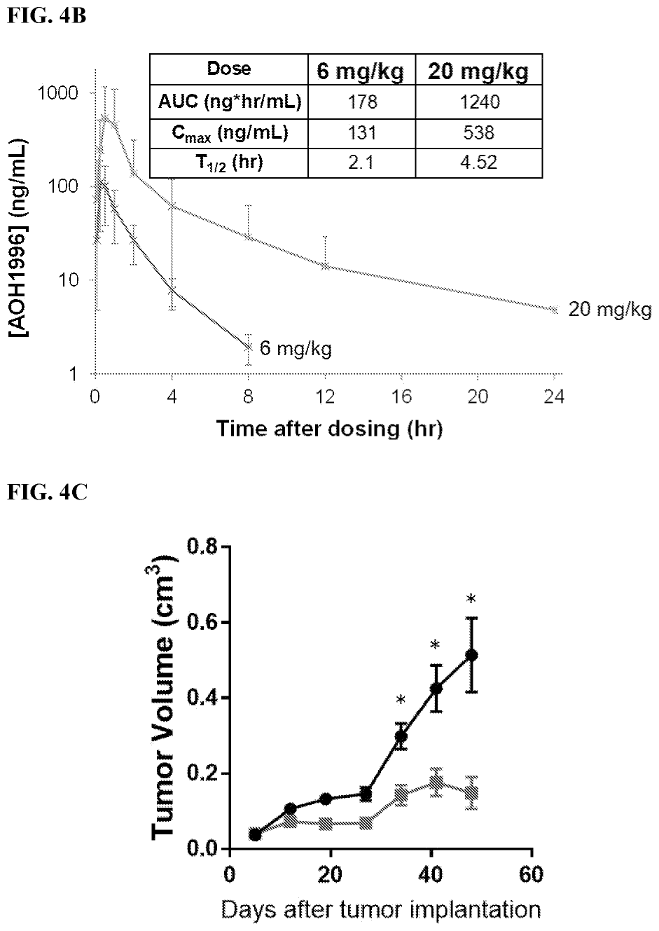

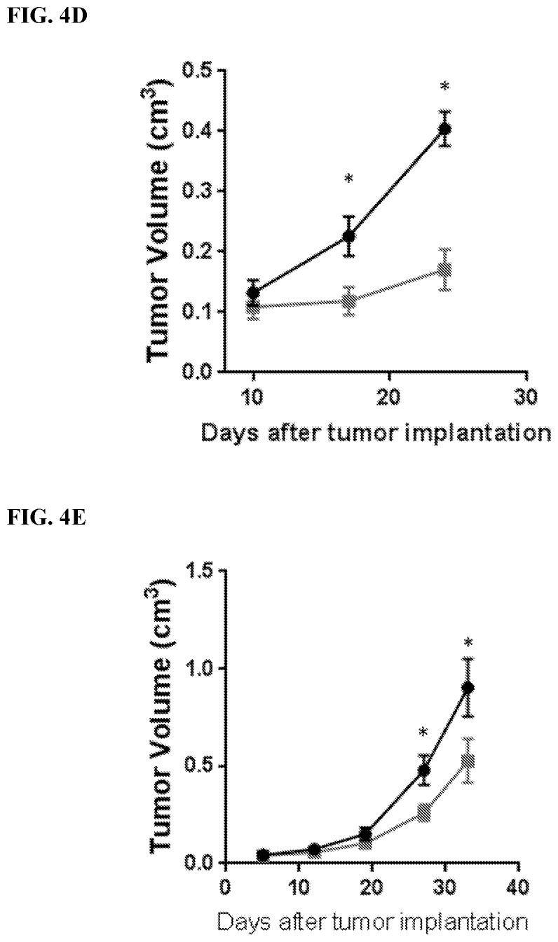

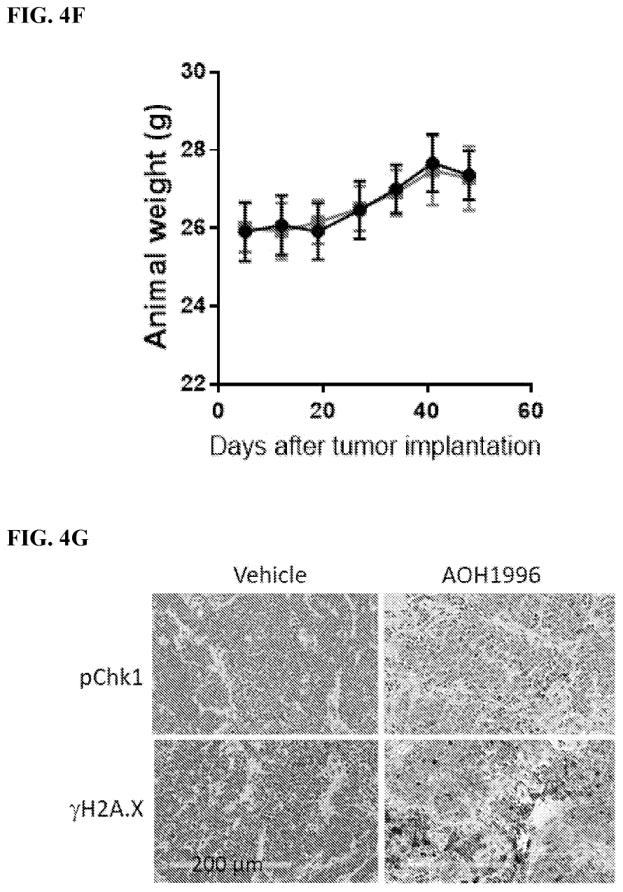

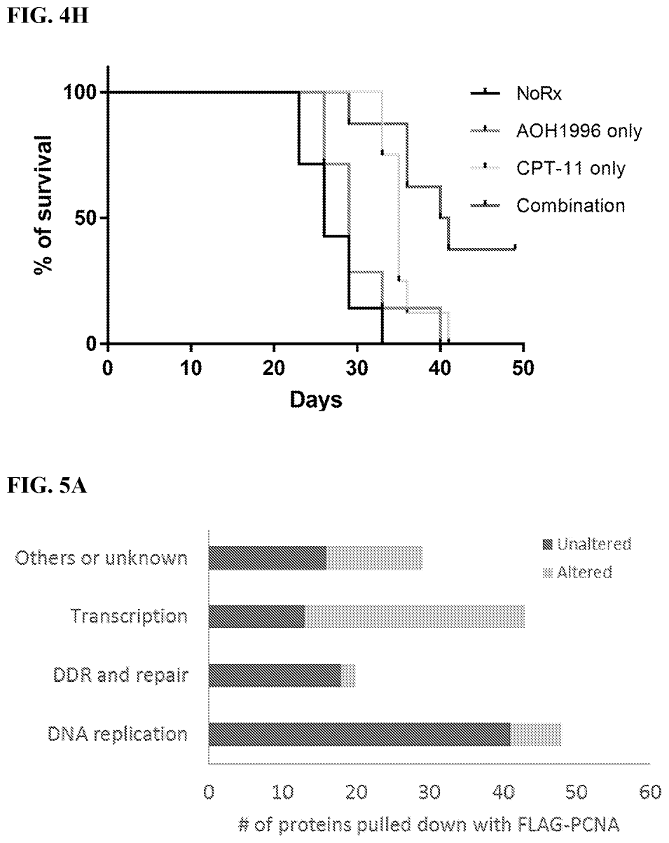

One purpose to synthesize and screen AOH1160 analogs is to identify drug candidate(s) that have similar therapeutic activity but are metabolically more stable than AOH1160 (Gu et al., 2018). The improved stability of AOH1996 (FIGS. 12A-12E) translated into a significant benefit in the pharmacokinetics (PK). In oral PK studies using FDA approved excipients (see methods), the compound half-life increased by ˜27% from 3.4 hr for AOH1160 to 4.33 hr for AOH1996 (FIG. 6A) following an identical dose of 40 mg/kg in ESI^{e} mice (Gu et al., 2018). The improvement in half-life is accompanied by ˜40% increase in peak concentration (Cₘₐₓ) and ˜4% increase in area under curve (AUC) (FIG. 4A & (Gu et al., 2018)). Comparable PK parameters of AOH1996 were observed in dog studies (FIG. 4B). These studies indicate that AOH1996 is orally available to animals (with almost 90% absorption into circulation, data not shown) in a formulation compatible with clinical applications.

We tested anti-cancer activity of AOH1996 in mice bearing xenograft tumors derived from neuroblastoma, breast cancer, and small cell lung cancer cells (FIG. 4C, FIG. 4D, and FIG. 4E, respectively). Daily AOH1996 treatment significantly reduced tumor burden in comparison with control groups that were given vehicle only in each of the tumor models (FIGS. 4C-4E) and did not cause any death or significant weight loss (FIG. 4F and data not shown). Furthermore, the no-observed-adverse-effect level (NOAEL) for AOH1996 was found to be ≥250 mg/kg/dose twice daily (BID) in mice and 75 mg/kg/dose BID in GLP-controlled toxicity studies consisting of 4-week chronical dosing and 2-week of post-dosing recovery periods. Based on 3:20 dose equivalency conversion between dogs and mice (Nair and Jacob, 2016), our study demonstrated that AOH1996 has a therapeutic window of more than 6 times its effective dose.

To identify pharmacodynamics (PD) markers, we analyzed xenograft tumors harvested from mice treated by AOH1996 or by vehicle only by immunohistochemistry. Focal staining of γH2A.X and phospho-Chk1 were observed in AOH1996 treated tumors (FIG. 4G). Cell disintegration was often observed at or around sites showing positive staining of γH2A.X and phospho-Chk1 (FIG. 4G). Overall, the tumors from AOH1996-treated mice were less dense than from the control mice. These results were consistent with the observation that AOH1996 causes DSBs (FIG. 2D) and G2/M arrest (FIG. 3A) in cancer cell and demonstrated the potential utility of γH2A.X and phosphor-Chk1 as PD markers in the clinic.

We further tested the effect of AOH1996 in combination with the topoisomerase I inhibitor CPT-11 (Ma et al., 2000) on xenograft tumors. Tumor bearing mice were either left untreated or were treated by AOH1996, CPT-11, or AOH1996 in combination with CPT-11. The AOH1996 treatment was given orally once daily for 8 consecutive days starting on the 8th day after tumor implantation. The CPT-11 was given by intraperitoneal injection once daily for 3 consecutive days starting on the 12th day after tumor implantation. After this single round of treatment, all animals were monitored without any further treatment until they died of tumor overgrowth. Median survival increased by ˜11.5%; however, a single round of treatment by AOH1996 alone failed to confer a statistically significant benefit on survival, probably due to the small cohort size and the short treatment duration. Treatment by CPT-11 only or by both AOH1996 and CPT-11 increased median survival by 34.6% and 55.4%, respectively (FIG. 6H). Comparing the group treated with AOH1996 in combination with CPT-11 with the untreated group or each of the groups treated with either agent alone, the survival benefit was statistically significant in favor of the combination treatment (FIG. 4H), demonstrating synergism between AOH1996 and CPT-11 in vivo.

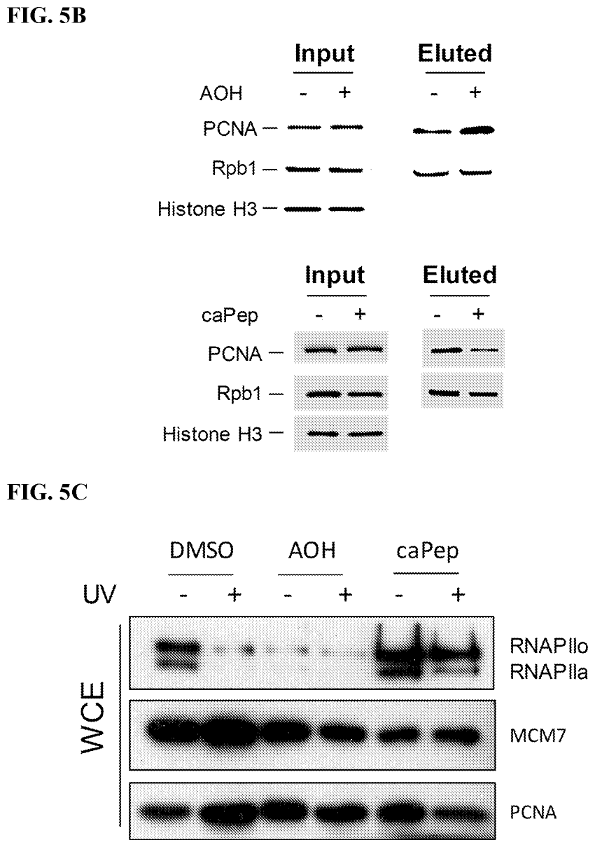

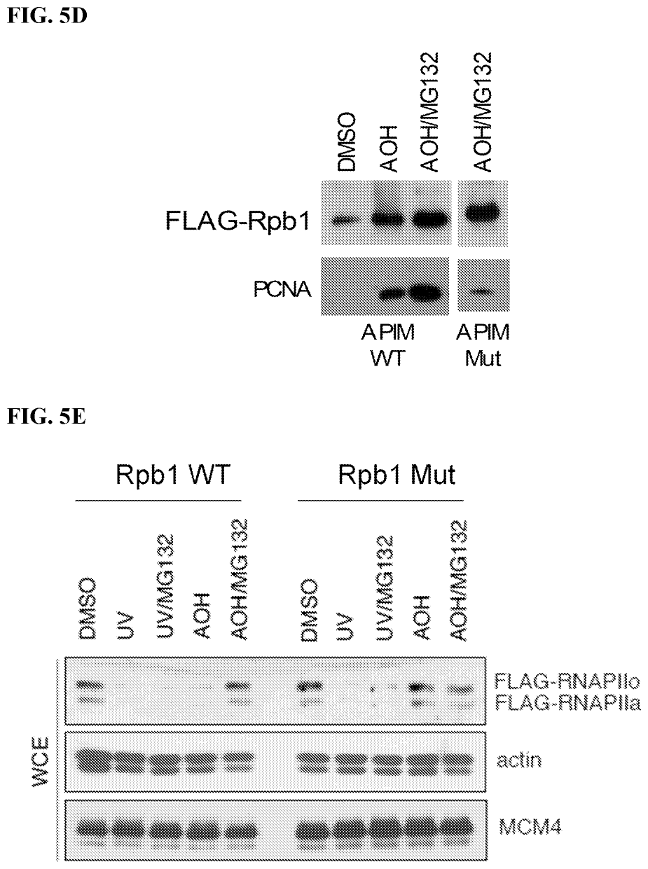

Modulating PCNA interaction with transcription machinery. Immunoprecipitation and mass spectrum analyses of PCNA interaction with its binding partners revealed that more than 50% of proteins whose association with the chromatin bound PCNA were altered by AOH1996 are components of the transcription processes (FIG. 5A). Further study of the effect of AOH1996 on transcription machinery components harboring the APIM motif, a known PCNA binding motif, discovered that AOH1996 enhanced interaction between PCNA and RPB1 (FIG. 5B) and depleted RPB1 in cancer cells (FIG. 5C). In contrast, treatment with caPep, which contains the L126-Y133 sequence of PCNA that overlaps APIM-interacting region of PCNA, blocked PCNA interaction with RPB1 (FIG. 5B) and increased RPB1 levels (FIG. 5C).

To determine whether effect of AOH1996 was mediated through RPB1 interaction with PCNA, we exogenously expressed FLAG-tagged wildtype RPB1 (AMIP WT) and FLAG-tagged RPB1 in which the Y418 within the APIM motif (Gilljam et al., 2009) was replaced by an alanine (AMIP mut). Immunoprecipitation of the chromatin-bound fraction of FLAG-tagged proteins revealed that AOH1996 increased both the amount of chromatin-bound wildtype RPB1 and the amount of PCNA co-precipitated with the FLAG-tagged wildtype RPB1 (FIG. 5D), indicating that AOH1996 increases TRC and enhances interaction between RPB1 and PCNA. These effects were further enhanced by MG132, suggesting AOH1996 can induce RPB1 degradation by proteasome, possibly by modulating RPB1 interaction with PCNA. In contrast, co-precipitation of PCNA with the FLAG-tagged AMIP mut RPB1 was only detectable at a reduced level in the presence of AOH1996 and MG132 (FIG. 5D), indicating a weakened interaction between PCNA and the mutation of Y418, which is partially compensated by the presence of AOH1996 and MG132. Examining the level of exogenously expressed RPB1 in whole cell extracts, we found that AOH1996 caused degradation of the wildtype RPB1, but not the APIM mut form, in a proteasome dependent manner (FIG. 5E). Interestingly, the APIM mut RPB1 was as susceptible to UV induced degradation as the wildtype RPB1, indicating that the mutant RPB1 selectively confers resistance to degradation triggered possibly by stalled transcription by replisomes, but not by UV-created DNA lesion.

We mutated Y418 of RPB1 to an alanine in the SK-N-AS cell line by CRISPR. Cells homozygous of the Y418A mutant alleles were significantly less sensitive to growth inhibition by AOH1996 than the parent cells (FIG. 6A). Mass spectrum and gene ontology (GO) enrichment analyses showed that proteins involved in cell cycle regulation and DNA replication and repair represent 49 out of 54 unique proteins whose levels were changed by AOH1996 treatment in either cell line by at least 2-fold with a p-value less than 0.05 (FIG. 6B). Most of the changes caused by AOH1996 treatment are much higher in the parent cells than in the RBP1 mutant cells (FIG. 6C), indicating that AOH1996 exerts its effects at least partially through modulating PCNA interaction with RPB1.

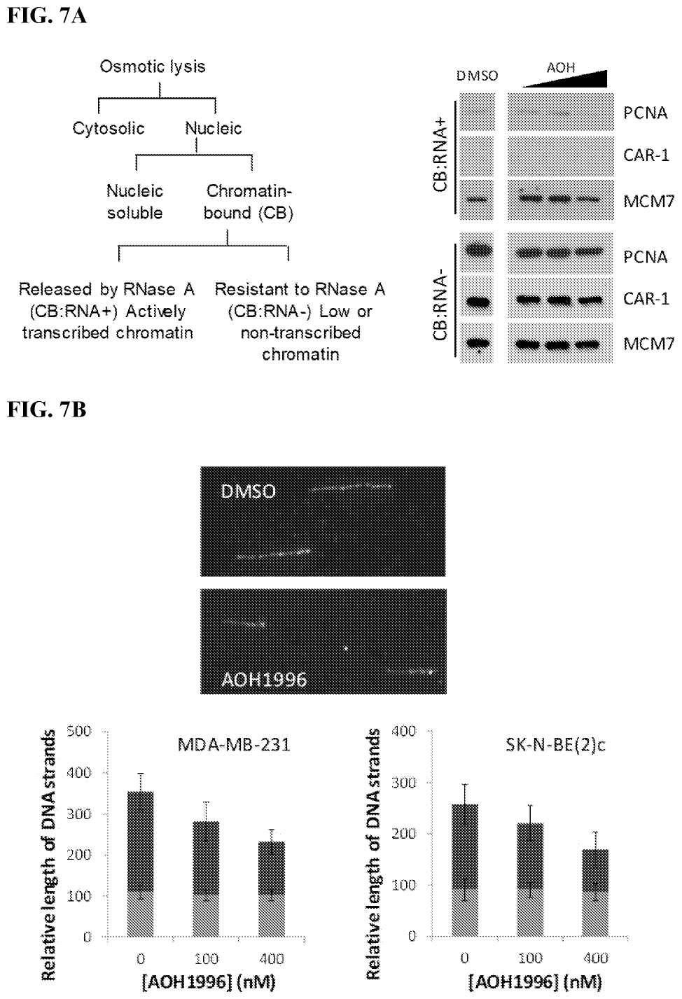

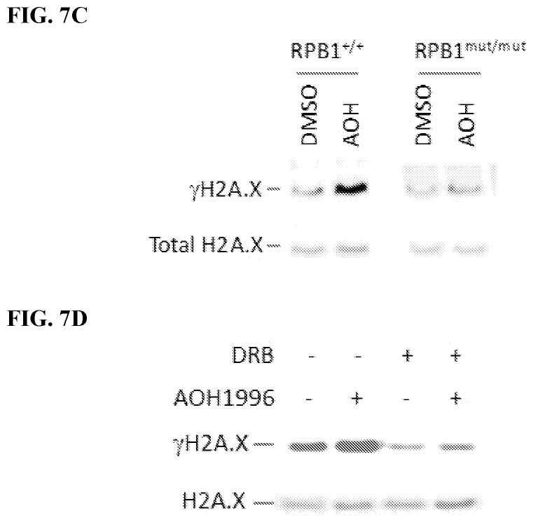

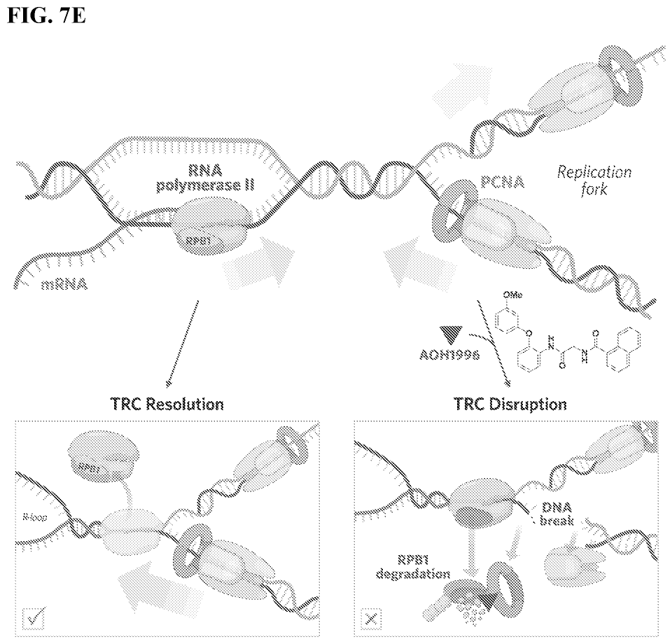

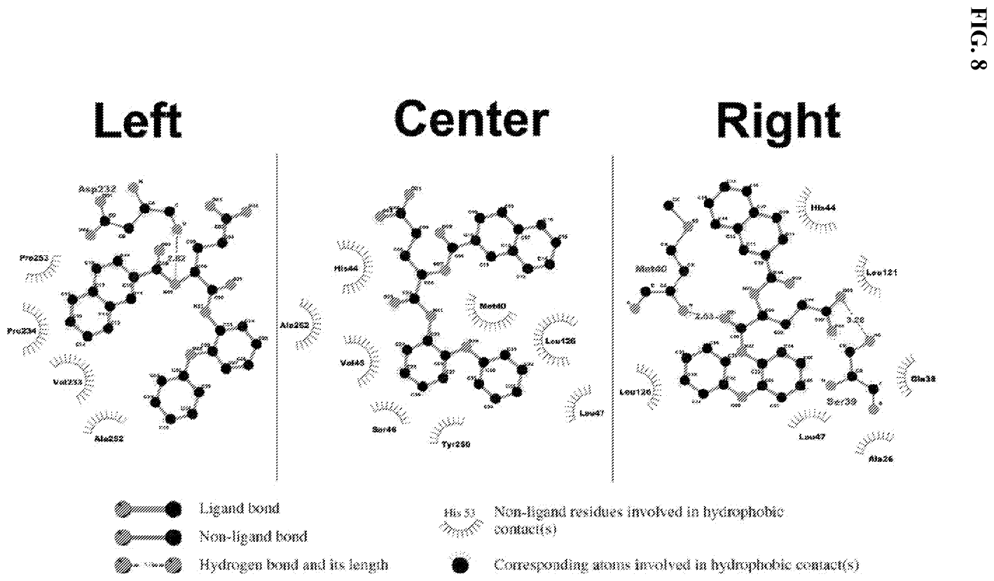

Transcription dependent dissociation of PCNA from chromatin. To determine the effect of altered PCNA and RBP1 interaction on DNA replication, we treated the chromatin pellet with RNase A to destabilize the open chromatin structures (Caudron-Herger et al., 2011; Li et al., 2011) and to solubilize active transcription factors, chromatin remodeling enzymes, and DNA replication factors from open chromatin (Li et al., 2015). After the solubilized fraction (CB:RNA+, FIG. 5A) was removed, the remaining insoluble pellet was further digested with benzonase to produce the protein fraction (CB:RNA−, FIG. 5A) enriched for the heterochromatin marker of CAF-1 (Li et al., 2018). Interestingly, AOH1996 caused dissociation of PCNA and MCM7 from the actively transcribed open chromatin regions (FIG. 7A), but did not cause much change in their presence at low or non-transcribed heterochromatin regions (FIG. 7A), suggesting that AOH1996 causes collapse of DNA replication and this effect occurs only in the presence of active transcription.

To measure the effect of AOH1996 on DNA replication fork extension directly, we pulsed synchronized S phase cells with a modified thymidine analog (CldU) in the absence of AOH1996. After washing away the unincorporated CldU, we incubated cells with a second thymidine analog (IdU) in the presence or absence of AOH1996. The DNA replication fork extension before and after AOH1996 treatment was quantified by measuring the relative length of CldU-incorporated DNA strands and adjacent IdU-incorporated DNA strands, respectively. Before AOH1996 treatment, the average lengths of the CldU-incorporated DNA strands (FIG. 7B, light grey strands or bars) were similar between the control and experimental cells, indicating similar DNA replication forks extension. In contrast, the IdU-incorporated DNA strands became significantly shorter in cells treated by AOH1996 than untreated control cells (FIG. 7B, dark grey strands and bars), indicating interference with DNA replication.

Consistent with our mechanistic model that AOH1996 exerts its effect by modulating PCNA interaction with RPB1, AOH1996 treatment caused substantially more DNA damages as measured by γH2A.X levels in cells containing wildtype RPB1 allele than in cells homozygous of the Y418A mutant allele (FIG. 7C). The transcription inhibitor DRB can suppress the DNA damage induced by AOH1996 (FIG. 7D), confirming the effect of the compound on DNA damage is transcription dependent.

A repertoire of synthetic approaches to AOH analogues. We have designed 3 different synthetic routes to prepare these AOH analogues (Schemes 1-3 in Example 2). AOH1160LV and AOH1160DV were synthesized in fair yield using method A (Scheme 1 in Example 2). In this approach, the active 1-naphthoyl chloride 1 was coupled with the corresponding tert-butyl L- or D)-valinate in presence of a base (DIPEA) in quantitative yield. Then, the tert-butyl protecting group was removed by TFA (quantitative yield) and the resulting free acid compound was coupled to 2-phenoxyaniline 4 (in fair yield) using PyPOB coupling agent (Coste et al., 1990) to afford final compound. In the case of less bulky amino acids, this method provided more inadvertent oxazolidinone intermediates which would be complicated to purify the desired product.

Moreover, the extended version of AOH1160 (named AOH1160e) with beta-alanine linker (in place of glycine linker in parent AOH1160) was synthesized in excellent yield using method B. As shown in Scheme 2 in Example 2, 1-naphthoyl chloride 1 was coupled with beta-alanine tert-butyl ester 5 in presence of a base (DIPEA) in quantitative yield. Then, the tert-butoxy group was replaced by active chlorine using a mixture of thionyl chloride and water (Greenberg and Sammakia, 2017) and coupled with 2-phenoxyaniline 4 to afford AOH1160e in excellent yield. This method was not compatible with N-Boc protected amino acids.

The superior results were obtained when N-Boc protected amino acid was used as described in method C. AOH1996 were synthesized by this approach in a higher yield and fewer side products. In this method (Scheme 3 in Example 2), the aniline derivatives (4, 10-12) were coupled with the corresponding N-Boc protected amino acid in very good yield using DCC (for glycine, 8) or HBTU (for alanine, 9) coupling agents (Dourtoglou et al., 1978). Then, N-Boc protecting group was removed by TFA/DCM (1:1) in quantitative yield and the resulting free amine product (13′-17′) was linked to 1-aroyl chloride/acid derivatives (1, 18-22) in very good yield. This new pathway resulted in AOH1996 and was extended to other AOH derivatives in very good yields as shown in Scheme 3 in Example 2. The 3-methoxyphenoxy phenylamine (10, 3-MOPA) moiety was synthesized in two steps in excellent yield (Bueno et al., 2019). All final compounds were obtained in 99.9% purity (confirmed by NMR) after purification by an automated Flash Chromatography system using gradient wash (hexane/ethyl acetate or DCM/MeOH).

Besides oncogenes, the survival of cancer cells depends on several stress response pathways including those for oxidative damage, DNA damage, and heat-shock, all of which play critical roles in normal and ubiquitous cellular functions (Luo et al., 2009). While not oncogenic themselves, many of the rate-limiting proteins in these pathways are essential for dealing with the increased stresses in cancer cells (Luo et al., 2009). Increasingly, cancer drug discovery has targeted these non-oncogenic pathways, and efforts have yielded a number of successful therapeutics (see Ashwell and Zabludoff, 2008 for review). Unlike oncogenes, target genes in these non-oncogenic pathways do not undergo oncogenic mutations or functionally significant genomic alterations in tumors. Therefore, they represent points of intervention less prone to the development of resistance. Acting as a central “hub” in the DNA replication/repair and interacting with many other cellular pathways, including mRNA transcription, PCNA is one of such non-oncogene proteins essential to growth and survival of cancer cells.

Our study reports two new AOH1160 based inhibitor analogs, with the readily soluble analog AOH1160-ILE clearly demonstrating binding to the PCNA PIP Box binding cavity. The second is a cell permeable and more metabolically stable compounds, AOH1996, that is lead compound with drug-like characteristics.

Our studies reveal that AOH1996 enhances the interaction between PCNA and RPB1. This leads to the overall degradation of RPB1, and collapse of DNA replication forks in actively transcribed chromatin region. Both transcription and replication are both highly active in fast growing cancer cells. Firing of dominant replication origins driven by oncogenes further dysregulates the spatio-temporal segregation of transcription and replication during S-phase in cancer cells (Jones et al., 2013). Thus, resolving TRC is paramount to the growth and survival of cancer cells, as the replication stress TRC causes can lead to genome instability and lethal DNA damage. Yet, the mechanisms to resolve TRC have yet to be fully elucidated, and TRC has not been previously targeted for therapeutic development.

Based on our findings, we now propose a working model to target TRC (FIG. 7E). When the transcription and replication machineries encounter each other on a chromosome, the RNA polymerase is temporarily removed from the collision site, leaving the unfinished RNA transcript forming an R-loop structure with the DNA template. It has been shown that PCNA plays a role in the process of dislodging RNA polymerase (Li et al., 2018). We now demonstrated that RPB1 interacts with PCNA through its AIMP motif, possibly by interacting with the outer hydrophobic surface adjacent to the inter-domain connector loop (IDCL) of PCNA, which interacts with AOH1996. The binding of AOH1996 to PCNA likely stabilizes the disordered IDCL region, as per the AOH1160LE crystal structure, and as noted in other PCNA co-complex structures (Li et al., 2017) and it enhances the interaction of PCNA with RPB1, thereby preventing the displacement of RNA polymerase and TRC resolution. The persistent presence of unresolved TRC not only leads to the lethal DSB, but also disrupts the transcription machinery by causing RPB1 degradation.

Overall, the presence of a cancer associated form of PCNA has allowed for the chemical disruption of the PCNA and TRC interface in cancer cells, enabling AOH1996 to exert potent, selective anticancer effects, while maintaining a remarkable safety profile. Thus, our study demonstrates both a therapeutic potential for AOH1996, and highlights its utility as a research tool to aid in the molecular characterization of the TRC in cancer cells.

### Example 2: Methods

Computer modeling. The computer modeling of AOH1996 binding to PCNA was based on the All-Around-Docking methodology and refined by 50 ns metadynamics simulation using NAMD software (Phillips et al., 2005). The free energy (ΔG) determined by the docking study was related to the compound's Ki by the Nernst equation at system equilibrium: ΔG=−RTln(Kᵢ), in which R=0.001987 kcal/K/mol. To model AOH1996 interaction with PCNA in complex with RPB1, we downloaded the protein structures: PDB 5iyd for the RPB1 APIM motif peptide and 5mlw for PCNA in complex with ZRANB3 APIM motif peptide, from the RCSB Protein Data Bank. The peptide structural alignment of RPB1 and ZRANB3 APIM motif peptides is carried out using PyMol (The PyMOL Molecular Graphics System, Version 2.0 Schrödinger, LLC). The best binding pocket of AOH1996 to the PCNA/RPB1 complex is predicted by using our in-house developed All-Around Docking method (Yu et al., 2016), which can automatically dock the ligand all-around the protein surface to search for the best sites by Glide (Friesner et al., 2006) and Induced Fitting docking (Sherman et al., 2006) methods. The 2-dimensional interaction diagram is drawn by Schrödinger Maestro software. The 3-dimensional interaction plot is generated by our in-house developed LiAn (Legion Interfaces Analysis) program (Guo et al., 2020), which can calculate and display protein-ligand or protein-protein interactions (such as hydrogen bond, salt-bridge, water-bridge, π-interactions, hydrophobic interactions, halogen bond, etc.) for single protein structure or massive structures from molecular dynamics simulations.

Plasmids, cell lines, and, transfection. Human neuroblastoma cell lines (SK-N-DZ, SK-N-BE(2) c, SK-N-AS, and SH-SY5Y) and breast cancer cell line (MDA-MB-468) were obtained from American Type Culture Collection (ATCC) and cultured in DMEM with 10% fetal bovine serum (FBS), 100 units/ml penicillin, and 100 μg/ml streptomycin. The HEK293T cells were cultured in DMEM with 10% fetal bovine serum (FBS), 100 units/ml penicillin, and 100 μg/ml streptomycin. Human embryonic progenitor cell line 7SM0032 was acquired from Millipore and cultured in the hEPM-1 Media Kit purchased from the same company. The plasmid expressing a FLAG-tagged PCNA were transfected by Lipofectamine 2000 (ThermoFisher Scientific).

Expression, purification and crystallization of PCNA. Human PCNA in a pET22b-hPCNA vector was transformed into E. coli Rosetta 2 (DE3) cells. PCNA expression was induced by 0.4 mM IPTG, OD₆₀₀=0.6, and cells were grown for 5 hrs at 37° C. Cells were harvested by centrifugation, 30 min at 5,000×g, and resuspended in lysis buffer, 25 mM Tris-HCl pH 8.5, 50 mM NaCl, 1 mM β-mercaptoethanol, 1 mM PMSF and 10% glycerol. Cells were sonicated, and soluble hPCNA in the cell supernatant was purified by HiTrap Q FF column (GE Healthcare) in lysis buffer with a 0.05-1.0 M NaCl gradient, followed by anion exchange chromatography with ENrichQ (BioRad) with lysis buffer over a 0.15-1.0 M NaCl gradient. Pooled PCNA fractions were loaded onto a HiLoad 26/60 Superdex 200 gel filtration column (GE Healthcare), in 10 mM HEPES pH 7.4, 100 mM NaCl and 1 mM β-mercaptoethanol. Purified hPCNA protein was incubated overnight with LE compound at 9 mg/mL PCNA (313 μM), 4 mM LE, in 9 mM HEPES pH 7.4, 90 mM NaCl, and 10% DMSO.

Thermal denaturation assay for PCNA-AOH1996 interaction. The assay was conducted using a BioRad CFX Connect Real-Time PCR Detection System. Protein (PCNA), inhibitors (AOH1996 and AOH1160LE), and 200×SYPRO orange dye (Sigma) were diluted into phosphate buffered saline (PBS). The final concentration of recombinant PCNA was 9 μM, and final compound concentrations were at 0, 10, or 30 μM. Sample plates were heated from 25° C. to 95° C. with heating increments of 0.5° C./min. Fluorescence intensity was measured within the excitation/emission ranges 470-505/540-700 nm.

Crystallization, X-ray data collection, processing, and refinement. Co-crystals were grown by vapor diffusion, with a reservoir solution of 100 mM sodium cacodylate pH 6.5, 200 mM NaCl and 2.0 M ammonium sulfate. Crystals after two weeks growth at 293 K were crushed using the Seed Bead Kit (Hampton Research) and a 10-5 seed dilution in a 1:1 ratio with pre-incubated PCNA:AOH1160LE was setup in hanging drop vapor diffusion, using the same reservoir solution. Seeded crystals grown at 293 K were collected and flash frozen in liquid N₂. X-ray data was collected at beamline 9-2 SSRL, Stanford, CA at 100 K. Images were collected at 0.2 sec, 0.15 deg per image, over 270 deg of data. Data was processed using XDS (Kabsch, 2010) to 2.85 Å in the H3 space group, with cell dimensions of a=b=197.14 Å, c=126.98 Å, a=b=900 and c=1200. Phasing was obtained using Phaser-MR (McCoy et al., 2007) with 3VKX.pdb as the search model. Model building and refinement was completed using Phenix (Adams et al., 2010) and Coot (Emsley et al., 2010). Images of molecular interactions were prepared utilizing the MOE (Molecular Operating Environment, version 2020.0101, Chemical Computing Group, Ontario, Canada).

Establishment of mutant cell lines by CRISPR. To introduce establish SK-N-AS cells heterozygous or homozygous of the mutant PCNA allele (L47V), specific guide RNAs (sgRNAs) were designed using the online tool CHOPCHOP (http://chopchop.cbu.uib.no). sgRNA sequences were selected close to the target sequence and with minimal identical genomic matches or near-matches to reduce risk of off-target effects. After confirming CRISPR editing efficiency, two sgRNAs were synthesized (PCNA-CR1: GGACTCGTCCCACGTCTCTT (SEQ NO ID: 5) and PCNA-CR4: CTTTGGTGCAGCTCACCCTG (SEQ ID NO:6)). According to the sgRNA cutting sites, two mutations were made in protospacer adjacent motif (PAM) sequence of the homology-directed repair (HDR) donor template to prevent re-cutting by CRISPR. The primer set (PCNA-SvF: CGGCATTAAACGGTTGCAGG (SEQ ID NO:7) and PCNA-SvR: CGTGGCAGGCCAATGAGAAG (SEQ ID NO:8)) was used to perform the surveyor assay and DNA amplification. The primer set (PCNA-FA-FP: ACGAGGCCTGCTGGGATATT (SEQ ID NO:9) and PCNA-FA-FP: TGAGGGCTAGGCTCGAAAGC (SEQ ID NO:10)) was used for DNA sequencing. The SK-N-AS neuroblastoma cells were seeded at a density of 5×10⁵/well in a 6-well plate and were co-transfected with: 1) pX458-PCNA-CR1/4 plasmid encoding CRISPR Sp-CAS9, a GFP selection marker, and the PCNA-CR1 and PCNA-CR4 sgRNAs, and 2) a plasmid containing the mCherry selection marker and the donor template. 48 h later, transfected cells were sorted for GFP and mCherry expression and enriched cells were seeded into 96-well plates by single cell limiting dilution. Single-cell clones were screened by DNA sequencing of the target site to identify cells homozygous (PCNA1.47V/1.47V) or heterozygous (PCNA+/1.47V) of the mutant allele. The RPB1 mutant cell lines were established by the same method using RPB1-CR1: ATTGTCTCGGATGATGTACT (SEQ ID NO:11) as sgRNA.

Cell Cycle Analysis. Cells were seeded in a 6-well plate at 1×10⁵/mL. After treatment with the compound, cells were fixed in 60% ethanol and stained with propidium iodide (PI). PI fluorescence intensity of the cells was measured by flow cytometry and the data was analyzed using the FlowJo program.

Cell growth and terminal deoxynucleotidyl transferase-mediated dUTP nick end labeling (TUNEL) assays. Cells were seeded at 5×10³/ml or 3×10⁴/ml into a 96-well plate, depending on the cell lines, and were treated with various concentrations of AOH1996 for 72 h after being allowed to attach overnight. Cell growth was measured by the CellTiter-Glo assay (Promega) according to the manufacturer's instruction. Alternatively, cell growth was analyzed by the IncuCyte® S3 Live-cell Analysis Systems (Sartorius), which measures cell confluence by periodic imaging. The effect of AOH1996 on the NCI60 panel of cell lines were analyzed in the standard 5-dose assay by a sulforhodamine B (SRB) assay by the National Cancer Institute (NCI) as described (https://dtp.cancer.gov/discovery_development/nci-60/methodology.htm). The 50% growth inhibition concentration (GI50) was calculated by the NCI.

Cell apoptosis was measured on a chamber slide at a seeding density of 1×10⁵/mL. After treatment with 500 nM AOH1996 for 24 h, cells were fixed and analyzed by a TUNEL assay using the TMR red in situ cell death detection kit (Roche Diagnostics).

DNA combing analysis. A DNA combing assay was performed as described (Frum et al., 2013). Briefly, synchronized neuroblastoma (SK-N-BE(2)-C) or breast cancer (MDA-MB-231) cells were incubated first with 5-Chloro-2′-deoxyuridine (CldU) for 10 minutes. After washing away the unincorporated CldU, cells were incubated with 5-Iodo-2′-deoxyuridine (IdU), in the presence or absence of AOH 1996 at the indicated concentrations for 20 minutes. The cells were spotted and lysed on microscope slides. The DNA fibers spread across the slides were immunologically stained with fluorophore-conjugated antibodies specific for CldU and IdU and were visualized under a fluorescent microscope. The length of CldU and IdU incorporated DNA fibers was measured using ImageJ software (National Institute of Health).

Clonogenic Assay. SK-N-DZ neuroblastoma cells were seeded and allowed to attach onto 60-mm plates (300 per plate). Cells were treated with cisplatin alone or with cisplatin and AOH1996 for 18 h. Cells were then cultured in fresh medium without cisplatin or AOH1996 for 18 d to allow the surviving cells to form colonies. The colonies were stained with 0.5% crystal violet and counted. Synergy between AOH1996 and cisplatin was evaluated using combination indices (Cl) based on the Bliss independence model [C]=(E_{A}+E_{B}−E_{A}*E_{B})/E_{AB}] (Foucquier and Guedj, 2015).

Western blot. Cells were dissolved into Laemmli sample buffer on the plate. Whole cell extracts were sonicated, resolved on a 4-12% Bis-Tris protein gel, and blotted onto a nitrocellulose membrane. The membrane was blocked with 5% nonfat dry milk and incubated individually with antibodies for H2A.X (Cell Signaling Technology, Danvers, MA), γH2A.X (Millipore), CAF-1 (Novus Biologicals), PCNA (Santa Cruz Biotechnology), and MCM7 (Abcam) diluted in blocking buffer. After incubation with peroxidase-conjugated secondary antibodies, the protein of interest was detected using an ECL kit purchased from ThermoFisher Scientific.

Cell fractionation and immunoprecipitation. Cells were fractionated as previously described (Li et al., 2018). Briefly, intact nuclei isolated following osmotic lysis were homogenized using a 21 G needle. Chromatin was pelleted by centrifugation and incubated overnight at 4° C. with benzonase in two volumes of nuclease buffer (20 mM HEPES pH 7.5, 1.5 mM MgCl₂, 1 mM EDTA, 150 mM KCl, 10% glycerol, 0.5 U μl-1 benzonase). The resulting supernatant was collected as the CB fraction. Alternatively, we sequentially incubated the chromatin pellet with RNase A and benzonase and collected the supernatants after each digestion as the CB:RNA+ and CB:RNA− fractions, respectively (Li et al., 2018). The chromatin extracts (CB) were incubated overnight with Anti-FLAG M2 affinity gel (Sigma) at 4° C. to pull down FLAG-tagged PCNA.

Measurement of compound metabolism in liver microsome. AOH1160 analogs were incubated in human liver microsomes in the presence or absence of NADPH at 37° C. An aliquot of the reaction mixture was taken after various incubation times. Compound concentration was determined by liquid chromatography-tandem mass spectrometry (LC-MS/MS) as previously described (Gu et al., 2018).

Proteomic analysis by mass spectrum. SK-N-AS cells homozygous of the wildtype or mutant RPB1 allele were treated with or without 0.5 μM AOH1996 overnight. Cell pellets were dissolved in 100 μL lysis buffer (0.5 M triethylammonium bicarbonate, 0.05% sodium dodecyl sulfate) and subjected to tip sonication. Protein lysates were quantified for protein content using the BCA protein assay kit (Thermo Fisher Scientific, Waltham, MA, USA) and equal amounts of protein were used per condition, adjusted to the highest volume with lysis buffer. Proteins were then reduced [4 μL of 100 mM methyl methanethiosulfonate (MMTS), 600 C for 1 hour], alkylated [2 μL of 100 mM tris(2-carboxyethyl) phosphine (TCEP), room temperature for 10 min) and enzymatically digested overnight [1:25 trypsin/LysC, 370 C in dark). Peptides were labelled using the 16-plex TMT reagents (TMT labels dissolved in 41 μL anhydrous acetonitrile and transferred to each sample, room temperature for 2 hr) (Thermo Fisher Scientific, Waltham, MA, USA). The labelling scheme was as follows: 126-untreated 1, 127N=treated 1, 127C=untreated 2, 128N=treated 2, 128C=untreated 3, 129N=treated 3, 129C=untreated 4, 130N=treated 4, 130C=26-0h-1, 131N=26-0h-2, 131C=26-24h-1, 132N=26-24h-2, 132C=31-0h-1, 133N=31-0h-2, 133C=31-24h-1, 134N=31-24h-2. The labelling reaction was stopped by adding 8 μL of 5% hydroxylamine in each sample and incubating at room temperature for 10 min. Peptides from all samples were then mixed and phospho-enrichment was performed using the Sequential enrichment of metal oxide affinity chromatography (SMOAC) protocol (Thermo Fisher Scientific, Waltham, MA, USA). Normalization was performed on total peptide amount and scaling was performed on all averages. The scaled abundance data was analyzed by the GO Process enrichment of MetaCore (Clarivate, Philadelphia, PA) with cutoff thresholds of p value (moderated t-test) less than 0.05 and fold of change (FC) greater than 2.

Pharmacokinetic (PK) study in animals. An oral dosing solution was prepared by dissolving AOH1996 (40 mg) in a mixture of Kolliphor EL (840 mg) and Poloxamer P124 (120 mg). For the mouse study, blood samples were collected from ES1^{e}/SCID mice (3 male and 3 female) per dosing group by cardiac puncture at 10, 20, and 30 min and 1, 2, 4, 6, and 24 h after dosing. For the dog study, blood samples were collected from 3 male beagle dogs per dosing group by venipuncture of peripheral veins at 5, 15, and 30 min and 1, 2, 4, 8, 12, and 24 h after dosing. Following removal of blood cells, plasma concentration of AOH1996 was determined by LC-MS/MS as described (Gu et al., 2018). Oral PK was determined using standard non-compartmental methods.

In vivo tumor model. All experiments involving live animals were carried out in strict accordance with the recommendations stated in the Guide for the Care and Use of Laboratory Animals, as adopted and promulgated by the National Institutes of Health. The protocol (#11034) was reviewed and approved by the City of Hope Institutional Animal Care and Use Committee. A breeding colony of ES1^{e}/SCID mice, originally provided by Dr. Philip M. Potter of the St. Jude Children's Research Hospital, was maintained at City of Hope. SK-N-BE(2)c and SK-N-AS neuroblastoma cells were suspended in Matrigel (BD Biosciences) at 5×10⁷/ml and 4×10⁷/ml, respectively, after being harvested and washed twice in PBS. Cell suspension (0.1 mL) was subcutaneously injected into the right flank of each ES1^{e}/SCID mouse. AOH1996 was dosed orally. CPT-11 was given by intraperitoneal injection. Tumor size and animal weight were measured weekly. At the end of the experiment, tumors were isolated from sacrificed mice and analyzed by immunohistochemistry staining with antibodies specific for phosphor-Chk1 and γH2A.X.

Protected amino acids were purchased from Chem-Impex International. Purification of designated intermediates and final compounds was performed using an ISCO CombiFlash chromatography system equipped with UV detector. All other reagents were purchased from Sigma-Aldrich, TCI, or Alfa Aesar (reagent grade) and used as received. ¹H, ¹⁹F, and ¹³C NMR spectra were obtained on Agilent 400 MHz spectrometer. All ¹H and ¹³C peak assignments were verified by COSY and HSQCAD. Multiplicities are quoted as singlet(s), doublet (d), triplet (t), unresolved multiplet (m), doublet of doublets (dd), doublet of doublet of doublets (ddd), doublet of triplets (dt), triplet of doublets (td), and broad (b). All chemical shifts (δ) are reported in parts per million (ppm) relative to residual CHCl₃ in CDCl₃ (δ 7.26, 1H NMR). Mass spectrometry (MS) was performed on a Thermo LTQ linear ion trap with a static nano-electrospray source in the positive ion mode (performed at COH core facility). MS m/z values were calculated using ChemDraw 20.1.1.125. Compound IUPAC names were assigned using ChemDraw 20.1.1.125. The molar yields of the final products were calculated weighing dry compounds. AOH1160LV and AOH1160DV were synthesized in according to method A (Scheme 1). The extended version of AOH1160 (named AOH1160e) with beta-alanine linker was synthesized using method B (Scheme 2). AOH1996, AOH1996LA, AOH1996t, AOH1160LA, AOH1160SO₂, AOH1160S, AOH1996S, AOH1996S-2F, AOH1996S-3F, AOH1996S-4F, AOH1996S-3CF₃, AOH1996S-4CF₃, AOH1996TMB, AOH-DMB-2CH3, AOH1160-2AB, AOH1996BINPh, AOH1996eNph and AOH1996eeNph were synthesized in using method C (Schemes 3-5).

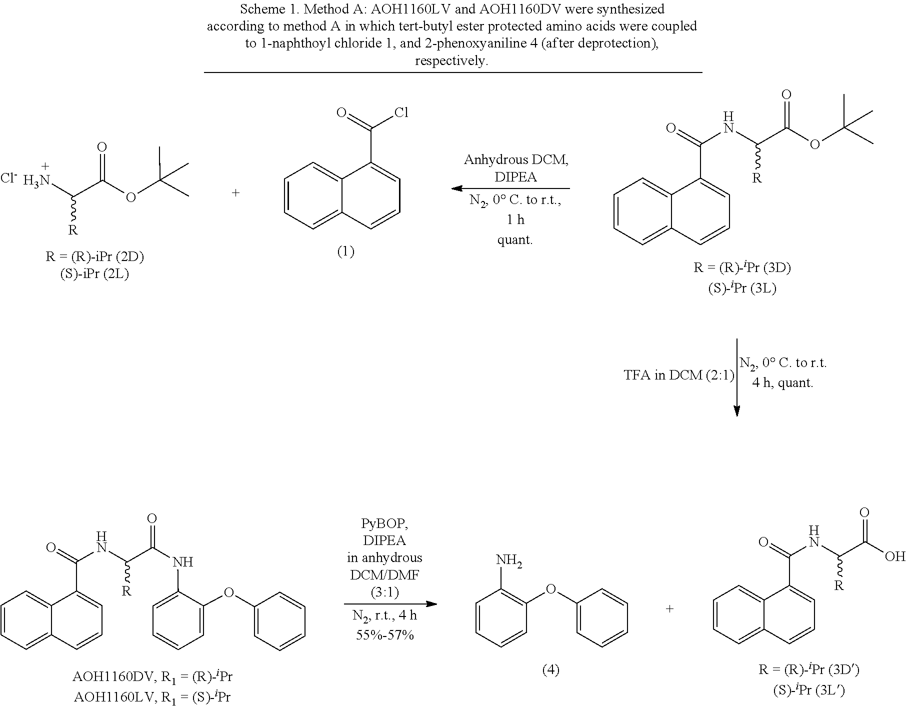

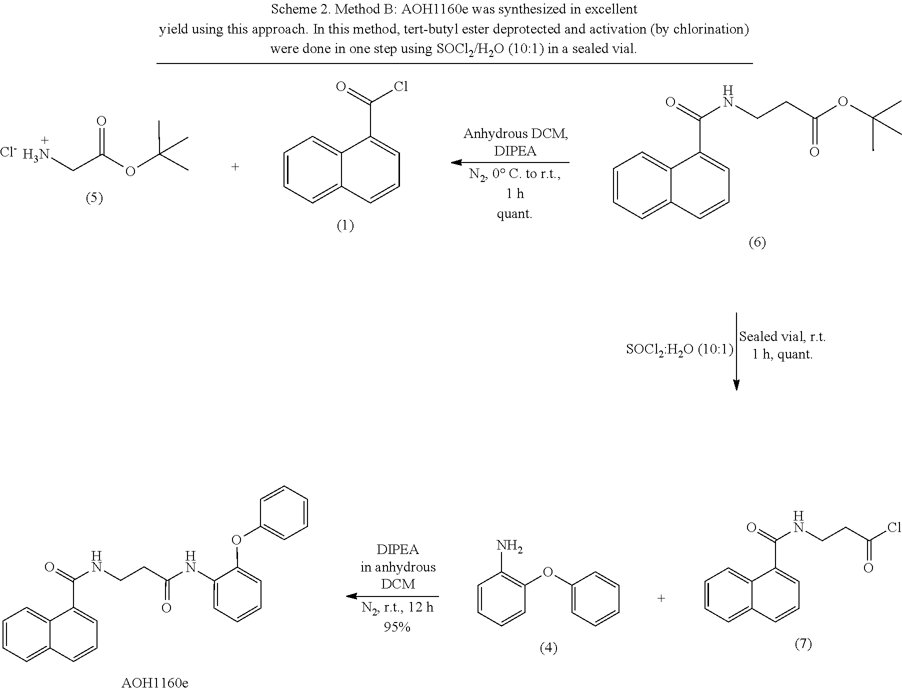

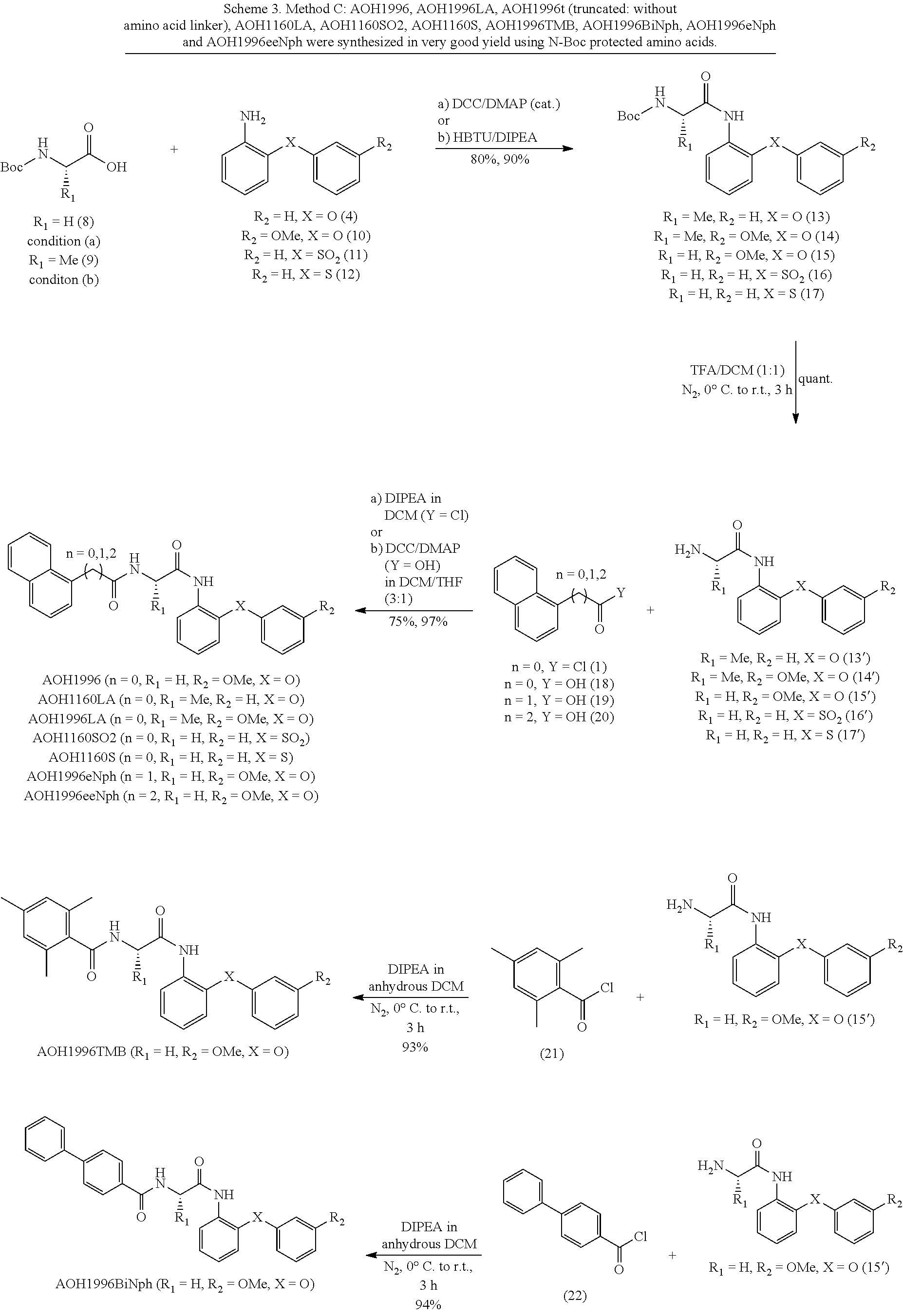

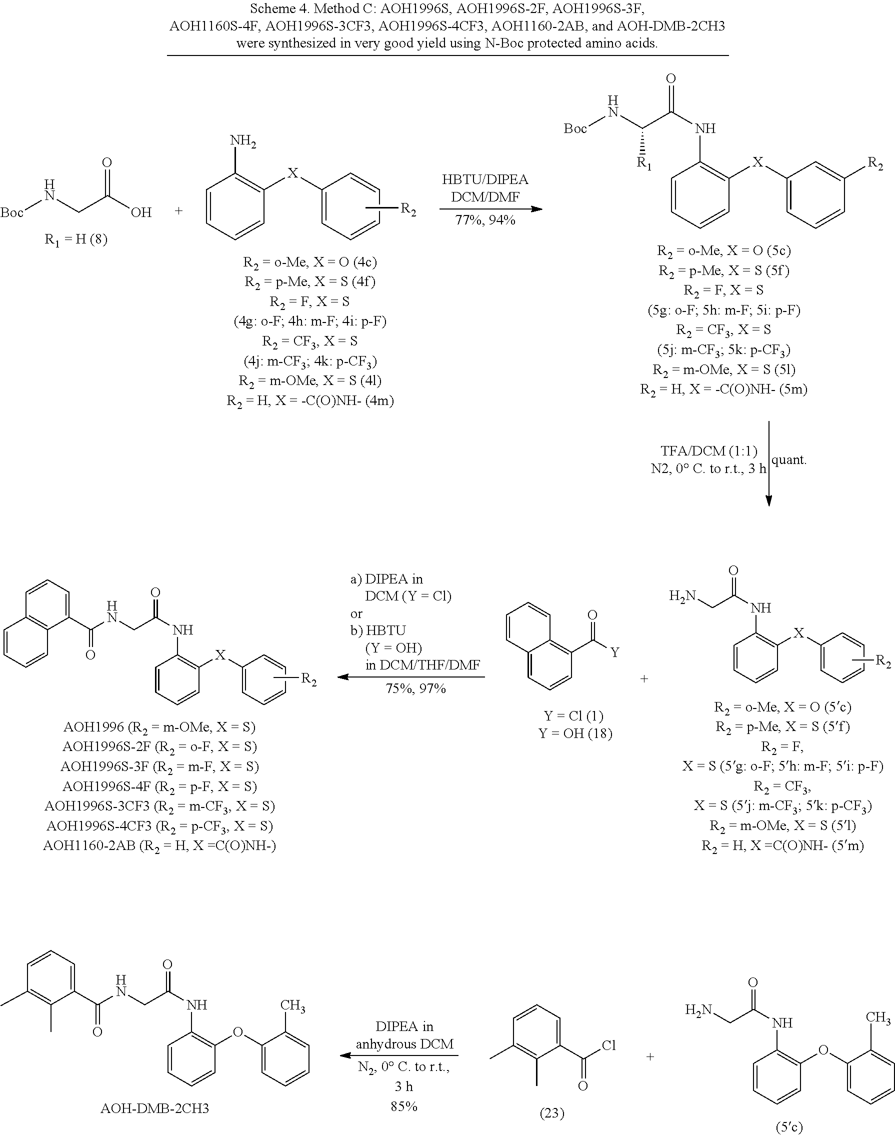

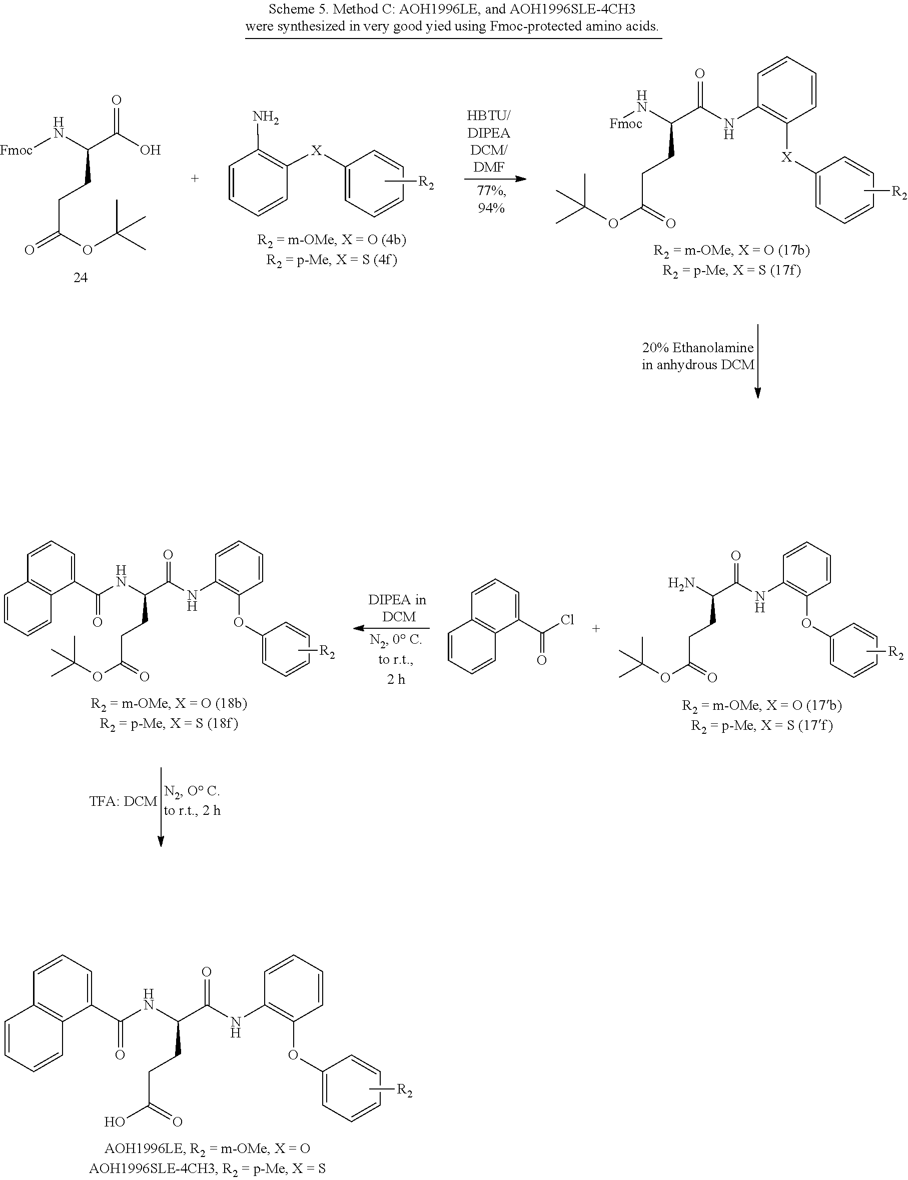

### Synthetic Procedures

General procedure 1: To a solution of the corresponding amine (1.0 equiv.) in anhydrous DCM (1.25 M amine) in iced bath, anhydrous DIPEA (3 equiv.) and 1-naphthyl chloride (1.0-1.2 equiv.) were added respectively. Then, the solution was stirred under nitrogen atmosphere at room temperature for 1-3 hours. After completion of the reaction (monitored by TLC), volatile was removed under reduced pressure, crude was redissolved in 50 mL DCM and washed with 20 mL saturated Na₂CO₃ solution, 20 mL HCl (0.1 M) and 20 mL brine solution.

General procedure 2: tert-butyl ester was dissolved in TFA/DCM (2:1) at 0° C. to reach the concentration of 0.5 M and the solution was brought to room temperature and was stirred for 4 hours. After completion of the reaction (monitored by TLC and ¹H NMR), volatile was removed under reduced pressure, co-evaporated with anhydrous DCM (3×2 mL) and used in the next step without further purification. To the corresponding free acid (1.0 equiv.) in anhydrous DCM/DMF (0.25M acid) in an ice bath, anhydrous DIPEA (3 equiv.), PyBOP (1.1 equiv.) and 2-phenoxyaniline (1.5 equiv.) were added respectively (Coste et al., 1990). The mixture was stirred under nitrogen atmosphere at room temperature for 4 hours. After completion of the reaction (monitored by TLC), volatile was removed under reduced pressure, crude was redissolved in 50 mL DCM and washed with 20 mL saturated Na₂CO₃ solution, 20 mL HCl (0.1 M) and 20 mL brine solution. Then, purified by CombiFlash chromatography, with a gradient of 0 to 40% ethyl acetate in hexane.

General procedure 3: Appropriate aniline derivatives (1.0 equiv.) was added to the solution of the corresponding dried N-Boc protected amino acid (1.1 equiv.), anhydrous DIPEA (3.3 equiv.) and HBTU (1.1 equiv.) in anhydrous DCM/MeCN (10:1) (0.2 M to aniline) in an ice bath (Dourtoglou et al., 1978). The mixture was brought to room temperature and stirred under nitrogen atmosphere for 2.5 hours. After completion of the reaction (monitored by TLC), volatile was removed under reduced pressure, crude was redissolved in 50 mL DCM and washed with 20 mL saturated Na₂CO₃ solution and 20 mL brine solution. Then, purified by CombiFlash chromatography, with a gradient wash (ethyl acetate/hexane).

General procedure 4: Appropriate amine derivatives (1.0 equiv.) was added to the solution of the corresponding dried acid (1.2 equiv.), DCC (1.2 equiv.) and catalytic amount of DMAP in anhydrous DCM (0.16 M to amine). The mixture was stirred at room temperature under nitrogen atmosphere overnight. After completion of the reaction the mixture was filtered by a fritted glass filter to remove the DCU byproduct and the filtrate was diluted with 50 mL DCM and washed with 20 mL saturated Na₂CO₃ solution (and 20 mL 0.1 M HCl in the absence of N-Boc group) and 20 mL brine solution. Then, purified by CombiFlash chromatography, with a gradient wash (ethyl acetate/hexane).

General procedure 5: The corresponding N-Boc protected derivative dissolved (0.6 M) in anhydrous TFA/DCM (1:1) in an ice-bath and the solution was placed at room temperature and stirred for 3 hours under nitrogen atmosphere. After completion of the reaction (monitored by TLC and ¹H NMR), volatile was removed under reduced pressure, co-evaporated with anhydrous DCM (3×2 mL), crude was redissolved in 50 mL DCM and washed with 5 mL saturated Na₂CO₃ (keep the pH to >11 by adding NaOH) to remove residual TFA salts. The corresponding free amine was used in the next step without further purification.

General procedure 6: Appropriate amine derivatives (1.0 equiv.) was added to the solution of the corresponding dried aromatic carboxylic acid derivatives (1.1 equiv.), anhydrous DIPEA (3.3 equiv.) and HBTU (1.1 equiv.) in anhydrous DCM/DMF (10:1) (0.2 M to amine) in an ice bath (Dourtoglou et al., 1978). The mixture was brought to room temperature and stirred under nitrogen atmosphere for 18 hours. After completion of the reaction (monitored by TLC), volatile was removed under reduced pressure, crude was redissolved in 50 mL DCM and washed with 20 mL saturated Na₂CO₃ solution, 20 mL 0.1 M HCl and 20 mL brine solution. Then, purified by CombiFlash chromatography, with a gradient wash (ethyl acetate/hexane).

General procedure 7: The corresponding Fmoc-protected derivative dissolved in 20% ethanolamine in DCM (0.3 M) in an ice bath and the solution was placed at room temperature and stirred for 1 hour under a nitrogen atmosphere. After completion of the reaction (monitored by TLC and ¹H NMR), the volatile was removed under reduced pressure. The corresponding free amine was then purified by CombiFlash chromatography (solid load), with a gradient wash (ethyl acetate/hexane).

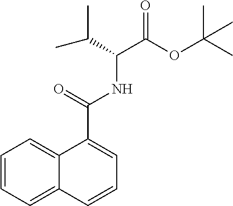 [SMILES: CC(C)[C@@H](NC(=O)c1cccc2ccccc12)C(=O)OC(C)(C)C]

Synthesis of tert-butyl (1-naphthoyl)-D-valinate (3D): According to general procedure 1, 2.2 mmol D-Valine tert-butyl ester hydrochloride 2D was reacted with 2.2 mmol of 1-naphthyl chloride 1 to provide 720 mg (2.2 mmol) title compound (3D) in quantitative yield as a white powder. ¹H NMR (400 MHZ, CDCl₃) δ 8.35 (dd, J=7.8, 1.8, Hz, 1H), 7.92 (dd, J=8.4, 1.1 Hz, 1H), 7.86 (dd, J=8.2, 1.4 Hz, 1H), 7.67 (dd, J=7.0, 1.3 Hz, 1H), 7.59-7.42 (m, 3H), 6.47 (d, J=8.9 Hz, 1H), 4.78 (dd, J=8.9, 4.5 Hz, 1H), 2.33 (pd, J=6.9, 4.5 Hz, 1H), 1.51 (s, 9H), 1.08 (d, J=6.9 Hz, 3H), 0.98 (d, J=6.9 Hz, 3H). ¹³C NMR (101 MHz, CDCl₃) δ 171.06, 169.21, 134.28, 133.69, 130.76, 130.17, 128.27, 127.15, 126.41, 125.43, 125.12, 124.71, 82.19, 57.80, 31.57, 28.09, 19.14, 17.71. MS (ESI+) m/z: [M+Na]⁺ calcd for C₂₀H₂₅NO₃Na⁺ 350.2; found 350.1.

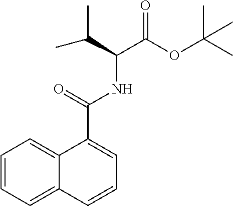 [SMILES: CC(C)[C@H](NC(=O)c1cccc2ccccc12)C(=O)OC(C)(C)C]

Synthesis of tert-butyl (1-naphthoyl)-L-valinate (3 L): According to general procedure 1, 2.6 mmol D-Valine tert-butyl ester hydrochloride 2 L was reacted with 2.6 mmol of 1-naphthyl chloride 1 to provide 851 mg (2.6 mmol) title compound (3L) in quantitative yield as a white powder. ¹H NMR (400 MHZ, CDCl₃) δ 8.37 (dd, J=8.2, 1.0 Hz, 1H), 7.91 (d, J=8.3 Hz, 1H), 7.86 (d, J=7.9 Hz, 1H), 7.68 (dd, J=7.1, 1.2 Hz, 1H), 7.60-7.42 (m, 3H), 6.51 (d, J=8.8 Hz, 1H), 4.79 (dd, J=8.8, 4.5 Hz, 1H), 2.33 (pd, J=6.9, 4.6 Hz, 1H), 1.52 (s, 9H), 1.09 (d, J=6.9 Hz, 3H), 0.99 (d, J=6.9 Hz, 3H). ¹³C NMR (101 MHZ, CDCl₃) δ 171.03, 169.19, 134.33, 133.73, 130.73, 130.21, 128.26, 127.11, 126.38, 125.46, 125.11, 124.69, 82.14, 57.90, 31.55, 28.10, 19.12, 17.76. MS (ESI+) m/z: [M+Na]⁺ calcd for C₂₀H₂₅NO₃Na⁺ 350.2; found 350.2.

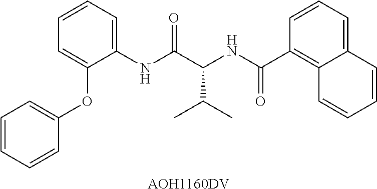 [SMILES: CC(C)[C@@H](NC(=O)c1cccc2ccccc12)C(=O)Nc1ccccc1Oc1ccccc1 ; "AOH1160DV"]

Synthesis of (R)—N-(3-methyl-1-oxo-1-((2-phenoxyphenyl)amino) butan-2-yl)-1-naphthamide (AOH1160DV): According to general procedure 2, 1.0 mmol 3D was deprotected and coupled to 2-phenoxyaniline 4 to yield brown solid of AOH1160DV (MW: 438.5 g/mol, 241 mg, 0.55 mmol, 55%). ¹H NMR (400 MHZ, CDCl₃) δ 8.40 (dd, J=8.1, 1.7 Hz, 1H), 8.34-8.25 (m, 2H), 7.90 (dd, J=8.3, 1.1 Hz, 1H), 7.88-7.81 (m, 1H), 7.61 (dd, J=7.1, 1.2 Hz, 1H), 7.54-7.46 (m, 2H), 7.40 (dd, J=8.3, 7.0 Hz, 1H), 7.36-7.30 (m, 2H), 7.16-7.09 (m, 2H), 7.08-6.99 (m, 3H), 6.88 (dd, J=8.1, 1.5 Hz, 1H), 6.70 (d, J=8.8 Hz, 1H), 4.75 (dd, J=8.8, 6.2 Hz, 1H), 2.30 (dq, J=13.5, 6.8 Hz, 1H), 1.04 (dd, J=18.3, 6.8 Hz, 6H). ¹³C NMR (101 MHz, CDCl₃) δ 169.55, 169.31, 156.26, 146.01, 133.73, 133.67, 130.96, 130.14, 129.98, 129.08, 128.29, 127.26, 126.46, 125.32, 125.20, 124.63, 124.58, 123.99, 123.96, 121.16, 118.64, 117.85, 59.60, 31.34, 19.36, 18.10. MS (ESI+) m/z: [M+Na]⁺ calcd for C₂₈H₂₆N₂O₃Na⁺ 461.2; found 461.2.

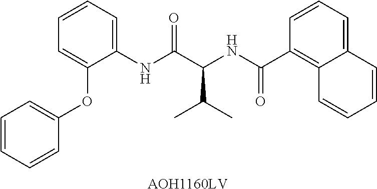 [SMILES: CC(C)[C@H](NC(=O)c1cccc2ccccc12)C(=O)Nc1ccccc1Oc1ccccc1 ; "AOH1160LV"]

Synthesis of(S)—N-(3-methyl-1-oxo-1-((2-phenoxyphenyl)amino) butan-2-yl)-1-naphthamide (AOH1160LV): According to general procedure 2, 1.0 mmol 3 L was deprotected and coupled to 2-phenoxyaniline 4 to yield brown solid of AOH1160LV (MW: 438.5 g/mol, 250 mg, 0.57 mmol, 57%). ¹H NMR (400 MHZ, CDCl₃) δ 8.42-8.33 (m, 2H), 8.32-8.24 (m, 1H), 7.90 (d, J=8.3 Hz, 1H), 7.88-7.81 (m, 1H), 7.61 (dd, J=7.0, 1.2 Hz, 1H), 7.55-7.44 (m, 2H), 7.43-7.27 (m, 3H), 7.17-6.74 (m, 8H), 5.20 (s, 1H), 4.77 (dd, J=8.8, 6.3 Hz, 1H), 2.29 (hept, J=6.8 Hz, 1H), 1.05 (dd, J=18.7, 6.8 Hz, 6H). ¹³C NMR (101 MHz, CDCl₃) δ 169.80, 169.41, 156.25, 146.14, 133.66, 133.50, 131.06, 130.10, 129.98, 129.75, 128.97, 128.32, 127.30, 126.48, 125.29, 125.25, 124.71, 124.63, 124.00, 123.94, 123.09, 121.31, 118.66, 117.88, 117.69, 59.70, 31.37, 19.34, 18.14. MS (ESI+) m/z: [M+Na]⁺ calcd for C₂₈H₂₆N₂O₃Na⁺ 461.2; found 461.2.

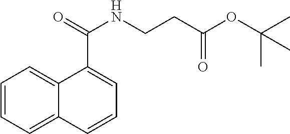 [SMILES: CC(C)(C)OC(=O)CCNC(=O)c1cccc2ccccc12]

Synthesis of tert-butyl 3-(1-naphthamido) propanoate (6): According to general procedure 1, 2.6 mmol beta-alanine tert-butyl ester hydrochloride 5 was reacted with 2.6 mmol of 1-naphthyl chloride 1 to provide 778 mg (2.6 mmol) title compound (6) in quantitative yield as a white powder. ¹H NMR (400 MHZ, CDCl₃) δ 8.31-8.23 (m, 1H), 8.03-7.91 (m, 2H), 7.63-7.48 (m, 4H), 7.05 (b, 1H), 3.64 (AB system appears as td, J=6.7, 5.9 Hz, 2H), 2.60 (t, J=6.6 Hz, 2H), 1.47 (s, 9H). ¹³C NMR (101 MHZ, CDCl₃) δ 171.14, 169.02, 134.89, 133.60, 130.06, 130.03, 128.22, 126.77, 126.33, 125.43, 125.00, 124.94, 80.27, 35.67, 35.10, 27.32. MS (ESI+) m/z: [M+Na]⁺ calcd for C₁₈H₂₁NO₃Na⁺ 322.1; found 322.2.

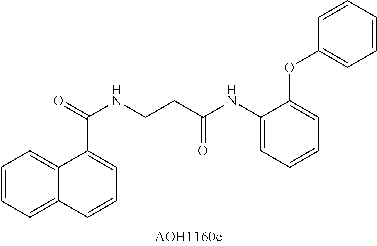 [SMILES: O=C(CCNC(=O)c1cccc2ccccc12)Nc1ccccc1Oc1ccccc1]

Synthesis of N-(3-oxo-3-((2-phenoxyphenyl)amino) propyl)-1-naphthamide (AOH1160e): according to literature procedure (Greenberg and Sammakia, 2017), 299 mg (1 mmol, 1 equiv.) tert-butyl 3-(1-naphthamido) propanoate 6 was dissolved in 0.7 mL SOCl₂ (10 mmol, 10 equiv.) in a 3 mL vial and 18 μL H₂O (1 mmol, 1 equiv.) was added to it at room temperature and the vial was capped immediately. The solution was stirred under sealed condition for 1 hour and then opened carefully to release the trapped gas. Volatile was removed under reduced pressure, co-evaporated with anhydrous DCM (2×2 mL) and activated acid chloride 7 was used in the next step without further purification. Then, to the freshly produced 7 (1.0 equiv.) in 2 mL anhydrous DCM in an ice bath, anhydrous DIPEA (3 equiv.) and 2-phenoxyaniline 4 (1.5 equiv.) were added respectively. The mixture was stirred under nitrogen atmosphere at room temperature for 12 hours. After completion of the reaction (monitored by TLC), volatile was removed under reduced pressure, crude was redissolved in 50 mL DCM and washed with 20 mL saturated Na₂CO₃ solution, 20 mL HCl (0.1 M) and 20 mL brine solution. After purification by CombiFlash chromatography, with a gradient of 0 to 10% methanol in DCM, the fractions containing the desired product combined and concentrated to yield AOH1160e as a beige powder (MW: 410.5 g/mol, 390 mg, 0.95 mmol, 95%). ¹H NMR (400 MHZ, CDCl₃) δ 8.29 (ddd, J=19.1, 7.8, 2.1 Hz, 2H), 8.05 (s, 1H), 7.81 (dd, J=12.2, 7.8 Hz, 2H), 7.55-7.38 (m, 3H), 7.37-7.23 (m, 3H), 7.15-6.93 (m, 6H), 6.82 (dd, J=8.1, 1.7 Hz, 1H), 3.75 (q, J=6.1 Hz, 2H), 2.66 (t, J=6.0 Hz, 2H). ¹³C NMR (101 MHz, CDCl₃) δ 170.07, 169.66, 156.29, 146.23, 134.24, 133.64, 130.52, 130.15, 129.97, 129.29, 128.23, 126.98, 126.30, 125.42, 125.12, 124.68, 124.46, 124.00, 123.82, 121.52, 118.76, 117.74, 36.67, 35.96. MS (ESI+) m/z: [M+Na]⁺ calcd for C₂₆H₂₂N₂O₃Na⁺ 433.1; found 433.0.

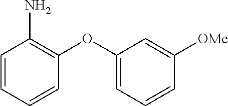 [SMILES: COc1cccc(Oc2ccccc2N)c1]

Synthesis of (3-methoxyphenoxy)aniline (10): According to literature procedure (Bueno et al., 2019), 10 mmol (1 equiv.) of 1-fluoro-2-nitrobenzene was added dropwise to the pre-warmed mixture of 11 mmol (1.1 equiv.) 3-methoxyphenol and 11 mmol Cs₂CO₃ in 30 mL anhydrous acetonitrile at 80° C. under nitrogen atmosphere. The mixture was rigorously stirred at 80° C. for 5.5 hours. After completion (monitored by TLC), the solution was cooled down to room temperature, volatile was removed under reduced pressure and 100 mL cold water was added and the mixture was extracted with ethyl acetate (3×100 mL). The organic layer was combined and dried over MgSO₄, concentrated, and purified by flash chromatography with a gradient of 0 to 10% ethyl acetate in hexane to provide 2.64 g (MW: 245.2 g/mol, 10.8 mmol, 98%) 1-(3-methoxyphenoxy)-2-nitrobenzene, 10′. 1H NMR (400 MHz, CDCl₃) δ 7.91 (dd, J=8.1, 1.2 Hz, 1H), 7.48 (dd, J=8.6, 7.4 Hz, 1H), 7.24 (t, J=8.1 Hz, 1H), 7.20-7.15 (m, 1H), 7.03 (dd, J=8.4, 1.3 Hz, 1H), 6.71 (dd, J=8.3, 2.2 Hz, 1H), 6.62-6.56 (m, 2H), 3.76 (s, 3H).

Then, it was reduced to the corresponding aniline derivative under balloon pressure of hydrogen gas in 5 mL anhydrous ethyl acetate/ethanol (4:1) using catalytic amount of 10% Pd/C. After completion (monitored by MS), solids were removed by filtration, and concentrated to yield of 2.32 g (MW: 215.2 g/mol, 10.8 mmol, 100%) title compound 10 as a pale-yellow oil. ¹H NMR (400 MHZ, CDCl₃) δ 7.22-7.15 (m, 1H), 6.98 (dd, J=7.9, 1.5 Hz, 1H), 6.89 (dd, J=8.0, 1.5 Hz, 1H), 6.81 (dd, J=7.9, 1.6 Hz, 1H), 6.72 (td, J=7.7, 1.6 Hz, 1H), 6.65-6.59 (m, 1H), 6.54 (appears as dq, J=8.1, 1.4, 0.8 Hz, 2H), 3.76 (s, 3H). ¹³C NMR (101 MHZ, CDCl₃) δ 160.97, 158.72, 142.84, 138.68, 130.12, 125.02, 120.47, 118.83, 118.81, 116.55, 109.19, 108.21, 103.26, 55.32. MS (ESI+) m/z: [M+Na]⁺ calcd for C₁₃H₁₄NO₂Na⁺ 216.1; found 216.1.

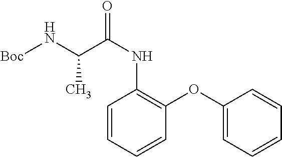 [SMILES: C[C@H](NC(=O)OC(C)(C)C)C(=O)Nc1ccccc1Oc1ccccc1]

Synthesis of tert-butyl(S)-(1-oxo-1-((2-phenoxyphenyl)amino) propan-2-yl)carbamate (13): According to general procedure 3, 2 mmol of 2-phenoxyaniline 4 was treated with 2.2 mmol N-Boc-L-alanine 9 and purified by CombiFlash chromatography, with a gradient of 0 to 30% ethyl acetate in hexane to provide 620 mg (MW: 356.4 g/mol, 1.7 mmol, 87%) title compound (13) as a solid powder. ¹H NMR (400 MHZ, CDCl₃) δ 8.52 (b, 1H), 8.41 (dd, J=8.1, 1.6 Hz, 1H), 7.37-7.26 (m, 2H), 7.15-7.04 (m, 2H), 7.04-6.93 (m, 3H), 6.84 (dd, J=8.1, 1.5 Hz, 1H), 4.94 (s, 1H), 4.27 (s, 1H), 1.37 (d, J=6.9 Hz, 3H), 1.36 (s, 9H). ¹³C NMR (101 MHz, CDCl₃) δ 170.78, 156.52, 145.84, 129.85, 129.55, 124.15, 123.99, 123.76, 120.94, 118.49, 117.93, 80.16, 51.12, 28.18, 18.01. MS (ESI+) m/z: [M+Na]⁺ calcd for C₂₀H₂₄N₂O₄Na⁺ 379.2; found 379.2.

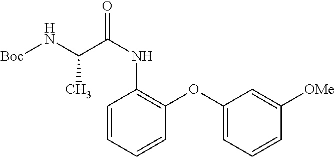 [SMILES: COc1cccc(Oc2ccccc2NC(=O)[C@H](C)NC(=O)OC(C)(C)C)c1]

Synthesis of tert-butyl(S)-(1-((2-(3-methoxyphenoxy)phenyl)amino)-1-oxopropan-2-yl)carbamate (14): According to general procedure 3, 2 mmol of 2-(3-methoxyphenoxy)aniline 10 was reacted with 2.4 mmol N-Boc-L-alanine 9 and purified by CombiFlash chromatography, with a gradient of 0 to 30% ethyl acetate in hexane to provide 695 mg (MW: 386.4 g/mol, 1.8 mmol, 90%) title compound (14) as a solid powder. ¹H NMR (400 MHZ, CDCl₃) δ 8.51 (s, 1H), 8.41 (dd, J=8.2, 1.7 Hz, 2H), 7.23-7.17 (m, 2H), 7.10 (td, J=7.5, 1.5 Hz, 2H), 7.00 (td, J=7.7, 1.7 Hz, 2H), 6.88 (dd, J=8.1, 1.5 Hz, 2H), 6.69-6.61 (m, 2H), 6.60-6.51 (m, 3H), 4.95 (s, 1H), 4.27 (s, 1H), 3.76 (s, 4H), 1.37 (d, J=7.2 Hz, 5H), 1.36 (s, 8H). ¹³C NMR (101 MHZ, CDCl₃) δ 170.80, 161.02, 157.65, 145.54, 130.27, 129.57, 124.16, 124.13, 120.86, 118.17, 110.51, 109.49, 104.52, 55.36, 51.02, 28.18, 18.01. MS (ESI+) m/z: [M+Na]⁺ calcd for C₂₁H₂₆N₂O₅Na⁺ 409.2; found 409.2.

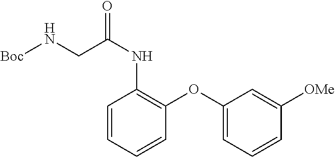 [SMILES: COc1cccc(Oc2ccccc2NC(=O)CNC(=O)OC(C)(C)C)c1]

Synthesis of tert-butyl (2-((2-(3-methoxyphenoxy)phenyl)amino)-2-oxoethyl)carbamate (15): According to general procedure 4, 2 mmol of 2-(3-methoxyphenoxy)aniline 10 was coupled with 2.4 mmol N-Boc glycine 8 and purified by CombiFlash chromatography, with a gradient of 0 to 50% ethyl acetate in hexane to provide 647 mg (MW: 372.4 g/mol, 1.7 mmol, 87%) title compound (15) as a white powder. ¹H NMR (400 MHz, CDCl₃) δ 8.41 (d, J=8.3 Hz, 1H), 8.38 (s, 1H), 7.22 (td, J=8.4, 0.7 Hz, 1H), 7.15-7.07 (m, 1H), 7.01 (t, J=7.7 Hz, 1H), 6.88 (dd, J=8.1, 1.5 Hz, 1H), 6.71-6.63 (m, 1H), 6.62-6.51 (m, 2H), 5.08 (s, 1H), 3.90 (d, J=6.0 Hz, 2H), 3.76 (s, 3H), 1.38 (s, 9H). ¹³C NMR was taken after Boc deprotection step (compound 15′). MS (ESI+) m/z: [M+Na]⁺ calcd for C₂₀H₂₄N₂O₅Na⁺ 395.2; found 395.2. (95% yield was obtained when using general procedure 3.)

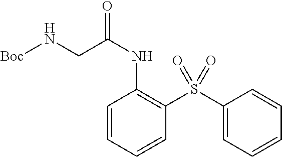 [SMILES: CC(C)(C)OC(=O)NCC(=O)Nc1ccccc1S(=O)(=O)c1ccccc1]

Synthesis of tert-butyl (2-oxo-2-((2-(phenylsulfonyl)phenyl)amino)ethyl)carbamate (16): According to general procedure 4, 2 mmol of 2-(phenylsulfonyl)aniline 11 was coupled with 2.4 mmol N-Boc glycine 8 and purified by CombiFlash chromatography, with a gradient of 0 to 70% ethyl acetate in hexane to provide 625 mg (MW: 390.5 g/mol, 1.6 mmol, 80%) title compound (16) as a solid powder. ¹H NMR (400 MHZ, CDCl₃) δ 7.96-7.88 (m, 2H), 7.58-7.44 (m, 5H), 7.32-7.14 (m, 2H), 5.16 (s, 2H), 4.60-4.52 (m, 2H), 1.43 (s, 9H). MS (ESI+) m/z: [M+Na]⁺ calcd for C₁₉H₂₂N₂O₅SNa⁺ 413.1; found 413.1. (89% yield was obtained when using general procedure 3.)

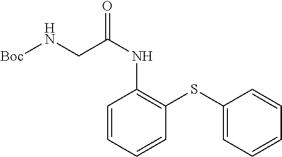 [SMILES: CC(C)(C)OC(=O)NCC(=O)Nc1ccccc1Sc1ccccc1]

Synthesis of tert-butyl (2-oxo-2-((2-(phenylthio)phenyl)amino)ethyl)carbamate (17): According to general procedure 4, 2 mmol of 2-(phenylthio)aniline 12 was coupled with 2.4 mmol N-Boc glycine 8 and purified by CombiFlash chromatography, with a gradient of 0 to 30% ethyl acetate in hexane to provide 674 mg (MW: 358.5 g/mol, 1.88 mmol, 94%) title compound (17) as a white powder. ¹H NMR (400 MHZ, CDCl₃) δ 8.88 (s, 1H), 8.45 (d, J=8.3 Hz, 1H), 7.57 (d, J=7.8 Hz, 1H), 7.47-7.38 (m, 1H), 7.27-7.19 (m, 2H), 7.19-7.04 (m, 4H), 4.83 (s, 1H), 3.82 (d, J=6.1 Hz, 2H), 1.44 (s, 9H). MS (ESI+) m/z: [M+Na]⁺ calcd for C₁₉H₂₂N₂O₃SNa⁺ 381.1; found 381.1. (97% yield was obtained when using general procedure 3.)

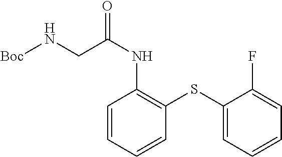 [SMILES: CC(C)(C)OC(=O)NCC(=O)Nc1ccccc1Sc1ccccc1F]

Synthesis of tert-butyl (2-((2-((2-fluorophenyl)thio)phenyl)amino)-2-oxoethyl)carbamate (5 g): According to general procedure 3, 3 mmol of 2-((2-fluorophenyl)thio)aniline 4 g was coupled with 3.3 mmol N-Boc glycine 8 and purified by CombiFlash chromatography, with a gradient of 0 to 25% ethyl acetate in hexane to provide 994 mg (MW: 376.4 g/mol, 2.64 mmol, 88%) title compound (4g) as pale yellow oil. ¹H NMR (400 MHZ, CDCl₃) δ 8.99 (s, 1H), 8.45 (d, J=8.3 Hz, 1H), 7.57 (dt, J=7.8, 1.7 Hz, 1H), 7.42 (ddt, J=8.6, 7.6, 1.8 Hz, 1H), 7.23-6.93 (m, 4H), 6.89 (d, J=7.9 Hz, 1H), 5.03 (s, 1H), 3.89 (d, J=6.0 Hz, 2H), 1.43 (s, 9H). ¹⁹F NMR (376 MHz, CDCl₃) δ −110.57. MS (ESI+) m/z: [M+Na]⁺ calcd for C₁₉H₂₁FN₂O₃SNa⁺ 399.1; found 399.2.

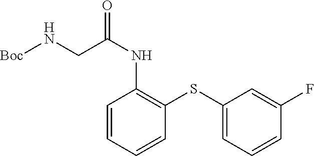 [SMILES: CC(C)(C)OC(=O)NCC(=O)Nc1ccccc1Sc1cccc(F)c1]

Synthesis of tert-butyl (2-((2-((3-fluorophenyl)thio)phenyl)amino)-2-oxoethyl)carbamate (5h): According to general procedure 3, 3 mmol of 2-((3-fluorophenyl)thio)aniline 4h was coupled with 3.3 mmol N-Boc glycine 8 and purified by CombiFlash chromatography, with a gradient of 0 to 25% ethyl acetate in hexane to provide 870 mg (MW: 376.4 g/mol, 2.31 mmol, 77%) title compound (5h) as a white solid. ¹H NMR (400 MHZ, CDCl₃) δ 8.94 (s, 1H), 8.49 (d, J=8.3 Hz, 1H), 7.58 (dd, J=7.7, 1.5 Hz, 1H), 7.46 (td, J=8.7, 8.1, 1.7 Hz, 1H), 7.23-7.12 (m, 2H), 6.83 (tdd, J=6.8, 2.9, 1.1 Hz, 2H), 6.75 (dt, J=9.2, 2.2 Hz, 1H), 4.98 (s, 1H), 3.85 (d, J=6.1 Hz, 2H), 1.44 (s, 9H). ¹⁹F NMR (376 MHz, CDCl₃) δ −111.17-−111.31 (m). MS (ESI+) m/z: [M+Na]⁺ calcd for C₁₉H₂₁FN₂O₃SNa⁺ 399.1; found 399.1.

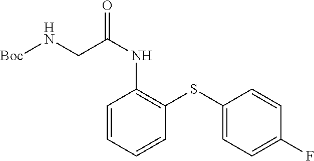 [SMILES: CC(C)(C)OC(=O)NCC(=O)Nc1ccccc1Sc1ccc(F)cc1]

Synthesis of tert-butyl (2-((2-((4-fluorophenyl)thio)phenyl)amino)-2-oxoethyl)carbamate (5i): According to general procedure 3, 3 mmol of 2-((4-fluorophenyl)thio)aniline 4i was coupled with 3.3 mmol N-Boc glycine 8 and purified by CombiFlash chromatography, with a gradient of 0 to 25% ethyl acetate in hexane to provide 1016 mg (MW: 376.4 g/mol, 2.70 mmol, 90%) title compound (51) as a colorless oil. 1H NMR (400 MHz, CDCl₃) δ 8.89 (s, 1H), 8.39 (d, J=8.2 Hz, 1H), 7.49 (d, J=7.6 Hz, 1H), 7.37 (td, J=7.9, 1.6 Hz, 1H), 7.11-7.03 (m, 3H), 6.94-6.87 (m, 2H), 4.92 (s, 1H), 3.83 (d, J=6.1 Hz, 2H), 1.40 (s, 9H). ¹⁹F NMR (376 MHZ, CDCl₃) δ −115.15 (tt, J=8.8, 5.1 Hz). MS (ESI+) m/z: [M+Na]⁺ calcd for C₁₉H₂₁FN₂O₃SNa⁺ 399.1; found 399.1.

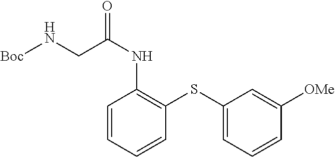 [SMILES: COc1cccc(Sc2ccccc2NC(=O)CNC(=O)OC(C)(C)C)c1]

Synthesis of tert-butyl (2-((2-((3-methoxyphenyl)thio)phenyl)amino)-2-oxoethyl)carbamate (51): According to general procedure 3, 4 mmol of 2-((3-methoxyphenyl)thio)aniline 10 was coupled with 4.4 mmol N-Boc glycine 8 and purified by CombiFlash chromatography, with a gradient of 0 to 35% ethyl acetate in hexane to provide 1.38 g (MW: 388.5 mol, 3.56 mmol, 89%) title compound (51). ¹H NMR (400 MHZ, CDCl₃) δ 8.88 (s, 1H), 8.44 (d, J=8.3 Hz, 1H), 7.56 (ddd, J=7.8, 5.1, 1.6 Hz, 1H), 7.43 (ddd, J=8.6, 7.6, 1.6 Hz, 1H), 7.17-7.08 (m, 2H), 6.71-6.64 (m, 3H), 3.82 (d, J=6.0 Hz, 2H), 3.69 (s, 3H), 1.43 (s, 9H). MS (ESI+) m/z: [M+Na]⁺ calcd for C₂₀H₂₄N₂O₄SNa⁺ 411.1; found 411.1.

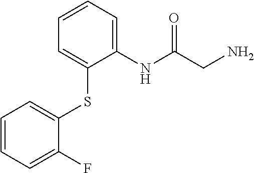 [SMILES: NCC(=O)Nc1ccccc1Sc1ccccc1F]

Synthesis of 2-amino-N-(2-((2-fluorophenyl)thio)phenyl) acetamide (5′g): According to general procedure 5, 2.64 mmol of compound 5g was deprotected in TFA/DCM (1:1) solution to produce 5′g in quantitative yield. ¹H NMR (400 MHZ, CDCl₃) δ 10.33 (s, 1H), 8.53 (d, J=8.1 Hz, 1H), 7.59 (dd, J=7.7, 1.6 Hz, 1H), 7.49-7.40 (m, 1H), 7.17-7.09 (m, 2H), 7.04 (ddd, J=9.6, 8.1, 1.2 Hz, 1H), 7.01-6.92 (m, 1H), 6.85 (td, J=7.7, 1.7 Hz, 1H), 3.41 (d, J=0.6 Hz, 2H). ¹⁹F NMR (376 MHz, CDCl₃) δ −111.10 (td, J=8.7, 5.2 Hz). MS (ESI+) m/z: [M+H]⁺ calcd for C₁₄H₁₄FN₂OS⁺ 277.1; found 277.1.

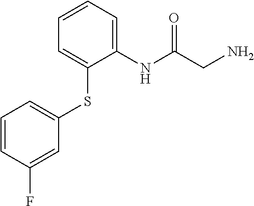 [SMILES: NCC(=O)Nc1ccccc1Sc1cccc(F)c1]

Synthesis of 2-amino-N-(2-((3-fluorophenyl)thio)phenyl) acetamide (5′h): According to general procedure 5, 2.31 mmol of compound 5h was deprotected in TFA/DCM (1:1) solution to produce 5′h in quantitative yield. MS (ESI+) m/z: [M+H]⁺ calcd for C₁₄H₁₄FN₂OS⁺ 277.1; found 277.2.

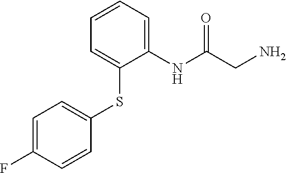 [SMILES: NCC(=O)Nc1ccccc1Sc1ccc(F)cc1]

Synthesis of 2-amino-N-(2-((4-fluorophenyl)thio)phenyl) acetamide (5′i): According to general procedure 5, 2.70 mmol of compound 5i was deprotected in TFA/DCM (1:1) solution to produce 5′i in quantitative yield (pale yellow oil). ¹H NMR (400 MHZ, CDCl₃) δ 7.69 (d, J=8.0 Hz, 1H), 7.35-7.20 (m, 5H), 7.16 (td, J=7.6, 1.5 Hz, 1H), 7.07-6.97 (m, 2H), 4.85 (s, 3H), 3.78 (s, 2H). ¹⁹F NMR (376 MHz, CD₃OD) δ −116.50 (tt, J=8.8, 4.9 Hz). MS (ESI+) m/z: [M+H]⁺ calcd for C₁₄H₁₄FN₂OS⁺ 277.1; found 277.1.

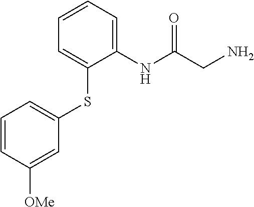 [SMILES: COc1cccc(Sc2ccccc2NC(=O)CN)c1]

Synthesis of 2-amino-N-(2-((3-methoxyphenyl)thio)phenyl) acetamide (5′1): According to general procedure 5, 3.56 mmol of compound 5l was deprotected in TFA/DCM (1:1) solution to produce 5′l in quantitative yield. ¹H NMR (400 MHZ, CDCl₃) δ 10.29 (s, 1H), 8.52 (dd, J=8.3, 1.4 Hz, 1H), 7.58 (dd, J=7.7, 1.6 Hz, 1H), 7.48-7.39 (m, 1H), 7.15-7.07 (m, 2H), 6.70-6.60 (m, 3H), 3.69 (s, 3H), 3.37 (s, 2H). ¹³C NMR (101 MHz, CDCl₃) δ 171.10, 160.01, 139.71, 137.33, 136.53, 130.86, 129.85, 124.31, 120.57, 120.44, 119.60, 112.89, 111.62, 55.20, 45.58. MS (ESI+) m/z: [M+H]⁺ calcd for C₁₅H₁₇N₂O₂S⁺ 289.1; found 289.1.

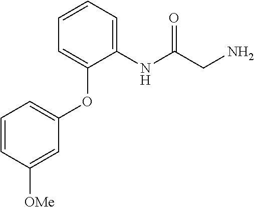 [SMILES: COc1cccc(Oc2ccccc2NC(=O)CN)c1]

Synthesis of 2-amino-N-(2-(3-methoxyphenoxy)phenyl) acetamide (15′): According to general procedure 5, 1 mmol of compound 15 (1 equiv.) was deprotected in TFA/DCM (1:1) solution to produce 15′ in quantitative yield. ¹H NMR (400 MHz, CDCl₃) δ 9.51 (s, 1H), 8.38 (dd, J=8.1, 1.6 Hz, 1H), 7.21-7.15 (m, 1H), 7.10-7.05 (m, 1H), 6.99 (td, J=7.8, 1.7 Hz, 1H), 6.88 (dd, J=8.1, 1.5 Hz, 1H), 6.66-6.61 (m, 1H), 6.56-6.52 (m, 2H), 3.96 (s, 2H), 3.73 (s, 3H), 3.53 (s, 2H). ¹³C NMR (101 MHz, CDCl₃) δ 169.28, 160.98, 157.82, 145.67, 130.22, 129.36, 124.32, 124.23, 121.03, 118.51, 110.37, 109.23, 104.42, 55.35, 44.40. MS (ESI+) m/z: [M+Na]⁺ calcd for C₁₅H₁₆N₂O₃Na⁺ 295.1; found 295.1.

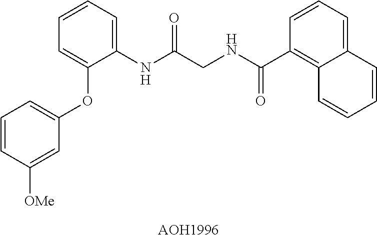 [SMILES: COc1cccc(Oc2ccccc2NC(=O)CNC(=O)c2cccc3ccccc23)c1 ; "AOH1996"]

Synthesis of N-(2-((2-(3-methoxyphenoxy)phenyl)amino)-2-oxoethyl)-1-naphthamide (AOH1996): According to general procedure 1, 1 mmol of compound 15′ (1 equiv.) was coupled with 1.2 mmol 1-naphthyl chloride 1 (1.2 equiv.) and purified by CombiFlash chromatography, with a gradient of 0 to 60% ethyl acetate in hexane to provide 392 mg (MW: 426.5 g/mol, 0.92 mmol, 92%) AOH1996 as a white powder. ¹H NMR (400 MHz, CDCl₃) δ 8.44 (s, 1H), 8.39 (dd, J=8.4, 1.5 Hz, 1H), 8.31 (dq, J=8.1, 0.8 Hz, 1H), 7.90 (d, J=8.3 Hz, 1H), 7.85-7.82 (m, 1H), 7.58 (dd, J=7.1, 1.2 Hz, 1H), 7.51-7.44 (m, 2H), 7.37 (dd, J=8.2, 7.0 Hz, 1H), 7.19 (t, J=8.6 Hz, 1H), 7.11 (td, J=7.8, 1.5 Hz, 1H), 7.03 (ddd, J=8.8, 7.6, 1.7 Hz, 1H), 6.90 (dd, J=8.1, 1.5 Hz, 1H), 6.83 (d, J=5.0 Hz, 1H), 6.67-6.64 (m, 1H), 6.57-6.53 (m, 2H), 4.32 (d, J=5.4 Hz, 2H), 3.70 (s, 3H). ¹³C NMR (101 MHz, CDCl₃) δ 169.92, 166.96, 161.05, 157.41, 145.57, 133.65, 133.01, 131.16, 130.35, 130.11, 128.28, 127.30, 126.47, 125.51, 125.32, 124.59, 124.50, 124.11, 120.97, 118.05, 110.54, 109.69, 104.65, 55.35, 44.68. MS (ESI+) m/z: [M+Na]⁺ calcd for C₂₆H₂₂N₂O₄Na⁺ 449.2; found 449.2.

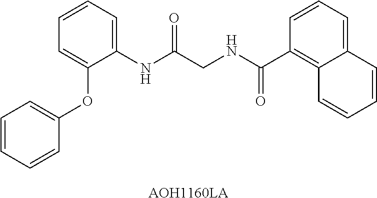 [SMILES: O=C(CNC(=O)c1cccc2ccccc12)Nc1ccccc1Oc1ccccc1 ; "AOH1160LA"]

Synthesis of(S)—N-(1-oxo-1-((2-phenoxyphenyl)amino) propan-2-yl)-1-naphthamide (AOH1160LA): According to general procedure 5, 1 mmol of compound 13 (1 equiv.) was deprotected in quantitative yield and based on general procedure 1 it was coupled with 1.2 mmol 1-naphthyl chloride 1 (1.2 equiv.). Final compound was purified by CombiFlash chromatography, with a gradient of 0 to 40% ethyl acetate in hexane to provide 398 mg (MW: 410.5 g/mol, 0.97 mmol, 97%) AOH1160LA as a white powder. ¹H NMR (400 MHZ, CDCl₃) δ 8.63 (s, 1H), 8.40 (d, J=8.1 Hz, 1H), 8.29 (d, J=8.2 Hz, 1H), 7.85 (dd, J=20.4, 8.1 Hz, 2H), 7.49 (dt, J=24.1, 7.8 Hz, 3H), 7.39-7.22 (m, 3H), 7.05 (dd, J=40.8, 7.1 Hz, 5H), 6.88 (d, J=8.1 Hz, 1H), 6.66 (d, J=7.9 Hz, 1H), 4.92 (appears as p, J=7.1 Hz, 1H), 1.53 (d, J=6.9 Hz, 3H). ¹³C NMR (101 MHz, CDCl₃) δ 170.22, 169.39, 156.39, 146.00, 133.66, 133.25, 131.01, 130.12, 129.90, 129.42, 128.27, 127.23, 126.41, 125.38, 125.25, 124.56, 124.45, 123.98, 123.84, 121.20, 118.56, 117.95, 50.13, 18.06. MS (ESI+) m/z: [M+Na]⁺ calcd for C₂₆H₂₂N₂O₃Na⁺ 433.1; found 432.9.

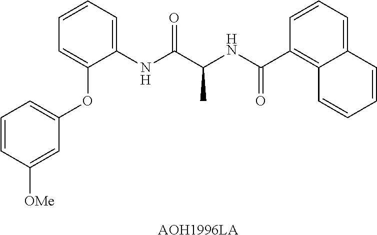 [SMILES: COc1cccc(Oc2ccccc2NC(=O)[C@H](C)NC(=O)c2cccc3ccccc23)c1 ; "AOH1996LA"]

Synthesis of(S)—N-(1-((2-(3-methoxyphenoxy)phenyl)amino)-1-oxopropan-2-yl)-1-naphthamide (AOH1996LA): According to general procedure 5, 1 mmol of compound 14 (1 equiv.) was deprotected in quantitative yield and based on general procedure 1 coupled with 1.2 mmol 1-naphthyl chloride 1 (1.2 equiv.). Final compound was purified by CombiFlash chromatography, with a gradient of 0 to 60% ethyl acetate in hexane to provide 384 mg (MW: 440.5 g/mol, 0.87 mmol, 87%) AOH1996LA as a beige powder. ¹H NMR (400 MHZ, CDCl₃) δ 8.71 (s, 1H), 8.38 (dd, J=8.1, 1.7 Hz, 1H), 8.26 (ddd, J=8.0, 1.6, 0.7 Hz, 1H), 7.87-7.76 (m, 2H), 7.52-7.36 (m, 3H), 7.29 (dd, J=8.2, 7.1 Hz, 1H), 7.19-7.14 (m, 1H), 7.09 (td, J=7.7, 1.6 Hz, 1H), 7.02 (td, J=7.8, 1.7 Hz, 1H), 6.91 (dd, J=8.0, 1.5 Hz, 1H), 6.85 (d, J=7.6 Hz, 1H), 6.66-6.59 (m, 1H), 6.58-6.52 (m, 2H), 4.90 (appears as p, J=7.1 Hz, 1H), 3.67 (s, 3H), 1.49 (d, J=7.1 Hz, 3H). ¹³C NMR (101 MHz, CDCl₃) δ 170.36, 169.50, 161.02, 157.58, 145.80, 133.59, 133.14, 130.98, 130.28, 130.09, 129.49, 128.26, 127.21, 126.39, 125.47, 125.28, 124.56, 124.48, 124.10, 121.27, 118.26, 110.48, 109.59, 104.58, 55.32, 50.11, 17.91. MS (ESI+) m/z: [M+Na]⁺ calcd for C₂₇H₂₄N₂O₄Na⁺ 463.2; found 463.3.

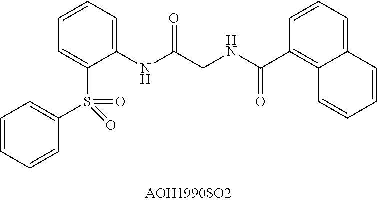 [SMILES: O=C(CNC(=O)c1cccc2ccccc12)Nc1ccccc1S(=O)(=O)c1ccccc1]

Synthesis of N-(2-oxo-2-((2-(phenylsulfonyl)phenyl)amino)ethyl)-1-naphthamide (AOH1160SO₂): According to general procedure 5, 1 mmol of compound 16 (1 equiv.) was deprotected in quantitative yield and based on general procedure 1 it was coupled with 1.2 mmol 1-naphthyl chloride 1 (1.2 equiv.). Final compound was purified by CombiFlash chromatography, with a gradient of 0 to 60% ethyl acetate in hexane to provide 333 mg (MW: 444.5 g/mol, 0.75 mmol, 75%) AOH1160SO₂ as a yellow oil. ¹H NMR (400 MHZ, CDCl₃) δ 10.14 (b, 1H), 8.44-8.34 (m, 2H), 8.01 (dd, J=8.0, 1.7 Hz, 1H), 7.94-7.84 (m, 5H), 7.64-7.35 (m, 7H), 7.29-7.20 (m, 1H), 6.72 (b, 1H), 4.41 (b, 2H). ¹³C NMR (101 MHz, CDCl₃) δ 170.07, 167.29, 140.73, 135.08, 133.79, 133.69, 131.24, 130.13, 129.78, 129.57, 128.36, 127.31, 127.08, 126.49, 125.69, 125.36, 124.77, 124.61, 122.80, 44.44. MS (ESI+) m/z: [M+Na]⁺ calcd for C₂₅H₂₀N₂O₄SNa⁺ 467.1; found 467.2. (88% yield was obtained when using general procedure 6.)

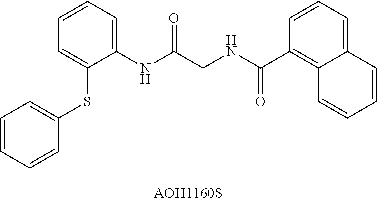 [SMILES: O=C(CNC(=O)c1cccc2ccccc12)Nc1ccccc1Sc1ccccc1]

Synthesis of N-(2-oxo-2-((2-(phenylthio)phenyl)amino)ethyl)-1-naphthamide (AOH1160S): According to general procedure 5, 1 mmol of compound 17 (1 equiv.) was deprotected in quantitative yield and based on general procedure 1 it was coupled with 1.2 mmol 1-naphthyl chloride 1 (1.2 equiv.). Final compound was purified by CombiFlash chromatography, with a gradient of 0 to 40% ethyl acetate in hexane to provide 384 mg (MW: 412.5 g/mol, 0.93 mmol, 93%) AOH1160S as a foamy powder. ¹H NMR (400 MHZ, CDCl₃) δ 8.75 (s, 1H), 8.40 (d, J=8.3 Hz, 1H), 8.37-8.30 (m, 1H), 7.91 (dt, J=8.1, 1.1 Hz, 1H), 7.88-7.80 (m, 1H), 7.57 (ddd, J=9.5, 7.4, 1.4 Hz, 2H), 7.53-7.47 (m, 2H), 7.45-7.34 (m, 2H), 7.18-7.04 (m, 4H), 6.97-6.90 (m, 2H), 6.66 (t, J=5.3 Hz, 1H), 4.20 (d, J=5.5 Hz, 2H). ¹³C NMR (101 MHZ, CDCl₃) δ 169.70, 166.94, 139.09, 136.46, 135.40, 133.66, 133.00, 131.20, 130.88, 130.13, 129.28, 128.31, 127.33, 127.16, 126.52, 126.31, 125.52, 125.42, 125.00, 124.60, 121.09, 120.76, 44.35. MS (ESI+) m/z: [M+Na]⁺ calcd for C₂₅H₂₀N₂O₂SNa⁺ 435.1; found 435.2. (95% yield was obtained when using general procedure 6.)

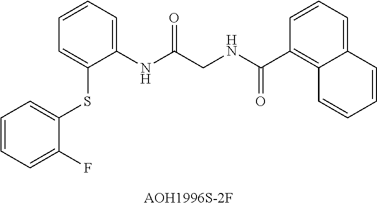 [SMILES: O=C(CNC(=O)c1cccc2ccccc12)Nc1ccccc1Sc1ccccc1F ; "AOH1996S-2F"]

Synthesis of N-(2-((2-((2-fluorophenyl)thio)phenyl)amino)-2-oxoethyl)-1-naphthamide (AOH1996S-2F): According to general procedure 6, 1 mmol of compound 5′g (1 equiv.) was coupled with 1.2 mmol 1-naphthoic acid 10 (1.2 equiv.). The final compound was purified by CombiFlash chromatography, with a gradient of 0 to 40% ethyl acetate in hexane to provide 383 mg (MW: 430.5 g/mol, 0.89 mmol, 89%) AOH1996S-2F as a powder. ¹H NMR (400 MHZ, CDCl₃) δ 8.84 (s, 1H), 8.44 (d, J=8.3 Hz, 1H), 8.40-8.34 (m, 1H), 7.94 (dt, J=8.2, 1.2 Hz, 1H), 7.90-7.85 (m, 1H), 7.69 (dd, J=7.1, 1.3 Hz, 1H), 7.60 (d, J=7.7 Hz, 1H), 7.58-7.48 (m, 2H), 7.45 (dd, J=8.3, 7.2 Hz, 2H), 7.20-7.11 (m, 2H), 7.04-6.91 (m, 3H), 6.69 (t, J=5.0 Hz, 1H), 4.35 (d, J=5.4 Hz, 2H). ¹⁹F NMR (376 MHz, CDCl₃) δ −110.17-−110.38 (m). ¹³C NMR (101 MHz, CDCl₃) δ 169.75, 166.93, 160.10 (d, J=244.6 Hz), 139.36, 136.59, 133.70, 133.06, 131.17 (d, J=9.8 Hz), 130.44, 130.14, 128.87, 128.79, 128.32, 127.31, 126.50, 125.51, 125.38, 125.09, 125.03, 125.00, 124.64, 122.36, 121.06, 119.79, 115.98, 115.77, 44.33. MS (ESI+) m/z: [M+Na]⁺ calcd for C₂₅H₁₉FN₂O₂SNa⁺ 453.1; found 453.1.

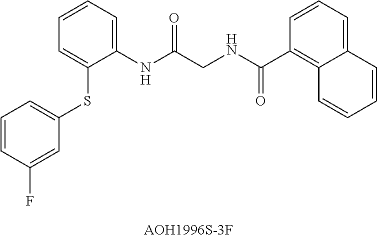 [SMILES: O=C(CNC(=O)c1cccc2ccccc12)Nc1ccccc1Sc1cccc(F)c1 ; "AOH1996S-3F"]

Synthesis of N-(2-((2-((3-fluorophenyl)thio)phenyl)amino)-2-oxoethyl)-1-naphthamide (AOH1996S-3F): According to general procedure 6, 1 mmol of compound 5′h (1 equiv.) was coupled with 1.2 mmol 1-naphthoic acid 18 (1.2 equiv.). The final compound was purified by CombiFlash chromatography, with a gradient of 0 to 40% ethyl acetate in hexane to provide 375 mg (MW: 430.5 g/mol, 0.87 mmol, 87%) AOH1996S-3F as a powder. ¹H NMR (400 MHZ, CDCl₃) δ 8.80 (s, 1H), 8.48 (d, J=8.3 Hz, 12H), 8.40-8.31 (m, 1H), 7.94 (d, J=8.3 Hz, 1H), 7.90-7.84 (m, 1H), 7.59 (td, J=7.3, 1.5 Hz, 2H), 7.55-7.46 (m, 3H), 7.42 (ddd, J=8.4, 6.9, 1.5 Hz, 1H), 7.17 (tt, J=7.5, 1.5 Hz, 1H), 7.08 (tdd, J=7.8, 5.8, 1.5 Hz, 1H), 6.81-6.65 (m, 3H), 6.63-6.52 (m, 1H), 4.26 (d, J=5.5 Hz, 2H). ¹⁹F NMR (376 MHz, CDCl₃) δ −111.08 (td, J=8.8, 5.8 Hz). ¹³C NMR (101 MHZ, CDCl₃) δ 169.75, 167.02, 163.05 (d, J=249.2 Hz), 139.49, 138.14, 138.06, 136.94, 133.70, 132.84, 131.53, 131.33, 130.56, 130.48, 130.13, 128.36, 127.37, 125.50, 125.39, 125.15, 124.59, 122.25, 121.11, 119.34, 113.75, 113.51, 113.29, 113.08, 44.54. MS (ESI+) m/z: [M+Na]⁺ calcd for C₂₅H₁₉FN₂O₂SNa⁺ 453.1; found 453.2.

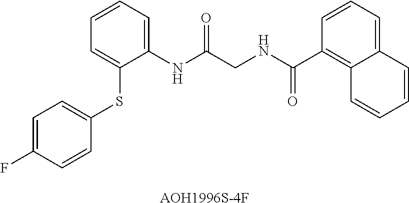 [SMILES: O=C(CNC(=O)c1cccc2ccccc12)Nc1ccccc1Sc1ccc(F)cc1 ; "AOH1996S-4F"]

Synthesis of N-(2-((2-((4-fluorophenyl)thio)phenyl)amino)-2-oxoethyl)-1-naphthamide (AOH1996S-4F): According to general procedure 6, 1 mmol of compound 5′i (1 equiv.) was coupled with 1.2 mmol 1-naphthoic acid 10 (1.2 equiv.). The final compound was purified by CombiFlash chromatography, with a gradient of 0 to 40% ethyl acetate in hexane to provide 409 mg (MW: 430.5 g/mol, 0.95 mmol, 95%) AOH1996S-4F as a powder. ¹H NMR (400 MHZ, CDCl₃) δ 8.78 (s, 1H), 8.42-8.31 (m, 2H), 7.92 (dt, J=8.3, 1.1 Hz, 1H), 7.86-7.82 (m, 1H), 7.61 (dd, J=7.1, 1.3 Hz, 1H), 7.57-7.46 (m, 3H), 7.41 (dd, J=8.3, 7.0 Hz, 2H), 7.12 (td, J=7.6, 1.4 Hz, 1H), 6.95 (t, J=6.9 Hz, 2H), 6.83 (t, J=8.6 Hz, 2H), 6.65 (s, 1H), 4.27 (d, J=5.6 Hz, 2H). ¹⁹F NMR (376 MHz, CDCl₃) δ −115.20 (tt, J=6.8, 6.2 Hz). ¹³C NMR (101 MHz, CDCl₃) δ 169.75, 166.94, 161.63 (d, J=247.1 Hz), 138.83, 136.10, 133.69, 132.89, 131.32, 130.85, 130.31, 130.11, 129.64, 129.57, 128.35, 127.38, 126.57, 125.50, 125.36, 125.29, 125.07, 124.59, 121.41, 121.15, 116.56, 116.34, 44.54. MS (ESI+) m/z: [M+Na]⁺ calcd for C₂₅H₁₉FN₂O₂SNa⁺ 453.1; found 453.1.

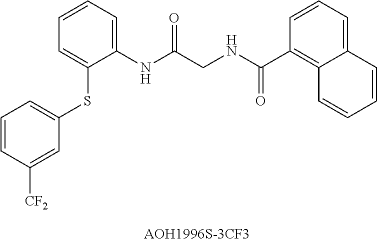

Synthesis of N-(2-oxo-2-((2-((3-(trifluoromethyl)phenyl)thio)phenyl)amino)ethyl)-1-naphthamide (AOH1996S-3CF₃): According to general procedure 6, 1 mmol of compound 5′j (1 equiv.) was coupled with 1.2 mmol 1-naphthoic acid 10 (1.2 equiv.). The final compound was purified by CombiFlash chromatography, with a gradient of 0 to 35% ethyl acetate in hexane to provide 389 mg (MW: 480.5 g/mol, 0.81 mmol, 81%) AOH1996S-3CF₃ as a white solid. ¹H NMR (400 MHZ, CDCl₃) δ 8.84 (s, 1H), 8.50 (d, J=8.3 Hz, 1H), 8.37 (dt, J=6.4, 3.5 Hz, 1H), 7.95 (d, J=8.6 Hz, 1H), 7.90-7.85 (m, 1H), 7.60 (ddd, J=10.3, 7.4, 1.4 Hz, 2H), 7.56-7.49 (m, 3H), 7.43 (dd, J=8.2, 7.1 Hz, 1H), 7.34 (d, J=7.8 Hz, 1H), 7.26-7.17 (m, 3H), 7.05 (d, J=8.0 Hz, 1H), 6.65 (t, J=4.9 Hz, 1H), 4.28 (d, J=5.6 Hz, 2H). ¹⁹F NMR (376 MHz, CDCl₃) δ −62.90. ¹³C NMR (101 MHz, CDCl₃) δ 169.81, 167.03, 139.53, 137.39, 136.86, 133.72, 132.77, 131.78, 131.64, 131.46, 131.39, 130.09, 129.77, 129.68, 128.38, 127.38, 126.57, 125.46, 125.33, 125.29, 124.86, 124.57, 123.37, 122.90 (q, J=3.9 Hz), 122.15, 121.26, 119.09, 44.65. MS (ESI+) m/z: [M+Na]⁺ calcd for C₂₆H₁₉F₃N₂O₂SNa⁺ 503.1; found 503.1.

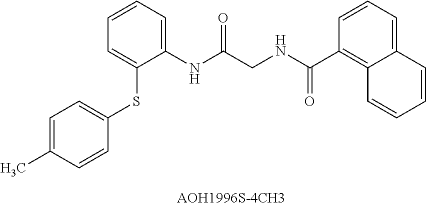 [SMILES: Cc1ccc(Sc2ccccc2NC(=O)CNC(=O)c2cccc3ccccc23)cc1 ; "AOH1996S-4CH3"]

Synthesis of N-(2-oxo-2-((2-(p-tolylthio)phenyl)amino)ethyl)-1-naphthamide (AOH1996S-4CH₃): According to general procedure 6, 1 mmol of compound 5′f (1 equiv.) was coupled with 1.2 mmol 1-naphthoic acid 18 (1.2 equiv.). The final compound was purified by CombiFlash chromatography, with a gradient of 0 to 70% ethyl acetate in hexane to provide 354 mg (MW: 426.5 g/mol, 0.83 mmol, 83%) AOH1996S-4CH₃ as a yellow solid. ¹H NMR (400 MHZ, CDCl₃) δ 8.73 (s, 1H), 8.43-8.33 (m, 2H), 7.94 (d, J=8.2 Hz, 1H), 7.91-7.84 (m, 1H), 7.64 (d, J=6.9 Hz, 1H), 7.59-7.48 (m, 3H), 7.47-7.38 (m, 2H), 7.13 (td, J=7.6, 1.2 Hz, 1H), 6.97 (d, J=7.9 Hz, 2H), 6.90 (d, J=7.9 Hz, 2H), 6.63 (s, 1H), 4.26 (d, J=4.5 Hz, 2H), 2.24 (s, 3H). ¹³C NMR (101 MHZ, CDCl₃) δ 169.69, 166.85, 138.79, 136.56, 136.08, 133.70, 133.08, 131.57, 131.22, 130.59, 130.15, 130.11, 128.34, 127.80, 127.35, 126.54, 125.55, 125.43, 124.95, 124.63, 121.64, 121.04, 44.39, 20.92. MS (ESI+) m/z: [M+Na]⁺ calcd for C₂₆H₂₂N₂O₂SNa⁺ 449.1; found 449.3.

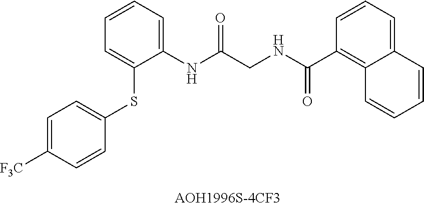 [SMILES: O=C(CNC(=O)c1cccc2ccccc12)Nc1ccccc1Sc1ccc(C(F)(F)F)cc1 ; "AOH1996S-4CF3"]

Synthesis of N-(2-oxo-2-((2-((4-(trifluoromethyl)phenyl)thio)phenyl)amino)ethyl)-1-naphthamide (AOH1996S-4CF₃): According to general procedure 6, 1 mmol of compound 5′k (1 equiv.) was coupled with 1.2 mmol 1-naphthoic acid 10 (1.2 equiv.). The final compound was purified by CombiFlash chromatography, with a gradient of 0 to 35% ethyl acetate in hexane to provide 375 mg (MW: 480.5 g/mol, 0.78 mmol, 78%) AOH1996S-4CF₃ as a white solid. ¹H NMR (400 MHZ, CDCl₃) δ 8.85 (s, 1H), 8.52 (d, J=8.3 Hz, 1H), 8.37 (dt, J=6.5, 3.7 Hz, 1H), 7.96 (d, J=8.2 Hz, 1H), 7.93-7.80 (m, 1H), 7.64-7.47 (m, 5H), 7.42 (dd, J=8.3, 7.1 Hz, 1H), 7.33 (d, J=8.2 Hz, 2H), 7.20 (td, J=7.6, 1.4 Hz, 1H), 6.93 (d, J=8.1 Hz, 2H), 6.58 (t, J=5.2 Hz, 1H), 4.28 (d, J=5.6 Hz, 2H). ¹⁹F NMR (376 MHz, CDCl₃) δ −62.52. ¹³C NMR (101 MHZ, CDCl₃) δ 169.75, 167.07, 140.95, 139.78, 137.18, 133.71, 132.69, 131.85, 131.43, 130.11, 128.38, 127.44, 126.62, 126.09, 125.96 (q, J=3.7 Hz), 125.46, 125.32, 125.26, 124.58, 122.51, 121.17, 118.45, 44.71. MS (ESI+) m/z: [M+Na]⁺ calcd for C₂₆H₁₉F₃N₂O₂SNa⁺ 503.1; found 503.2.

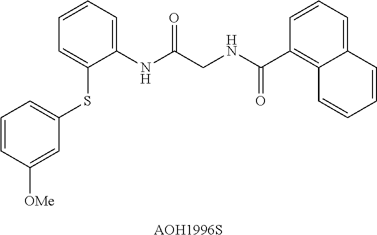 [SMILES: COc1cccc(Sc2ccccc2NC(=O)CNC(=O)c2cccc3ccccc23)c1 ; "AOH1996S"]

Synthesis of N-(2-((2-((3-methoxyphenyl)thio)phenyl)amino)-2-oxoethyl)-1-naphthamide (AOH1996S): According to general procedure 6, 1 mmol of compound 5′1 (1 equiv.) was coupled with 1.2 mmol 1-naphthoic acid 10 (1.2 equiv.). The final compound was purified by CombiFlash chromatography, with a gradient of 0 to 45% ethyl acetate in hexane to provide 376 mg (MW: 442.5 g/mol, 0.85 mmol, 85%) AOH1996S as pale yellow oil. ¹H NMR (400 MHZ, CDCl₃) δ 8.74 (s, 1H), 8.42 (d, J=8.3 Hz, 1H), 8.36-8.29 (m, 1H), 7.92 (dt, J=8.2, 1.1 Hz, 1H), 7.87-7.83 (m, 1H), 7.59 (ddd, J=10.8, 7.4, 1.4 Hz, 2H), 7.53-7.49 (m, 2H), 7.46-7.39 (m, 2H), 7.18-7.12 (m, 1H), 7.04 (t, J=7.9 Hz, 1H), 6.71-6.61 (m, 2H), 6.51 (d, J=8.4 Hz, 2H), 4.23 (d, J=5.4 Hz, 2H), 3.61 (s, 3H). ¹³C NMR (101 MHZ, CDCl₃) δ 169.69, 166.90, 160.16, 139.23, 136.76, 136.65, 133.68, 133.02, 131.20, 131.05, 130.12, 130.10, 128.31, 127.33, 126.52, 125.50, 125.42, 124.99, 124.60, 121.07, 120.34, 119.17, 112.50, 111.99, 55.15, 44.38. MS (ESI+) m/z: [M+Na]⁺ calcd for C₂₆H₂₂N₂O₃SNa⁺ 465.1; found 465.2.

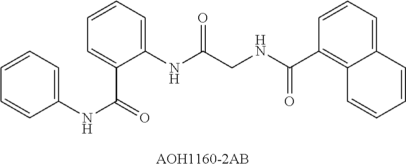

Synthesis of N-(2-oxo-2-((2-(phenylcarbamoyl)phenyl)amino)ethyl)-1-naphthamide (AOH1160-2AB): According to general procedure 1, 1 mmol of compound 5′m (1 equiv.) was coupled with 1.2 mmol 1-naphthyl chloride 1 (1.2 equiv.). The final compound was purified by CombiFlash chromatography, with a gradient of 0 to 10% MeOH in DCM to provide 385 mg (MW: 423.5 g/mol, 0.91 mmol, 91%) AOH1160-2AB as a white powder. 1H NMR (400 MHZ, DMSO) δ 11.32 (s, 1H), 10.49 (s, 1H), 9.17 (t, J=5.8 Hz, 1H), 8.54 (dd, J=8.4, 1.1 Hz, 1H), 8.32-8.22 (m, 1H), 8.02 (d, J=7.7 Hz, 2H), 7.98-7.93 (m, 1H), 7.87 (dd, J=7.9, 1.5 Hz, 1H), 7.61-7.47 (m, 5H), 7.43 (dd, J=8.2, 7.1 Hz, 1H), 7.24 (dtd, J=9.4, 7.5, 1.6 Hz, 3H), 7.12-7.05 (m, 1H), 4.08 (d, J=5.8 Hz, 2H). ¹³C NMR (101 MHZ, DMSO) δ 170.00, 168.74, 167.47, 138.87, 138.77, 133.98, 133.55, 132.76, 130.70, 130.23, 129.30, 128.96, 128.57, 127.14, 126.62, 126.48, 126.01, 125.19, 124.60, 123.41, 122.27, 121.30, 120.85, 44.82. MS (ESI+) m/z: [M+Na]⁺ calcd for C₂₆H₂₁N₃O₃Na⁺ 446.1; found 446.2.

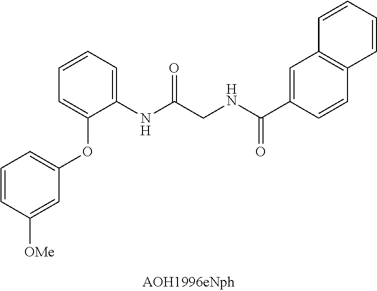 [SMILES: COc1cccc(Oc2ccccc2NC(=O)CNC(=O)c2ccc3ccccc3c2)c1]

Synthesis of N-(2-(3-methoxyphenoxy)phenyl)-2-(2-(naphthalen-1-yl) acetamido) acetamide (AOH1996eNph): According to general procedure 4, 1 mmol of compound 15′ (1 equiv.) was coupled with 1.2 mmol 2-(naphthalen-1-yl) acetic acid 19 (1.2 equiv.) using DCC (cat. DMAP) and purified by CombiFlash chromatography, with a gradient of 0 to 50% ethyl acetate in hexane to provide 405 mg (MW: 440.5 g/mol, 0.92 mmol, 92%) AOH1996eNph as a pale-yellow powder. ¹H NMR (400 MHZ, CDCl₃) δ 8.27 (d, J=8.1 Hz, 1H), 8.20 (b, 1H), 7.90-7.80 (m, 3H), 7.50-7.45 (m, 2H), 7.42-7.37 (m, 1H), 7.32 (d, J=6.9 Hz, 1H), 7.25 (d, J=0.5 Hz, 1H), 7.08 (td, J=7.7, 1.3 Hz, 1H), 7.01 (td, J=7.7, 1.6 Hz, 1H), 6.90 (dt, J=8.1, 1.3 Hz, 1H), 6.69 (ddt, J=8.3, 2.2, 1.1 Hz, 1H), 6.62-6.52 (m, 2H), 5.95 (b, 1H), 3.98 (b, 2H), 3.91 (dd, J=5.6, 1.1 Hz, 2H), 3.77 (s, 3H). ¹³C NMR (101 MHz, CDCl₃) δ 171.62, 166.89, 161.08, 157.68, 145.39, 133.95, 131.92, 130.37, 129.34, 128.84, 128.63, 128.42, 126.87, 126.19, 125.65, 124.47, 124.21, 123.53, 121.07, 118.41, 110.36, 109.41, 104.48, 55.41, 44.44, 41.11. MS (ESI+) m/z: [M+Na]⁺ calcd for C₂₇H₂₄N₂O₄Na⁺ 463.2; found 463.2.

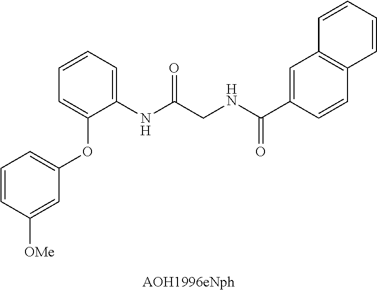 [SMILES: COc1cccc(Oc2ccccc2NC(=O)CNC(=O)c2ccc3ccccc3c2)c1]

Synthesis of N-(2-((2-(3-methoxyphenoxy)phenyl)amino)-2-oxoethyl)-3-(naphthalen-1-yl) propanamide (AOH1996eeNph): According to general procedure 4, 1 mmol of compound 15′ (1 equiv.) was coupled with 1.2 mmol 3-(naphthalen-1-yl) propanoic acid 20 (1.2 equiv.) using DCC (cat. DMAP) and was purified by CombiFlash chromatography, with a gradient of 0 to 50% ethyl acetate in hexane to provide 413 mg (MW: 454.5 g/mol, 0.91 mmol, 91%) AOH1996eeNph as a pale-yellow oil. ¹H NMR (400 MHZ, CDCl₃) δ 8.34 (d, J=8.1 Hz, 1H), 8.18 (s, 1H), 8.00-7.97 (m, 1H), 7.86-7.82 (m, 1H), 7.70 (d, J=8.0 Hz, 1H), 7.51-7.44 (m, 2H), 7.36-7.28 (m, 2H), 7.22-7.17 (m, 1H), 7.14-7.10 (m, 1H), 7.04 (t, J=7.7 Hz, 1H), 6.93-6.88 (m, 1H), 6.65-6.62 (m, 1H), 6.58-6.52 (m, 2H), 6.02 (s, 1H), 4.02 (dd, J=5.4, 0.8 Hz, 2H), 3.72 (d, J=0.7 Hz, 3H), 3.40-3.36 (m, 2H), 2.62 (dd, J=9.0, 6.8 Hz, 2H). MS (ESI+) m/z: [M+Na]⁺ calcd for C₂₈H₂₆N₂O₄Na⁺ 477.2; found 477.2.

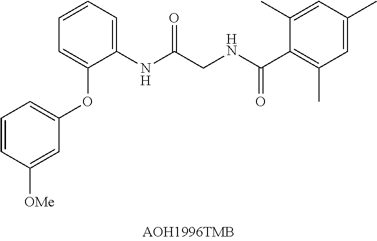 [SMILES: COc1cccc(Oc2ccccc2NC(=O)CNC(=O)c2c(C)cc(C)cc2C)c1]

Synthesis of N-(2-((2-(3-methoxyphenoxy)phenyl)amino)-2-oxoethyl)-2,4,6-trimethylbenzamide (AOH1996TMB): According to general procedure 1, 1 mmol of compound 15′ (1 equiv.) was coupled with 1.2 mmol 2,4,6-trimethylbenzoyl chloride 21 (1.2 equiv.) in 3 hours and purified by CombiFlash chromatography, with a gradient of 0 to 40% ethyl acetate in hexane to provide 389 mg (MW: 418.5 g/mol, 0.93 mmol, 93%) AOH1996TMB as a yellow oil. ¹H NMR (400 MHZ, CDCl₃) δ 8.36 (dd, J=8.2, 1.6 Hz, 1H), 8.30 (s, 1H), 7.24-7.19 (m, 1H), 7.10 (td, J=7.8, 1.5 Hz, 1H), 7.02 (td, J=7.8, 1.7 Hz, 1H), 6.87 (dd, J=8.1, 1.5 Hz, 1H), 6.81 (h, J=0.6 Hz, 2H), 6.68 (ddd, J=8.3, 2.2, 1.2 Hz, 1H), 6.61-6.52 (m, 2H), 6.39 (d, J=5.7 Hz, 1H), 4.24 (d, J=5.5 Hz, 2H), 3.75 (s, 3H), 2.24 (dt, J=3.7, 0.7 Hz, 9H). ¹³C NMR (101 MHz, CDCl₃) δ 171.21, 166.85, 161.06, 157.28, 145.76, 138.77, 134.30, 133.86, 130.36, 128.99, 128.22, 124.46, 123.93, 121.04, 117.79, 110.85, 109.74, 104.89, 55.38, 44.24, 21.05, 19.16. MS (ESI+) m/z: [M+Na]⁺ calcd for C₂₅H₂₆N₂O₄Na⁺ 441.2; found 441.2.

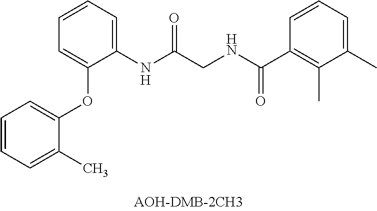 [SMILES: Cc1ccccc1Oc1ccccc1NC(=O)CNC(=O)c1cccc(C)c1C]

Synthesis of 2,3-dimethyl-N-(2-oxo-2-((2-(o-tolyloxy)phenyl)amino)ethyl)benzamide (AOH-DMB-2CH3): According to general procedure 1, 1 mmol of compound 5′c (1 equiv.) was coupled with 1.2 mmol 2,3-dimethylbenzoyl chloride 23 (1.2 equiv.) in 3 hours and purified by CombiFlash chromatography, with a gradient of 0 to 20% ethyl acetate in hexane to provide 330 mg (MW: 388.5 g/mol, 0.85 mmol, 85%) AOH-DMB-2CH3. ¹H NMR (400 MHZ, CDCl₃) δ 8.53 (s, 1H), 8.39 (dd, J=8.1, 1.7 Hz, 1H), 7.24 (dd, J=1.8, 0.9 Hz, 1H), 7.18 (ddd, J=5.2, 2.9, 1.4 Hz, 3H), 7.10 (td, J=7.4, 1.4 Hz, 1H), 7.09-6.93 (m, 3H), 6.89 (dd, J=7.9, 1.3 Hz, 1H), 6.67-6.59 (m, 2H), 4.28 (d, J=5.5 Hz, 2H), 2.25 (s, 6H), 2.21 (s, 3H). ¹³C NMR (101 MHz, CDCl₃) δ 171.24, 167.02, 153.47, 146.53, 137.96, 136.06, 134.42, 131.68, 131.53, 129.95, 128.08, 127.38, 125.47, 124.77, 124.53, 124.37, 122.93, 120.71, 119.77, 115.40, 44.53, 20.22, 16.24, 16.06. MS (ESI+) m/z: [M+H]⁺ calcd for C₂₆H₂₃N₂O₃+411.2; found 411.5.

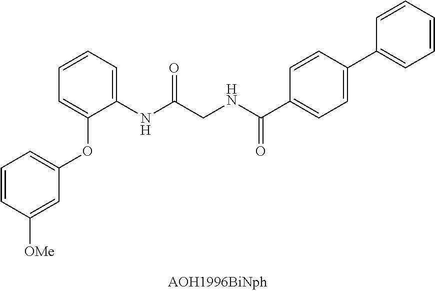 [SMILES: COc1cccc(Oc2ccccc2NC(=O)CNC(=O)c2ccc(-c3ccccc3)cc2)c1]

Synthesis of N-(2-((2-(3-methoxyphenoxy)phenyl)amino)-2-oxoethyl)-[1,1′-biphenyl]-4-carboxamide (AOH1996BiNph): According to general procedure 1, 1 mmol of compound 15′ (1 equiv.) was coupled with 1.2 mmol [1,1′-biphenyl]-4-carbonyl chloride 22 (1.2 equiv.) in 3 hours and purified by CombiFlash chromatography, with a gradient of 0 to 50% ethyl acetate in hexane to provide 403 mg (MW: 452.5 g/mol, 0.89 mmol, 89%) AOH1996BiNph as a white powder. ¹H NMR (400 MHZ, CDCl₃) δ 8.50 (s, 1H), 8.38 (d, J=8.1 Hz, 1H), 7.84-7.78 (m, 2H), 7.66-7.55 (m, 4H), 7.53-7.32 (m, 3H), 7.22-7.00 (m, 4H), 6.91 (dd, J=8.1, 1.5 Hz, 1H), 6.66-6.58 (m, 1H), 6.54-6.47 (m, 2H), 4.28 (d, J=5.3 Hz, 2H), 3.70 (s, 3H). ¹³C NMR (101 MHz, CDCl₃) δ 167.55, 167.32, 161.00, 157.62, 145.39, 144.65, 139.89, 131.95, 130.28, 129.44, 128.91, 128.05, 127.67, 127.19, 127.18, 124.59, 124.29, 121.10, 118.56, 110.12, 109.38, 104.30, 55.31, 44.75. MS (ESI+) m/z: [M+Na]⁺ calcd for C₂₈H₂₄N₂O₄Na⁺ 475.2; found 475.3.

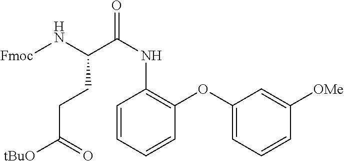

Synthesis of tert-butyl(S)-4-((((9H-fluoren-9-yl) methoxy) carbonyl)amino)-5-((2-(3-methoxyphenoxy)phenyl)amino)-5-oxopentanoate (17b): According to general procedure 3, 2 mmol of 2-(3-methoxyphenoxy)aniline 10 was coupled with 2.2 mmol Fmoc-L-glutamic acid 5-tert-butyl ester 24 and only purified by CombiFlash chromatography (without aqueous wash), with a gradient of 0 to 35% ethyl acetate in hexane to provide 1.14 g (MW: 622.7 g/mol, 1.84 mmol, 92%) title compound (17b). ¹H NMR (400 MHZ, CDCl₃) δ 8.67 (s, 1H), 8.39 (dd, J=8.1, 1.6 Hz, 1H), 7.75 (t, J=6.8 Hz, 2H), 7.54 (d, J=7.6 Hz, 2H), 7.43-7.26 (m, 5H), 7.14-6.98 (m, 3H), 6.89 (dt, J=8.1, 1.8 Hz, 1H), 6.59-6.47 (m, 3H), 4.39-4.26 (m, 3H), 4.17-4.09 (m, 1H), 3.66 (s, 3H), 2.50-2.27 (m, 2H), 2.15 (dq, J=14.8, 7.9, 7.0 Hz, 1H), 1.97 (dt, J=14.5, 7.3 Hz, 1H), 1.42 (s, 9H). MS (ESI+) m/z: [M+Na]⁺ calcd for C₃₇H₃₈N₂O₇Na⁺ 645.3; found 645.4.

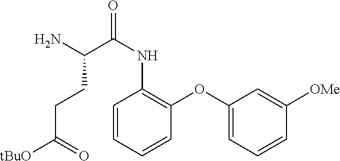 [SMILES: COc1cccc(Oc2ccccc2NC(=O)[C@@H](N)CCC(=O)OC(C)(C)C)c1]

Synthesis of tert-butyl(S)-4-amino-5-((2-(3-methoxyphenoxy)phenyl)amino)-5-oxopentanoate (17′b): According to general procedure 7, 1.84 mmol of compound 17b was deprotected in 20% ethanolamine in DCM solution and purified by CombiFlash chromatography, with a gradient of 0 to 40% ethyl acetate in hexane to provide 692 mg (MW: 400.5 g/mol, 1.73 mmol, 94%) of the title compound. ¹H NMR (400 MHz, CDCl₃) δ 9.85 (s, 1H), 8.44 (dd, J=8.1, 1.7 Hz, 1H), 7.19 (tt, J=7.6, 1.2 Hz, 1H), 7.12 (td, J=7.7, 1.5 Hz, 1H), 7.01 (td, J=7.8, 1.6 Hz, 1H), 6.92 (dd, J=8.1, 1.5 Hz, 1H), 6.67-6.59 (m, 1H), 6.58-6.51 (m, 2H), 3.75 (s, 3H), 3.49 (dd, J=7.6, 4.9 Hz, 1H), 2.33 (td, J=7.3, 1.6 Hz, 2H), 2.20-2.07 (m, 1H), 1.90-1.77 (m, 1H), 1.41 (s, 9H). ¹³C NMR (101 MHz, CDCl₃) δ 172.61, 172.58, 160.98, 158.02, 145.47, 130.19, 129.82, 124.41, 124.09, 120.81, 118.89, 110.08, 109.05, 104.15, 80.59, 55.35, 55.28, 31.93, 30.04, 28.03. MS (ESI+) m/z: [M+H]⁺ calcd for C₂₂H₂₉N₂O₅+401.2; found 401.1.

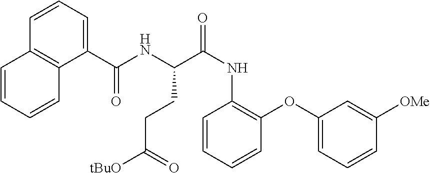 [SMILES: COc1cccc(Oc2ccccc2NC(=O)[C@H](CCC(=O)OC(C)(C)C)NC(=O)c2cccc3ccccc23)c1]

Synthesis of tert-butyl(S)-4-(1-naphthamido)-5-((2-(3-methoxyphenoxy)phenyl)amino)-5-oxopentanoate (18b): According to general procedure 1, 1 mmol of amino acid derivative 17′b (1 equiv.) was coupled with 1.2 mmol 1-naphthyl chloride 1 (1.2 equiv.) and purified by CombiFlash chromatography, with a gradient of 0 to 25% ethyl acetate in hexane to provide 554.6 mg (MW: 554.6 g/mol, 1.0 mmol, quant. yield) 18b. ¹H NMR (400 MHZ, CDCl₃) δ 8.89 (s, 1H), 8.40 (dd, J=8.2, 1.6 Hz, 1H), 8.37-8.31 (m, 1H), 7.91-7.87 (m, 1H), 7.87-7.81 (m, 1H), 7.58 (dd, J=7.1, 1.2 Hz, 1H), 7.52-7.43 (m, 2H), 7.37 (dd, J=8.2, 7.1 Hz, 1H), 7.21-7.00 (m, 4H), 6.92 (dd, J=8.1, 1.5 Hz, 1H), 6.63 (ddd, J=8.3, 2.3, 1.0 Hz, 1H), 6.60-6.53 (m, 2H), 4.91 (td, J=7.9, 5.1 Hz, 1H), 3.69 (s, 3H), 2.60 (ddd, J=16.9, 7.9, 6.2 Hz, 1H), 2.41 (ddd, J=16.9, 7.0, 6.1 Hz, 1H), 2.34-2.21 (m, 1H), 2.11 (dddd, J=14.4, 8.1, 7.1, 6.2 Hz, 1H), 1.37 (s, 9H). ¹³C NMR (101 MHZ, CDCl₃) δ 172.94, 169.65, 169.45, 161.00, 157.57, 145.90, 133.67, 133.15, 131.08, 130.21, 130.19, 129.40, 128.24, 127.25, 126.40, 125.40, 125.37, 124.56, 124.51, 124.02, 121.23, 118.17, 110.58, 109.65, 104.62, 81.19, 55.31, 53.97, 31.87, 27.95, 27.66. MS (ESI+) m/z: [M+Na]⁺ calcd for C₃₃H₃₄N₂O₆Na⁺ 577.2; found 577.2.

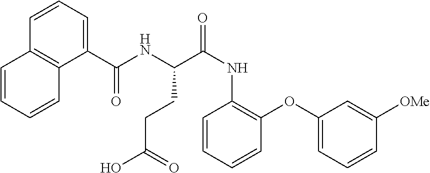 [SMILES: COc1cccc(Oc2ccccc2NC(=O)[C@H](CCC(=O)O)NC(=O)c2cccc3ccccc23)c1]

Synthesis of(S)-4-(1-naphthamido)-5-((2-(3-methoxyphenoxy)phenyl)amino)-5-oxopentanoic acid (AOH1996LE): According to general procedure 2, 1 mmol of tert-butyl ester 18b was deprotected by TFA. Volatiles were removed under reduced pressure and coevaporated with HCl 0.1 M (2×2 mL to remove TFA salts) to provide 498 mg (MW: 498.5 g/mol, 1.0 mmol, quant. yield) AOH1996LE as a white powder. This compound was not purified by CombiFlash chromatography. ¹H NMR (400 MHZ, CDCl₃) δ 9.89 (s, 1H), 8.99 (s, 1H), 8.31-8.18 (m, 2H), 7.90 (d, J=8.4 Hz, 1H), 7.85-7.79 (m, 1H), 7.59 (dd, J=7.1, 1.2 Hz, 1H), 7.52-7.45 (m, 2H), 7.38 (dd, J=8.3, 7.1 Hz, 1H), 7.32 (d, J=8.4 Hz, 1H), 7.22-7.03 (m, 3H), 6.93-6.86 (m, 1H), 6.62 (ddd, J=8.3, 2.3, 0.9 Hz, 1H), 6.58-6.50 (m, 2H), 5.16 (td, J=8.5, 5.3 Hz, 1H), 3.68 (s, 3H), 2.58 (ddd, J=17.0, 8.6, 5.5 Hz, 1H), 2.40 (ddd, J=17.1, 6.9, 5.4 Hz, 1H), 2.22-1.94 (m, 2H). ¹H NMR (400 MHZ, DMSO) δ 12.13 (s, 1H), 9.53 (s, 1H), 8.86 (d, J=7.6 Hz, 1H), 8.18 (d, J=7.4 Hz, 1H), 8.09 (dd, J=7.8, 1.9 Hz, 1H), 7.96 (dd, J=20.0, 7.7 Hz, 2H), 7.64-7.41 (m, 4H), 7.23 (t, J=8.2 Hz, 1H), 7.12 (dtd, J=17.6, 7.5, 1.7 Hz, 2H), 6.92 (dd, J=7.9, 1.7 Hz, 1H), 6.68 (dd, J=8.2, 2.5 Hz, 1H), 6.60-6.47 (m, 2H), 4.74 (td, J=8.3, 7.7, 5.0 Hz, 1H), 3.66 (s, 3H), 3.39 (s, 1H), 2.36 (t, J=7.9 Hz, 2H), 2.12-1.76 (m, 2H). ¹³C NMR (101 MHz, DMSO) δ 174.33, 170.90, 169.56, 161.06, 158.04, 147.30, 134.41, 133.51, 130.83, 130.47, 130.21, 129.99, 128.60, 127.17, 126.66, 125.97, 125.80, 125.35, 125.28, 124.26, 123.32, 119.09, 110.78, 109.75, 105.02, 55.66, 53.88, 30.89, 26.84. MS (ESI+) m/z: [M+Na]⁺ calcd for C₂₉H₂₆N₂O₆Na⁺ 521.2; found 521.3.

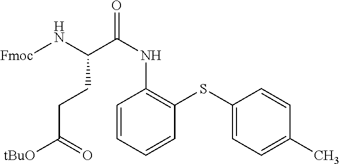

Synthesis of tert-butyl(S)-4-((((9H-fluoren-9-yl) methoxy) carbonyl)amino)-5-oxo-5-((2-(p-tolylthio)phenyl)amino) pentanoate (17f): According to general procedure 3, 2 mmol of 2-(p-tolylthio)aniline 4f was coupled with 2.2 mmol Fmoc-L-glutamic acid 5-tert-butyl ester 24 and only purified by CombiFlash chromatography (without aqueous wash), with a gradient of 0 to 30% ethyl acetate in hexane to provide 872 mg (MW: 622.8 g/mol, 1.4 mmol, 70%) title compound (17f). ¹H NMR (400 MHZ, CDCl₃) δ 9.02 (s, 1H), 8.46-8.39 (m, 1H), 7.77 (d, J=7.6 Hz, 2H), 7.63-7.49 (m, 3H), 7.36 (dt, J=36.2, 8.5 Hz, 5H), 7.11 (t, J=7.3 Hz, 1H), 6.95-6.85 (m, 4H), 5.70 (d, J=7.4 Hz, 1H), 4.38-4.26 (m, 3H), 4.18-4.10 (m, 1H), 2.39-2.18 (m, 2H), 2.16 (s, 3H), 2.11-1.79 (m, 2H), 1.45 (s, 9H). ¹³C NMR (101 MHz, CDCl₃) δ 172.66, 169.47, 156.18, 143.79, 143.71, 141.33, 141.30, 138.88, 136.46, 135.98, 131.70, 130.46, 130.00, 128.03, 127.74, 127.11, 127.10, 125.09, 124.84, 122.07, 120.89, 119.98, 119.96, 81.09, 67.11, 55.86, 47.08, 31.68, 28.07, 27.21, 20.81. MS (ESI+) m/z: [M+Na]⁺ calcd for C₃₇H₃₈N₂O₅SNa⁺ 645.2; found 645.3.

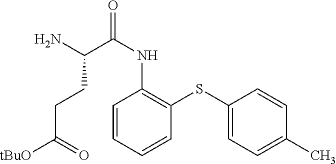 [SMILES: Cc1ccc(Sc2ccccc2NC(=O)[C@@H](N)CCC(=O)OC(C)(C)C)cc1]

Synthesis of tert-butyl(S)-4-amino-5-oxo-5-((2-(p-tolylthio)phenyl)amino) pentanoate (17′f): According to general procedure 7, 1.4 mmol of compound 17f was deprotected in 20% ethanolamine in DCM solution and purified by CombiFlash chromatography, with a gradient of 0 to 30% ethyl acetate in hexane to provide 504 mg (MW: 400.5 g/mol, 1.26 mmol, 90%) of the title compound. ¹H NMR (400 MHZ, CDCl₃) δ 10.26 (s, 1H), 8.47 (dd, J=8.2, 1.4 Hz, 1H), 7.55 (dd, J=7.7, 1.5 Hz, 1H), 7.45-7.35 (m, 1H), 7.14-7.04 (m, 1H), 7.05-7.00 (m, 4H), 3.47 (dd, J=7.5, 4.9 Hz, 1H), 2.32-2.24 (m, 5H), 2.06 (dtd, J=14.2, 7.6, 4.9 Hz, 1H), 1.84-1.73 (m, 3H), 1.43 (s, 9H). ¹³C NMR (101 MHz, CDCl₃) δ 172.53, 172.49, 139.23, 136.23, 135.96, 132.06, 130.37, 129.86, 128.01, 124.34, 121.85, 120.64, 80.62, 55.38, 31.81, 29.88, 28.06, 20.93. MS (ESI+) m/z: [M+H]⁺ calcd for C₂₂H₂₉N₂O₃S⁺ 401.1; found 401.1.

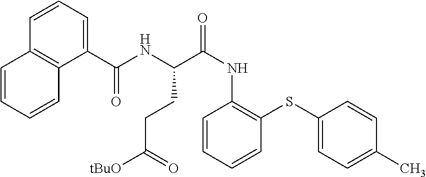 [SMILES: Cc1ccc(Sc2ccccc2NC(=O)[C@H](CCC(=O)OC(C)(C)C)NC(=O)c2cccc3ccccc23)cc1]

Synthesis of tert-butyl(S)-4-(1-naphthamido)-5-oxo-5-((2-(p-tolylthio)phenyl)amino) pentanoate (18f): According to general procedure 1, 1 mmol of amino acid derivative 17′f (1 equiv.) was coupled with 1.2 mmol 1-naphthyl chloride 1 (1.2 equiv.) and purified by CombiFlash chromatography, with a gradient of 0 to 20% ethyl acetate in hexane to provide 527 mg (MW: 554.7 g/mol, 0.95 mmol, 95%) 18f as a white solid. 1H NMR (400 MHZ, CDCl₃) δ 8.99 (s, 1H), 8.39 (dddd, J=6.7, 3.1, 2.1, 0.9 Hz, 2H), 7.93 (dt, J=8.2, 0.7 Hz, 1H), 7.90-7.83 (m, 1H), 7.64 (dd, J=7.1, 1.2 Hz, 1H), 7.57-7.50 (m, 3H), 7.42 (dddd, J=9.1, 8.2, 3.8, 1.3 Hz, 2H), 7.13 (td, J=7.6, 1.4 Hz, 1H), 7.01-6.91 (m, 5H), 4.86 (tdd, J=8.0, 5.1, 2.7 Hz, 1H), 2.48 (ddd, J=16.9, 7.8, 6.7 Hz, 1H), 2.34 (dt, J=16.9, 6.7 Hz, 1H), 2.22 (s, 3H), 2.21-2.14 (m, 1H), 2.03-1.92 (m, 1H), δ 1.40 (s, 9H). ¹³C NMR (101 MHZ, CDCl₃) δ 172.64, 169.51, 169.42, 138.82, 136.54, 135.86, 133.71, 133.11, 131.62, 131.16, 130.32, 130.24, 130.05, 128.27, 128.18, 127.30, 126.46, 125.53, 125.42, 124.96, 124.56, 122.40, 121.31, 81.09, 54.10, 31.67, 28.00, 27.42, 20.91. MS (ESI+) m/z: [M+Na]⁺ calcd for C₃₃H₃₄N₂O₄SNa⁺ 577.2; found 577.3.

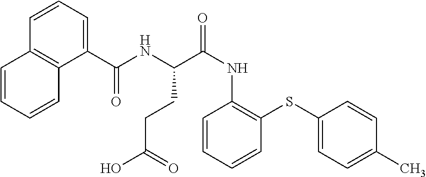 [SMILES: Cc1ccc(Sc2ccccc2NC(=O)[C@H](CCC(=O)O)NC(=O)c2cccc3ccccc23)cc1]

Synthesis of(S)-4-(1-naphthamido)-5-oxo-5-((2-(p-tolylthio)phenyl)amino) pentanoic acid (AOH1996SLE-4CH₃): According to general procedure 2, 0.95 mmol of tert-butyl ester 18f was deprotected by TFA. Volatiles were removed under reduced pressure and coevaporated with HCl 0.1 M (2×2 mL to remove TFA salts) to provide 449 mg (MW: 498.6 g/mol, 0.95 mmol, quant. yield) AOH1996SLE-4CH₃ as a white powder. ¹H NMR (400 MHz, DMSO) δ 12.16 (s, 1H), 9.62 (s, 1H), 8.89 (d, J=7.5 Hz, 1H), 8.25 (dd, J=8.4, 1.5 Hz, 1H), 8.08-7.92 (m, 2H), 7.85 (dd, J=8.2, 1.4 Hz, 1H), 7.65 (dd, J=7.1, 1.3 Hz, 1H), 7.59-7.45 (m, 3H), 7.40-7.32 (m, 1H), 7.27 (dd, J=7.9, 1.6 Hz, 1H), 7.19-7.08 (m, 5H), 4.71 (ddd, J=9.3, 7.4, 4.9 Hz, 1H), 2.40 (t, J=7.7 Hz, 2H), 2.23 (s, 3H), 2.16-1.85 (m, 2H). ¹³C NMR (101 MHz, DMSO) δ 174.34, 170.88, 169.54, 138.03, 137.56, 134.36, 133.55, 133.47, 131.23, 131.05, 130.65, 130.53, 130.26, 129.08, 128.61, 128.02, 127.14, 126.68, 126.21, 126.06, 125.95, 125.32, 124.41, 53.94, 30.88, 26.85, 21.04. MS (ESI−) m/z: [M−H]⁻ calcd for C₂₉H₂₅N₂O₄S⁻ 497.2; found 497.3.

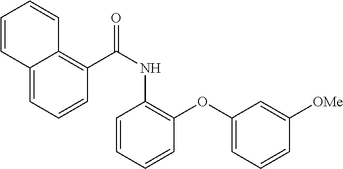 [SMILES: COc1cccc(Oc2ccccc2NC(=O)c2cccc3ccccc23)c1]

Synthesis of N-(2-(3-methoxyphenoxy)phenyl)-1-naphthamide (AOH1996t): According to general procedure 1, 1 mmol of compound 10 (1 equiv.) was coupled with 1.2 mmol 1-naphthyl chloride 1 (1.2 aequiv.). and purified by CombiFlash chromatography, with a gradient of 0 to 20% ethyl acetate in hexane to provide 358 mg (MW: 369.4 g/mol, 0.97 mmol, 97%) title compound (AOH1996t) as a colorless oil. ¹H NMR (400 MHZ, CDCl₃) δ 8.72 (d, J=8.1 Hz, 1H), 8.41-8.36 (m, 1H), 8.29 (s, 1H), 7.94 (dt, J=8.5, 1.0 Hz, 1H), 7.89-7.85 (m, 1H), 7.63 (dd, J=7.1, 1.3 Hz, 1H), 7.54-7.49 (m, 2H), 7.45 (dd, J=8.2, 7.1 Hz, 1H), 7.26-7.21 (m, 2H), 7.10 (ddd, J=8.2, 7.5, 1.6 Hz, 1H), 6.96 (dd, J=8.1, 1.5 Hz, 1H), 6.71-6.66 (m, 1H), 6.61-6.55 (m, 2H), 3.75 (s, 3H). ¹³C NMR (101 MHz, CDCl₃) δ 167.37, 161.11, 157.62, 145.67, 134.33, 133.77, 131.17, 130.41, 130.11, 130.06, 128.40, 127.34, 126.54, 125.35, 125.29, 124.76, 124.50, 124.38, 121.20, 118.33, 110.47, 109.60, 104.56, 55.41. MS (ESI+) m/z: [M+Na]⁺ calcd for C₂₄H₁₉NO₃Na⁺ 369.1; found 369.2.

### Example 3: In Silico Data

The in silico structures were produced using Chimera (UCSF Chimera—a visualization system for exploratory research and analysis. Pettersen E F, Goddard T D, Huang C C, Couch G S, Greenblatt D M, Meng E C, Ferrin T E. J Comput Chem. 2004 October; 25 (13): 1605-12).

In performing the in silico analyses, the following assumptions were being made in determining what compounds would make good drug candidates from our in silico data:
(1) The compound's lowest energy configuration must bind the Primary Targeted Site with within the PIP binding interaction domain of PCNA with an energy similar to or lower than AOH1996/1160.
(2) The compound must bind significantly to both regions A and B of the PIP binding domain.
(3) Even if the conditions set out in (1) and (2) are met for the lowest energy conformations of the compound, if the compound should be calculated to have several slightly stronger energy configurations that do violate those conditions, it would be considered a poorer candidate on the grounds that unfavorable equilibria may result in an undesired decrease in efficacy/increase in detrimental side effects.

Other factors considered in our in silico analyses associated with identifying highly cancer selective therapeutics with enhanced potency relative to AOH1996 that target caPCNA included: reagent reactivity, ease of synthesis, cost of synthesis, relative affinity, interaction with the PIP box binding domain, etc.

In Silico Analysis Results involving substituting D- and L-amino acids for the Glycine Linker

The in silico analysis began by substituting the glycine linker between the “A” section (or Proximal section of AOH1996 (the methoxy amino diphenyl ether section)), and the “C” section (distal section of AOH1996 (the napthoic acid section)) (FIG. 14). We performed extensive in silico docking experiments to identify compounds with potentially equivalent or better binding affinity for the PIP interaction domain of the cancer isoform of PCNA, (caPCNA), which incorporated the A and C sections of AOH1996. Part of the simulations involved the substitution of the D- or L-isomers of all nineteen remaining amino acids, using Autodock VINA Projections to prepare the representations and determine the structure associated with the lowest energy configuration of the compound within the desired binding pocket. A table of energies for the 10 lowest energy configurations was prepared for each compound. The binding energies of the most stable configurations are included in Table 3 for each of the D- and L-isoforms of the analogs containing the glycine substitutions. It should be noted that while the strongest binding energy is listed in Table 3 for each analog, the other 9 conformations have a binding energy ranging from −8.7 to −7.1 Kcal/mole depending upon the docking configuration. The more negative the number, the stronger the binding. The table lists the best docking energy for each of the analogs with the PIP interaction domain on PCNA.

TABLE 3
Binding energies of the most stable configuration for
AOH1996 analogs using D- or L-amino acids to replace
the glycine linking the A and C sections of AOH1996.

| Amino acid linker | L- | D- |
|---|---|---|
| Glycine | −8.7 | n/a |
| Alanine | −9.1 | −8.3 |
| Valine | −7.2 | −7.7 |
| Leucine | −7.4 | −7.6 |
| Isoleucine | −7.4 | −8.0 |
| Proline | −8.5 | −8.5 |
| Methionine | −6.0 | −7.5 |
| Serine | −9.0 | −7.8 |
| Threonine | −8.3 | −7.4 |
| Cysteine | −7.8 | −7.8 |
| Aspartic acid | −8.4 | −8.4 |
| Glutamic acid | −7.4 | −7.6 |
| Asparagine | −8.3 | −7.8 |
| Glutamine | −7.2 | −7.7 |
| Histidine | −8.0 | −7.7 |
| Phenylalanine | −8.4 | −7.0 |
| Tyrosine | −7.7 | −8.4 |
| Tryptophan | −8.6 | −8.7 |
| Arginine | −6.1 | −8.5 |
| Lysine | −7.6 | −7.3 |

At least one or more of the alternate 9 configurations identified in our top 10 configurations for each analog interact with some other region on PCNA not in the PIP box domain. Additionally, in attempting to substitute other amino acids (beta, gamma, etc.) for the glycine, we identified a number of different linkers such as isonipecotic acid which enables the analog to bind even more tightly to the PIP box domain of PCNA; perhaps by extending the length of the amino acid “bridge” which seems to place the naphthalene into a better binding pocket or positioning the far amide in a more favorable position relative to the polar portions of the PCNA binding pocket. With respect to the substitution of the glycine with the other common alpha amino acids, and perhaps not too unexpectedly, substituting Alanine and Serine for the glycine produce slightly stronger binding interactions with the PIP domain. This is likely due in part to their similarity in size to the glycine linker in AOH1996. Substituting proline for the glycine results in almost the same predicted binding energy as for AOH1996, regardless of whether we used the D- or L-amino acid. The L-configuration amino acids Threonine, Aspartic Acid, Asparagine, Phenylalanine, and Tryptophan resulted in a slightly weaker interaction with the PIP box domain on PCNA, but nevertheless comparable to that of AOH1996. The L-configuration Histidine, Lysine, Arginine, Tyrosine, Glutamine, Glutamic acid, Cysteine Methionine, Isoleucine, Leucine, and Valine amino acid substitutions resulted in considerably weaker binding to this site. The L-configuration amino acids for Methionine, cysteine, Glutamic acid, Glutamine, Tyrosine, Arginine, and Lysine had lower binding affinities for the PIP box domain, with Arginine and Methionine having the lowest calculated binding strength of the analogs analyzed. The D-configuration of the Arginine, Tyrosine, Aspartic acid, Proline, Alanine, and Isoleucine resulted in estimated binding energies that were similar to that of AOH1996 containing the Glycine linker. When used as a molecular probe, the Lysine and Glutamic acid containing analogs bound caPCNA tightly and helped capture several of PCNA's interacting binding partners in vitro.

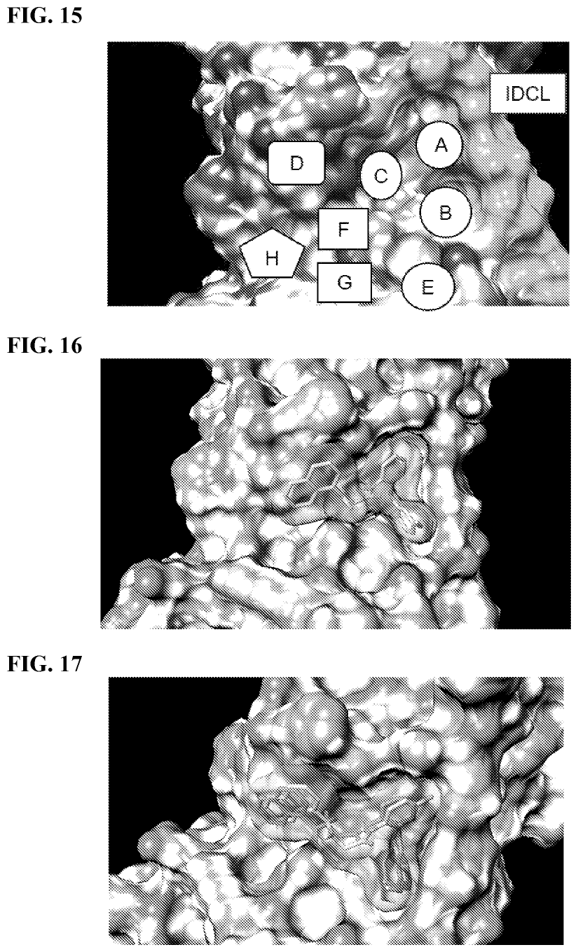

Analysis of the binding pocket within the PIP box domain of PCNA contains 8 readily identifiable features which can be used to model compounds “resembling” AOH1996 and its parent molecule AOH1160 (FIG. 15).

FIG. 16 shows the representation for the lowest energy configuration of AOH1996 (E=−8.7). The B pocket is well utilized, while the A pocket is somewhat less utilized, and the naphthalene unit associates with the Wall, D. The amide bonds on each side of the glycine associate with some polar features of C, but do not fill the pocket. There is also a predicted hydrogen bond between the methoxy oxygen attached to the B-pocket phenyl residue and a positive charge adjacent to the lower portion of the B-pocket. Addition of a fluorine to the adjacent phenyl ring introduces some polarity to the ring, which more deeply penetrates the A pocket, but decreases the binding (E=−7.5) despite the molecule having the same configuration (FIG. 17). In addition, adding a weakly acidic alkyne to this site completely disrupts binding into the A pocket (data not shown).

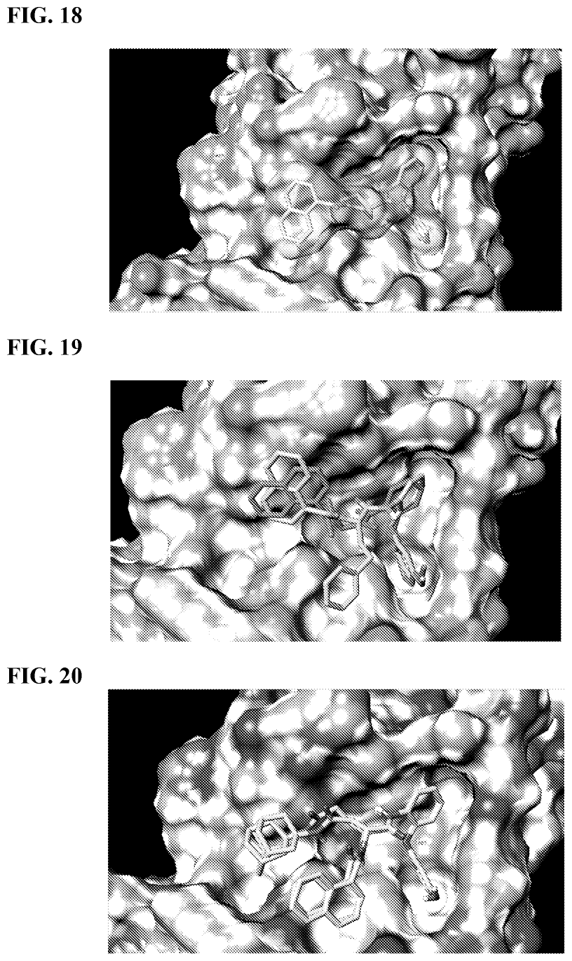

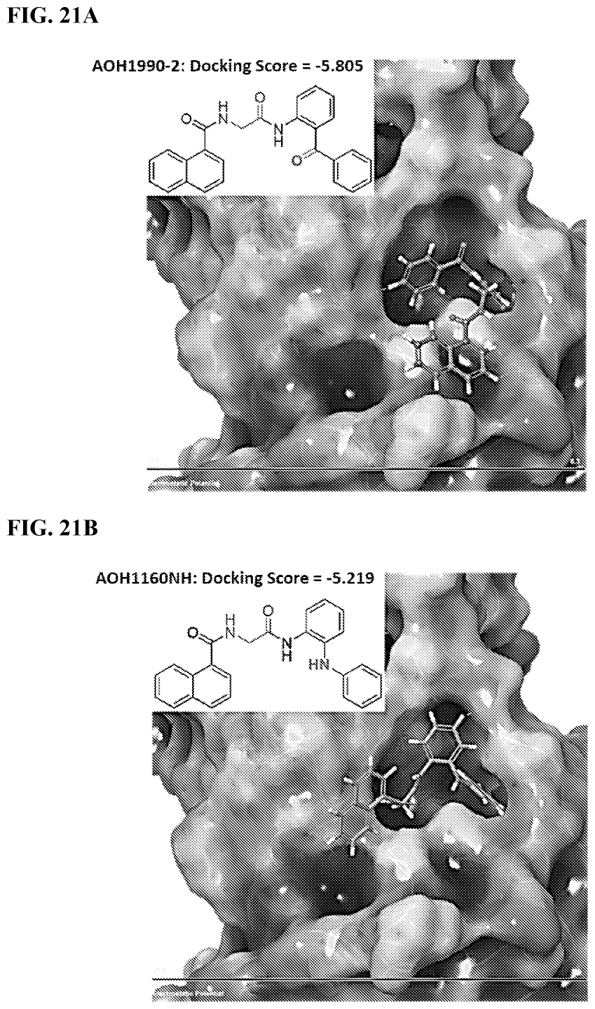

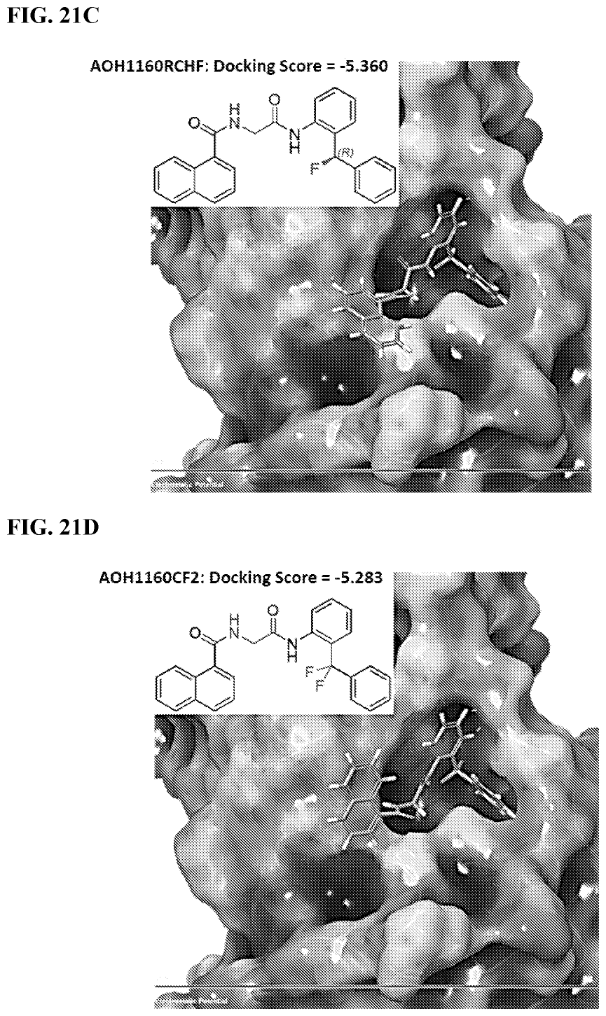

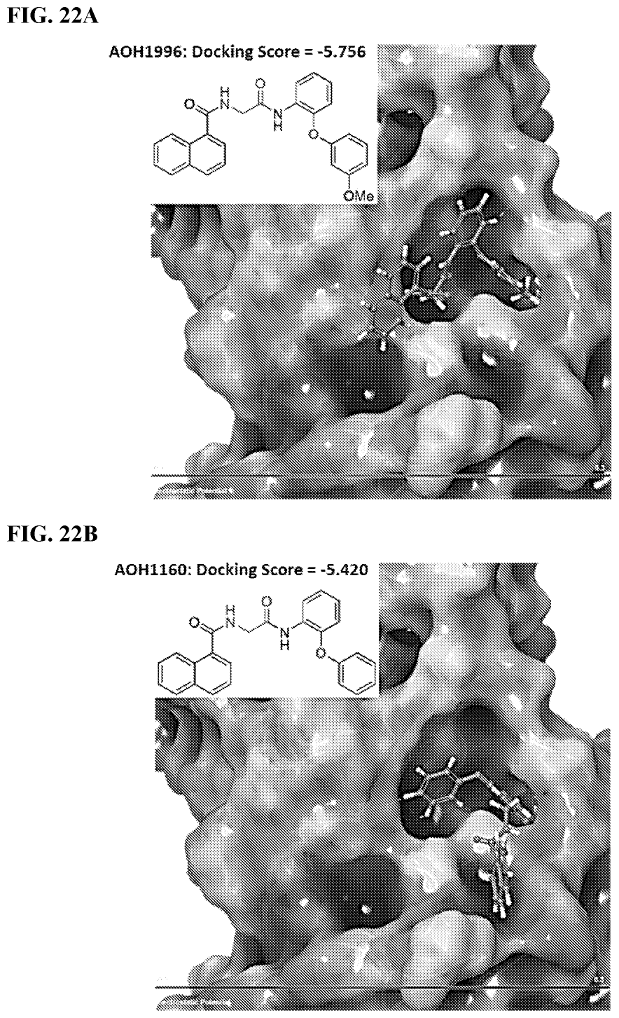

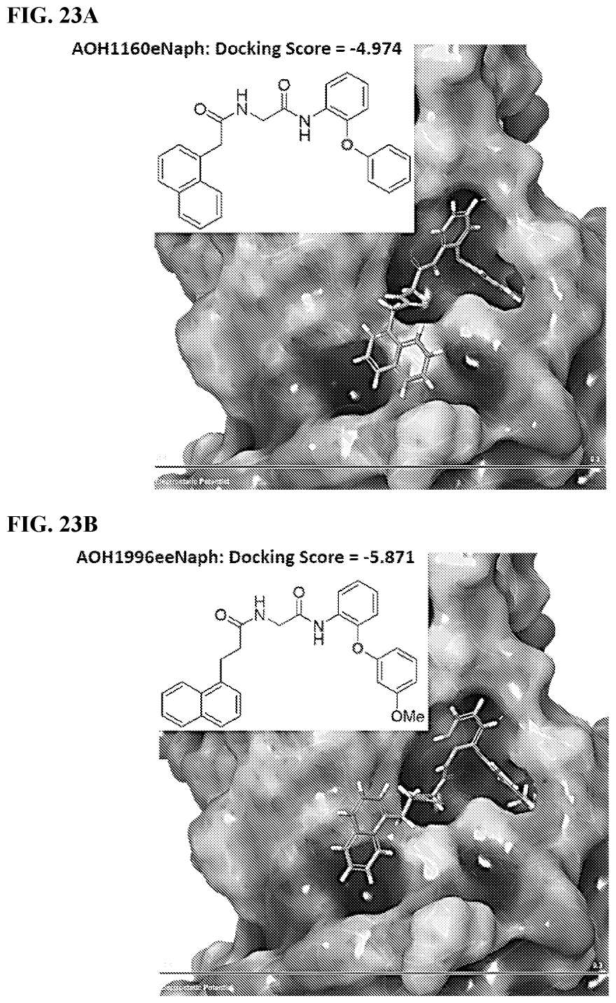

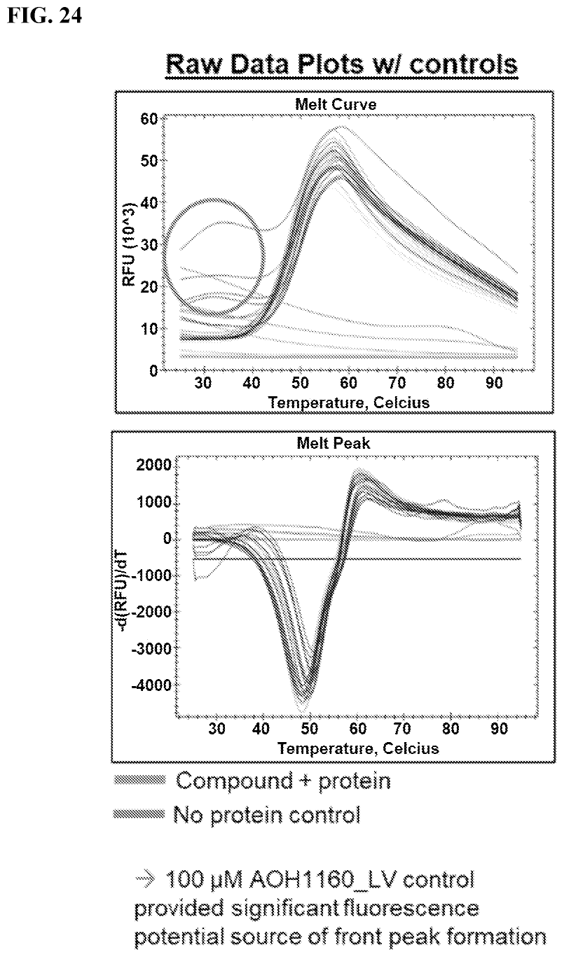

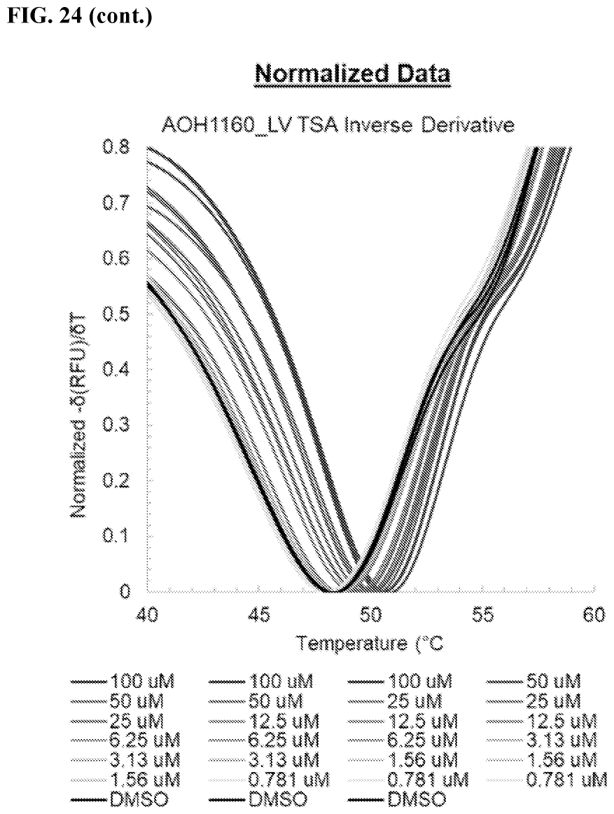

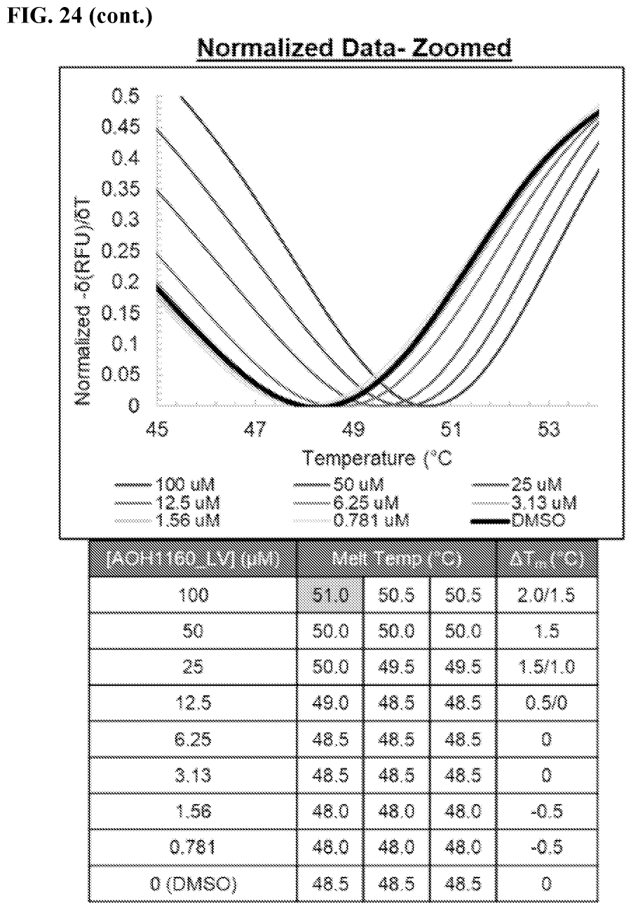

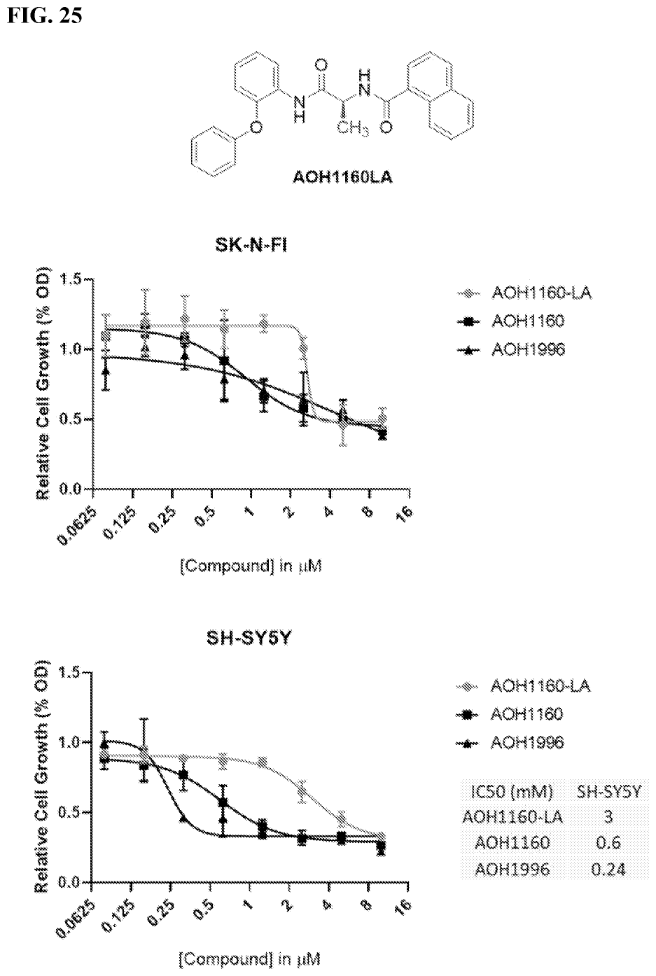

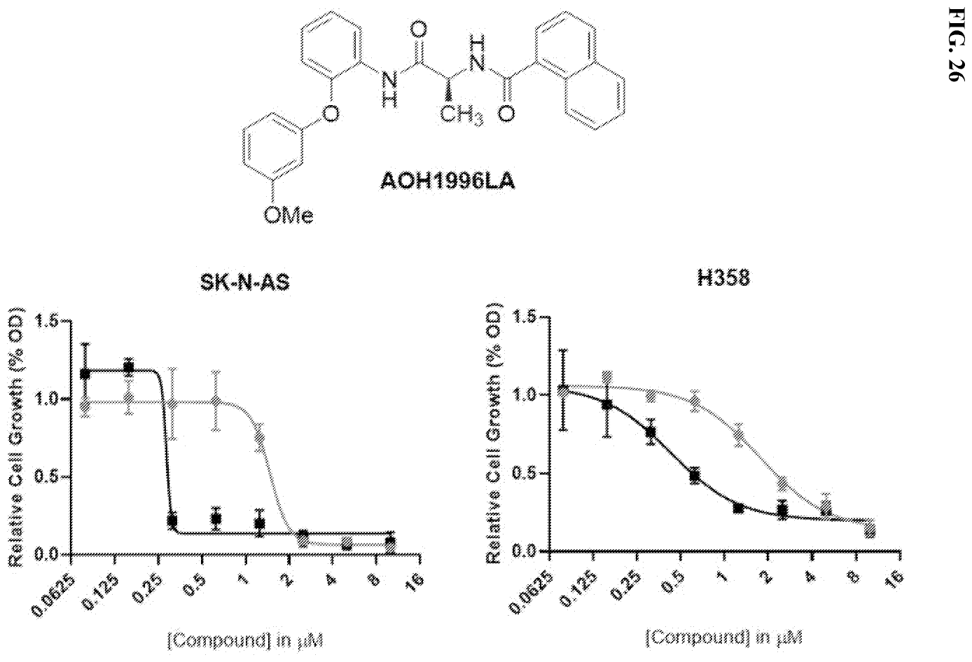

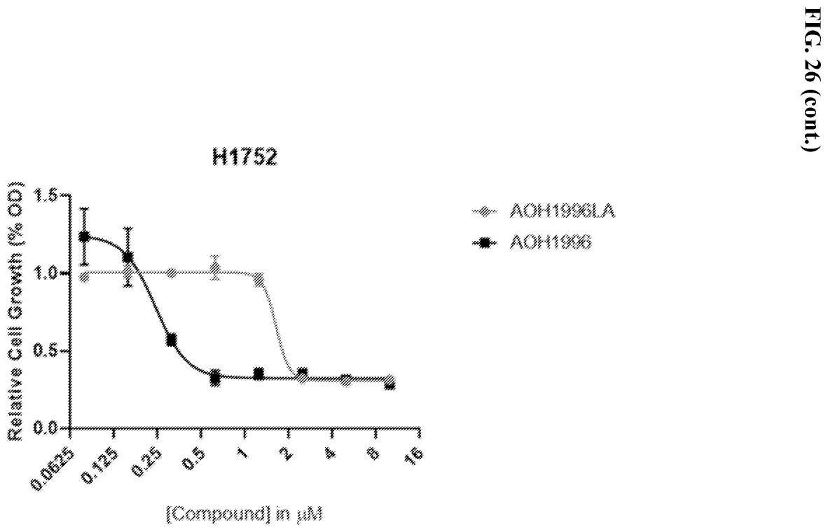

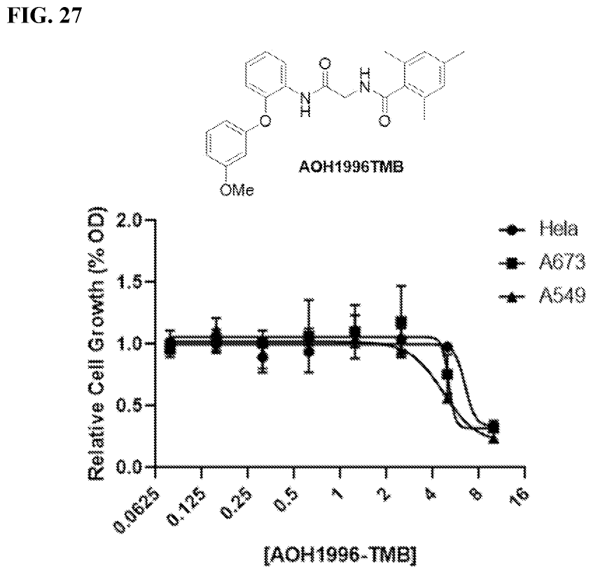

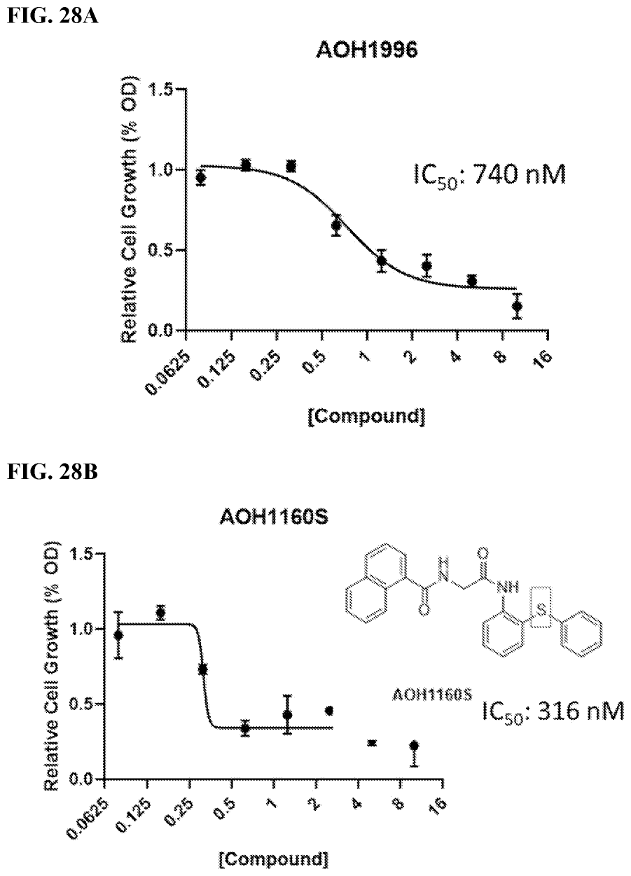

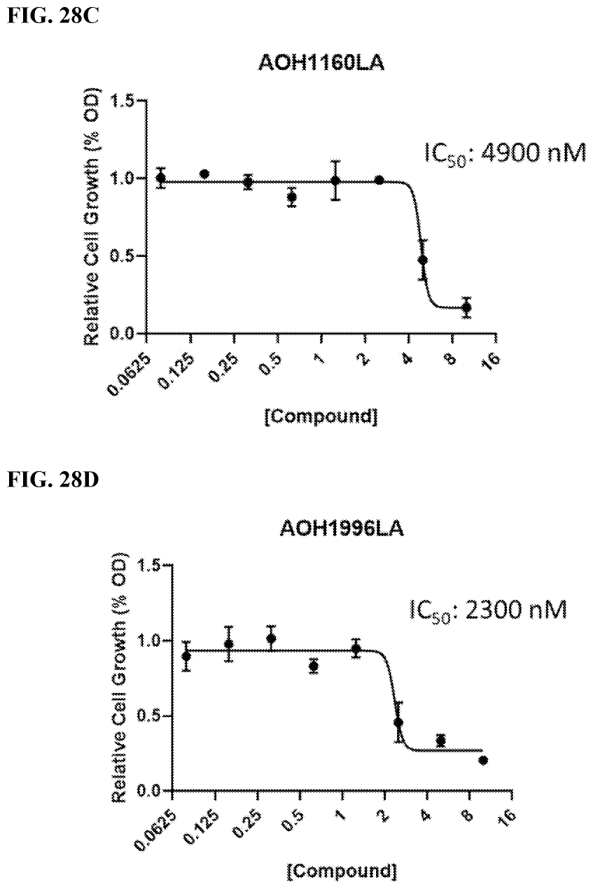

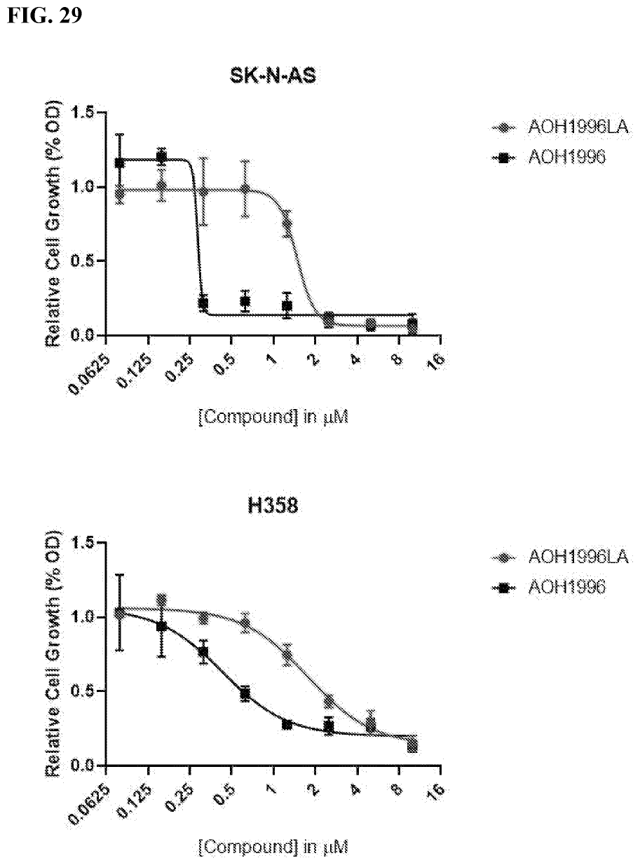

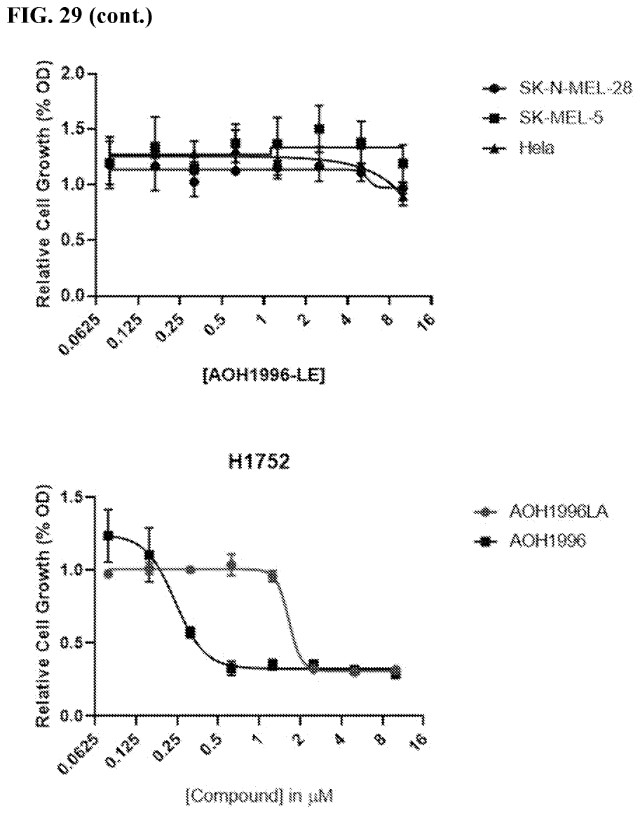

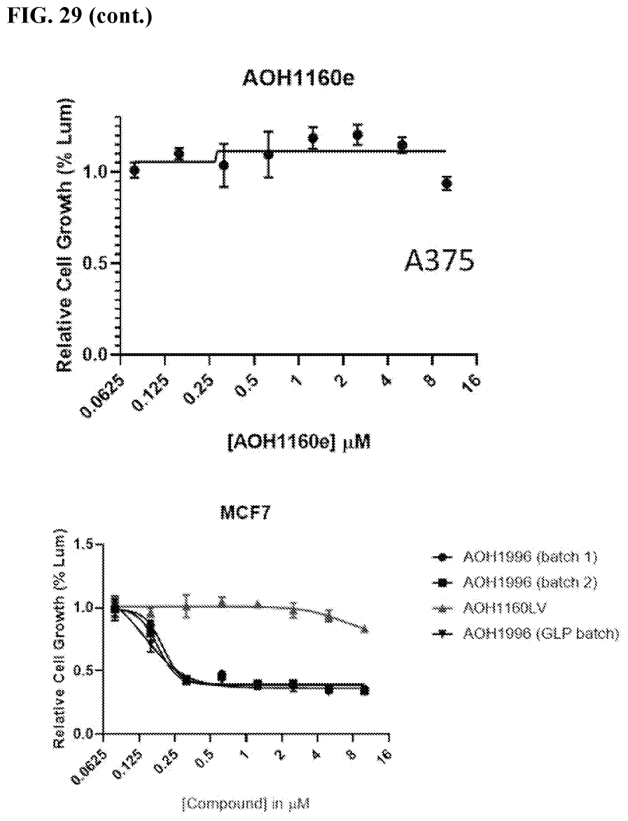

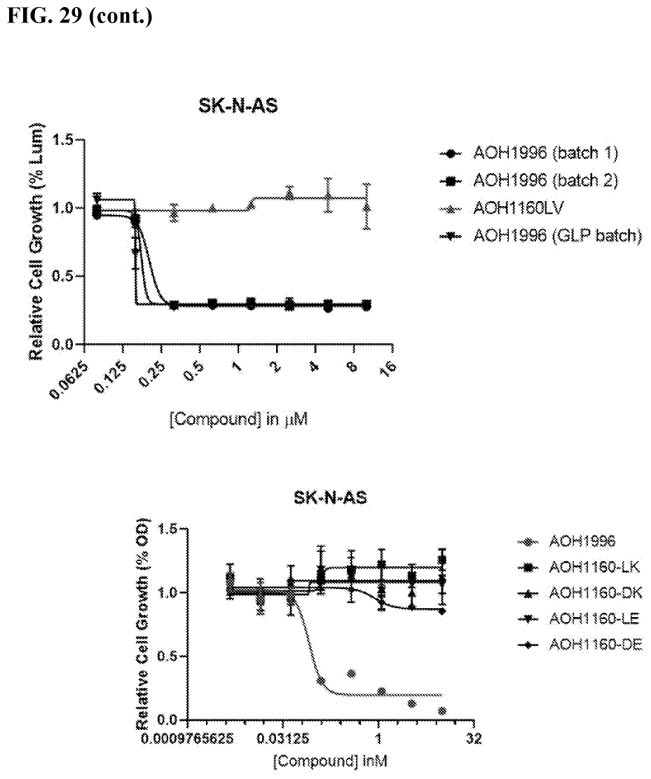

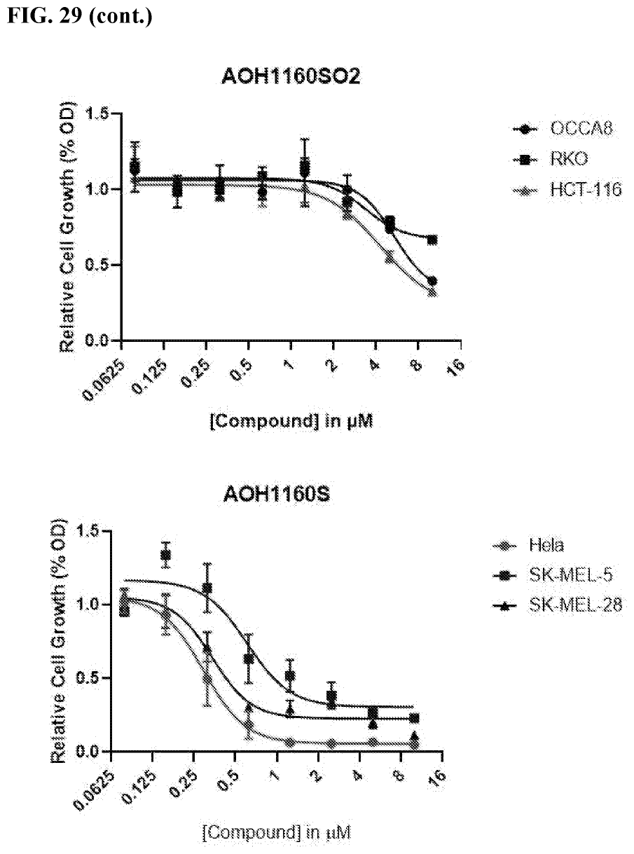

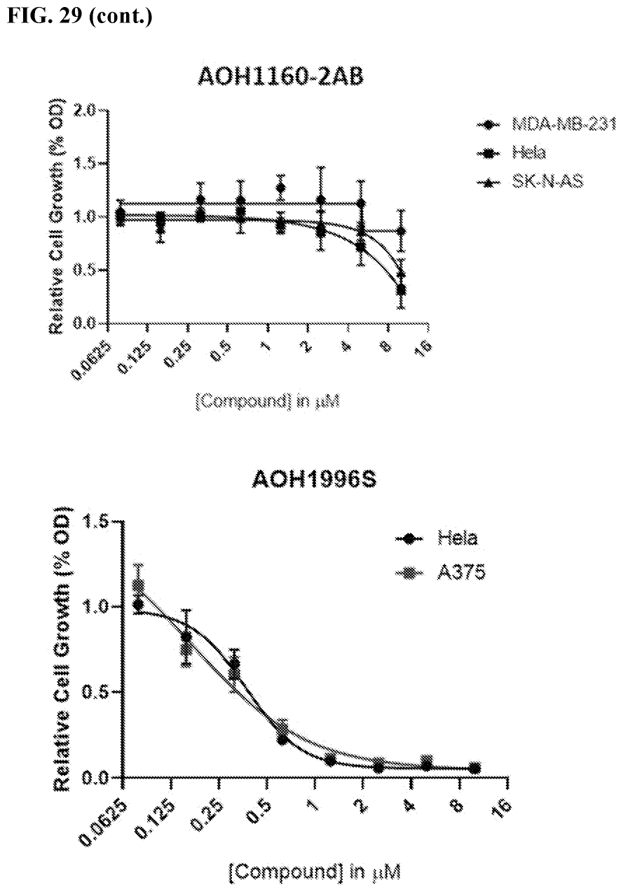

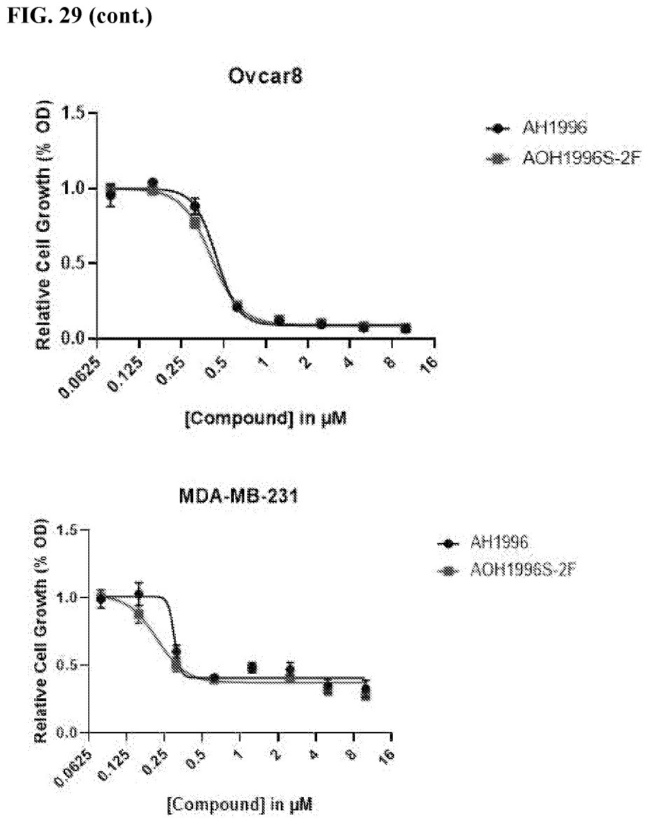

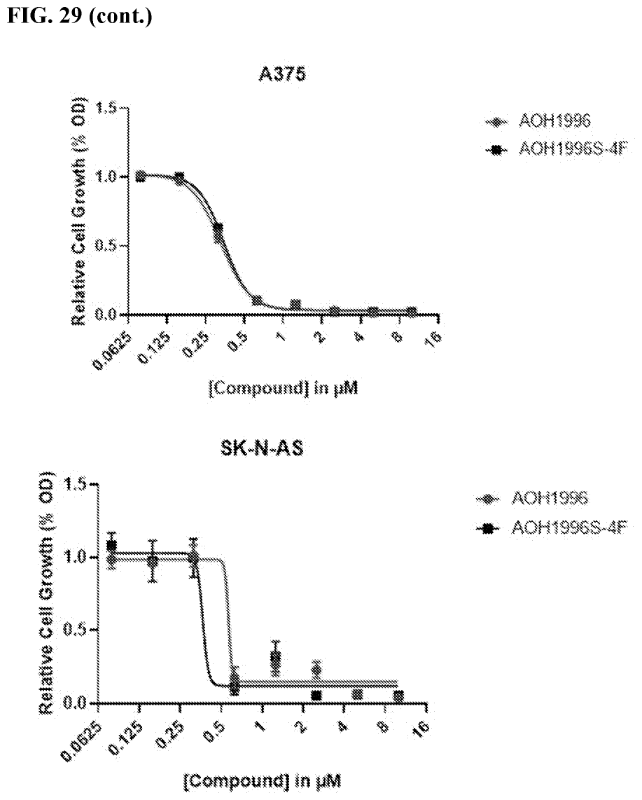

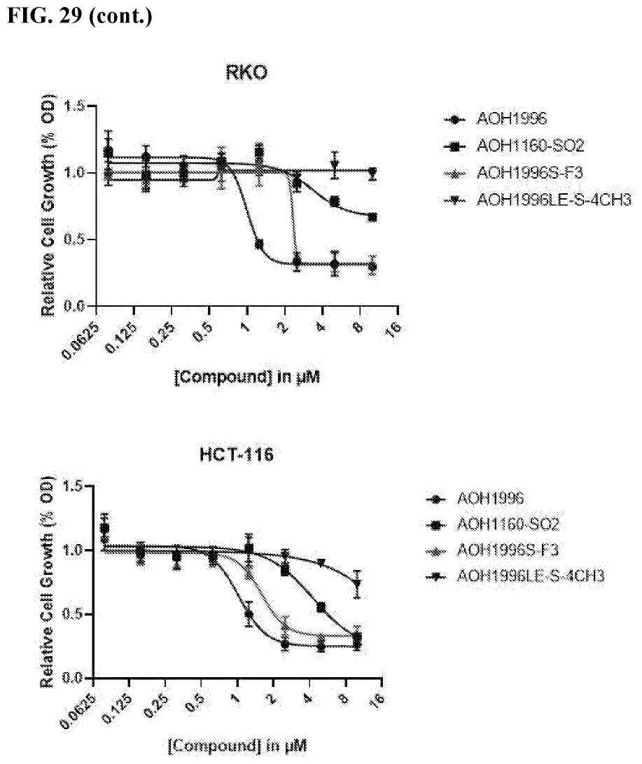

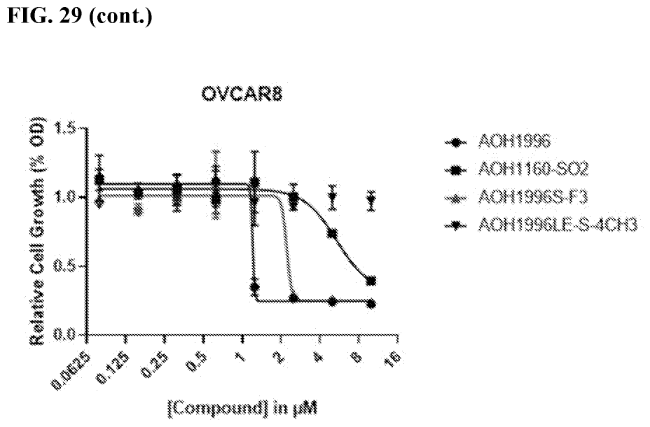

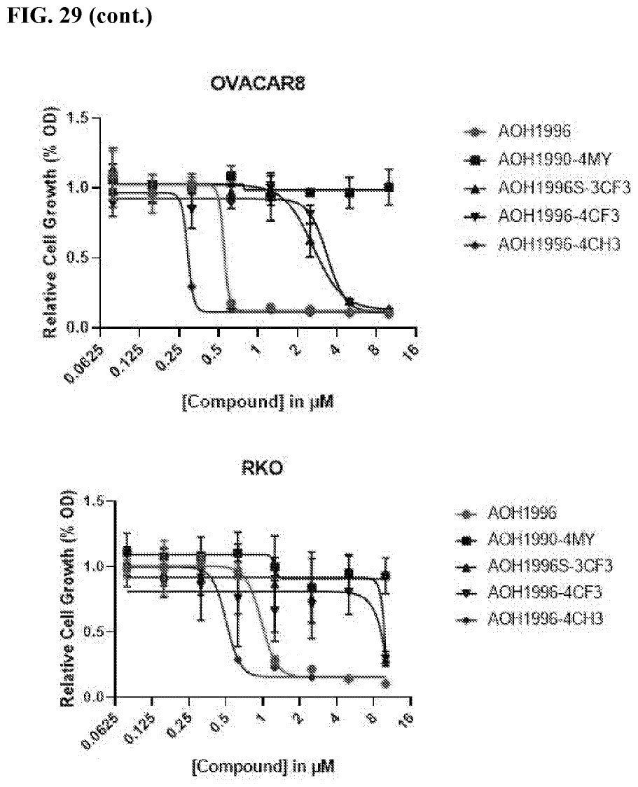

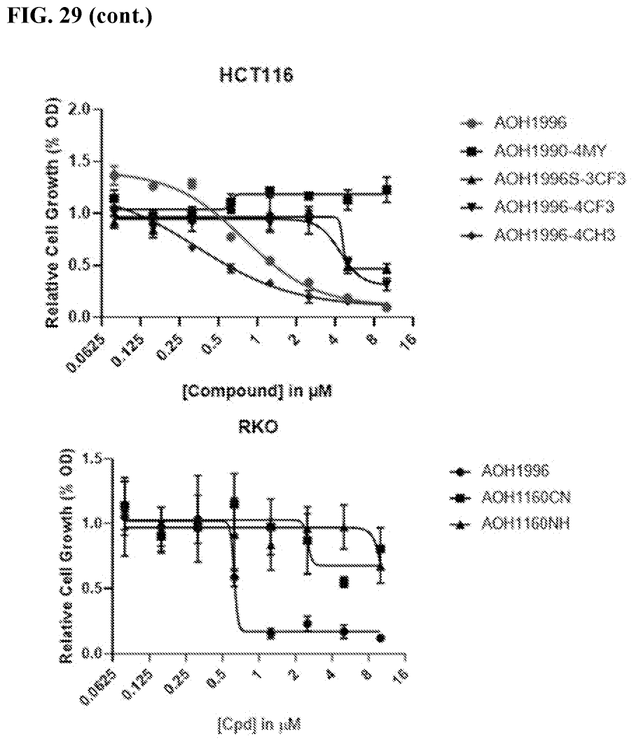

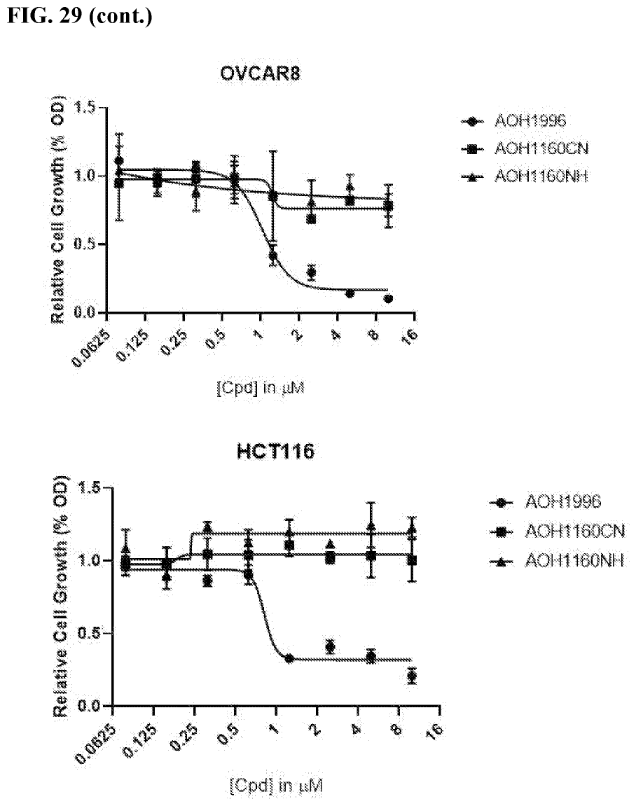

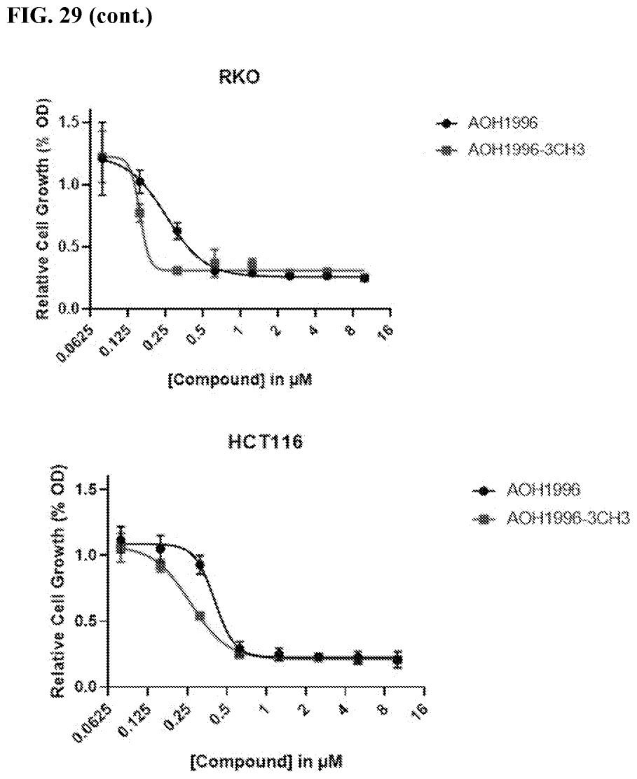

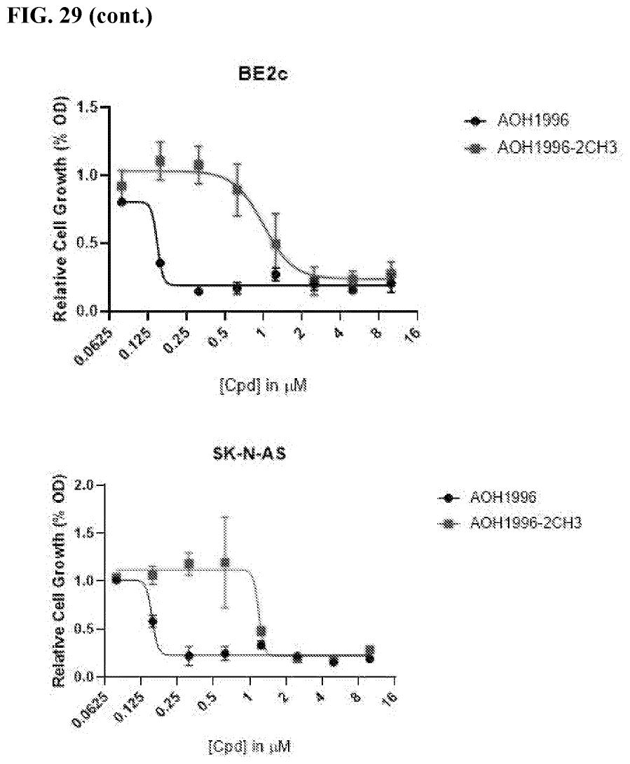

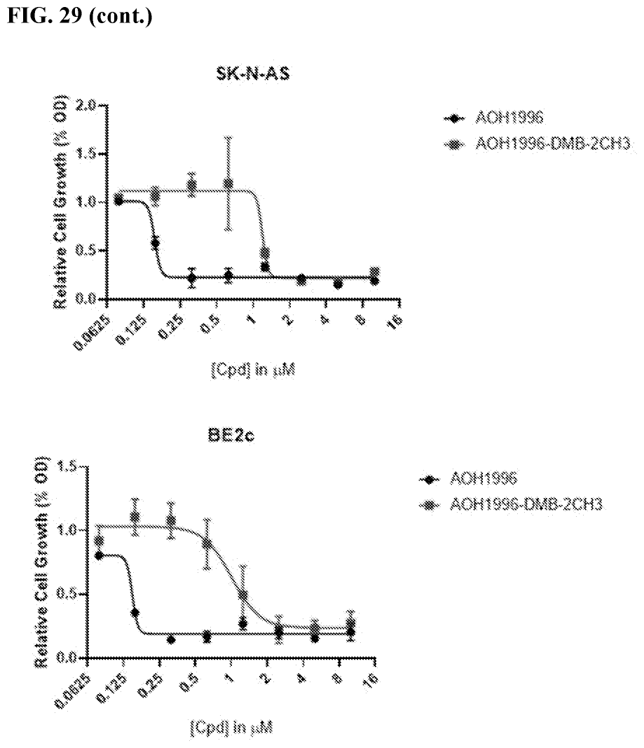

We next examined whether extending the length of the linker between the A and C portions of AOH1160 (the parent of AOH1996) enhanced binding to the PIP box domain (FIG. 12). FIG. 18 shows the binding representation, and predicts a somewhat stronger binding to the PIP box (E=−9.1); presumably because the isonipecotic ring partially fills and mostly caps the C region, which extends the overall length of the compound so the naphthalene ring partially utilizes the F, G, and H, regions for binding.

Additionally, we simulated docking of analogs containing either diphenylglycine around the alpha carbon of the glycine and examined whether analogs containing the diphenyl ether bound better to the A and B segments of the PIP box domain than analogs containing the diphenyl methane. 8 of 10 configurations place the diphenylmethane into the A and B segments of the PIP box vs. only 1 configuration for the diphenyl ether.

When we examined the unnatural amino acid D-homophenylalanine as the linker in place of the glycine, we found that the configuration for the homophenylalanine analog was almost identical to that of AOH1996 docking with this site, and the binding strength was almost the same (E=−8.5 vs. E=−8.6 for AOH1996) (FIG. 19).

To improve overall residence time in the PIP binding domain, we modeled AOH1996 analogs containing the adamantyl amides of D-aspartic and D-glutamic acids (E=−9.1 and E=−9.3 respectively) (FIG. 20). The two analogs effectively superimposed upon one another in minimized conformation, and exhibited somewhat higher affinity for the PIP binding domain than AOH1996. However, the adamantyl group does not reside in the targeted location and both aspartyl and glutamyl analogs are prone to highly varied alternate conformations of higher energy, which may affect selectivity of the analogs for the PIP binding domain. Additionally, the naphthalene moiety which appears to reside within the C and D segments of the PIP box domain appears to reside over the E segment of the PIP box domain.

### REFERENCES

1. Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). Acta crystallographica Section D, Biological crystallography 66, 213-221. 2. Ashwell, S., and Zabludoff, S. (2008). Clinical cancer research: an official journal of the American Association for Cancer Research 14, 4032-4037. 3. Caudron-Herger, M., Muller-Ott, K., Mallm, J. P., Marth, C., Schmidt, U., Fejes-Toth, K., and Rippe, K. (2011). Nucleus 2, 410-424. 4. Chapados, B. R., Hosfield, D. J., Han, S., Qiu, J., Yelent, B., Shen, B., and Tainer, J. A. (2004). Cell 116, 39-50. 5. de la Fuente-Nunez, C., and Lu, T. K. (2017). Integrative biology: quantitative biosciences from nano to macro 9, 109-122. 6. Dijt, F. J., Fichtinger-Schepman, A. M., Berends, F., and Reedijk, J. (1988). Cancer Res 48, 6058-6062. 7. Emsley, P., Lohkamp, B., Scott, W. G., and Cowtan, K. (2010). Acta crystallographica Section D, Biological crystallography 66, 486-501. 8. Foucquier, J., and Guedj, M. (2015). Pharmacology research & perspectives 3, e00149. 9. Friesner, R. A., Murphy, R. B., Repasky, M. P., Frye, L. L., Greenwood, J. R., Halgren, T. A., Sanschagrin, P. C., and Mainz, D. T. (2006). Journal of medicinal chemistry 49, 6177-6196. 10. Frum, R. A., Deb, S., and Deb, S. P. (2013). Methods in molecular biology 962, 147-155. 11. Gaillard, H., and Aguilera, A. (2016). Annu Rev Biochem 85, 291-317. 12. Gilljam, K. M., Feyzi, E., Aas, P. A., Sousa, M. M., Muller, R., Vagbo, C. B., Catterall, T. C., Liabakk, N. B., Slupphaug, G., Drablos, F., et al. (2009). The Journal of cell biology 186, 645-654. 13. Gu, L., Chu, P., Lingeman, R., McDaniel, H., Kechichian, S., Hickey, R. J., Liu, Z., Yuan, Y. C., Sandoval, J. A., Fields, G. B., and Malkas, L. H. (2015). EBioMedicine 2, 1923-1931. 14. Gu, L., Lingeman, R., Yakushijin, F., Sun, E., Cui, Q., Chao, J., Hu, W., Li, H., Hickey, R. J., Stark, J. M., et al. (2018). Clinical cancer research: an official journal of the American Association for Cancer Research 24, 6053-6065. 15. Gu, L., Smith, S., Li, C., Hickey, R. J., Stark, J. M., Fields, G. B., Lang, W. H., Sandoval, J. A., and Malkas, L. H. (2014). PLOS One 9, e94773. 16. Guo, X., Chen, Z., Xia, Y., Lin, W., and Li, H. (2020). Journal of translational medicine 18, 321. 17. Han, C., Srivastava, A. K., Cui, T., Wang, Q. E., and Wani, A. A. (2016). Carcinogenesis 37, 129-138. 18. Hanahan, D., and Weinberg, R. A. (2000). Cell 100, 57-70. 19. Hanahan, D., and Weinberg, R. A. (2011). Cell 144, 646-674. 20. Helmrich, A., Ballarino, M., Nudler, E., and Tora, L. (2013). Nat Struct Mol Biol 20, 412-418. 21. Hsu, P. D., Lander, E. S., and Zhang, F. (2014). Cell 157, 1262-1278. 22. Inoue, A., Kikuchi, S., Hishiki, A., Shao, Y., Heath, R., Evison, B. J., Actis, M., Canman, C. E., Hashimoto, H., and Fujii, N. (2014). J Biol Chem 289, 7109-7120. 23. Jones, R. M., Mortusewicz, O., Afzal, I., Lorvellec, M., Garcia, P., Helleday, T., and Petermann, E. (2013). Oncogene 32, 3744-3753. 24. Kabsch, W. (2010). Acta crystallographica Section D, Biological crystallography 66, 133-144. 25. Koshland, D. E. (1958). Proc Natl Acad Sci USA 44, 98-104. 26. Krishna, T. S., Kong, X. P., Gary, S., Burgers, P. M., and Kuriyan, J. (1994). Cell 79, 1233-1243. 27. Lei, Q., Gao, F., Liu, T., Ren, W., Chen, L., Cao, Y., Chen, W., Guo, S., Zhang, Q., Chen, W., et al. (2021). Science translational medicine 13. 28. Li, H., Sandhu, M., Malkas, L. H., Hickey, R. J., and Vaidehi, N. (2017). Journal of chemical information and modeling 57, 3011-3021. 29. Li, M., Pokharel, S., Wang, J. T., Xu, X., and Liu, Y. (2015). Nature communications 6, 6720. 30. Li, M., Xu, X., Chang, C. W., Zheng, L., Shen, B., and Liu, Y. (2018). Nature communications 9, 2706. 31. Li, M., Xu, X., and Liu, Y. (2011). Mol Cell Biol 31, 2090-2099. 32. Luo, J., Solimini, N. L., and Elledge, S. J. (2009). Cell 136, 823-837. 33. Ma, M. K., Zamboni, W. C., Radomski, K. M., Furman, W. L., Santana, V. M., Houghton, P. J., Hanna, S. K., Smith, A. K., and Stewart, C. F. (2000). Clinical cancer research: an official journal of the American Association for Cancer Research 6, 813-819. 34. Malkas, L. H., Herbert, B. S., Abdel-Aziz, W., Dobrolecki, L. E., Liu, Y., Agarwal, B., Hoelz, D., Badve, S., Schnaper, L., Arnold, R. J., et al. (2006). Proc Natl Acad Sci USA 103, 19472-19477. 35. McCoy, A. J., Grosse-Kunstleve, R. W., Adams, P. D., Winn, M. D., Storoni, L. C., and Read, R. J. (2007). Journal of applied crystallography 40, 658-674. 36. Muller, R., Misund, K., Holien, T., Bachke, S., Gilljam, K. M., Vatsveen, T. K., Ro, T. B., Bellacchio, E., Sundan, A., and Otterlei, M. (2013). PLOS One 8, e70430. 37. Nair, A. B., and Jacob, S. (2016). Journal of basic and clinical pharmacy 7, 27-31. 38. Phillips, J. C., Braun, R., Wang, W., Gumbart, J., Tajkhorshid, E., Villa, E., Chipot, C., Skeel, R. D., Kale, L., and Schulten, K. (2005). Journal of computational chemistry 26, 1781-1802. 39. Pommier, Y. (2006). Nat Rev Cancer 6, 789-802. 40. Punchihewa, C., Inoue, A., Hishiki, A., Fujikawa, Y., Connelly, M., Evison, B., Shao, Y., Heath, R., Kuraoka, I., Rodrigues, P., et al. (2012). J Biol Chem 287, 14289-14300. 41. Sebesta, M., Cooper, C. D. O., Ariza, A., Carnie, C. J., and Ahel, D. (2017). Nature communications 8, 15847. 42. Sherman, W., Day, T., Jacobson, M. P., Friesner, R. A., and Farid, R. (2006). Journal of medicinal chemistry 49, 534-553. 43. Smith, S. J., Gu, L., Phipps, E. A., Dobrolecki, L. E., Mabrey, K. S., Gulley, P., Dillehay, K. L., Dong, Z., Fields, G. B., Chen, Y. R., et al. (2015). Mol Pharmacol 87, 263-276. 44. Tan, Z., Wortman, M., Dillehay, K. L., Seibel, W. L., Evelyn, C. R., Smith, S. J., Malkas, L. H., Zheng, Y., Lu, S., and Dong, Z. (2012). Mol Pharmacol 81, 811-819. 45. Tsutakawa, S. E., Van Wynsberghe, A. W., Freudenthal, B. D., Weinacht, C. P., Gakhar, L., Washington, M. T., Zhuang, Z., Tainer, J. A., and Ivanov, I. (2011). Proc Natl Acad Sci USA 108, 17672-17677. 46. Tsutakawa, S. E., Yan, C., Xu, X., Weinacht, C. P., Freudenthal, B. D., Yang, K., Zhuang, Z., Washington, M. T., Tainer, J. A., and Ivanov, I. (2015). Structure 23, 724-733. 47. Waga, S., Hannon, G. J., Beach, D., and Stillman, B. (1994). Nature 369, 574-578. 48. Yu, D. D., Andrali, S. S., Li, H., Lin, M., Huang, W., and Forman, B. M. (2016). Bioorganic & medicinal chemistry 24, 3986-3993. 49. Yu, Y. L., Chou, R. H., Liang, J. H., Chang, W. J., Su, K. J., Tseng, Y. J., Huang, W. C., Wang, S. C., and Hung, M. C. (2013). PLOS One 8, e61362. 50. Zhao, H., Lo, Y. H., Ma, L., Waltz, S. E., Gray, J. K., Hung, M. C., and Wang, S. C. (2011). Mol Cancer Ther 10, 29-36. 51. Bueno, Oskia, et al. European journal of medicinal chemistry 171 (2019): 195-208. 52. Coste, J., D. Le-Nguyen, and B. Castro. 31.2 (1990): 205-208. 53. Dourtoglou, Vassilis, Jean-Claude Ziegler, and Bernard Gross. Tetrahedron letters 19.15 (1978): 1269-1272. 54. Greenberg, Jacob A., and Tarek Sammakia. The Journal of organic chemistry 82.6 (2017): 3245-3251.

What is claimed is:

1. A compound, or a pharmaceutically acceptable salt thereof, having the formula: 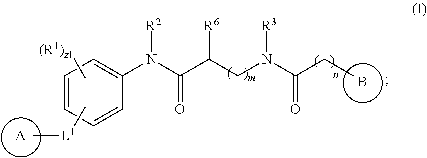
wherein
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
Ring B is substituted or unsubstituted phenyl, substituted or unsubstituted naphthyl, substituted or unsubstituted quinolinyl, or substituted or unsubstituted isoquinolinyl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOₙ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F;
m is an integer from 0 to 5; and
n is an integer from 0 to 10;
provided when L¹ is —O—, m is 0, n is 0, and Ring B is substituted or unsubstituted naphthyl, substituted or unsubstituted quinolinyl, or substituted or unsubstituted isoquinolinyl, then R⁶ is not hydrogen.

2. The compound of claim 1, having the formula: 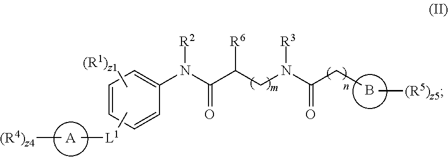
wherein
Ring A is phenyl or 5 to 6 membered heteroaryl;
Ring B is phenyl, naphthyl, quinolinyl, or isoquinolinyl;
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{1C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX₅³, —CHX⁵₂, —CH₂X⁵, —OCX₅³, —OCHX⁵₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom May optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
z4 is an integer from 0 to 5;
z5 is an integer from 0 to 7;
m4, m5, v4, and v5 are independently 1 or 2;
n4 and n5 are independently an integer from 0 to 4; and
X⁴ and X⁵ are independently —CI, —Br, —I, or —F.

3. The compound of claim 2, having the formula:

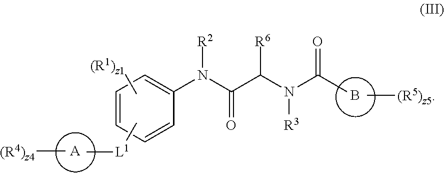

4. The compound of claim 3, having the formula:

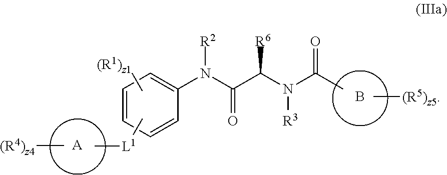

5. The compound of claim 3, having the formula:

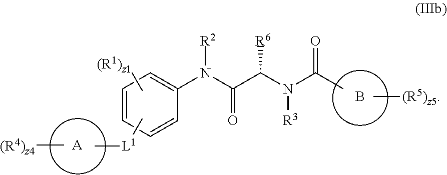

6. The compound of claim 3, having the formula:

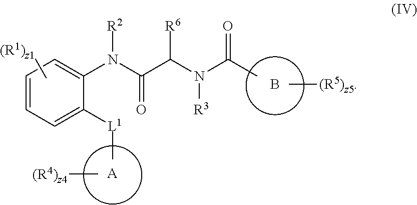

7. The compound of claim 3, having the formula:

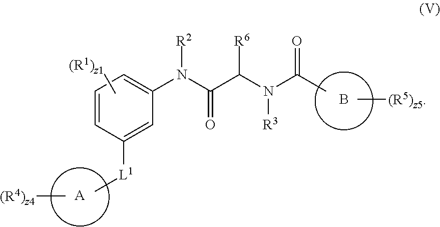

8. The compound of claim 3, having the formula:

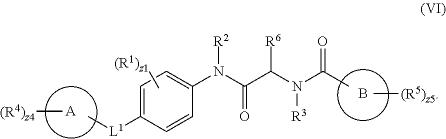

9. The compound of claim 1, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

10. The compound of claim 1, wherein L¹ is —O—.

11. The compound of claim 1, wherein L¹ is —S—.

12. The compound of claim 1, wherein L¹ is —S(O)₂—.

13. The compound of claim 1, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

14. The compound of claim 1, wherein R¹ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

15. The compound of claim 1, wherein R¹ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

16. The compound of claim 1, wherein R¹ is independently halogen, —OH, —CF₃, —CHF₂, —CH₂F, —OCF₃, —OCHF₂, —OCH₂F, unsubstituted methyl, or unsubstituted methoxy.

17. The compound of claim 1, wherein z1 is 1.

18. The compound of claim 1, wherein z1 is 0.

19. The compound of claim 1, wherein R² is hydrogen, —CX²₃, —CHX²₂, —CH₂X², —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

20. The compound of claim 1, wherein R² is hydrogen, unsubstituted methyl, unsubstituted ethyl, or unsubstituted isopropyl.

21. The compound of claim 1, wherein R² is hydrogen.

22. The compound of claim 1, wherein R³ is hydrogen, —CX³₃, —CHX₃², —CH₂X³, —CN, —C(O)H, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

23. The compound of claim 1, wherein R³ is hydrogen, unsubstituted methyl, unsubstituted ethyl, or unsubstituted isopropyl.

24. The compound of claim 1, wherein R³ is hydrogen.

25. The compound of claim 1, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

26. The compound of claim 1, wherein R⁶ is substituted or unsubstituted C₁-C₆ alkyl or substituted or unsubstituted 2 to 6 membered heteroalkyl.

27. The compound of claim 1, wherein R⁶ is hydrogen, unsubstituted methyl, unsubstituted isopropyl,

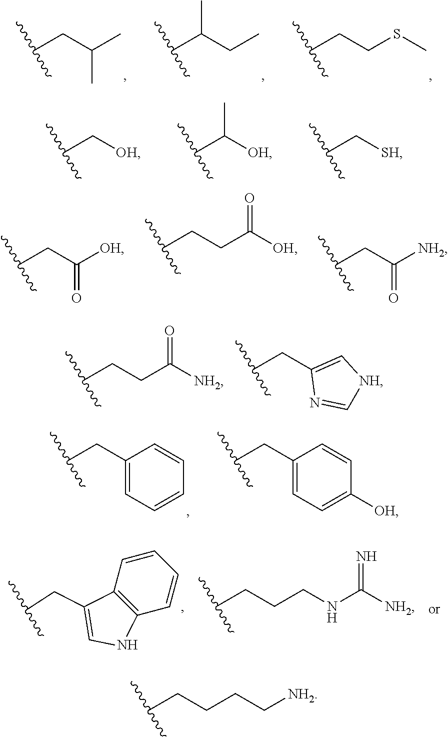

28. The compound of claim 1, wherein R³ and R⁶ are joined to form a substituted or unsubstituted heterocycloalkyl or substituted or unsubstituted heteroaryl.

29. The compound of claim 1, wherein R³ and R⁶ are joined to form a substituted or unsubstituted 4 to 8 membered heterocycloalkyl.

30. The compound of claim 1, wherein R³ and R⁶ are joined to form an unsubstituted pyrrolidinyl.

31. The compound of claim 1, wherein R³ and R⁶ are joined to form an unsubstituted piperidinyl.

32. The compound of claim 2, wherein R⁴ is independently halogen, —CF₃, —CHF₂, CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

33. The compound of claim 2, wherein R⁴ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

34. The compound of claim 2, wherein R⁴ is independently halogen, —CF₃, —CHF₂, —CH₂F, —OCF₃, —OCHF₂, —OCH₂F, —OH, —NH₂, —SH, unsubstituted C₁-C₄ alkyl, or unsubstituted 2 to 4 membered heteroalkyl.

35. The compound of claim 2, wherein R⁴ is independently halogen, —CF₃, —CHF₂, —CH₂F, —OCF₃, —OCHF₂, —OCH₂F, —OH, unsubstituted methyl, or unsubstituted methoxy.

36. The compound of claim 2, wherein R⁴ is independently —OR^{4D}.

37. The compound of claim 36, wherein R^{4D} is hydrogen or substituted or unsubstituted alkyl.

38. The compound of claim 36, wherein R^{4D} is hydrogen or unsubstituted alkyl.

39. The compound of claim 36, wherein R^{4D} is hydrogen or unsubstituted C₁-C₅ alkyl.

40. The compound of claim 36, wherein R^{4D} is hydrogen or unsubstituted methyl.

41. The compound of claim 36, wherein R^{4D} is unsubstituted methyl.

42. The compound of claim 2, wherein z4 is 1.

43. The compound of claim 2, wherein z4 is 0.

44. The compound of claim 2, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

45. The compound of claim 2, wherein R⁵ is independently halogen, —CF₃, —OH, —NH₂, —SH, substituted or unsubstituted C₁-C₄ alkyl, substituted or unsubstituted 2 to 4 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl.

46. The compound of claim 2, wherein R⁵ is independently halogen, —CF₃, —CHF₂, —CH₂F, —OCF₃, —OCHF₂, —OCH₂F, —OH, —NH₂, —SH, unsubstituted C₁-C₄ alkyl, unsubstituted 2 to 4 membered heteroalkyl, or unsubstituted phenyl.

47. The compound of claim 2, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

48. The compound of claim 2, wherein z5 is 1.

49. The compound of claim 2, wherein z5 is 0.

50. The compound of claim 1, wherein Ring A is a substituted or unsubstituted phenyl.

51. The compound of claim 1, wherein Ring A is a substituted or unsubstituted 5 to 6 membered heteroaryl.

52. The compound of claim 1, wherein Ring A is a substituted or unsubstituted thienyl.

53. The compound of claim 1, wherein Ring A is a substituted or unsubstituted 2-thienyl.

54. The compound of claim 1, wherein Ring A is a substituted or unsubstituted 3-thienyl.

55. The compound of claim 1, wherein Ring A is a substituted or unsubstituted pyridyl.

56. The compound of claim 1, wherein Ring A is a substituted or unsubstituted 2-pyridyl.

57. The compound of claim 1, wherein Ring A is a substituted or unsubstituted 3-pyridyl.

58. The compound of claim 1, wherein Ring A is a substituted or unsubstituted 4-pyridyl.

59. The compound of claim 1, wherein Ring B is a substituted or unsubstituted phenyl.

60. The compound of claim 1, wherein Ring B is a substituted or unsubstituted naphthyl.

61. The compound of claim 1, wherein Ring B is a substituted or unsubstituted 1-naphthyl.

62. The compound of claim 1, wherein Ring B is a substituted or unsubstituted 2-naphthyl.

63. The compound of claim 1, wherein Ring B is a substituted or unsubstituted quinolinyl.

64. The compound of claim 1, wherein Ring B is a substituted or unsubstituted 2-quinolinyl.

65. The compound of claim 1, wherein Ring B is a substituted or unsubstituted 3-quinolinyl.

66. The compound of claim 1, wherein Ring B is a substituted or unsubstituted 4-quinolinyl.

67. The compound of claim 1, wherein Ring B is a substituted or unsubstituted isoquinolinyl.

68. The compound of claim 1, wherein Ring B is a substituted or unsubstituted 1-isoquinolinyl.

69. The compound of claim 1, wherein Ring B is a substituted or unsubstituted 3-isoquinolinyl.

70. The compound of claim 1, wherein Ring B is a substituted or unsubstituted 4-isoquinolinyl.

71. The compound of claim 2, having the formula:

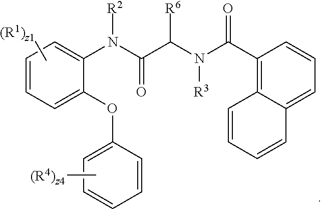

72. The compound of claim 2, having the formula:

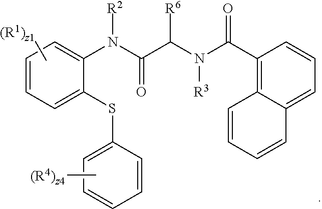

73. The compound of claim 2, having the formula:

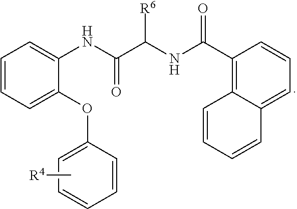

74. The compound of claim 2, having the formula:

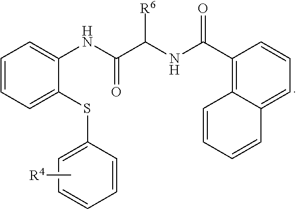

75. The compound of claim 1, having the formula:

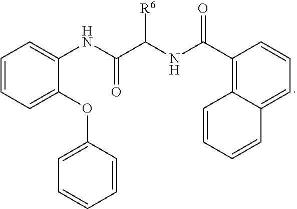

76. The compound of claim 1, having the formula:

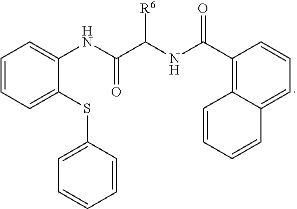

77. The compound of claim 2, having the formula:

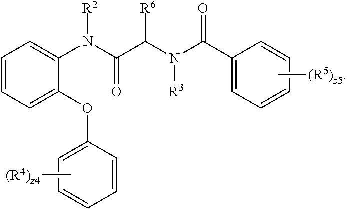

78. The compound of claim 2, having the formula:

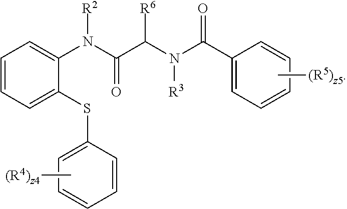

79. The compound of claim 2, having the formula:

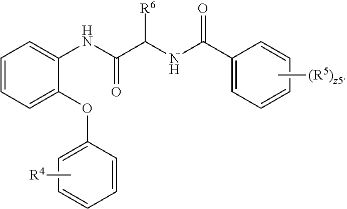

80. The compound of claim 1, having the formula:

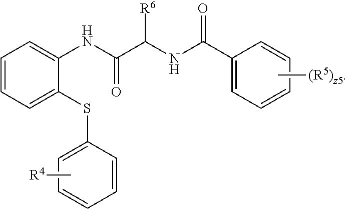

81. The compound of claim 2, having the formula:

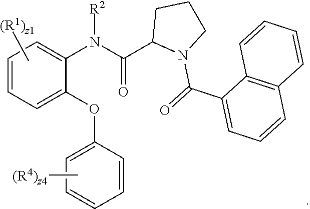

82. The compound of claim 2, having the formula:

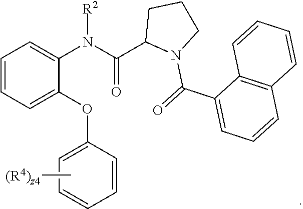

83. The compound of claim 2, having the formula:

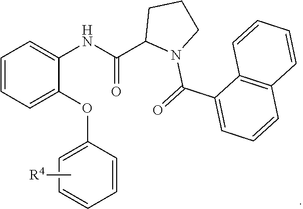

84. The compound of claim 2, having the formula:

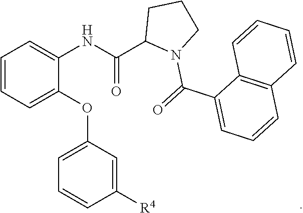

85. The compound of claim 1, having the formula:

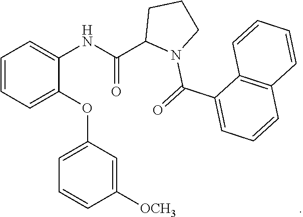 [SMILES: COc1cccc(Oc2ccccc2NC(=O)C2CCCN2C(=O)c2cccc3ccccc23)c1]

86. The compound of claim 1, having the formula:

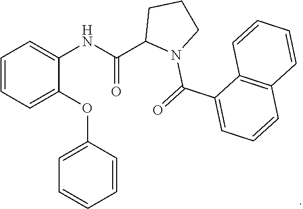 [SMILES: O=C(Nc1ccccc1Oc1ccccc1)C1CCCN1C(=O)c1cccc2ccccc12]

87. The compound claim 1, having the formula:

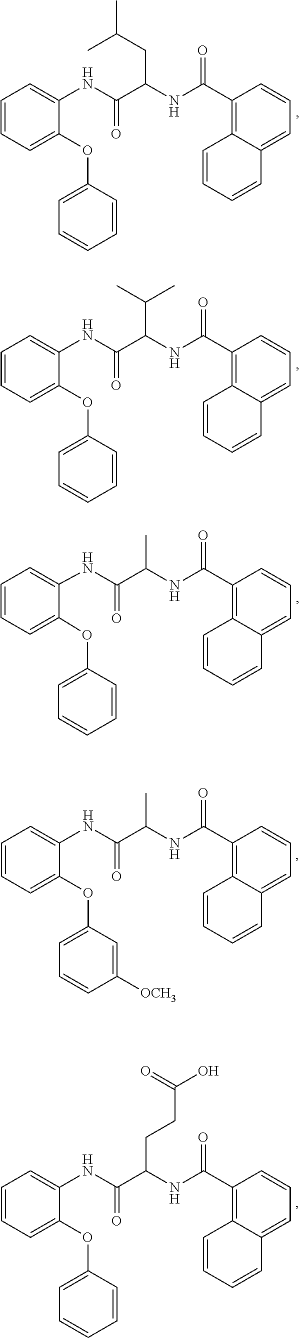

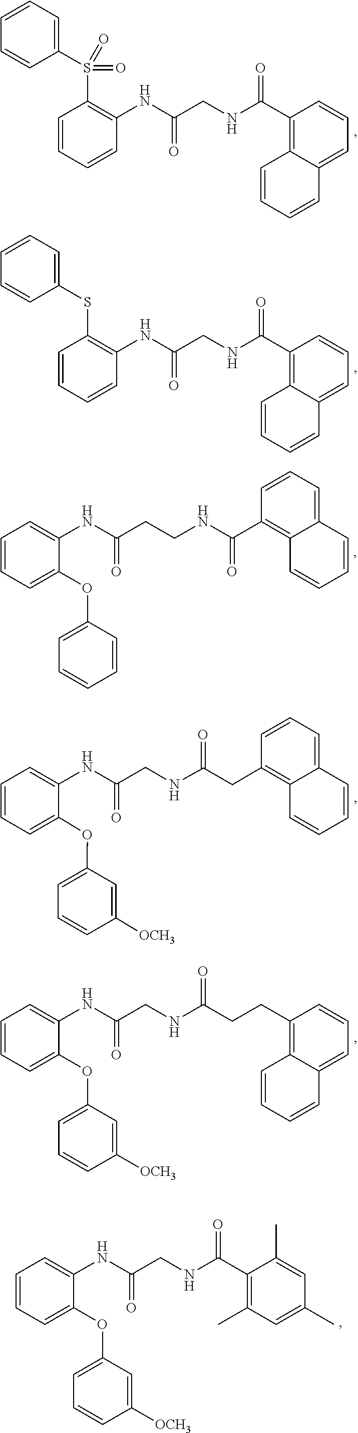

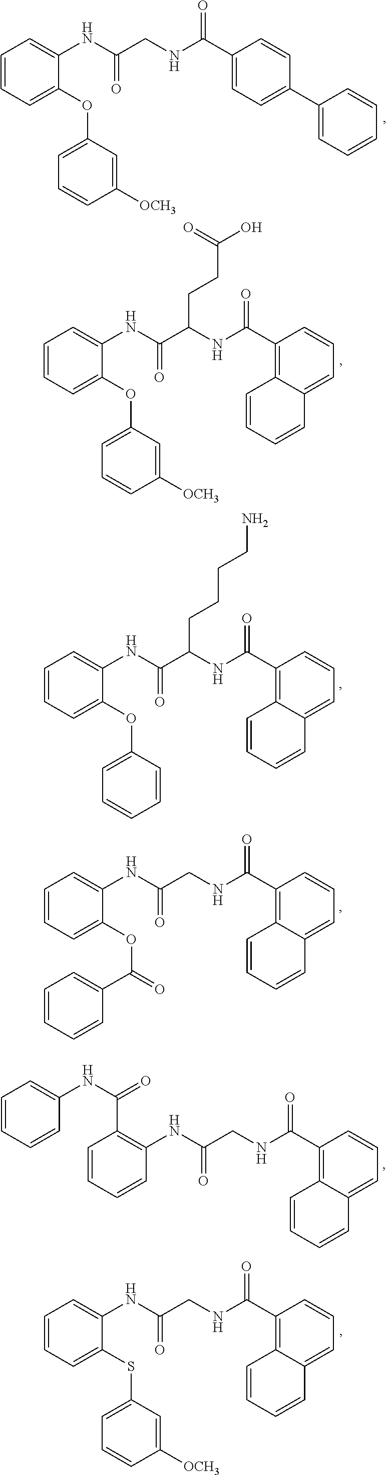

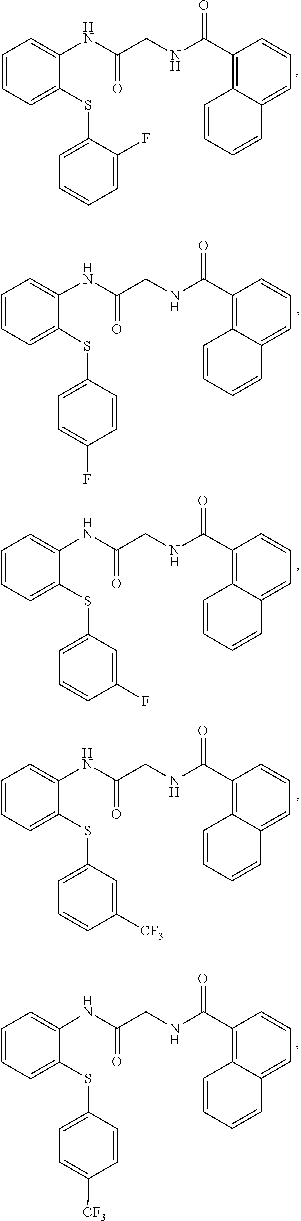 [SMILES: O=C(CNC(=O)c1cccc2ccccc12)Nc1ccccc1Sc1ccccc1F ; O=C(CNC(=O)c1cccc2ccccc12)Nc1ccccc1Sc1ccc(F)cc1 ; O=C(CNC(=O)c1cccc2ccccc12)Nc1ccccc1Sc1cccc(F)c1 ; O=C(CNC(=O)c1cccc2ccccc12)Nc1ccccc1Sc1cccc(C(F)(F)F)c1 ; O=C(CNC(=O)c1cccc2ccccc12)Nc1ccccc1Sc1ccc(C(F)(F)F)cc1]

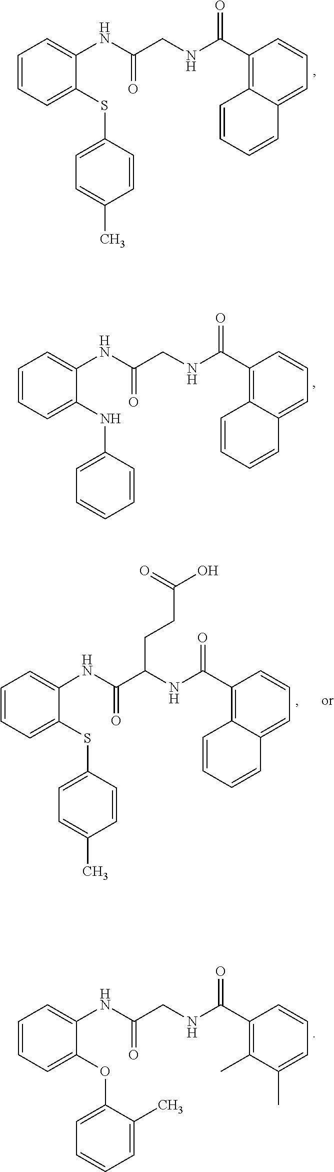 [SMILES: Cc1ccc(Sc2ccccc2NC(=O)CNC(=O)c2cccc3ccccc23)cc1 ; O=C(CNC(=O)c1cccc2ccccc12)Nc1ccccc1Nc1ccccc1 ; Cc1ccc(Sc2ccccc2NC(=O)C(CCC(=O)O)NC(=O)c2cccc3ccccc23)cc1 ; Cc1ccccc1Oc1ccccc1NC(=O)CNC(=O)c1cccc(C)c1C]

88. A pharmaceutical composition comprising a compound of one of claims 1 to 87 or a pharmaceutically acceptable salt thereof, and a pharmaceutically acceptable excipient.

89. The pharmaceutical composition of claim 88, further comprising an anti-cancer agent.

90. The pharmaceutical composition of claim 89, wherein the anti-cancer agent is a platinum-based compound, topoisomerase inhibitor, or Chk1 inhibitor.

91. The pharmaceutical composition of claim 89, wherein the anti-cancer agent is a cisplatin.

92. The pharmaceutical composition of claim 89, wherein the anti-cancer agent is etoposide, SN-38, camptothecin, or gemcitabine.

93. The pharmaceutical composition of claim 89, wherein the anti-cancer agent is CHIR-124, debromohymenialdisine, SB 218078, LY2603618, SCH900776, TCS 2312, PF 477736, UCN-01, or AZD7762.

94. A method of treating a disease associated with PCNA activity in a subject in need of such treatment, said method comprising administering a therapeutically effective amount of a compound of one of claims 1 to 87, or a pharmaceutically acceptable salt thereof.

95. A method of treating cancer in a subject in need of such treatment, said method comprising administering a therapeutically effective amount of a compound of one of claims 1 to 87, or a pharmaceutically acceptable salt thereof.

96. The method of claim 95, further comprising administering radiation.

97. The method of claim 95, wherein said cancer is a sarcoma, adenocarcinoma, leukemia, or lymphoma.

98. The method of claim 95, wherein said cancer is a lung cancer, colon cancer, central nervous system cancer, brain cancer, neuroblastoma, skin cancer, head and neck cancer, melanoma, ovarian cancer, renal cancer, prostate cancer, breast cancer, mesothelioma, liver cancer, stomach cancer, esophageal cancer, bladder cancer, cervical cancer, osteosarcoma, pancreatic cancer, adrenal cortical cancer, adrenal gland cancer, colorectal cancer, testicular cancer, myeloma, B-acute lymphoblastic lymphoma, non-Hodgkin's lymphoma, Hodgkin's lymphoma, chronic leukemia, acute leukemia, glandular carcinoma, or hematoid carcinoma.

99. A method of inhibiting PCNA activity, said method comprising contacting PCNA with an effective amount of a compound of one of claims 1 to 87, or a pharmaceutically acceptable salt thereof.

100. The method of claim 99, wherein the compound binds to His44, Val45, Leu47, Pro234, Tyr250, Leu251, Ala252, Met40, Leu47, Leu126, Leu128, Val233, Pro234, Ala252, Pro253, or Asp 232 of PCNA.

101. The method of claim 99, wherein the compound binds noncovalently to His44, Val45, Leu47, Pro234, Tyr250, Leu251, Ala252, Met40, Leu47, Leu126, Leu128, Val233, Pro234, Ala252, Pro253, or Asp 232 of PCNA.

102. A compound, or a pharmaceutically acceptable salt thereof, having the formula: 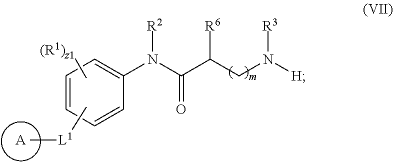
wherein
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or 29 unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)R^{6C}, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F; and
m is an integer from 0 to 5.

103. The compound of claim 102, having the formula: 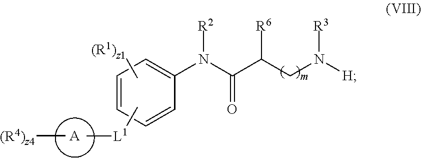
wherein
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{4C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{4C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{1C}, and R^{4D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
z4 is an integer from 0 to 5;
m4 and v4 are independently 1 or 2;
n4 is an integer from 0 to 4; and
X⁴ is independently —Cl, —Br, —I, or —F.

104. The compound of claim 103, having the formula:

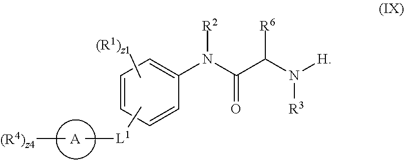

105. The compound of claim 102, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

106. The compound of claim 102, wherein L¹ is —O—.

107. The compound of claim 102, wherein L¹ is —S—.

108. The compound of claim 102, wherein L¹ is —S(O)₂—.

109. The compound of claim 102, wherein R¹ is independently halogen, —CF₃, CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

110. The compound of claim 102, wherein R¹ is independently halogen, —OH, —CF₃, —CHF₂, —CH₂F, —OCF₃, —OCHF₂, —OCH₂F, unsubstituted methyl, or unsubstituted methoxy.

111. The compound of claim 102, wherein z1 is 1.

112. The compound of claim 102, wherein z1 is 0.

113. The compound of claim 102, wherein R² is hydrogen, unsubstituted methyl, unsubstituted ethyl, or unsubstituted isopropyl.

114. The compound of claim 102, wherein R³ is hydrogen, unsubstituted methyl, unsubstituted ethyl, or unsubstituted isopropyl.

115. The compound of claim 102, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

116. The compound of claim 102, wherein R⁶ is substituted or unsubstituted C₁-C₆ alkyl or substituted or unsubstituted 2 to 6 membered heteroalkyl.

117. The compound of claim 102, wherein R⁶ is hydrogen, unsubstituted methyl, unsubstituted isopropyl,

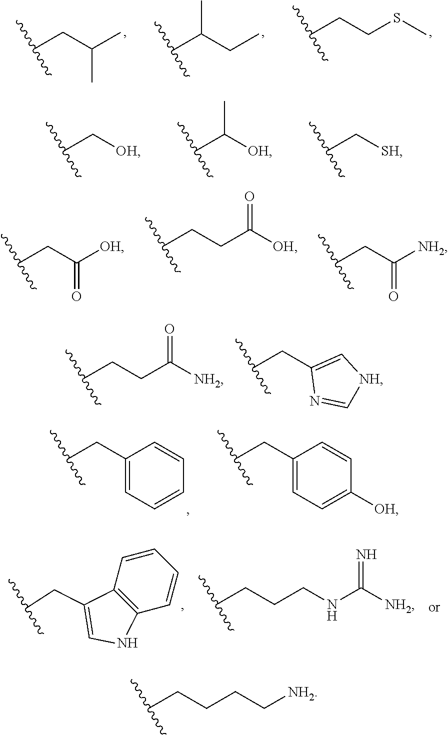

118. The compound of claim 103, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

119. The compound of claim 103, wherein R⁴ is independently halogen, —CF₃, —CHF₂, —CH₂F, —OCF₃, —OCHF₂, —OCH₂F, —OH, unsubstituted methyl, or unsubstituted methoxy.

120. The compound of claim 103, wherein R⁴ is independently —OR^{4D}.

121. The compound of claim 120, wherein R^{4D} is hydrogen or substituted or unsubstituted alkyl.

122. The compound of claim 120, wherein R^{4D} is hydrogen or unsubstituted C₁-C₅ alkyl.

123. The compound of claim 120, wherein R^{4D} is unsubstituted methyl.

124. The compound of claim 103, wherein z4 is 1.

125. The compound of claim 103, wherein z4 is 0.

126. The compound of claim 103, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

127. The compound of claim 103, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

128. The compound of claim 103, wherein z5 is 1.

129. The compound of claim 103, wherein z5 is 0.

130. The compound of claim 102, wherein Ring A is a substituted or unsubstituted phenyl.

131. The compound of claim 102, wherein Ring A is a substituted or unsubstituted 5 to 6 membered heteroaryl.

132. The compound of claim 102, wherein Ring A is a substituted or unsubstituted thienyl.

133. The compound of claim 102, wherein Ring A is a substituted or unsubstituted pyridyl.

134. A method of making compound (I), or a pharmaceutically acceptable salt thereof, said method comprising mixing compound (VII) and compound (X) together in a reaction vessel; wherein
compound (I) has the formula: 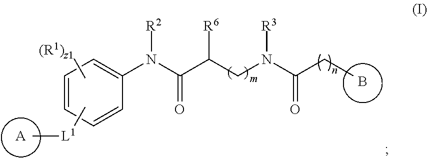
compound (VII) has the formula: 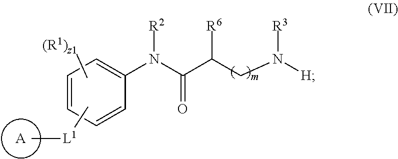
compound (X) has the formula: 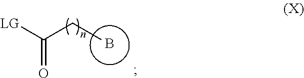
L¹ is —O—, —NR⁷—, —S—, —C(O)—, —C(O)O—, —OC(O)—, —NR⁷C(O)—, —C(O)NR⁷—, —NR⁷C(O)NR⁸—, —NR⁷S(O)₂O—, —OS(O)₂NR⁷—, —NR⁷S(O)₂—, —S(O)₂NR⁷—, —S(O)—, —S(O)₂—, —OS(O)₂O—, —S(O)₂O—, —OS(O)₂—, —P(O)(OR⁷)—, —OP(O)(OR⁷)O—, —OP(O)(OR⁷)—, —P(O)(OR⁷)O—, or —CR⁸R⁹—;
R⁷, R⁸, and R⁹ are independently hydrogen, halogen, —OH, —N₃, or substituted or unsubstituted alkyl;
Ring A is substituted or unsubstituted phenyl or substituted or unsubstituted 5 to 6 membered heteroaryl;
Ring B is substituted or unsubstituted phenyl, substituted or unsubstituted naphthyl, substituted or unsubstituted quinolinyl, or substituted or unsubstituted isoquinolinyl;
R¹ is independently halogen, —CX¹₃, —CHX¹₂, —CH₂X¹, —OCX¹₃, —OCHX¹₂, —OCH₂X¹, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —OC(O)NR^{1A}R^{1B}, —NR^{1A}OR^{1C}, —P(O)R^{1A}R^{1B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is hydrogen, halogen, —CX⁶₃, —CHX⁶₂, —CH₂X⁶, —OCX⁶₃, —OCHX⁶₂, —OCH₂X⁶, —CN, —SOₙ₆R^{6D}, —SOᵥ₆NR^{6A}R^{6B}, —NR^{6C}NR^{6A}R^{6B}, —ONR^{6A}R^{6B}, —NHC(O)NR^{6C}NR^{6A}R^{6B}, —NR^{6C}C(O)NR^{6A}R^{6B}, —N(O)ₘ₆, —NR^{6A}R^{6B}, —C(O)R^{6C}, —C(O)OR^{6C}, —OC(O)R^{6C}, —OC(O)OR^{6C}, —C(O)NR^{6A}R^{6B}, —OR^{6D}, —SR^{6D}, —NR^{6A}SO₂R^{6D}, —NR^{6A}C(O)ROC, —NR^{6A}C(O)OR^{6C}, —OC(O)NR^{6A}R^{6B}, —NR^{6A}OR^{6C}, —P(O)R^{6A}R^{6B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ and R⁶ may optionally be joined to form a substituted or unsubstituted heterocycloalkyl or substituted or unsubstituted heteroaryl;
R^{1A}, R^{1B}, R^{1C}, R^{1D}, R^{6A}, R^{6B}, R^{6C}, and R^{6D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl;
z1 is an integer from 0 to 4;
m1, m6, v1, and v6 are independently 1 or 2;
n1 and n6 are independently an integer from 0 to 4;
X, X¹, X², X³, and X⁶ are independently —Cl, —Br, —I, or —F;
m is an integer from 0 to 5;
n is an integer from 0 to 10; and
LG is a leaving group.

135. The method of claim 134, wherein LG is halogen.

136. The method of claim 134, wherein LG is —Cl.

137. The method of claim 134, further comprising a base.

138. The method of claim 137, wherein the base is N,N-diisopropylethylamine.

139. The method of claim 134, wherein LG is —OH.

140. The method of claim 134, further comprising a peptide coupling agent.

141. The method of claim 140, wherein the peptide coupling agent is dicyclohexylcarbodiimide.

142. The method of claim 140, wherein the peptide coupling agent is HBTU.

143. The method of claim 140, wherein the peptide coupling agent is HOBt.

144. The method of claim 140, wherein the peptide coupling agent is PyBOP.

145. The method of claim 134, wherein
compound (I) is a compound of formula (II): 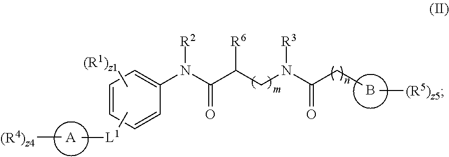
compound (VII) is a compound of formula (VIII): 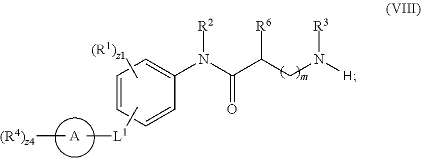
compound (X) is a compound of formula (XI): 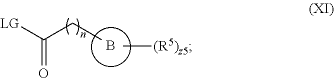
R⁴ is independently a halogen, —CX⁴₃, —CHX⁴₂, —CH₂X⁴, —OCX⁴₃, —OCHX⁴₂, —OCH₂X⁴, —CN, —SOₙ₄R^{4D}, —SOᵥ₄NR^{4A}R^{4B}, —NR^{4C}NR^{4A}R^{4B}, —ONR^{4A}R^{4B}, —NHC(O)NR^{4C}NR^{4A}R^{4B}, —NR^{4C}C(O)NR^{4A}R^{4B}, —N(O)ₘ₄, —NR^{4A}R^{4B}, —C(O)R^{1C}, —C(O)OR^{4C}, —OC(O)R^{4C}, —OC(O)OR^{4C}, —C(O)NR^{4A}R^{4B}, —OR^{4D}, —SR^{4D}, —NR^{4A}SO₂R^{4D}, —NR^{4A}C(O)R^{4C}, —NR^{4A}C(O)OR^{4C}, —OC(O)NR^{4A}R^{4B}, —NR^{4A}OR^{1C}, —P(O)R^{4A}R^{4B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁴ substituents may optionally be joined to form a substituted or unsubstituted cycloalkyl, substituted or unsubstituted heterocycloalkyl, substituted or unsubstituted aryl, or substituted or unsubstituted heteroaryl;
R⁵ is independently a halogen, —CX₅³, —CHX⁵₂, —CH₂X⁵, —OCX₅³, —OCHX⁵₂, —OCH₂X⁵, —CN, —SOₙ₅R^{5D}, —SOᵥ₅NR^{5A}R^{5B}, —NR^{5C}NR^{5A}R^{5B}, —ONR^{5A}R^{5B}, —NHC(O)NR^{5C}NR^{5A}R^{5B}, —NR^{5C}C(O)NR^{5A}R^{5B}, —N(O)ₘ₅, —NR^{5A}R^{5B}, —C(O)R^{5C}, —C(O)OR^{5C}, —OC(O)R^{5C}, —OC(O)OR^{5C}, —C(O)NR^{5A}R^{5B}, —OR^{5D}, —SR^{5D}, —NR^{5A}SO₂R^{5D}, —NR^{5A}C(O)R^{5C}, —NR^{5A}C(O)OR^{5C}, —OC(O)NR^{5A}R^{5B}, —NR^{5A}OR^{5C}, —P(O)R^{5A}R^{5B}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R⁵ substituents may optionally be joined to form a substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, R^{4D}, R^{5A}, R^{5B}, R^{5C}, and R^{5D} are independently hydrogen, halogen, —CX³, —CHX², —CH₂X, —CN, —COOH, —CONH₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
z4 is an integer from 0 to 5;
z5 is an integer from 0 to 7;
m4, m5, v4, and v5 are independently 1 or 2;
n4 and n5 are independently an integer from 0 to 4; and
X⁴ and X⁵ are independently —Cl, —Br, —I, or —F.

146. The method of claim 134, wherein
L¹ is —O—, —NH—, —NCH₃—, —S—, —C(O)—, —C(O)O—, —OC(O)—, —NHC(O)—, —C(O)NH—, —NHC(O)NH—, —NHS(O)₂O—, —OS(O)₂NH—, —NHS(O)₂—, —S(O)₂NH—, —S(O)—, —S(O)₂—, —OS(O)₂O—, —S(O)₂O—, —OS(O)₂—, —P(O)(OH)—, —OP(O)(OH)O—, —OP(O)(OH)—, —P(O)(OH)O—, —CHR⁹—, or —CR⁸R⁹—; and
R⁸ and R⁹ are independently halogen or unsubstituted methyl.

147. The method of claim 134, wherein L¹ is —O—.

148. The method of claim 134, wherein L¹ is —S—.

149. The method of claim 134, wherein L¹ is —S(O)₂—.

150. The method of claim 134, wherein R¹ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

151. The method of claim 134, wherein R¹ is independently halogen, —OH, —CF₃, —CHF₂, —CH₂F, —OCF₃, —OCHF₂, —OCH₂F, unsubstituted methyl, or unsubstituted methoxy.

152. The method of claim 134, wherein z1 is 1.

153. The method of claim 134, wherein z1 is 0.

154. The method of claim 134, wherein R² is hydrogen, unsubstituted methyl, unsubstituted ethyl, or unsubstituted isopropyl.

155. The method of claim 134, wherein R³ is hydrogen, unsubstituted methyl, unsubstituted ethyl, or unsubstituted isopropyl.

156. The method of claim 134, wherein R⁶ is hydrogen, halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

157. The method of claim 134, wherein R⁶ is substituted or unsubstituted C₁-C₆ alkyl or substituted or unsubstituted 2 to 6 membered heteroalkyl.

158. The method of claim 134, wherein R⁶ is hydrogen, unsubstituted methyl, unsubstituted isopropyl,

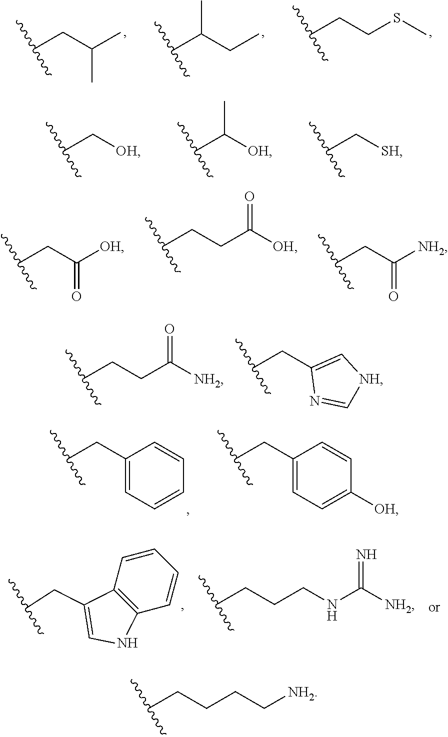

159. The method of claim 145, wherein R⁴ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

160. The method of claim 145, wherein R⁴ is independently halogen, —CF₃, —CHF₂, —CH₂F, —OCF₃, —OCHF₂, —OCH₂F, —OH, unsubstituted methyl, or unsubstituted methoxy.

161. The method of claim 145, wherein R⁴ is independently —OR^{4D}.

162. The method of claim 161, wherein R^{4D} is hydrogen or substituted or unsubstituted alkyl.

163. The method of claim 161, wherein R^{4D} is hydrogen or unsubstituted 2 C₁-C₈ alkyl.

164. The method of claim 161, wherein R^{4D} is unsubstituted methyl.

165. The method of claim 145, wherein z4 is 1.

166. The method of claim 145, wherein z4 is 0.

167. The method of claim 145, wherein R⁵ is independently halogen, —CF₃, —CHF₂, —CH₂F, —CN, —OH, —NH₂, —COOH, —CONH₂, —NO₂, —SH, —OCF₃, —OCHF₂, —OCH₂F, substituted or unsubstituted C₁-C₈ alkyl, substituted or unsubstituted 2 to 8 membered heteroalkyl, substituted or unsubstituted C₃-C₈ cycloalkyl, substituted or unsubstituted 3 to 8 membered heterocycloalkyl, substituted or unsubstituted C₆-C₁₀ aryl, or substituted or unsubstituted 5 to 10 membered heteroaryl.

168. The method of claim 145, wherein R⁵ is independently halogen, —CF₃, —CHF₂, —CH₂F, —OCF₃, —OCHF₂, —OCH₂F, —OH, unsubstituted methyl, unsubstituted methoxy, or unsubstituted phenyl.

169. The method of claim 145, wherein z5 is 1.

170. The method of claim 145, wherein z5 is 0.

171. The method of claim 134, wherein Ring A is a substituted or unsubstituted phenyl.

172. The method of claim 134, wherein Ring A is a substituted or unsubstituted 5 to 6 membered heteroaryl.

173. The method of claim 134, wherein Ring A is a substituted or unsubstituted thienyl.

174. The method of claim 134, wherein Ring A is a substituted or unsubstituted pyridyl.

175. The method of claim 134, wherein Ring B is a substituted or unsubstituted phenyl.

176. The method of claim 134, wherein Ring B is a substituted or unsubstituted naphthyl.

177. The method of claim 134, wherein Ring B is a substituted or unsubstituted quinolinyl.

178. The method of claim 134, wherein Ring B is a substituted or unsubstituted isoquinolinyl.


SEQUENCE LISTING (11 sequences)

SEQ ID NO: 1 - DNA, 23 bp, synthetic construct
  misc_feature 1..23: note = Synthetic polynucleotide
  source 1..23: mol_type = other DNA
acgtctctttggtgcagctcacc

SEQ ID NO: 2 - DNA, 23 bp, synthetic construct
  misc_feature 1..23: note = Synthetic polynucleotide
  source 1..23: mol_type = other DNA
acgtctctgtcgtgcagctcacc

SEQ ID NO: 3 - DNA, 24 bp, synthetic construct
  misc_feature 1..24: note = Synthetic polynucleotide
  source 1..24: mol_type = other DNA
acgtctctgtcgtgcagctcaccc

SEQ ID NO: 4 - protein, 261 aa, synthetic construct
  REGION 1..261: note = Synthetic polypeptide
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 5 - DNA, 20 bp, synthetic construct
  misc_feature 1..20: note = Synthetic polynucleotide
  source 1..20: mol_type = other DNA
ggactcgtcccacgtctctt

SEQ ID NO: 6 - DNA, 20 bp, synthetic construct
  misc_feature 1..20: note = Synthetic polynucleotide
  source 1..20: mol_type = other DNA
ctttggtgcagctcaccctg

SEQ ID NO: 7 - DNA, 20 bp, synthetic construct
  misc_feature 1..20: note = Synthetic polynucleotide
  source 1..20: mol_type = other DNA
cggcattaaacggttgcagg

SEQ ID NO: 8 - DNA, 20 bp, synthetic construct
  misc_feature 1..20: note = Synthetic polynucleotide
  source 1..20: mol_type = other DNA
cgtggcaggccaatgagaag

SEQ ID NO: 9 - DNA, 20 bp, synthetic construct
  misc_feature 1..20: note = Synthetic polynucleotide
  source 1..20: mol_type = other DNA
acgaggcctgctgggatatt

SEQ ID NO: 10 - DNA, 20 bp, synthetic construct
  misc_feature 1..20: note = Synthetic polynucleotide
  source 1..20: mol_type = other DNA
tgagggctaggctcgaaagc

SEQ ID NO: 11 - DNA, 20 bp, synthetic construct
  misc_feature 1..20: note = Synthetic polynucleotide
  source 1..20: mol_type = other DNA
attgtctcggatgatgtact